(19) United States
(12) Patent Application Publication
(10) Pub. No.: US 2020/0095226 A1
(43) Pub. Date: Mar. 26, 2020

(54) TRIAZINE FUSED RING DERIVATIVE AND APPLICATION THEREOF IN ORGANIC ELECTRONIC DEVICE

(71) Applicant: GUANGZHOU CHINARAY OPTOELECTRONIC MATERIALS LTD., Guangzhou, Guangdong (CN)

(72) Inventors: Junyou PAN, Guangzhou, Guangdong (CN); Guang HU, Guangzhou, Guangdong (CN)

(73) Assignee: GUANGZHOU CHINARAY OPTOELECTRONIC MATERIALS LTD., Guangzhou, Guangdong (CN)

(21) Appl. No.: 16/467,422

(22) PCT Filed: Dec. 8, 2017
(86) PCT No.: PCT/CN2017/115313

§ 371 Date: Jun. 6, 2019

(30) Foreign Application Priority Data
Dec. 8, 2016 (CN) 201611122513.5

(51) Int. Cl. C07D 401/14; C07D 401/10; C07D 401/04; H01L 51/00

(52) U.S. Cl. CPC C07D 401/14; C07D 401/10; C07D 401/04; H01L 51/0067; H01L 51/0054; H01L 2251/552; H01L 51/0072; H01L 51/0073; H01L 51/0074; H01L 51/0055; H01L 51/0058

(57) ABSTRACT

Provided are a triazine fused ring compound, and an organic mixture, formulation and organic electronic device comprising the same and application thereof. The triazine fused ring compound comprises a triazine structure having three strong electron-withdrawing nitrogen atoms, and an aromatic fused ring structure. As the triazine structure has favorable optoelectronic properties and the fused ring structure contains an aryl group or heteroaryl group, the compound has good carrier transport performance and optoelectronic response. Thus, by connecting triazine with the fused ring aromatic structure having a large planar-conjugated system, the present invention facilitates better carrier transport and optoelectronic response and better energy level matching, improves the optoelectronic properties and stability of the triazine fused ring compound, and thereby enables a light-emitting device having high manufacturing efficiency and long operating life.

### TECHNICAL FIELD

The present disclosure relates to a triazine fused ring compound, a mixture and a formulation comprising the triazine fused ring compound, and application thereof in organic electronic devices.

### BACKGROUND

Organic semiconductor materials have great application potential in many optoelectronic apparatuses, such as an organic light-emitting diode (OLED), an organic photovoltaic cell (OPV), and an organic field effect transistor (OFET), due to properties of the organic semiconductor materials such as the diversity of molecular structure design, relatively low manufacturing cost, and superior optoelectronic performance, etc. Especially, since Deng Qingyun et al. (C. W. Tang and S. A. Van Slyke, Appl. Phys. Lett., 1987, 51, 913) reported the double-layer OLED structure in 1987, the organic semiconductor materials have developed rapidly in the fields of flat panel display and illumination.

The organic thin film light-emitting element must satisfy the requirements of improvement in luminous efficiency, reduction in driving voltage, improvement in durability, etc. However, currently there are still many technical issues, among which the coexistence of high efficiency and long lifetime of the element is one of many challenges.

In order to accelerate the process of promoting the large-scale industrialization of OLED and improve its optoelectronic performance, various novel organic optoelectronic material systems have been widely designed, developed and produced. Organic semiconductor materials of triazines containing three strong electron-accepting nitrogen atoms have wide applications in optoelectronic devices due to their superior optoelectronic performance. In addition, the aromatic group or the heteroaromatic group of the fused ring structure, such as fluoranthene, anthracene, pyrene, phenanthrene, phenanthroline, benzofluoranthene, etc., generally has good carrier transport performance and optoelectronic response due to the planar structure of the molecule thereof. However, currently reported organic semiconductor materials of triazines or having fused ring structures have certain limitations in carrier transport capability, stability, lifetime, and the like in photovoltaic devices.

KR 20150120875 A disclosed a class of triazine biphenyl fused ring compounds characterized in that a fused ring group is linked at two meta positions derived from triazine, and a nitrogen atom-containing aromatic ring is linked at two meta positions derived from benzene ring. Such compounds are used as electron transport layers for blue OLED devices, which improves the whole performance of the device.

Rather, no research and disclosure have been made on a class of compounds which link the nitrogen atom-containing aromatic ring containing electron-accepting system at two meta positions of triazine, and link large planar conjugated fused ring structure of the electron-donating system at two meta positions derived from the benzene ring. The novel design combination of such triazines with fused rings may impart superior optoelectronic performance to such structures.

In addition, in order to reduce the production costs and realize the large-area OLED devices, printing OLED is becoming one of the most promising technology options. In this regard, materials for printing OLEDs are the key. However, the currently developed small-molecule OLED materials based on evaporation technology have poor solubility and film-forming performance due to their lower molecular weight and rigid aromatic molecular structure. Particularly, it is difficult to form a non-hollow amorphous film with a regular appearance. Therefore, currently there is no corresponding material solution to the problem of printing OLEDs, and small molecule organic light-emitting diodes with high performance are still prepared by vacuum evaporation. Therefore, designing and synthesizing small molecule organic functional compounds with good solubility and film-forming performance is especially important for realizing the high-performance solution-processing organic light-emitting diodes.

### SUMMARY

In view of the above deficiencies of the prior art, the object of the present disclosure is to provide a class of novel organic optoelectronic materials, in particular a triazine fused ring compound, a mixture and a formulation containing the triazine fused ring compound, and application thereof in organic electronic devices, aimed to reduce driving voltage, improve luminous efficiency, stability and lifetime of the devices, and provide a material solution for printing OLED at the same time.

A technical solution of the present disclosure is described below.

A class of triazine fused ring compound as represented by following general formula (1):

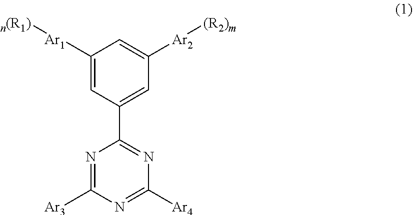

wherein

Ar₁ and Ar₂ are aromatic or heteroaromatic groups, and at least one of Ar₁ and Ar₂ is a fused aromatic ring or fused heteroaromatic ring containing 13 to 60 ring atoms;

Ar₃ and Ar₄ are selected from the group consisting of H, D, F, —CN, —NO₂, —CF₃, alkenyl, alkynyl, amino, acyl, amide group, cyano, isocyano, alkoxy, hydroxy, carbonyl, sulfonyl, an alkyl group containing 1 to 60 carbon atoms, a cycloalkyl group containing 3 to 60 carbon atoms, an aromatic group containing 6 to 60 carbon atoms, a heterocyclic aromatic group containing 3 to 60 carbon atoms, a fused ring aromatic group containing 7 to 60 carbon atoms, and a fused heterocyclic aromatic group containing 4 to 60 carbon atoms, or Ar₃ and Ar₄ form a monocyclic or polycyclic aliphatic or aromatic ring system with each other;

at least one of Ar₃ and Ar₄ comprises an aromatic heterocyclic ring having an N atom;

R₁ and R₂ are selected from the group consisting of H, D, F, —CN, —NO₂, —CF₃, alkenyl, alkynyl, amino, acyl, amide group, cyano, isocyano, alkoxy, hydroxy, carbonyl, sulfonyl, an alkyl group containing 1 to 60 carbon atoms, a cycloalkyl group containing 3 to 60 carbon atoms, an aromatic group containing 6 to 60 carbon atoms, a heterocyclic aromatic group containing 3 to 60 carbon atoms, a fused ring aromatic group containing 7 to 60 carbon atoms, and a fused heterocyclic aromatic group containing 4 to 60 carbon atoms, or R₁ and R₂ form a monocyclic or polycyclic aliphatic or aromatic ring system with each other;

n is an integer from 0 to 20; and

m is an integer from 0 to 20.

In one embodiment, at least one of Ar₃ and Ar₄ comprises the following structures Ts:

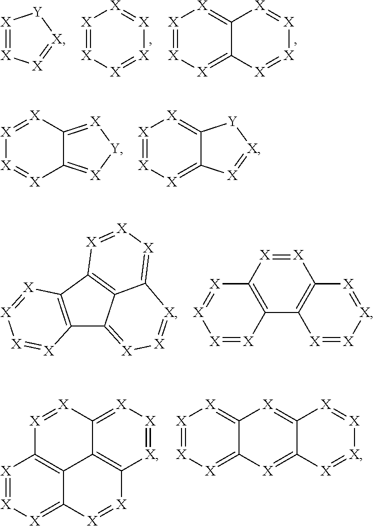

wherein

X is CR₃ or N, at least one of Xs in the structures Ts is N, and two adjacent Xs are not N simultaneously;

Y is selected from the group consisting of CR₄R₅, SiR₆R₇, NR₈, C(═O), S(═O)₂, O, and S; and

R₃ to R₈ are selected from the group consisting of H, D, F, —CN, —NO₂, —CF₃, alkenyl, alkynyl, amino, acyl, amide group, cyano, isocyano, alkoxy, hydroxy, carbonyl, sulfonyl, an alkyl group containing 1 to 60 carbon atoms, a cycloalkyl group containing 3 to 60 carbon atoms, an aromatic group containing 6 to 60 carbon atoms, a heterocyclic aromatic group containing 3 to 60 carbon atoms, a fused ring aromatic group containing 7 to 60 carbon atoms, and a fused heterocyclic aromatic group containing 4 to 60 carbon atoms, or R₃ to R₈ form a monocyclic or polycyclic aliphatic or aromatic ring system with each other.

In one embodiment, at least one of Ar₁ and Ar₂ described in the general formula (1) of the triazine fused ring compound is selected from the following structures Ds:

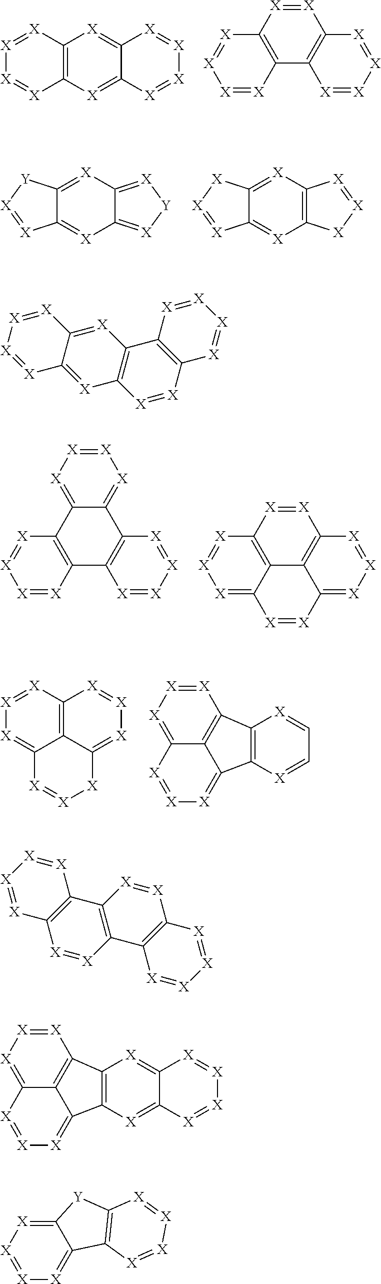

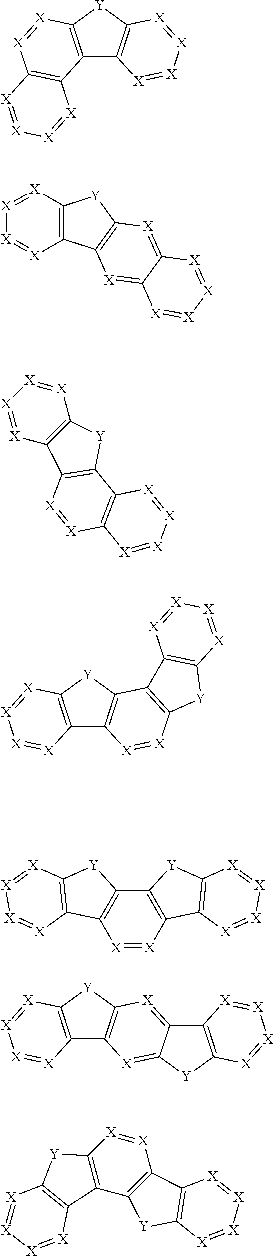

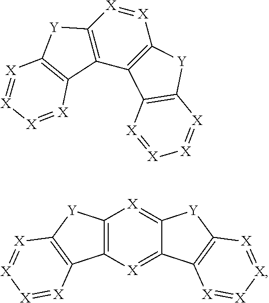

wherein

X is CR₉ or N, but two adjacent Xs are not N simultaneously;

Y is selected from the group consisting of CR₁₀R₁₁, SiR₁₂R₁₃, NR₁₄, C(═O), S(═O)₂, O, and S; and

R₉ to R₁₄ are selected from the group consisting of H, D, F, —CN, —NO₂, —CF₃, alkenyl, alkynyl, amino, acyl, amide group, cyano, isocyano, alkoxy, hydroxy, carbonyl, sulfonyl, an alkyl group containing 1 to 60 carbon atoms, a cycloalkyl group containing 3 to 60 carbon atoms, an aromatic group containing 6 to 60 carbon atoms, a heterocyclic aromatic group containing 3 to 60 carbon atoms, a fused ring aromatic group containing 7 to 60 carbon atoms, and a fused heterocyclic aromatic group containing 4 to 60 carbon atoms, or R₉ to R₁₄ form a monocyclic or polycyclic aliphatic or aromatic ring system with each other.

A polymer comprises at least two repeating structural units of the triazine fused ring compound as described in the general formula (1).

A mixture comprises at least one of the triazine fused ring compound or the polymer as described above, and at least one organic functional material, and the organic functional material may be selected from the group consisting of a hole (also called electron hole) injection or transport material (HIM/HTM), a hole blocking material (HBM), an electron injection or transport material (EIM/ETM), an electron blocking material (EBM), an organic matrix material (Host), a singlet emitter (fluorescent emitter), a triplet emitter (phosphorescent emitter), a thermally activated delayed fluorescent material (TADF material) and an organic dye.

A formulation comprises at least one of the triazine fused ring compound or the polymer as described above, and at least one organic solvent.

An organic electronic device comprises at least one of the triazines fused ring compound or the polymer as described above, or is prepared from the formulation as described above, and the organic electronic device can be selected from the group consisting of an organic light-emitting diode (OLED), an organic photovoltaic cell (OPV), an organic light-emitting electrochemical cell (OLEEC), an organic field effect transistor (OFET), an organic light-emitting field effect transistor, organic laser, an organic spintronic device, organic sensor, and an organic plasmon emitting diode.

A functional layer containing the triazine fused ring compound, prepared by: forming a functional layer on a substrate by evaporation of the triazine fused ring compound as described above; or forming a functional layer on a substrate by co-evaporation of the triazine fused ring compound together with the an organic functional material; or forming a functional layer by coating the formulation as described above on a substrate via printing or coating method. The printing or coating method can be selected from inkjet printing, nozzle printing, letterpress printing, screen printing, dip coating, spin coating, blade coating, roller printing, torsion roll printing, lithography, flexographic printing, rotary printing, spray coating, brush coating or pad printing, slot die coating, and the like.

The triazine fused ring compound of the present disclosure comprises a triazine structure containing three strong electron-accepting nitrogen atoms and a fused aromatic ring structure. The triazine structure has superior optoelectronic performance, and the fused ring structure contains an aromatic group or a heteroaromatic group in a planar structure, and has good carrier transport performance and optoelectronic response. Therefore the large planar conjugated structure formed by linking the triazine to the fused aromatic ring structure is beneficial to achieve better carrier transport and optoelectronic response, better energy level matching, and improves optoelectronic performance and stability of this triazine fused ring compound, so that a light-emitting device with high manufacturing efficiency and long lifetime can be finally obtained.

### DETAILED DESCRIPTION OF THE DISCLOSURE

The present disclosure provides a class of novel organic optoelectronic materials and application thereof in organic electronic devices. In order to make the purpose, technical solution and effects of the present disclosure clearer and more specific, the present disclosure will be further described in detail below. It should be noted that, the specific embodiment illustrated herein is merely for the purpose of explanation, and should not be deemed to limit the present disclosure.

In the present disclosure, formulation and printing ink, or ink, have the same meaning and they can be used interchangeably.

In the present disclosure, the host material or the matrix material, Host or Matrix have the same meaning and they are interchangeable.

In the present disclosure, the metal organic clathrate, the metal organic complexes, and organometallic complexes have the same meaning and are interchangeable.

According to the triazine fused ring compound of the present disclosure, the concepts of aromatic and heteroaromatic will be involved for many times, and are specifically defined as follows:

The aromatic group refers to a hydrocarbyl containing at least one aromatic ring. The heterocyclic aromatic group refers to an aromatic hydrocarbyl containing at least one heteroatom. The fused ring aromatic group refers to an aromatic group whose ring may have two or more rings, wherein two carbon atoms are shared by two adjacent rings, i.e., fused ring. The fused heterocyclic aromatic group refers to a fused ring aromatic hydrocarbyl containing at least one heteroatom. For the purposes of the present disclosure, the aromatic group contains a fused ring aromatic group, and the heterocyclic aromatic group contains a fused heterocyclic aromatic group. For the purposes of the present disclosure, the aromatic group or the heterocyclic aromatic group includes not only an aromatic ring system but also a non-aromatic ring system. Therefore, systems such as pyridine, thiophene, pyrrole, pyrazole, triazole, imidazole, oxazole, oxadiazole, thiazole, tetrazole, pyrazine, pyridazine, pyrimidine, triazine, carbene, etc., are also considered to be an aromatic group or a heterocyclic aromatic group is also included, for the purpose of the disclosure. For the purpose of the present disclosure, the fused aromatic or heteroaromatic ring systems not only include aromatic or heteroaromatic systems, but also have a plurality of aryl groups or heteroaryl groups spaced by short non-aromatic units (<10% of non-H atoms, preferably less than 5% of non-H atoms, such as C, N or O atoms). Therefore, systems such as 9,9′-spirobifluorene, 9,9-diarylfluorene, triarylamine, diaryl ether and the like are also considered to be fused aromatic ring systems for the purpose of this disclosure.

Specifically, examples of the aromatic group include: benzene, biphenyl, terphenyl, toluene, chlorobenzene, and derivatives thereof.

Specifically, examples of the fused ring aromatic group include: naphthalene, anthracene, fluoranthene, phenanthrene, benzophenanthrene, perylene, tetracene, pyrene, benzopyrene, acenaphthene, fluorene, and derivatives thereof.

Specifically, examples of heterocyclic aromatic group are: pyridine, thiophene, pyrrole, pyrazole, triazole, imidazole, oxazole, oxadiazole, thiazole, tetrazole, pyrazine, pyridazine, pyrimidine, triazine, carbene, and derivatives thereof.

Specifically, examples of the fused heterocyclic aromatic group include: benzofuran, benzothiophene, indole, carbazole, pyrroloimidazole, pyrrolopyrrole, thienopyrrole, thienothiophene, furopyrrole, furofuran, thienofuran, benzisoxazole, benzisothiazole, benzimidazole, quinoline, isoquinoline, cinnoline, quinoxaline, phenanthridine, perimidine, quinazoline, quinazolinone, and derivatives thereof.

The present disclosure provides a fused ring compound as represented by general formula (I):

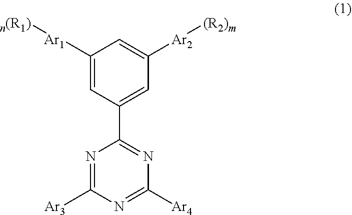

wherein

Ar₁ and Ar₂ are the same or different, are aromatic or heteroaromatic groups, and at least one of Ar₁ and Ar₂ is a fused aromatic ring or fused heteroaromatic ring containing 13 to 60 ring atoms;

R₁, R₂, Ar₃, and Ar₄ are the same or different in multiple occurrences, and are each independently selected from the group consisting of H, D, F, —CN, —NO₂, —CF₃, alkenyl, alkynyl, amino, acyl, amide group, cyano, isocyano, alkoxy, hydroxy, carbonyl, sulfonyl, a substituted or unsubstituted alkyl containing 1 to 60 carbon atoms, a substituted or unsubstituted cycloalkyl containing 3 to 60 carbon atoms, a substituted or unsubstituted aromatic group containing 6 to 60 carbon atoms, a substituted or unsubstituted heterocyclic aromatic group containing 3 to 60 carbon atoms, a substituted or unsubstituted fused ring aromatic group containing 7 to 60 carbon atoms, or a fused heterocyclic aromatic group containing 4 to 60 carbon atoms, or one or more of the groups may form a monocyclic or polycyclic aliphatic or aromatic ring system with each other and/or with a ring bonded to the groups,

at least one of Ar₃ and Ar₄ comprises an aromatic heterocyclic ring having an N atom;

m is an integer from 0 to 20, further, m is an integer from 0 to 10, still further, m is an integer from 0 to 5, and even further, m is an integer from 0 to 3; and

n is an integer from 0 to 20, further, n is an integer from 0 to 10, still further, n is an integer from 0 to 5, and even further, n is an integer from 0 to 3.

In some embodiments, Ar₁ or Ar₂ is a fused aromatic ring or fused heteroaromatic ring containing 13 to 50 ring atoms, further, Ar₁ or Ar₂ is a fused aromatic ring or fused heteroaromatic ring containing 13 to 40 ring atoms, still further, Ar₁ or Ar₂ is a fused aromatic ring or fused heteroaromatic ring containing 13 to 30 ring atoms, even further, Ar₁ or Ar₂ is a fused aromatic ring or fused heteroaromatic ring containing 13 to 20 ring atoms.

In some embodiments, Ar₁ and Ar₂ are independently selected from a fused aromatic ring or fused heteroaromatic ring containing 13 to 50 ring atoms, further, Ar₁ and Ar₂ are selected from a fused aromatic ring or fused heteroaromatic ring containing 13 to 40 ring atoms, still further, Ar₁ and Ar₂ are selected from a fused aromatic ring or fused heteroaromatic ring containing 13 to 30 ring atoms, even further, Ar₁ and Ar₂ are selected from a fused aromatic ring or fused heteroaromatic ring containing 13 to 20 ring atoms.

In certain embodiments, the heteroatom of the fused heteroaromatic ring is selected from Si, N, P, O, S and/or Ge, particularly selected from Si, N, P, O and/or S, and even particularly selected from N, O or S.

In an embodiment, Ar₁ or Ar₂ in the general formula (1) is a fused ring with a ring number of 3 to 30, wherein the ring may be selected from the group consisting of a three-membered ring, a four-membered ring, a five-membered ring, and a six-membered ring, and particularly a five-membered ring and a six-membered ring. In one embodiment, Ar₁ is a fused ring with a ring number of 3 to 20, in another embodiment, Ar₁ is a fused ring with a ring number of 3 to 10, and in yet another embodiment, Ar₁ is a fused ring with a ring number of 3 to 5.

In one embodiment, Ar₁ and Ar₂ in the general formula (1) are independently selected from a fused ring with a ring number of 3 to 30, wherein the ring may be selected from the group consisting of a three-membered ring, a four-membered ring, a five-membered ring, and a six-membered ring, and particularly a five-membered ring and a six-membered ring. In an embodiment, Ar₁ and Ar₂ are independently selected from a fused ring with a ring number of 3 to 20, in another embodiment, Ar₁ and Ar₂ are a fused ring with a ring number of 3 to 10, and in yet another embodiment, Ar₁ and Ar₂ are a fused ring with a ring number of 3 to 5.

In some embodiments, R₁, R₂, Ar₃, and Ar₄ are the same or different in multiple occurrences, and are each independently selected from the group consisting of H, D, F, —CN, —NO₂, —CF₃, alkenyl, alkynyl, amino, acyl, amide group, cyano, isocyano, alkoxy, hydroxy, carbonyl, sulfonyl, a substituted or unsubstituted alkyl containing 1 to 30 carbon atoms, a substituted or unsubstituted cycloalkyl containing 3 to 30 carbon atoms, a substituted or unsubstituted aromatic group containing 6 to 30 carbon atoms, a substituted or unsubstituted heterocyclic aromatic group containing 3 to 30 carbon atoms, a substituted or unsubstituted fused ring aromatic group containing 7 to 30 carbon atoms, and a fused heterocyclic aromatic group containing 4 to 30 carbon atoms, or one or more of the groups may form a monocyclic or polycyclic aliphatic or aromatic ring system with each other and/or with a ring bonded to the group.

In some embodiments, Ar₃ comprises an aromatic heterocyclic ring having at least one N atom;

In other embodiments, Ar₄ comprises an aromatic heterocyclic ring having at least one N atom;

In some embodiments, Ar₃ and Ar₄ both comprise an aromatic heterocyclic ring having at least one N atom;

In an embodiment, at least one of Ar₃ and Ar₄ comprises an aromatic heterocyclic ring having an N atom, and the aromatic heterocyclic ring is selected from the following structures:

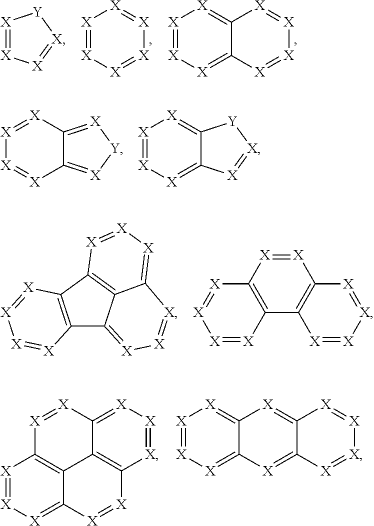

wherein

X is CR₃ or N, at least one Xs in each structure is N, but two adjacent Xs are not N simultaneously; and in an embodiment, the number of N is 1 to 6, further, the number of N is 1 to 3, even further, the number of N is 1 to 2;

Y is selected from the group consisting of CR₄R₅, SiR₆R₇, NR₈, C(═O), S(═O)₂, O, and S; particularly, Y is CR₃R₄ or O; and

R₃ to R₈ are the same or different in multiple occurrences, and may be a linking site to other groups, or are selected from the group consisting of H, D, F, —CN, —NO₂, —CF₃, alkenyl, alkynyl, amino, acyl, amide group, cyano, isocyano, alkoxy, hydroxy, carbonyl, sulfonyl, a substituted or unsubstituted alkyl containing 1 to 60 carbon atoms, a substituted or unsubstituted cycloalkyl containing 3 to 60 carbon atoms, a substituted or unsubstituted aromatic group containing 6 to 60 carbon atoms, a substituted or unsubstituted heterocyclic aromatic group containing 3 to 60 carbon atoms, a substituted or unsubstituted fused ring aromatic group containing 7 to 60 carbon atoms, and a fused heterocyclic aromatic group containing 4 to 60 carbon atoms, or one or more of the groups may form a monocyclic or polycyclic aliphatic or aromatic ring system with each other and/or with a ring bonded to the groups.

In some embodiments, R₃ to R₈ are the same or different in multiple occurrences, and may be a linking site to other groups, or are selected from the group consisting of H, D, F, —CN, —NO₂, —CF₃, alkenyl, alkynyl, amino, acyl, amide group, cyano, isocyano, alkoxy, hydroxy, carbonyl, sulfonyl, a substituted or unsubstituted alkyl containing 1 to 30 carbon atoms, a substituted or unsubstituted cycloalkyl containing 3 to 30 carbon atoms, a substituted or unsubstituted aromatic group containing 6 to 30 carbon atoms, a substituted or unsubstituted heterocyclic aromatic group containing 3 to 30 carbon atoms, a substituted or unsubstituted fused ring aromatic group containing 7 to 30 carbon atoms, and a fused heterocyclic aromatic group containing 4 to 30 carbon atoms, or one or more of the groups may form a monocyclic or polycyclic aliphatic or aromatic ring system with each other and/or with a ring bonded to the groups.

In some embodiments, at least one of Ar₃ and Ar₄ comprises an aromatic heterocyclic ring having an N atom, and the aromatic heterocyclic ring is selected from the following structures:

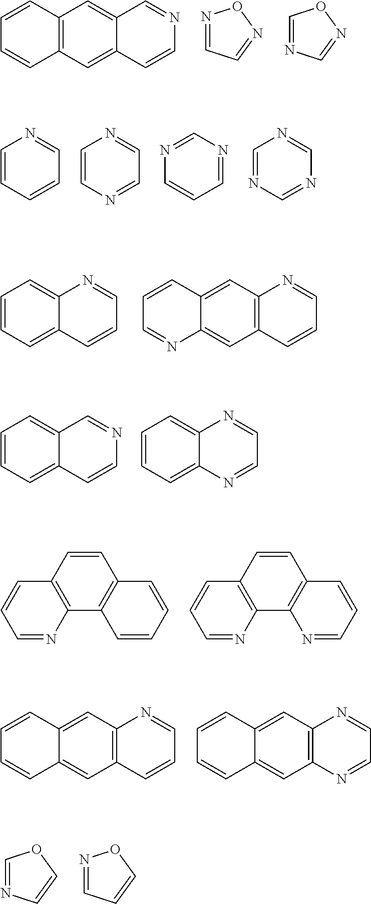

In certain embodiments, Ar₁ and/or Ar₂ in the general formula (1) are each independently selected from the following structures:

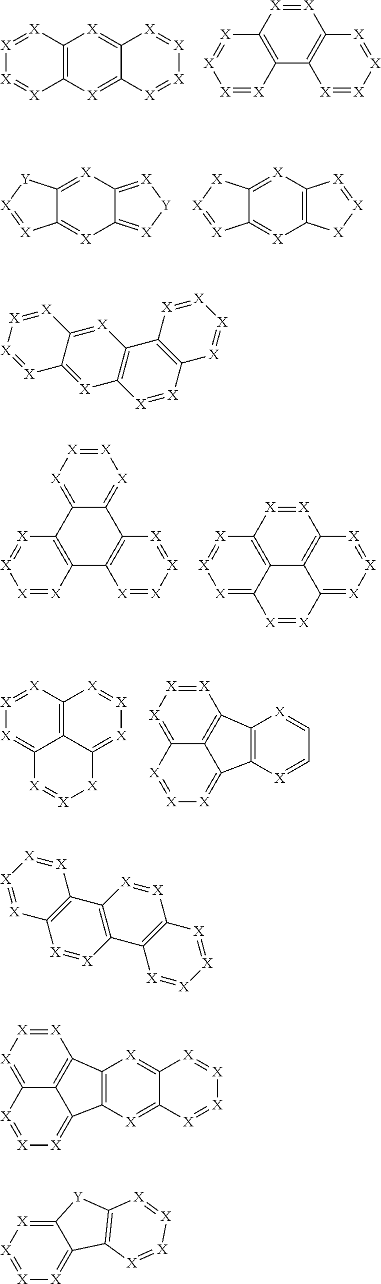

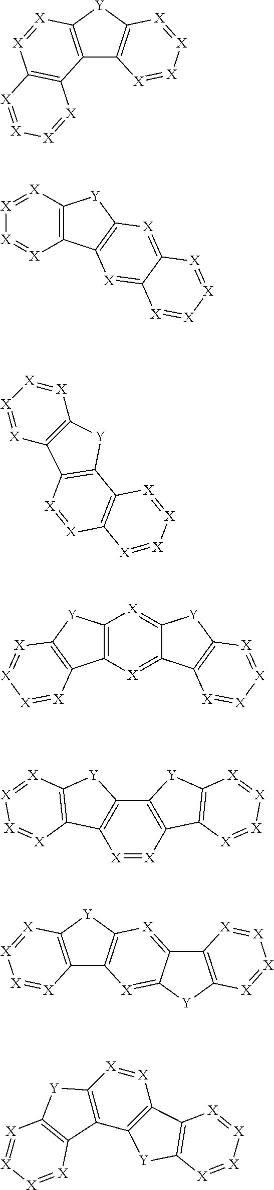

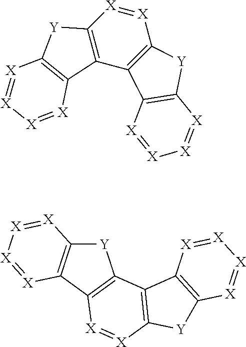

wherein

X is CR₉ or N, but two adjacent Xs are not simultaneously N; in some embodiments, X is CR₉. In some embodiments, all Xs in the above structural formulas are CR₉, particularly, R₉ is H or D;

Y is selected from the group consisting of CR₁₀R₁₁, SiR₁₂R₁₃, NR₁₄, C(═O), S(═O)₂, O, and S; particularly, Y is CR₃R₄ or O; and

R₉ to R₁₄ are the same or different in multiple occurrences, and may be a linking site to other groups, or are selected from the group consisting of H, D, F, —CN, —NO₂, —CF₃, alkenyl, alkynyl, amino, acyl, amide group, cyano, isocyano, alkoxy, hydroxy, carbonyl, sulfonyl, a substituted or unsubstituted alkyl containing 1 to 60 carbon atoms, a substituted or unsubstituted cycloalkyl containing 3 to 60 carbon atoms, a substituted or unsubstituted aromatic group containing 6 to 60 carbon atoms, a substituted or unsubstituted heterocyclic aromatic group containing 3 to 60 carbon atoms, a substituted or unsubstituted fused ring aromatic group containing 7 to 60 carbon atoms, and a fused heterocyclic aromatic group containing 4 to 60 carbon atoms, or in which one or more of the groups may form a monocyclic or polycyclic aliphatic or aromatic ring system with each other and/or with a ring bonded to the groups.

In some embodiments, R₉ to R₁₄ are the same or different in multiple occurrences, and may be a linking site to other groups, or are selected from the group consisting of H, D, F, —CN, —NO₂, —CF₃, alkenyl, alkynyl, amino, acyl, amide group, cyano, isocyano, alkoxy, hydroxy, carbonyl, sulfonyl, a substituted or unsubstituted alkyl containing 1 to 30 carbon atoms, a substituted or unsubstituted cycloalkyl containing 3 to 30 carbon atoms, a substituted or unsubstituted aromatic group containing 6 to 30 carbon atoms, a substituted or unsubstituted heterocyclic aromatic group containing 3 to 30 carbon atoms, a substituted or unsubstituted fused ring aromatic group containing 7 to 30 carbon atoms, and a fused heterocyclic aromatic group containing 4 to 30 carbon atoms, or in which one or more of the groups may form a monocyclic or polycyclic aliphatic or aromatic ring system with each other and/or with a ring bonded to the groups.

In some embodiments, suitable examples of aromatic or heteroaromatic groups which may be used as Ar₁ and Ar₂ are each independently selected from, but not limited to, the group consisting of anthracene, fluoranthene, phenanthrene, benzophenanthrene, perylene, tetracene, pyrene, benzopyrene, acenaphthene, fluorene, carbazole, dibenzofuran, dibenzothiophene and the like.

In some embodiments, Ar₁ and/or Ar₂ are each independently selected from, but not limited to, the following structures:

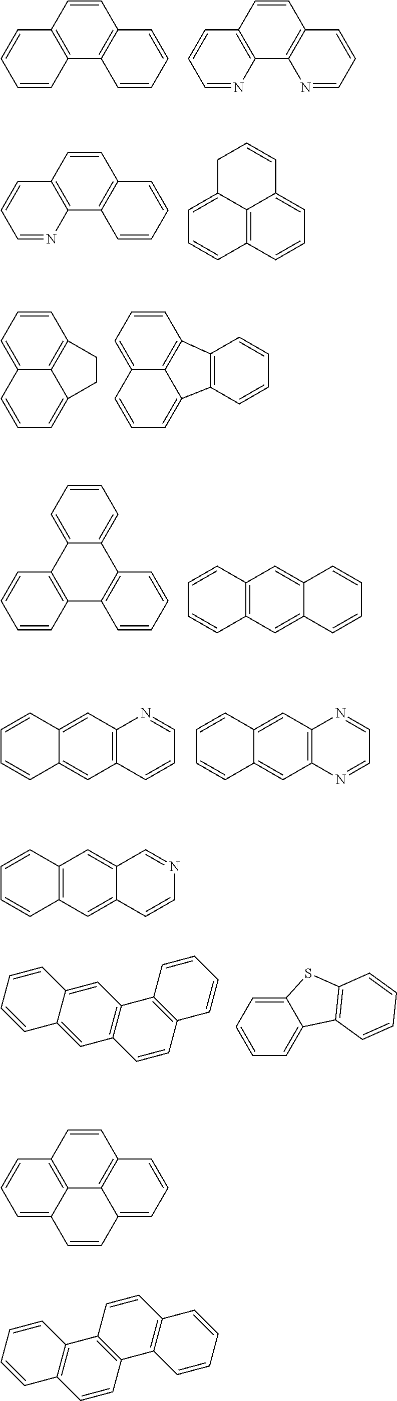

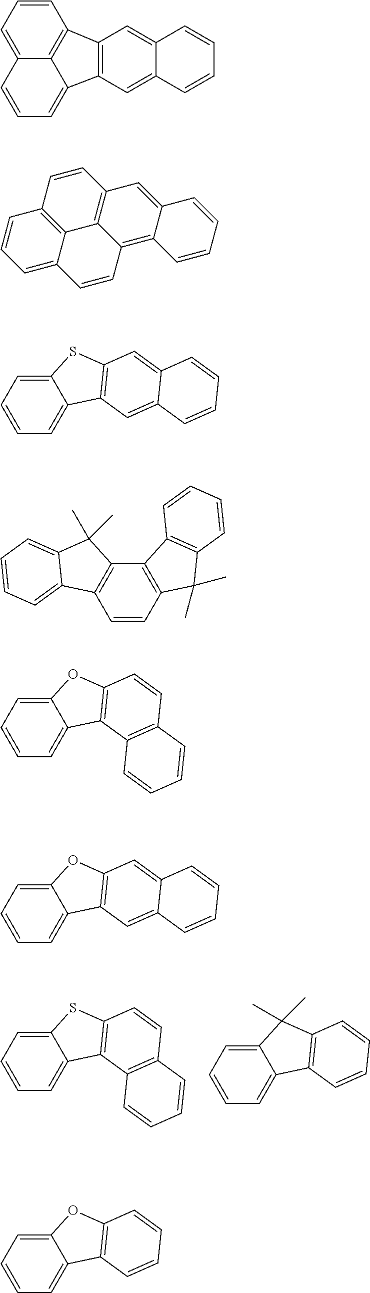

Further, Ar₁, Ar₂, Ar₃, and Ar₄ may be the same or different and contain the following structural units or a combination thereof:

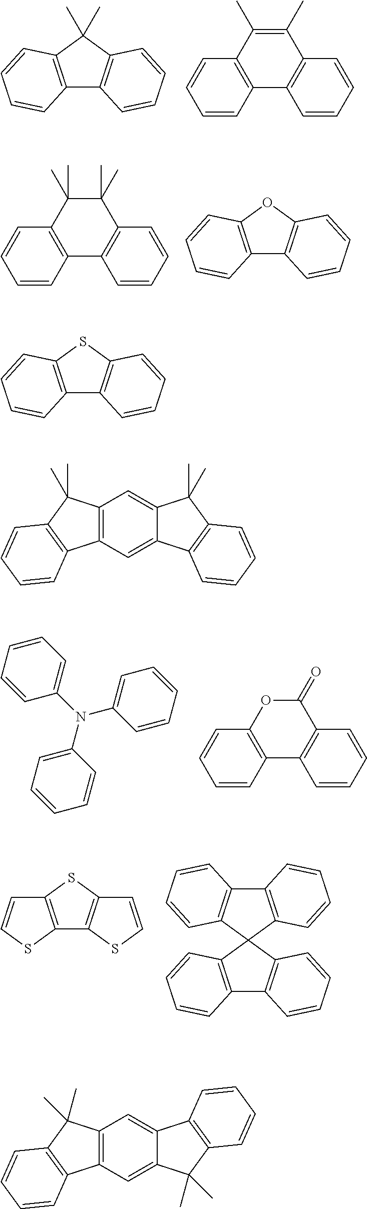

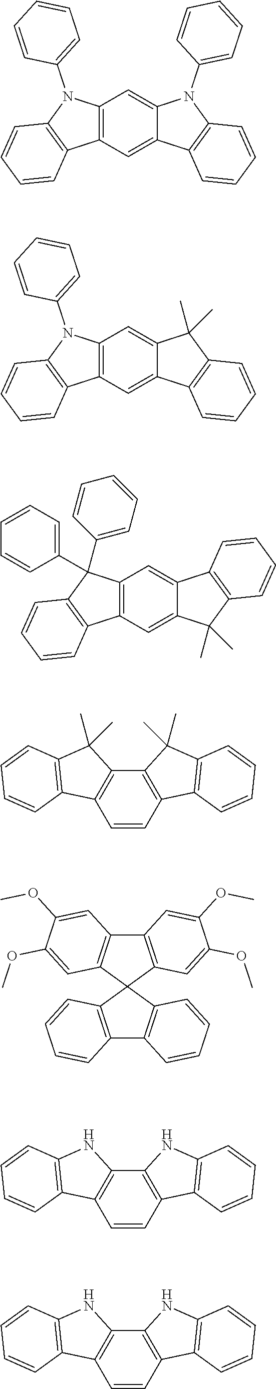

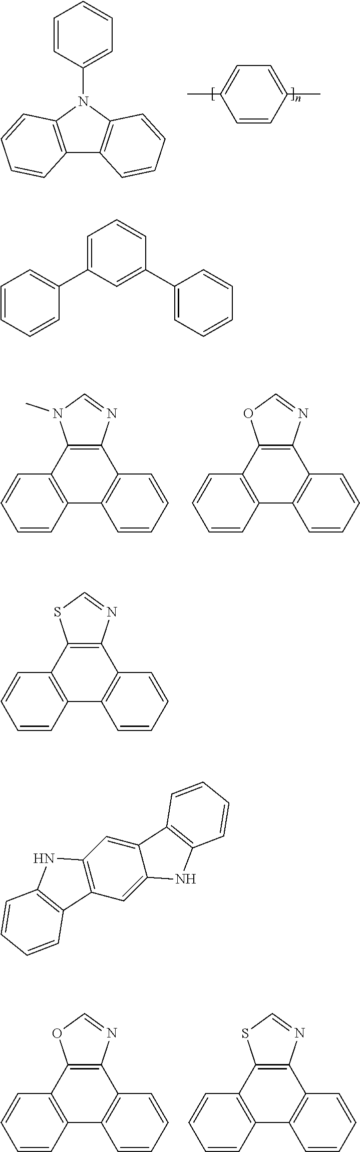

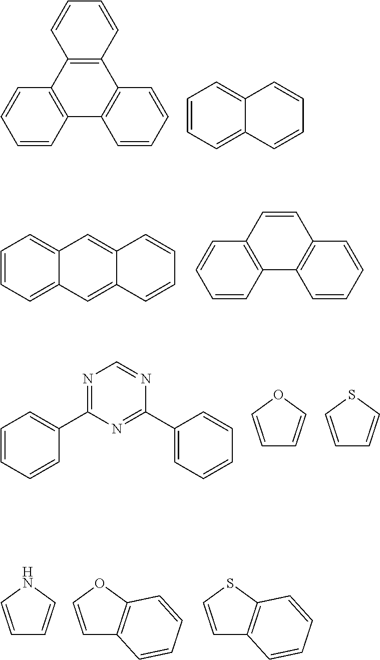

wherein n is 1, 2, 3 or 4.

In some embodiments, the compound according to the present disclosure has a relatively high electron mobility, typically greater than or equal to 10⁻⁵ cm²/V·s, further greater than or equal to 10⁻⁴ cm²/V·s, even further greater than or equal to 10⁻³ cm²/V·s.

In other embodiments, the compound according to the present disclosure has a glass transition temperature greater than or equal to 100° C., further greater than or equal to 110° C., still further greater than or equal to 120° C., even further greater than or equal to 140° C.

In other embodiments, the lowest unoccupied molecular orbital energy level LUMO of the compound according to the present disclosure is less than or equal to −2.7 eV, further less than or equal to −2.8 eV, still further less than or equal to −2.9 eV, even further less than or equal to −3.0 eV.

In other embodiments, the highest occupied molecular orbital energy level HOMO of the compound according to the present disclosure is less than or equal to −5.65 eV, further less than or equal to −5.8 eV, still further less than or equal to −5.9 eV, even further less than or equal to −6.0 eV.

In other embodiments, the triplet excited state energy level T1 of the compound according to the present disclosure is greater than or equal to 1.70 eV, further greater than or equal to 1.90 eV, still further greater than or equal to 2.10 eV, even further greater than or equal to 2.2 eV.

In the embodiments of the present disclosure, the energy level structure, i.e. the triplet excited state energy level E_{T}, HOMO and LUMO of the organic compound play a key role. The determination of these energy levels is described below.

The HOMO and LUMO energy levels can be measured by photoelectric effect, such as XPS (X-ray Photoelectron Spectroscopy) and UPS (Ultraviolet Photoelectron Spectroscopy) or by Cyclic Voltammetry (hereinafter referred to as CV). Recently, quantum chemistry method such as density functional theory (hereinafter referred to as DFT), has also become a feasible method for calculating molecular orbital energy levels.

The triplet excited state energy level E_{T} of organic materials can be measured by low temperature time-resolved luminescence spectroscopy, or obtained by quantum simulation calculation (e.g., by Time-dependent DFT), such as by the commercial software Gaussian 03W (Gaussian Inc.), and the specific simulation method may refer to WO2011141110 or may be as described in the embodiments below.

It should be noted that, the absolute values of HOMO, LUMO, and E_{T} depend on the measurement method or calculation method used, even for the same method, different HOMO/LUMO value may be obtained by different evaluation method, such as different HOMO/LUMO value can be obtained by starting point and peak point on the CV curve. Therefore, reasonable and meaningful comparisons should be made by using same measurement method and same evaluation method. In the description of the embodiments of the present disclosure, the values of HOMO, LUMO and E_{T} are based on the simulations of Time-dependent DFT, but this does not affect the application of other measurement or calculation methods. The energy level values determined by different methods should be calibrated against each other.

In an embodiment, the compound according to the present disclosure is at least partially deuterated, further 10% of H is deuterated, still further 20% of H is deuterated, even further 30% of H is deuterated, and further 40% of H is deuterated.

Suitable examples of the compound according to the present disclosure are listed below, but are not limited to the following structures:

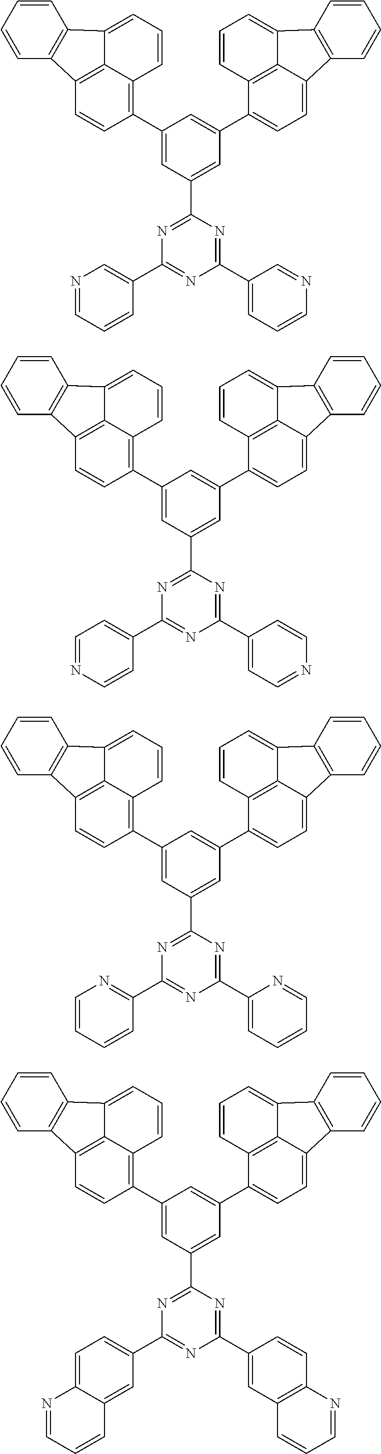 [SMILES: c1cncc(-c2nc(-c3cccnc3)nc(-c3cc(-c4ccc5c6c(cccc46)-c4ccccc4-5)cc(-c4ccc5c6c(cccc46)-c4ccccc4-5)c3)n2)c1 ; c1ccc2c(c1)-c1cccc3c(-c4cc(-c5nc(-c6ccncc6)nc(-c6ccncc6)n5)cc(-c5ccc6c7c(cccc57)-c5ccccc5-6)c4)ccc-2c13 ; c1ccc(-c2nc(-c3cc(-c4ccc5c6c(cccc46)-c4ccccc4-5)cc(-c4ccc5c6c(cccc46)-c4ccccc4-5)c3)nc(-c3ccccn3)n2)nc1 ; c1ccc2c(c1)-c1cccc3c(-c4cc(-c5nc(-c6ccc7ncccc7c6)nc(-c6ccc7ncccc7c6)n5)cc(-c5ccc6c7c(cccc57)-c5ccccc5-6)c4)ccc-2c13]

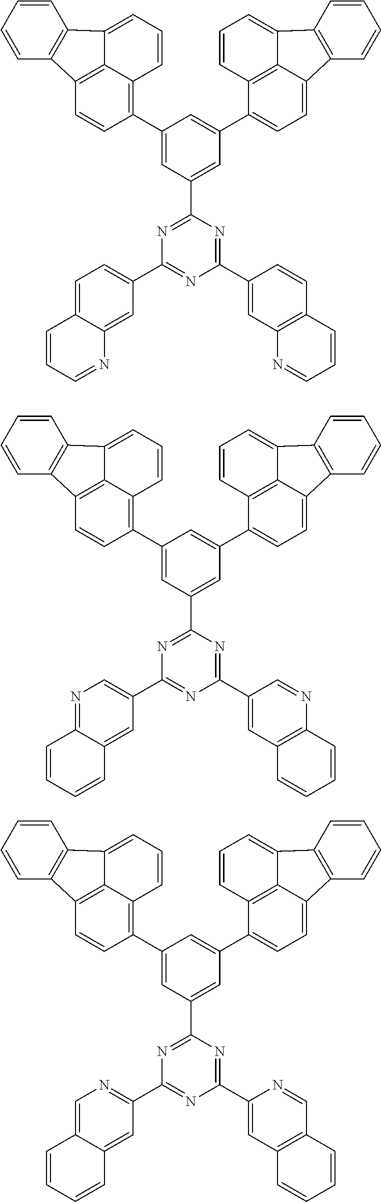

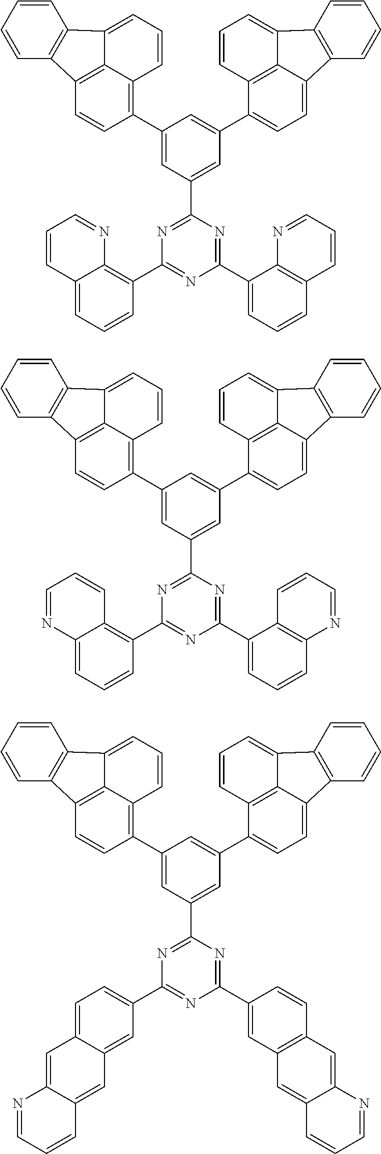 [SMILES: c1ccc2c(c1)-c1cccc3c(-c4cc(-c5nc(-c6cccc7cccnc67)nc(-c6cccc7cccnc67)n5)cc(-c5ccc6c7c(cccc57)-c5ccccc5-6)c4)ccc-2c13 ; c1ccc2c(c1)-c1cccc3c(-c4cc(-c5nc(-c6cccc7ncccc67)nc(-c6cccc7ncccc67)n5)cc(-c5ccc6c7c(cccc57)-c5ccccc5-6)c4)ccc-2c13 ; c1ccc2c(c1)-c1cccc3c(-c4cc(-c5nc(-c6ccc7cc8ncccc8cc7c6)nc(-c6ccc7cc8ncccc8cc7c6)n5)cc(-c5ccc6c7c(cccc57)-c5ccccc5-6)c4)ccc-2c13]

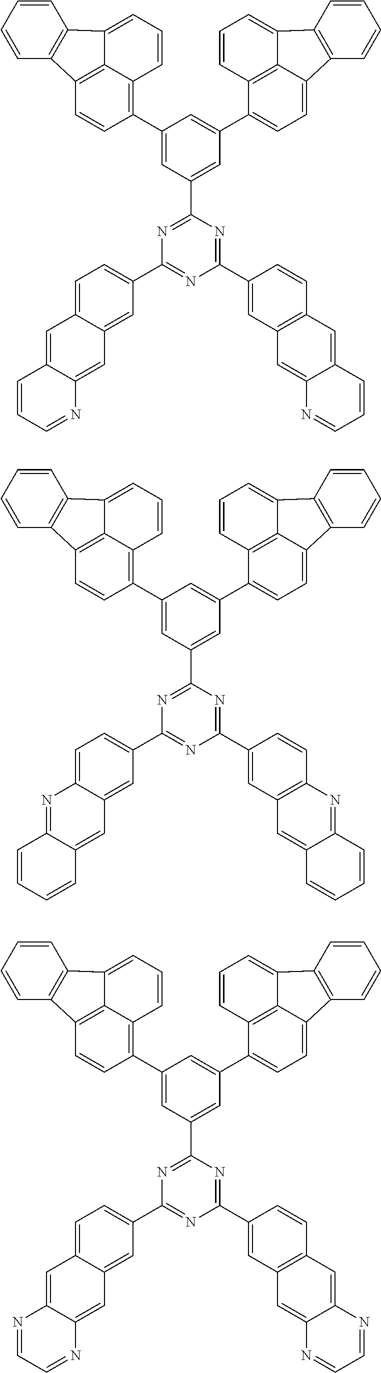

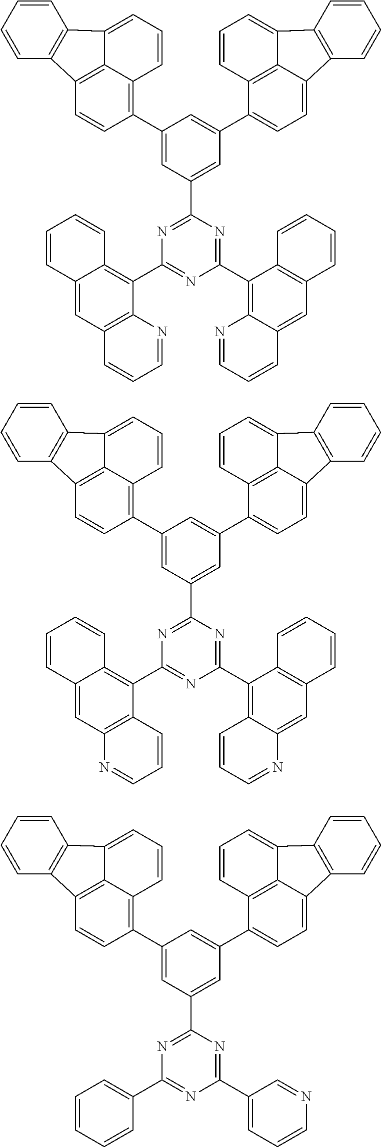

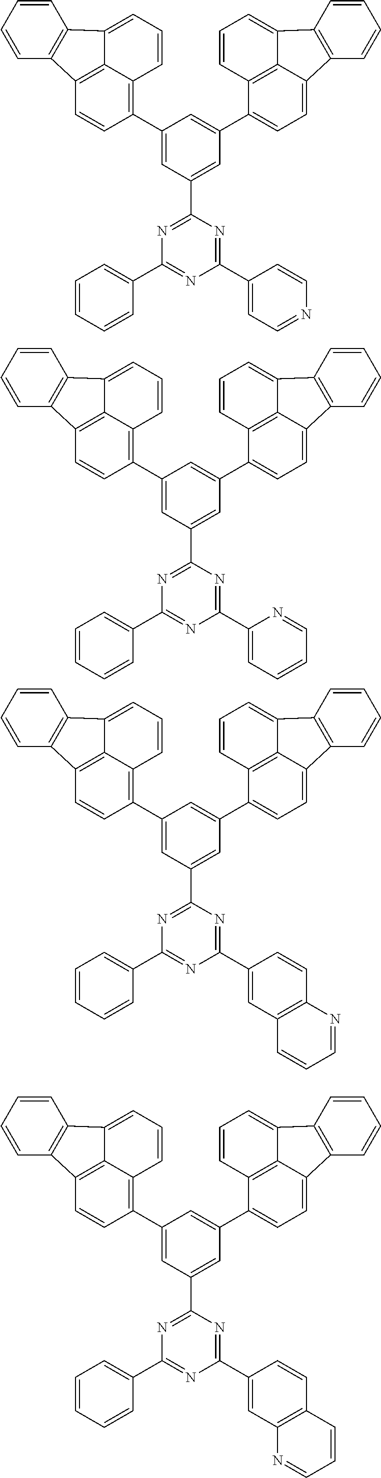

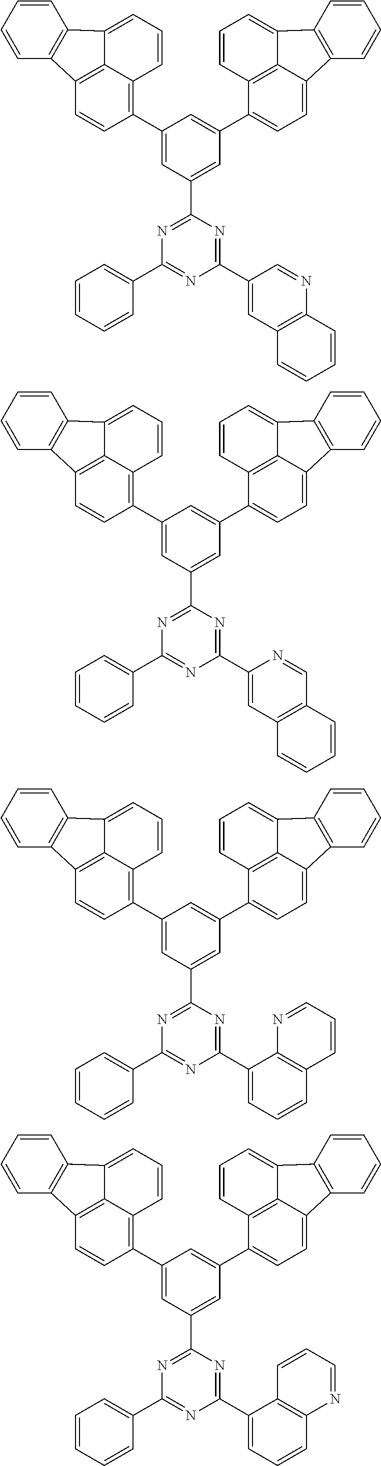

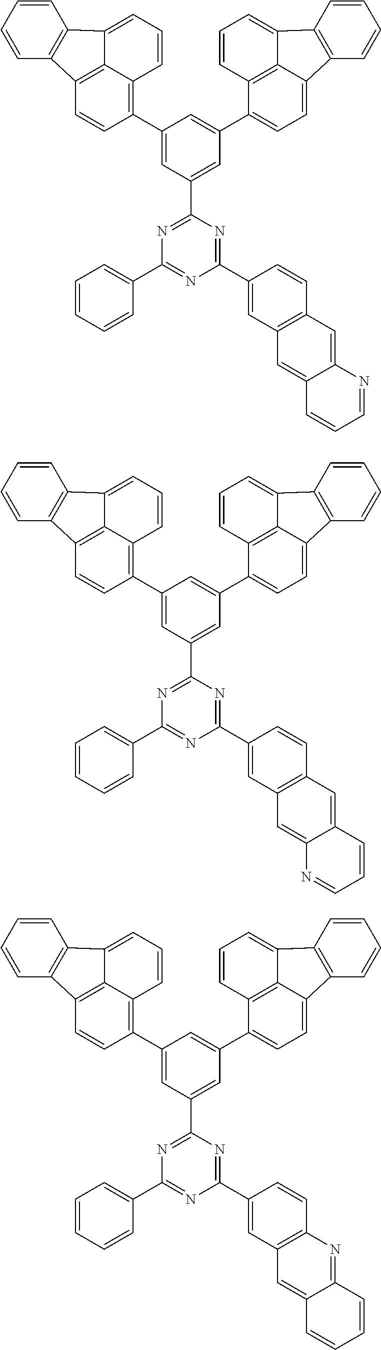

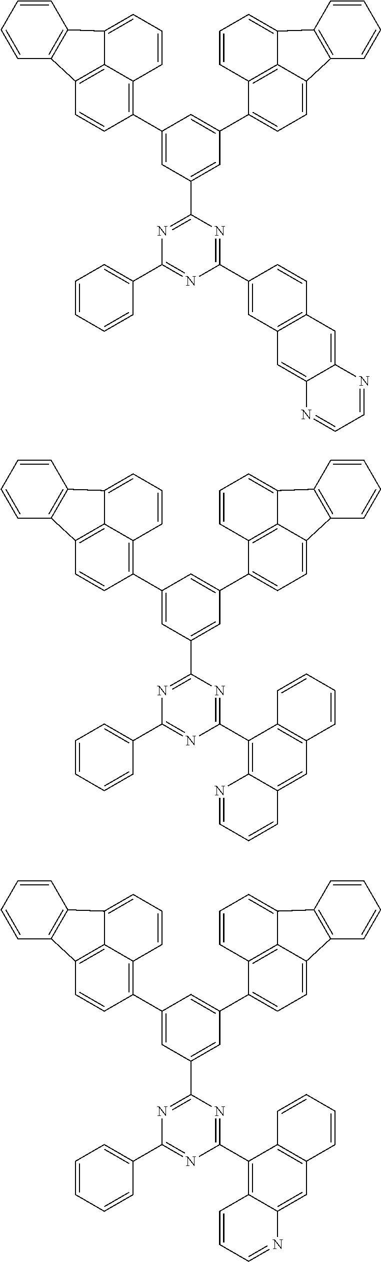

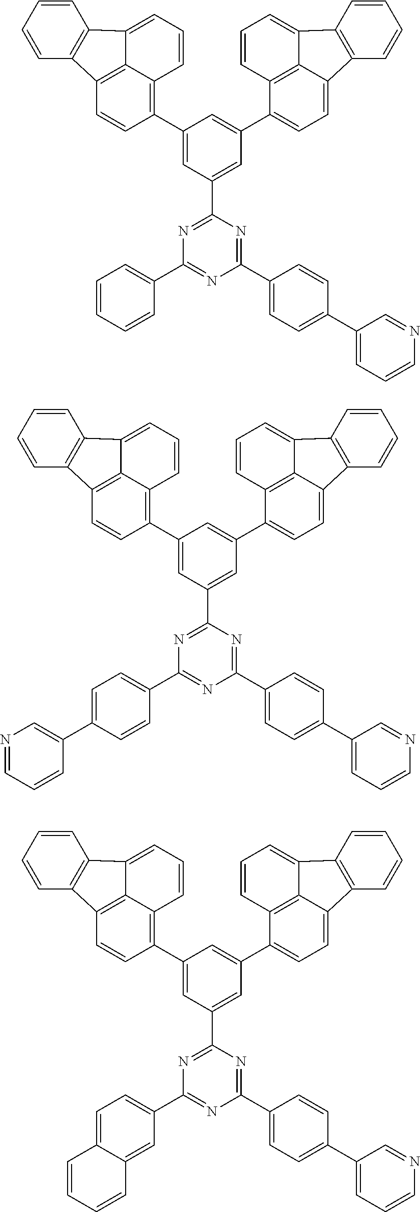

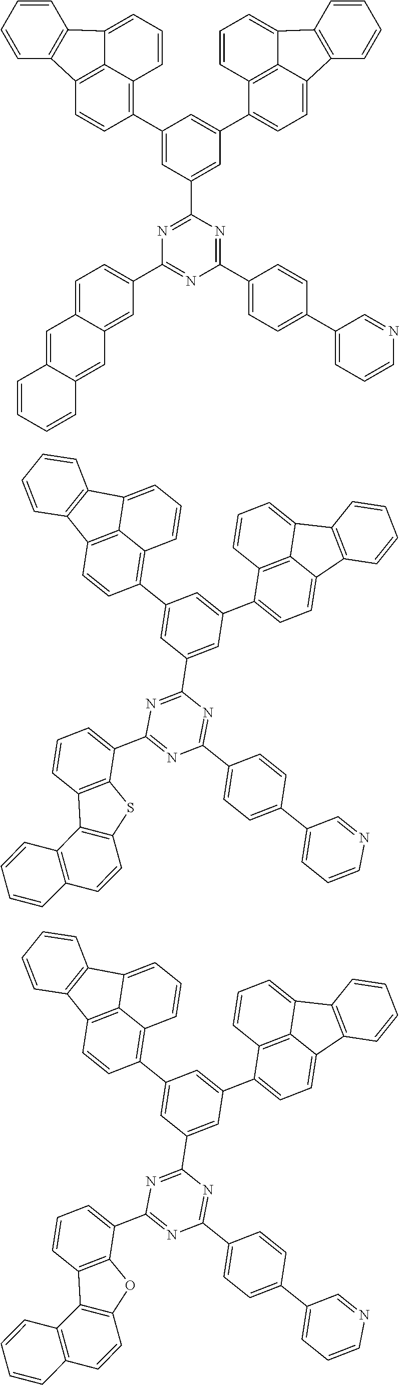

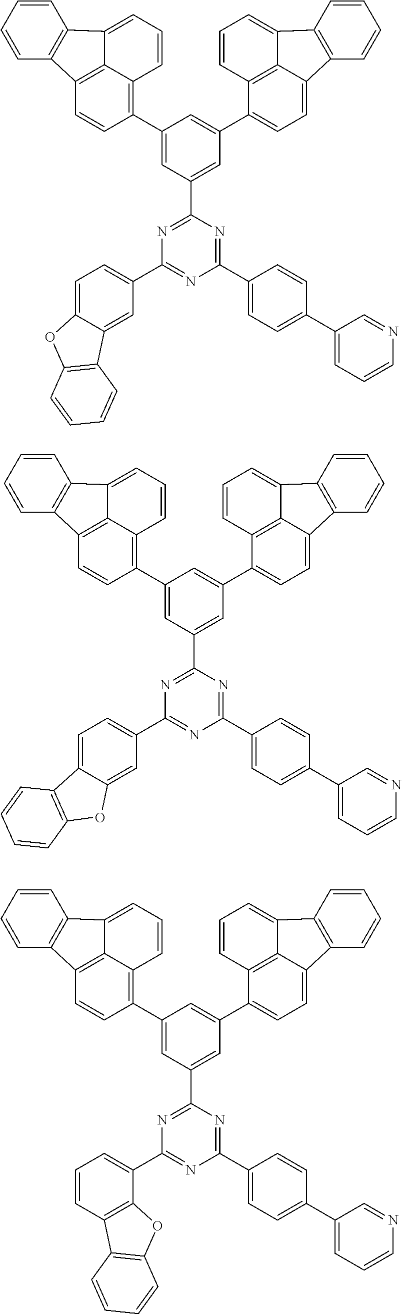

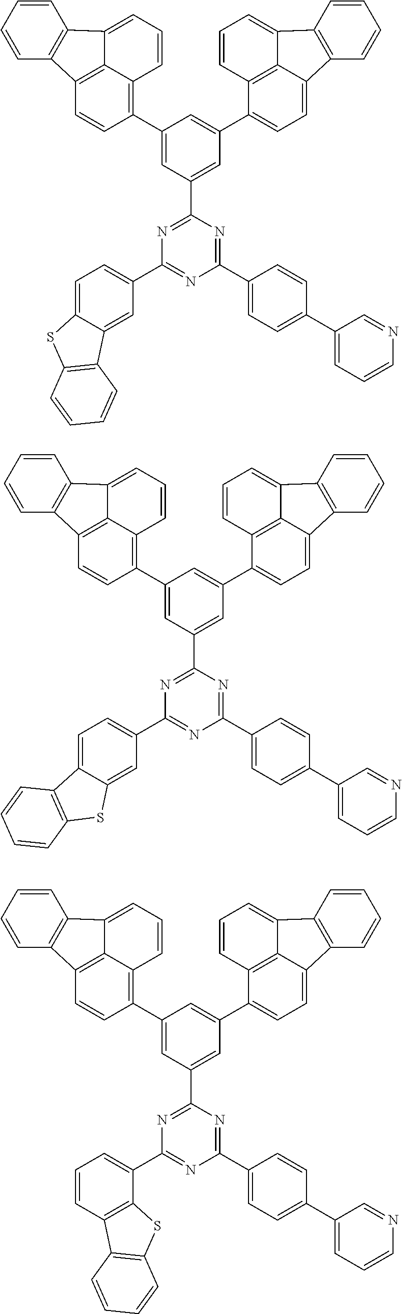

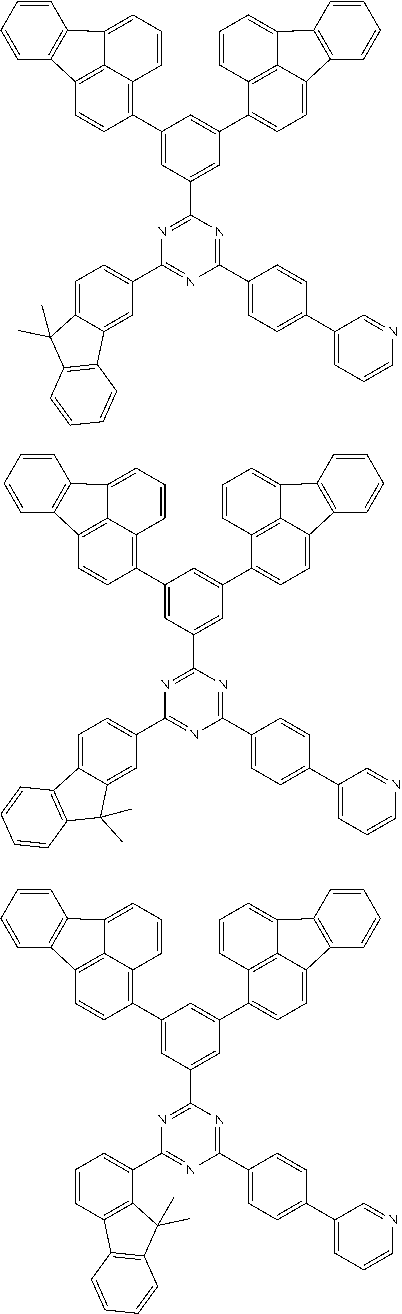

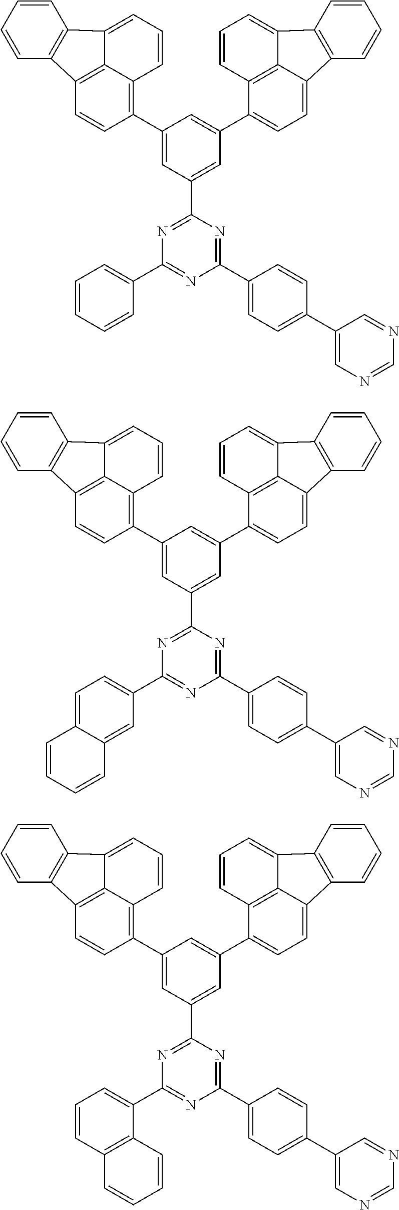

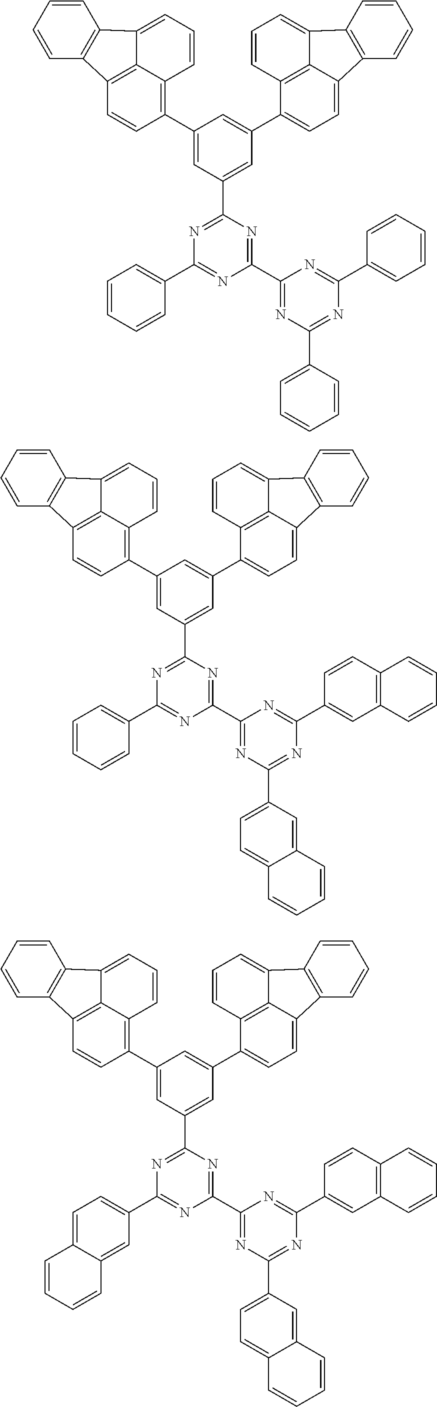

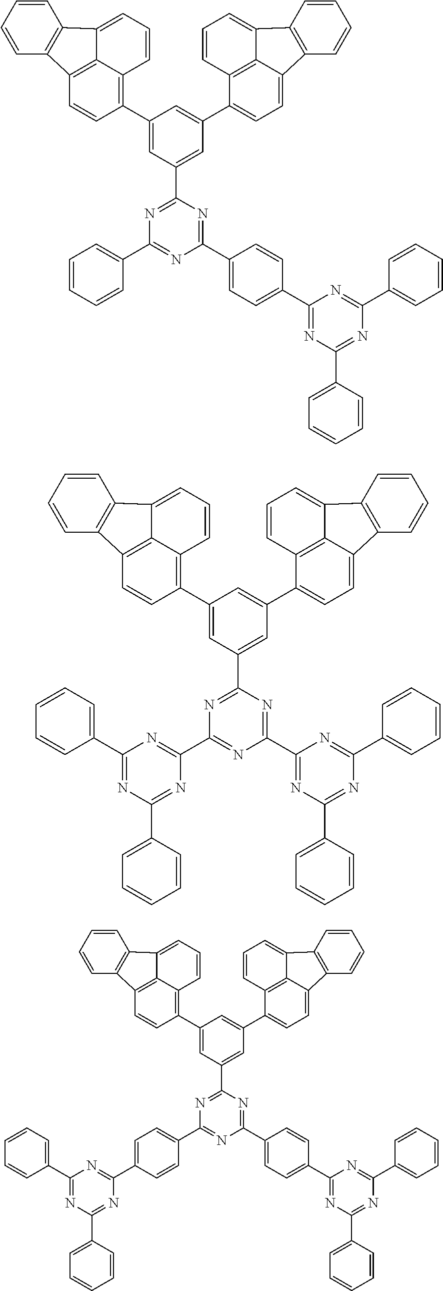

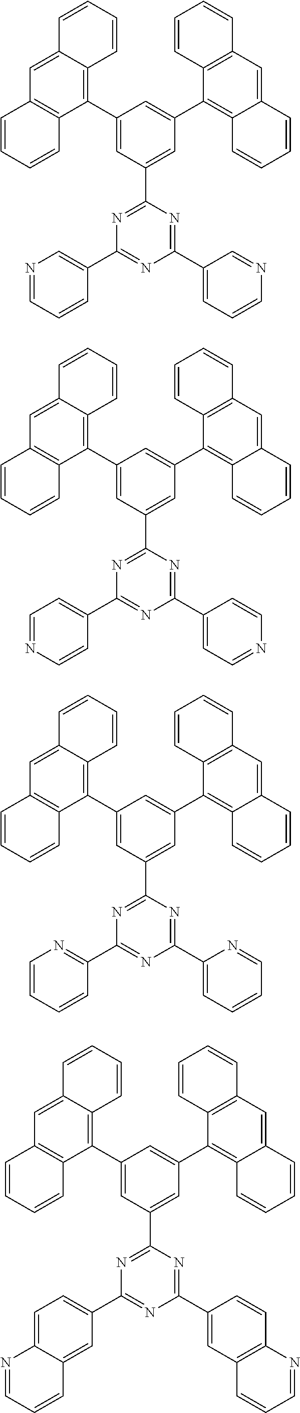

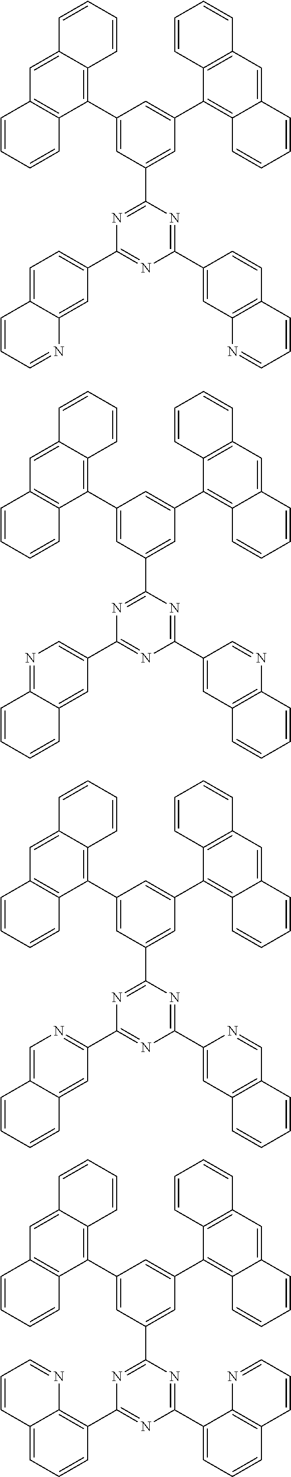

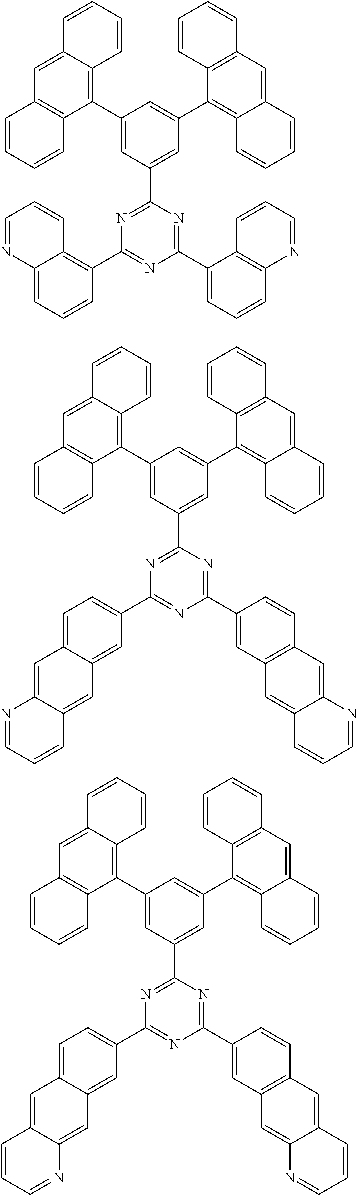

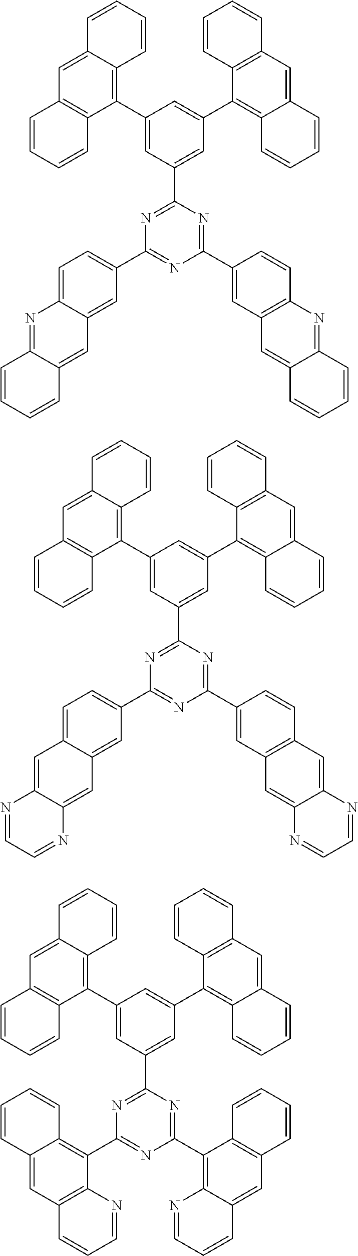 [SMILES: c1ccc2nc3ccc(-c4nc(-c5cc(-c6c7ccccc7cc7ccccc67)cc(-c6c7ccccc7cc7ccccc67)c5)nc(-c5ccc6nc7ccccc7cc6c5)n4)cc3cc2c1 ; c1ccc2c(-c3cc(-c4nc(-c5ccc6cc7nccnc7cc6c5)nc(-c5ccc6cc7nccnc7cc6c5)n4)cc(-c4c5ccccc5cc5ccccc45)c3)c3ccccc3cc2c1 ; c1ccc2c(-c3cc(-c4nc(-c5c6ccccc6cc6cccnc56)nc(-c5c6ccccc6cc6cccnc56)n4)cc(-c4c5ccccc5cc5ccccc45)c3)c3ccccc3cc2c1]

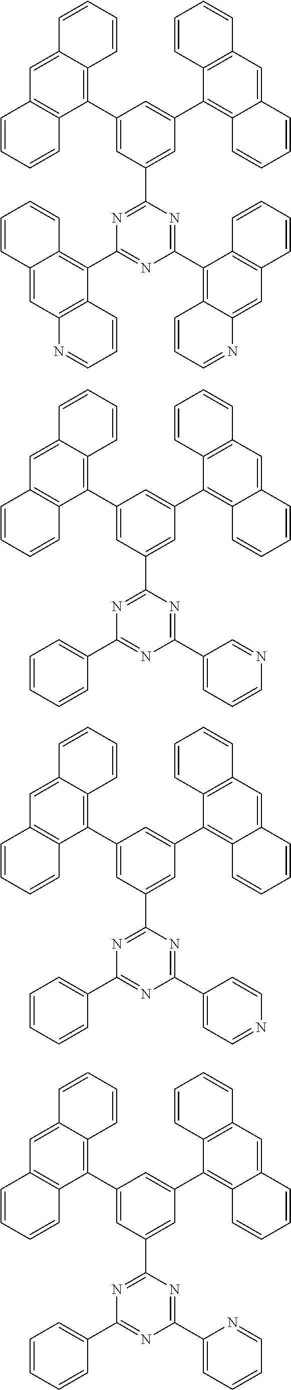

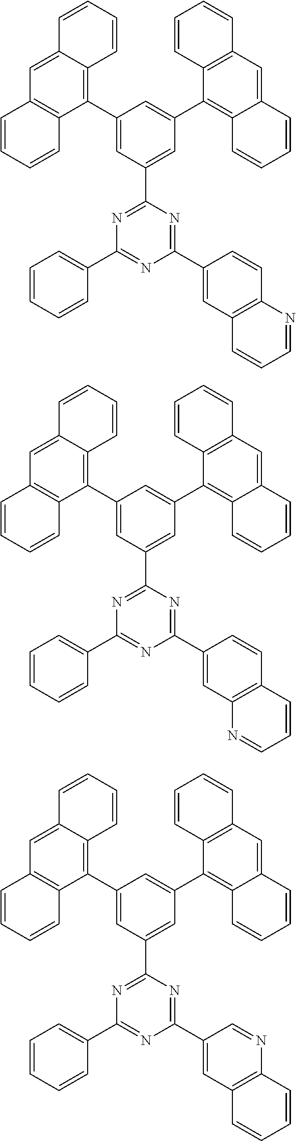

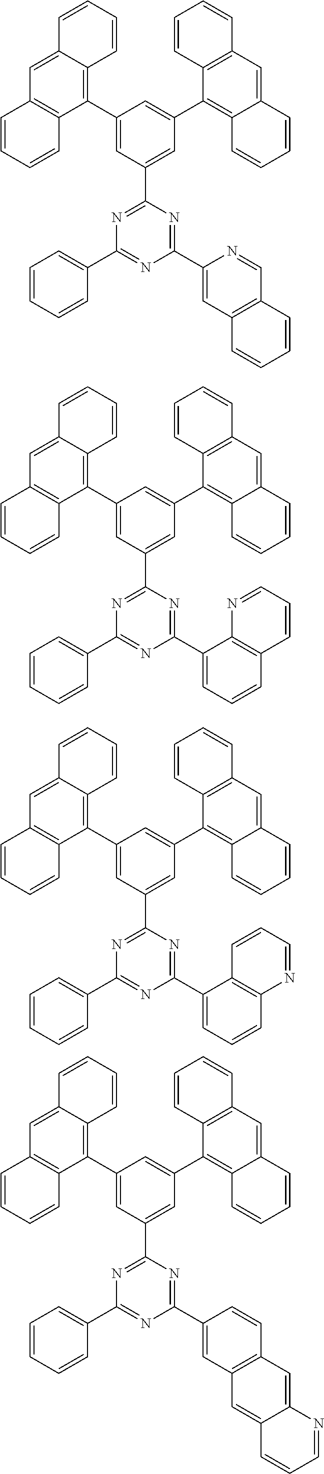

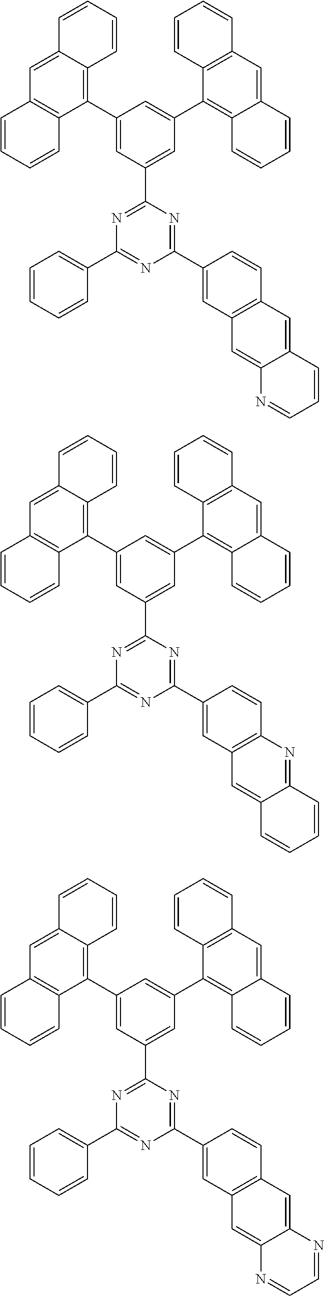 [SMILES: c1ccc(-c2nc(-c3cc(-c4c5ccccc5cc5ccccc45)cc(-c4c5ccccc5cc5ccccc45)c3)nc(-c3ccc4cc5cccnc5cc4c3)n2)cc1 ; c1ccc(-c2nc(-c3cc(-c4c5ccccc5cc5ccccc45)cc(-c4c5ccccc5cc5ccccc45)c3)nc(-c3ccc4nc5ccccc5cc4c3)n2)cc1 ; c1ccc(-c2nc(-c3cc(-c4c5ccccc5cc5ccccc45)cc(-c4c5ccccc5cc5ccccc45)c3)nc(-c3ccc4cc5nccnc5cc4c3)n2)cc1]

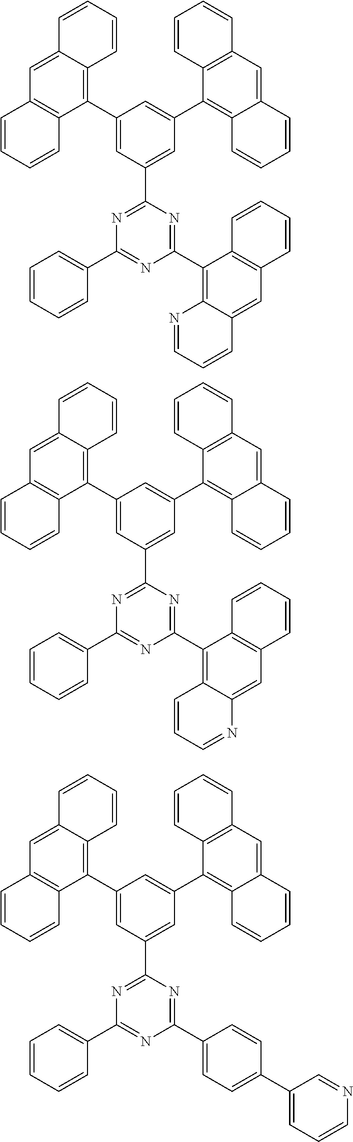

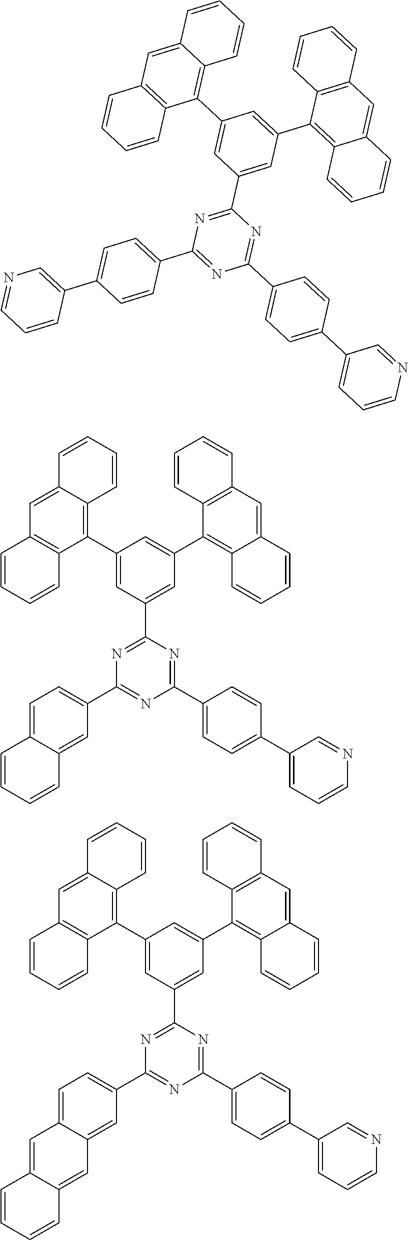 [SMILES: c1cncc(-c2ccc(-c3nc(-c4ccc(-c5cccnc5)cc4)nc(-c4cc(-c5c6ccccc6cc6ccccc56)cc(-c5c6ccccc6cc6ccccc56)c4)n3)cc2)c1 ; c1cncc(-c2ccc(-c3nc(-c4cc(-c5c6ccccc6cc6ccccc56)cc(-c5c6ccccc6cc6ccccc56)c4)nc(-c4ccc5ccccc5c4)n3)cc2)c1 ; c1cncc(-c2ccc(-c3nc(-c4cc(-c5c6ccccc6cc6ccccc56)cc(-c5c6ccccc6cc6ccccc56)c4)nc(-c4ccc5cc6ccccc6cc5c4)n3)cc2)c1]

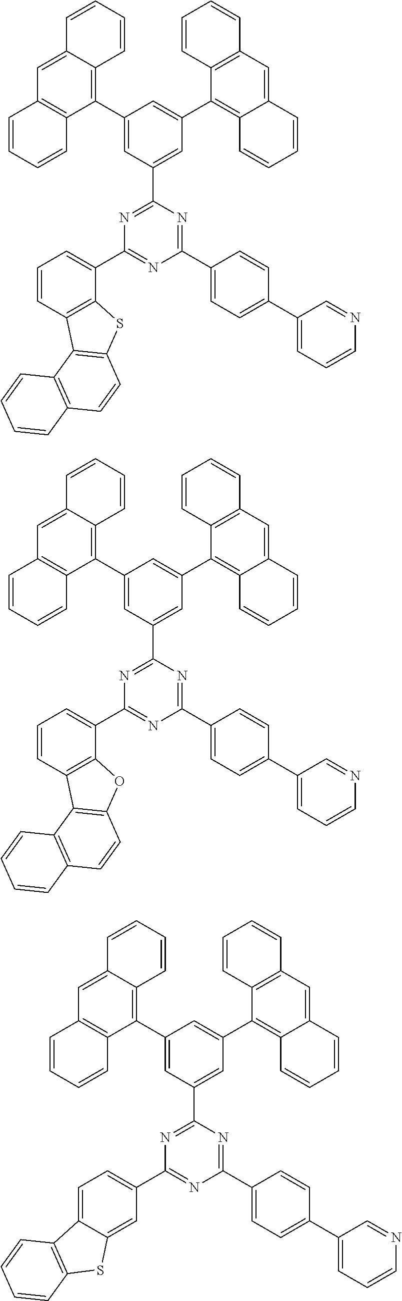

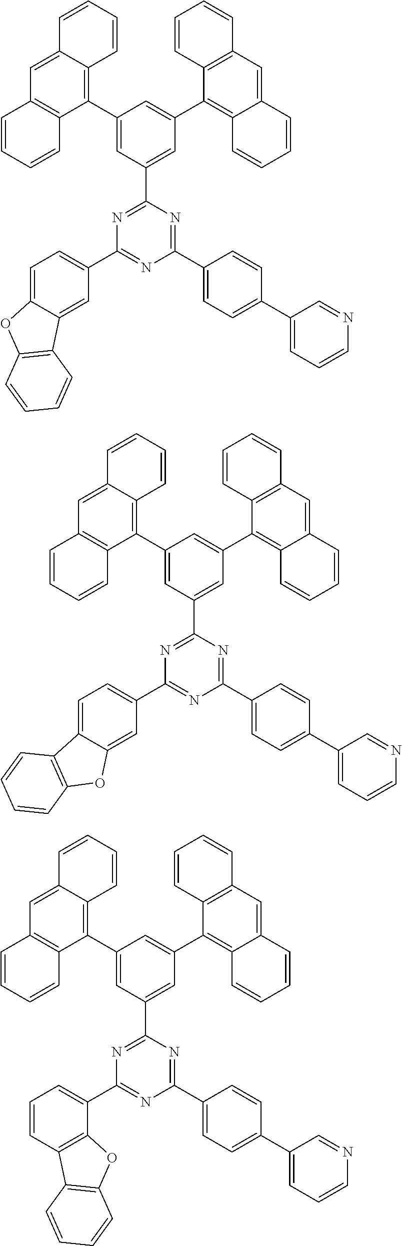

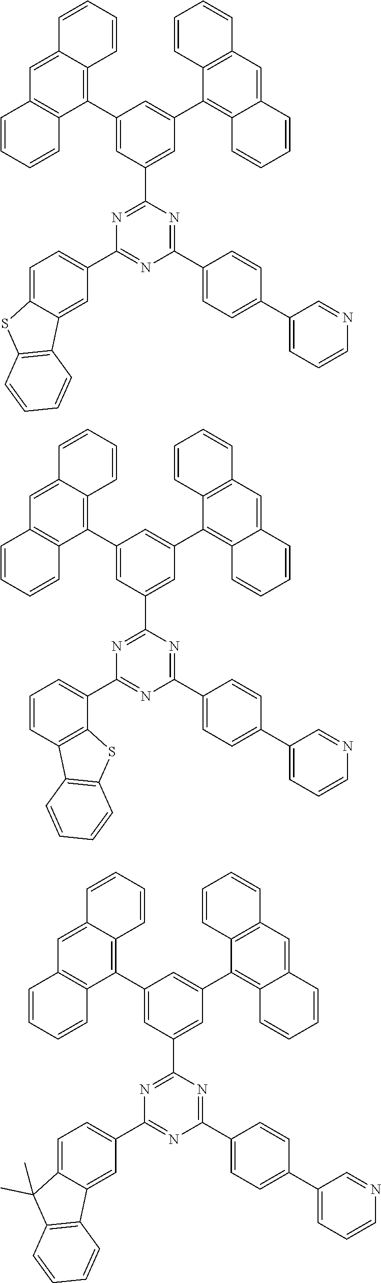

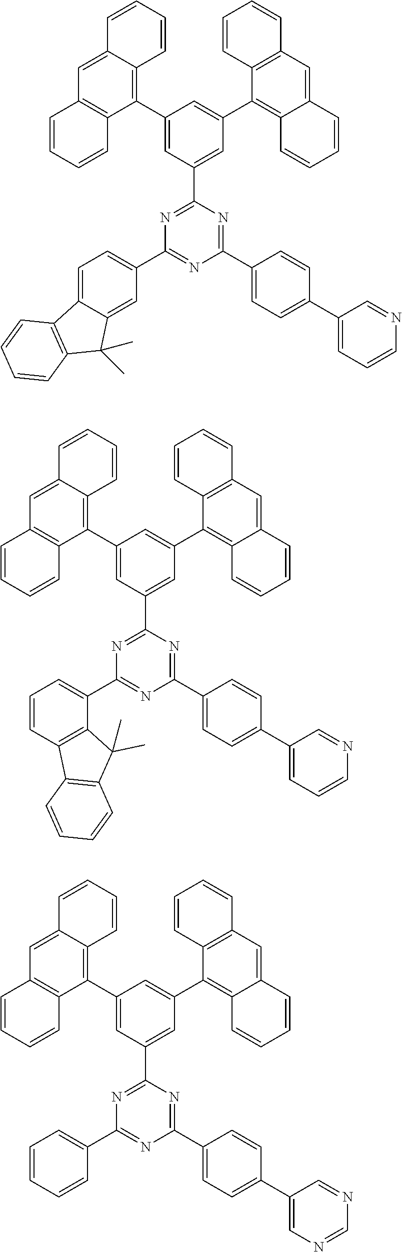

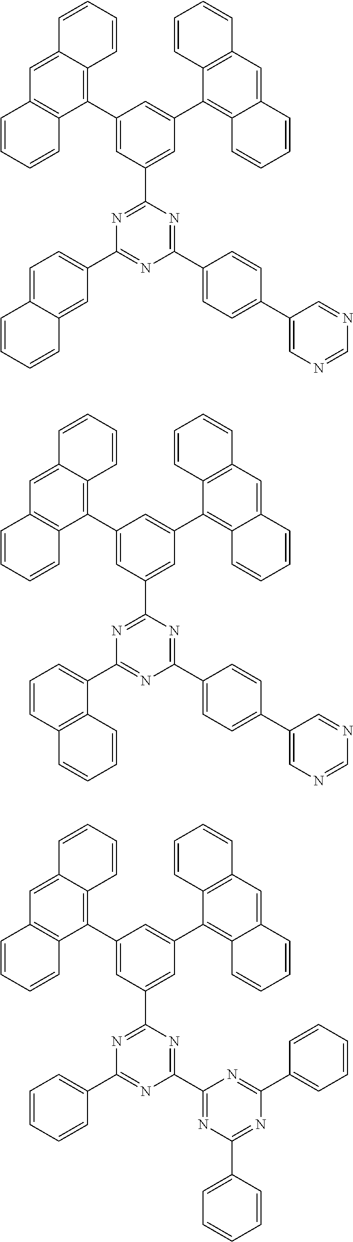

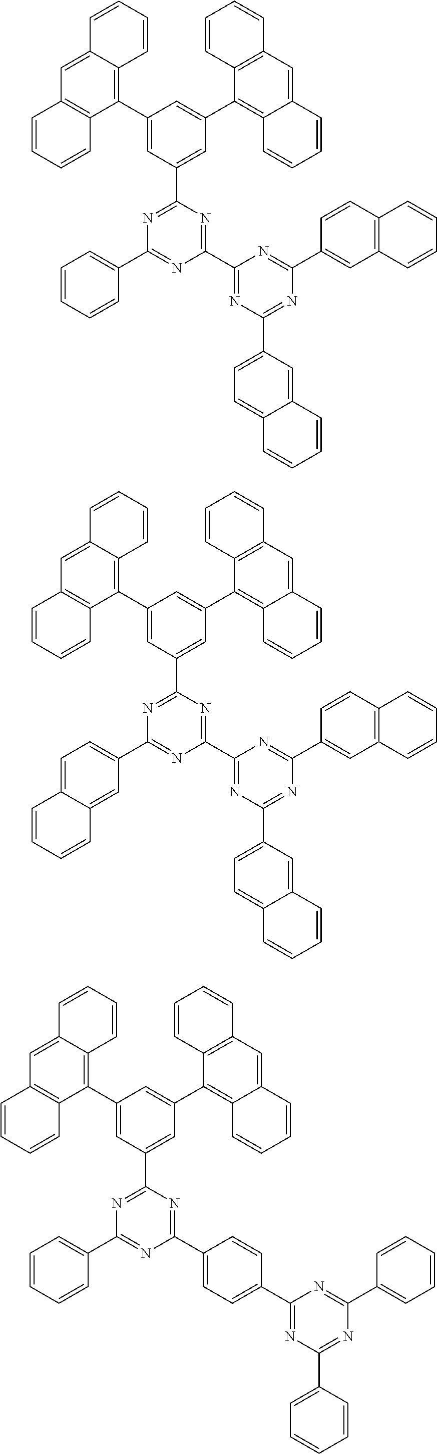 [SMILES: c1ccc(-c2nc(-c3cc(-c4c5ccccc5cc5ccccc45)cc(-c4c5ccccc5cc5ccccc45)c3)nc(-c3nc(-c4ccc5ccccc5c4)nc(-c4ccc5ccccc5c4)n3)n2)cc1 ; c1ccc2cc(-c3nc(-c4cc(-c5c6ccccc6cc6ccccc56)cc(-c5c6ccccc6cc6ccccc56)c4)nc(-c4nc(-c5ccc6ccccc6c5)nc(-c5ccc6ccccc6c5)n4)n3)ccc2c1 ; c1ccc(-c2nc(-c3ccccc3)nc(-c3ccc(-c4nc(-c5ccccc5)nc(-c5cc(-c6c7ccccc7cc7ccccc67)cc(-c6c7ccccc7cc7ccccc67)c5)n4)cc3)n2)cc1]

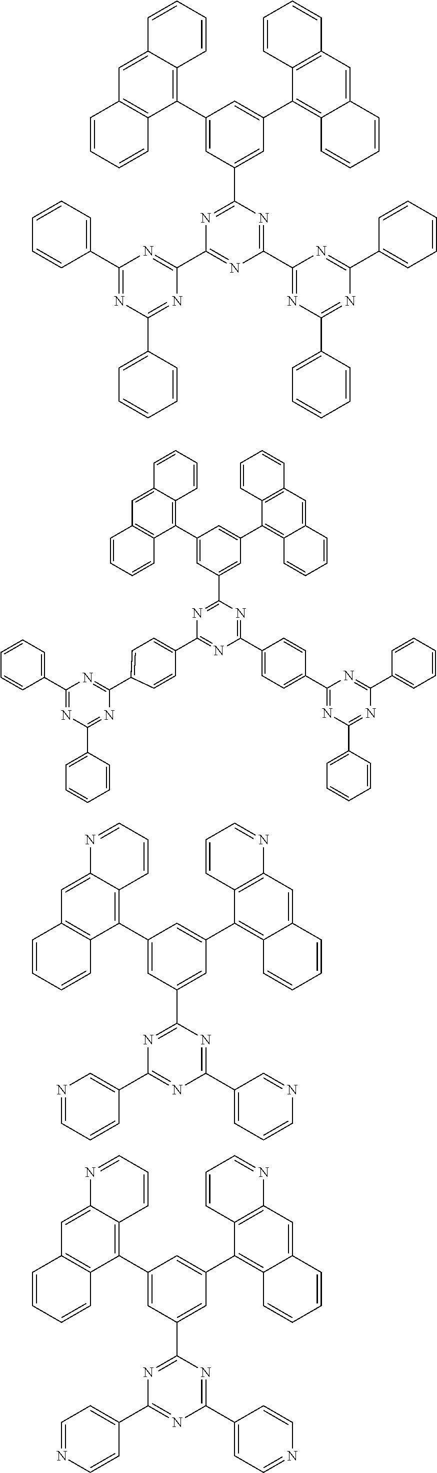

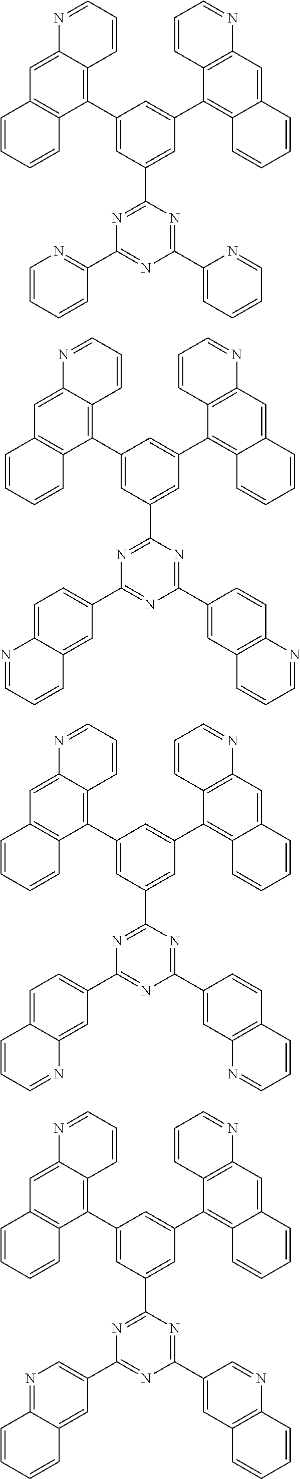

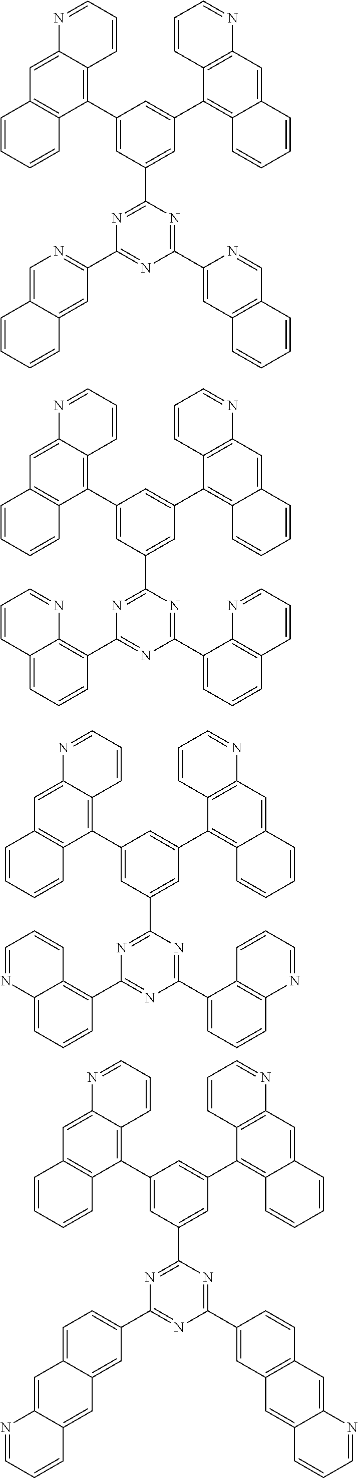

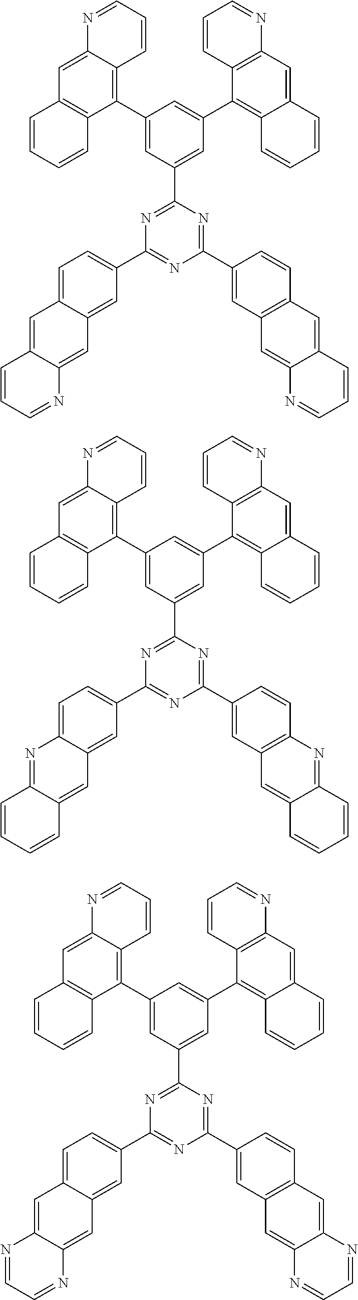 [SMILES: c1cnc2cc3cc(-c4nc(-c5cc(-c6c7ccccc7cc7ncccc67)cc(-c6c7ccccc7cc7ncccc67)c5)nc(-c5ccc6cc7cccnc7cc6c5)n4)ccc3cc2c1 ; c1ccc2nc3ccc(-c4nc(-c5cc(-c6c7ccccc7cc7ncccc67)cc(-c6c7ccccc7cc7ncccc67)c5)nc(-c5ccc6nc7ccccc7cc6c5)n4)cc3cc2c1 ; c1ccc2c(-c3cc(-c4nc(-c5ccc6cc7nccnc7cc6c5)nc(-c5ccc6cc7nccnc7cc6c5)n4)cc(-c4c5ccccc5cc5ncccc45)c3)c3cccnc3cc2c1]

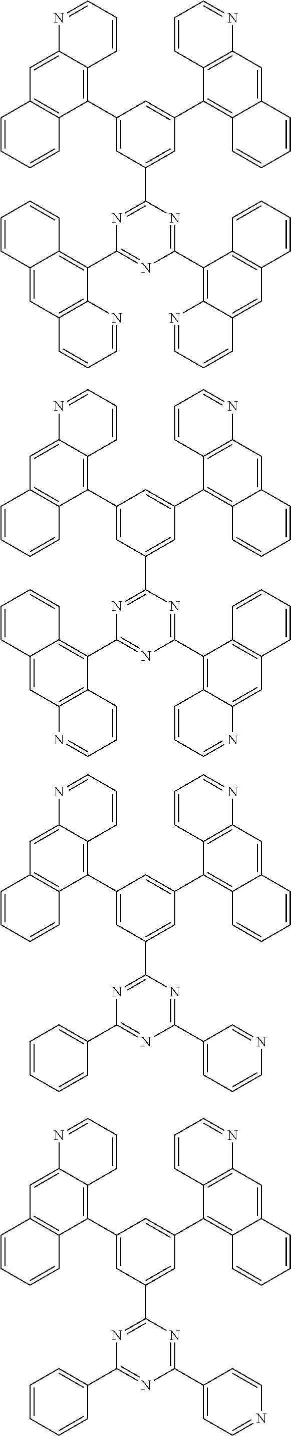

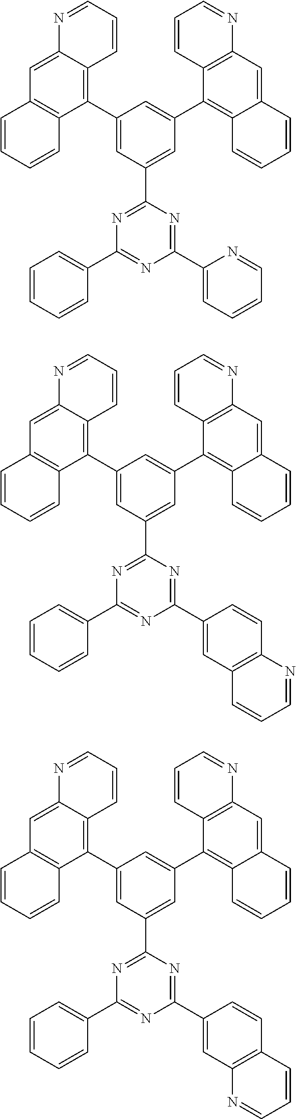

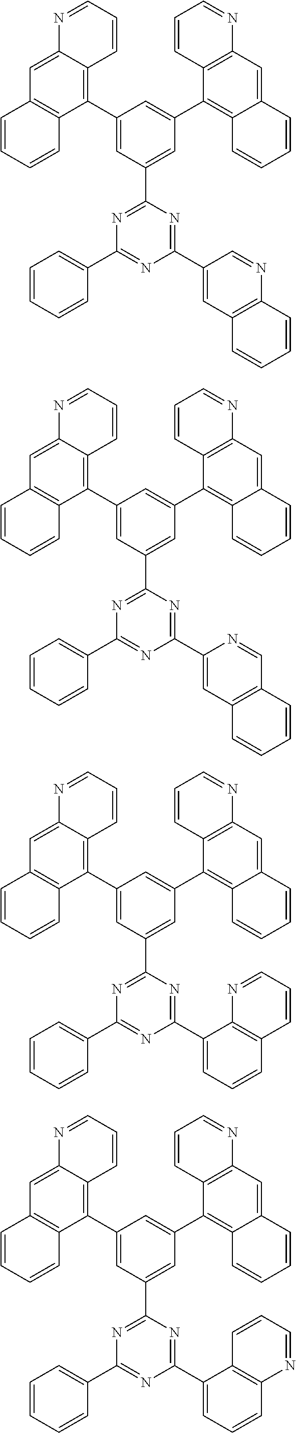

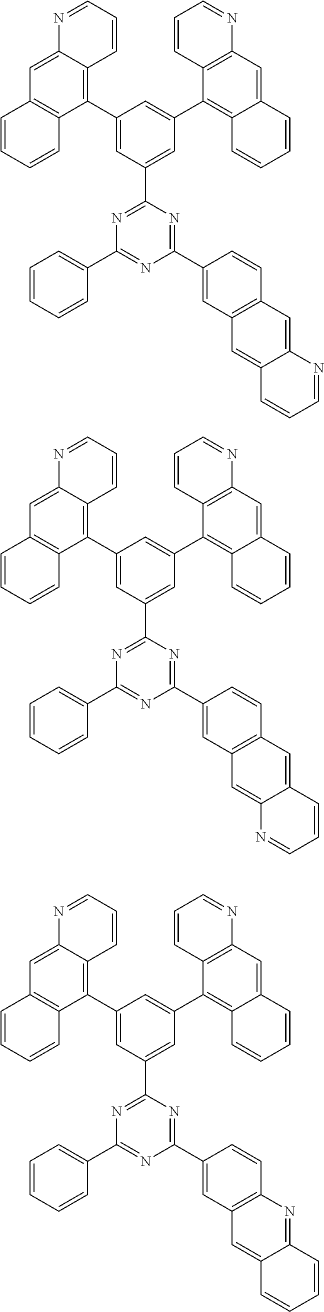 [SMILES: c1ccc(-c2nc(-c3cc(-c4c5ccccc5cc5ncccc45)cc(-c4c5ccccc5cc5ncccc45)c3)nc(-c3ccc4cc5ncccc5cc4c3)n2)cc1 ; c1ccc(-c2nc(-c3cc(-c4c5ccccc5cc5ncccc45)cc(-c4c5ccccc5cc5ncccc45)c3)nc(-c3ccc4cc5cccnc5cc4c3)n2)cc1 ; c1ccc(-c2nc(-c3cc(-c4c5ccccc5cc5ncccc45)cc(-c4c5ccccc5cc5ncccc45)c3)nc(-c3ccc4nc5ccccc5cc4c3)n2)cc1]

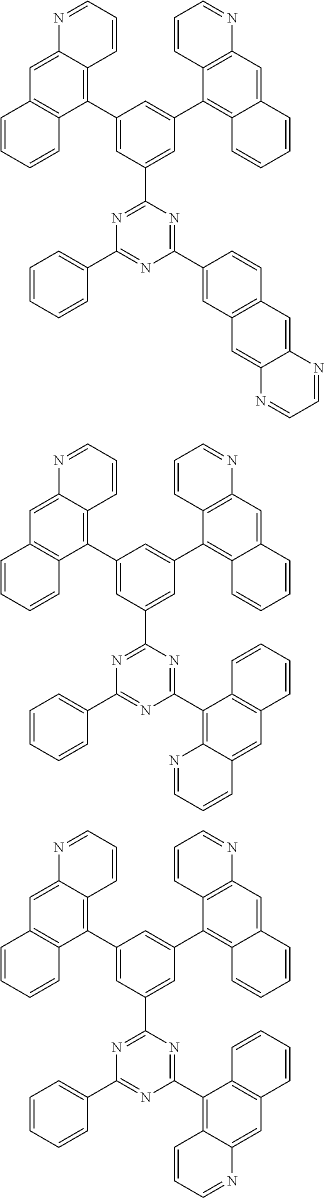

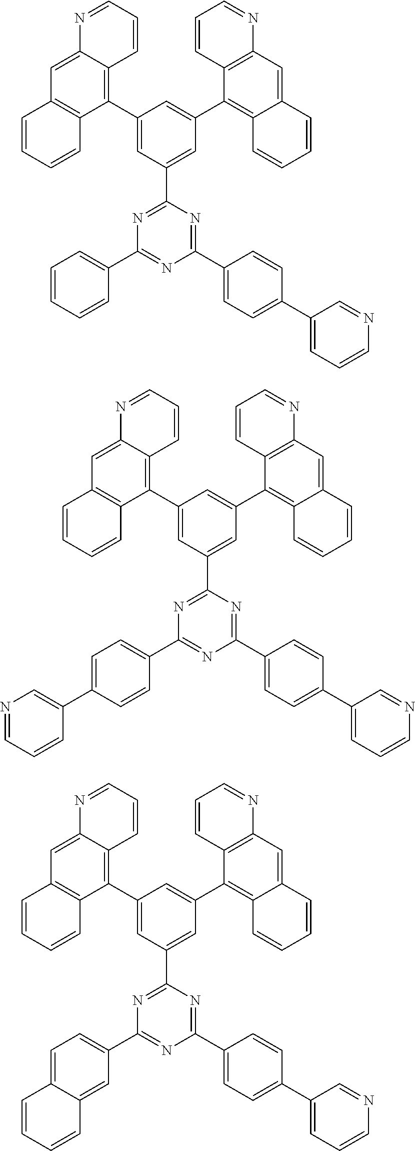 [SMILES: c1ccc(-c2nc(-c3ccc(-c4cccnc4)cc3)nc(-c3cc(-c4c5ccccc5cc5ncccc45)cc(-c4c5ccccc5cc5ncccc45)c3)n2)cc1 ; c1cncc(-c2ccc(-c3nc(-c4ccc(-c5cccnc5)cc4)nc(-c4cc(-c5c6ccccc6cc6ncccc56)cc(-c5c6ccccc6cc6ncccc56)c4)n3)cc2)c1 ; c1cncc(-c2ccc(-c3nc(-c4cc(-c5c6ccccc6cc6ncccc56)cc(-c5c6ccccc6cc6ncccc56)c4)nc(-c4ccc5ccccc5c4)n3)cc2)c1]

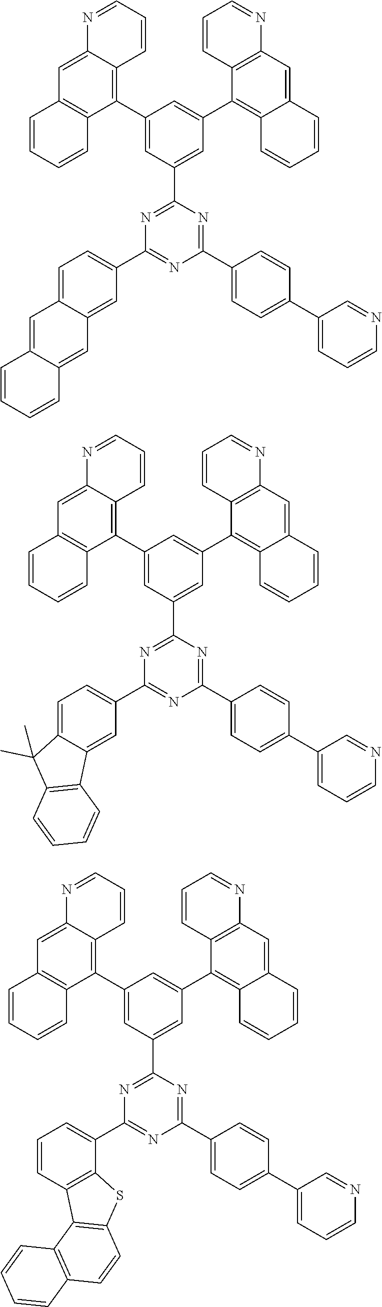

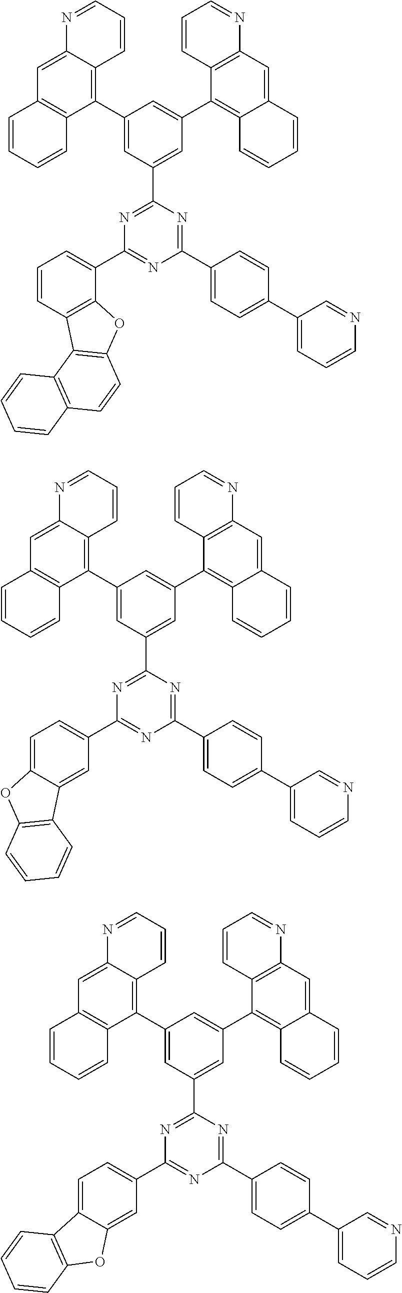 [SMILES: c1cncc(-c2ccc(-c3nc(-c4cc(-c5c6ccccc6cc6ncccc56)cc(-c5c6ccccc6cc6ncccc56)c4)nc(-c4cccc5c4oc4ccc6ccccc6c45)n3)cc2)c1 ; c1cncc(-c2ccc(-c3nc(-c4cc(-c5c6ccccc6cc6ncccc56)cc(-c5c6ccccc6cc6ncccc56)c4)nc(-c4ccc5oc6ccccc6c5c4)n3)cc2)c1 ; c1cncc(-c2ccc(-c3nc(-c4cc(-c5c6ccccc6cc6ncccc56)cc(-c5c6ccccc6cc6ncccc56)c4)nc(-c4ccc5c(c4)oc4ccccc45)n3)cc2)c1]

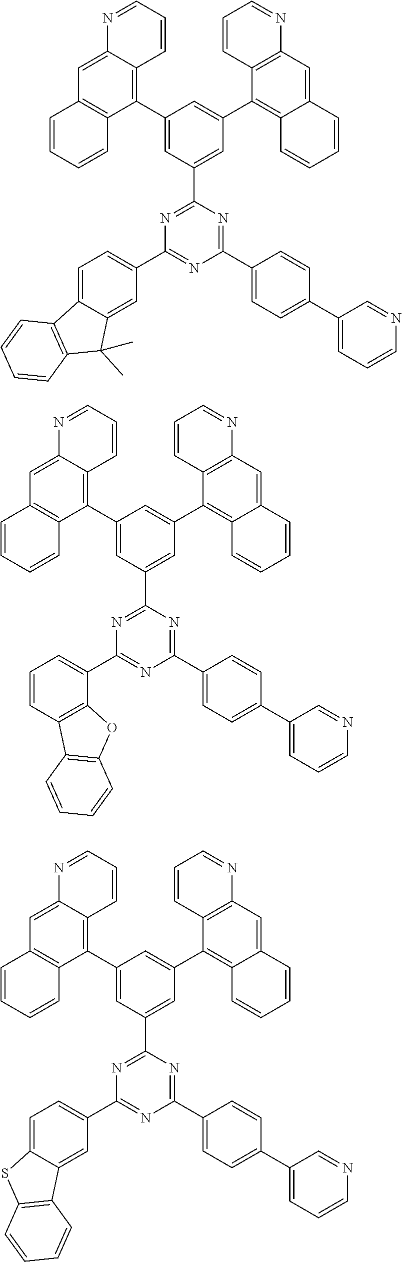

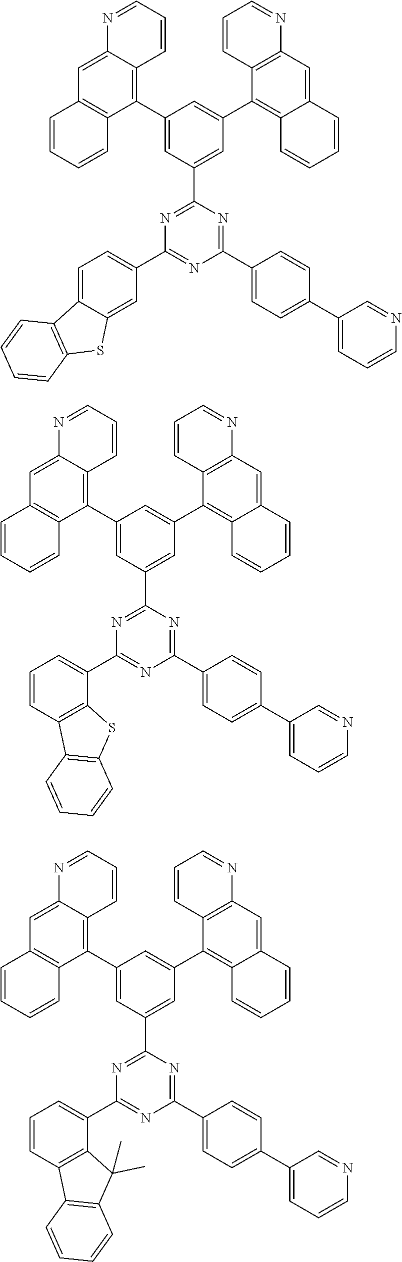

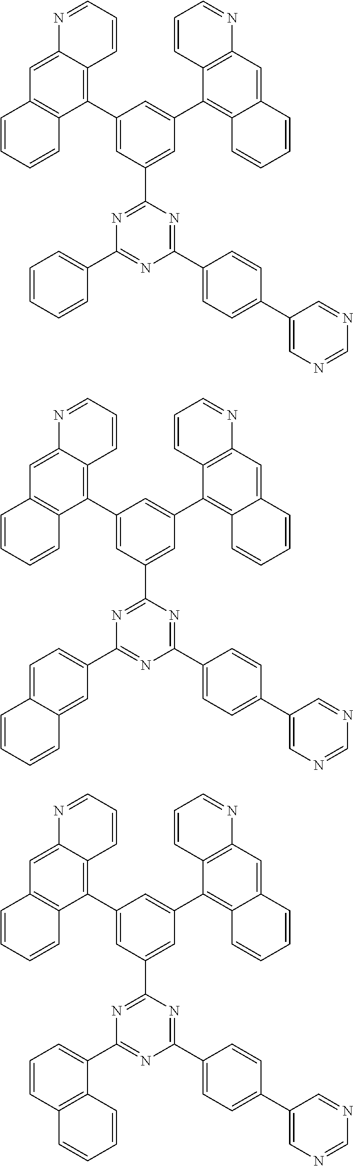 [SMILES: c1ccc(-c2nc(-c3ccc(-c4cncnc4)cc3)nc(-c3cc(-c4c5ccccc5cc5ncccc45)cc(-c4c5ccccc5cc5ncccc45)c3)n2)cc1 ; c1ccc2cc(-c3nc(-c4ccc(-c5cncnc5)cc4)nc(-c4cc(-c5c6ccccc6cc6ncccc56)cc(-c5c6ccccc6cc6ncccc56)c4)n3)ccc2c1 ; c1ccc2c(-c3nc(-c4ccc(-c5cncnc5)cc4)nc(-c4cc(-c5c6ccccc6cc6ncccc56)cc(-c5c6ccccc6cc6ncccc56)c4)n3)cccc2c1]

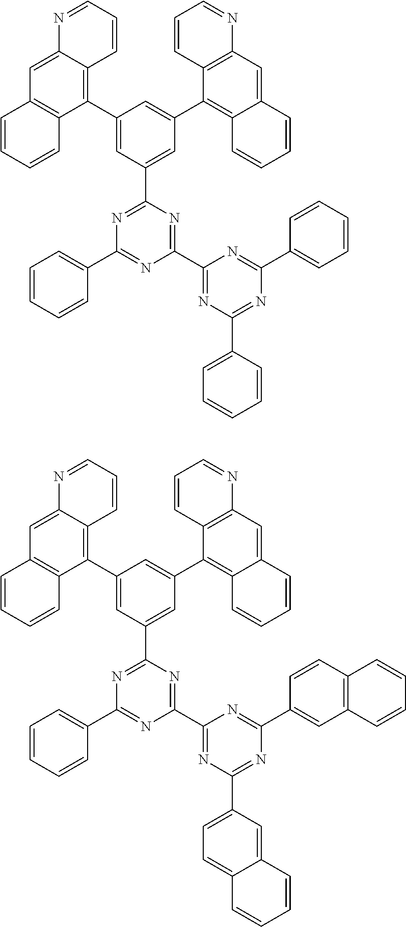 [SMILES: c1ccc(-c2nc(-c3ccccc3)nc(-c3nc(-c4ccccc4)nc(-c4cc(-c5c6ccccc6cc6ncccc56)cc(-c5c6ccccc6cc6ncccc56)c4)n3)n2)cc1 ; c1ccc(-c2nc(-c3cc(-c4c5ccccc5cc5ncccc45)cc(-c4c5ccccc5cc5ncccc45)c3)nc(-c3nc(-c4ccc5ccccc5c4)nc(-c4ccc5ccccc5c4)n3)n2)cc1]

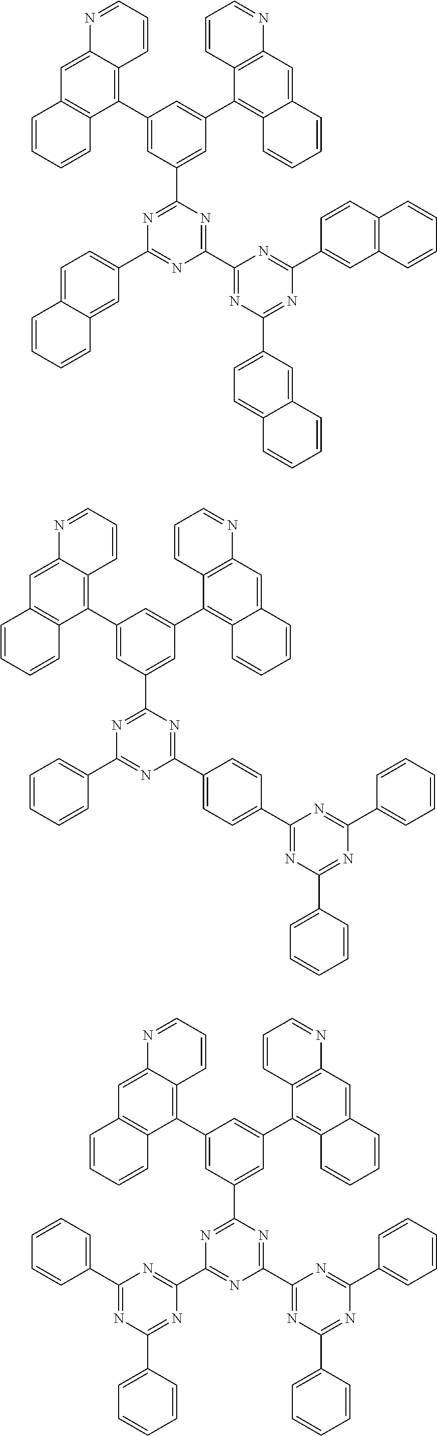 [SMILES: c1ccc2cc(-c3nc(-c4cc(-c5c6ccccc6cc6ncccc56)cc(-c5c6ccccc6cc6ncccc56)c4)nc(-c4nc(-c5ccc6ccccc6c5)nc(-c5ccc6ccccc6c5)n4)n3)ccc2c1 ; c1ccc(-c2nc(-c3ccccc3)nc(-c3ccc(-c4nc(-c5ccccc5)nc(-c5cc(-c6c7ccccc7cc7ncccc67)cc(-c6c7ccccc7cc7ncccc67)c5)n4)cc3)n2)cc1 ; c1ccc(-c2nc(-c3ccccc3)nc(-c3nc(-c4cc(-c5c6ccccc6cc6ncccc56)cc(-c5c6ccccc6cc6ncccc56)c4)nc(-c4nc(-c5ccccc5)nc(-c5ccccc5)n4)n3)n2)cc1]

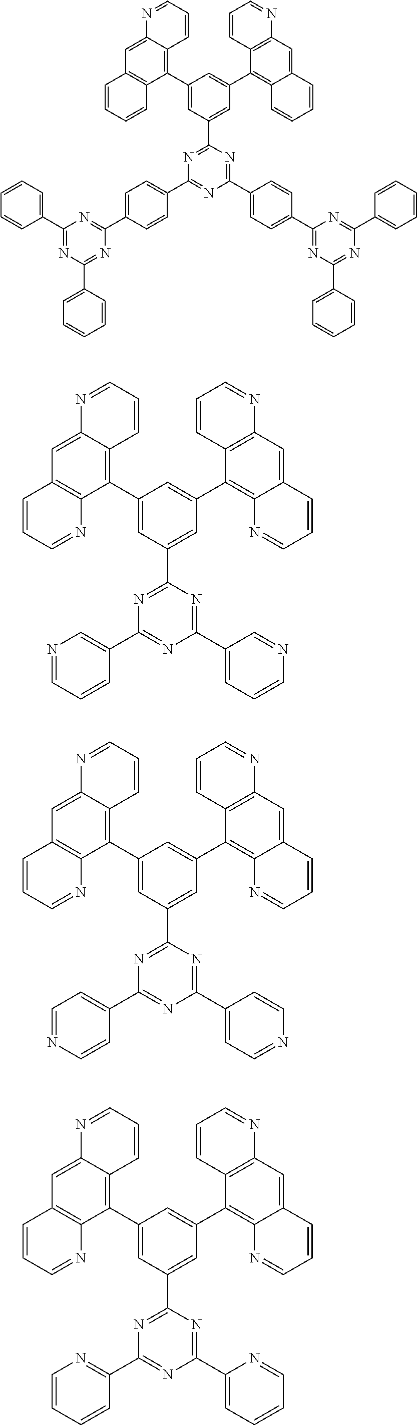 [SMILES: c1ccc(-c2nc(-c3ccccc3)nc(-c3ccc(-c4nc(-c5ccc(-c6nc(-c7ccccc7)nc(-c7ccccc7)n6)cc5)nc(-c5cc(-c6c7ccccc7cc7ncccc67)cc(-c6c7ccccc7cc7ncccc67)c5)n4)cc3)n2)cc1 ; c1cncc(-c2nc(-c3cccnc3)nc(-c3cc(-c4c5cccnc5cc5cccnc45)cc(-c4c5cccnc5cc5cccnc45)c3)n2)c1 ; c1cnc2c(-c3cc(-c4nc(-c5ccncc5)nc(-c5ccncc5)n4)cc(-c4c5cccnc5cc5cccnc45)c3)c3cccnc3cc2c1 ; c1ccc(-c2nc(-c3cc(-c4c5cccnc5cc5cccnc45)cc(-c4c5cccnc5cc5cccnc45)c3)nc(-c3ccccn3)n2)nc1]

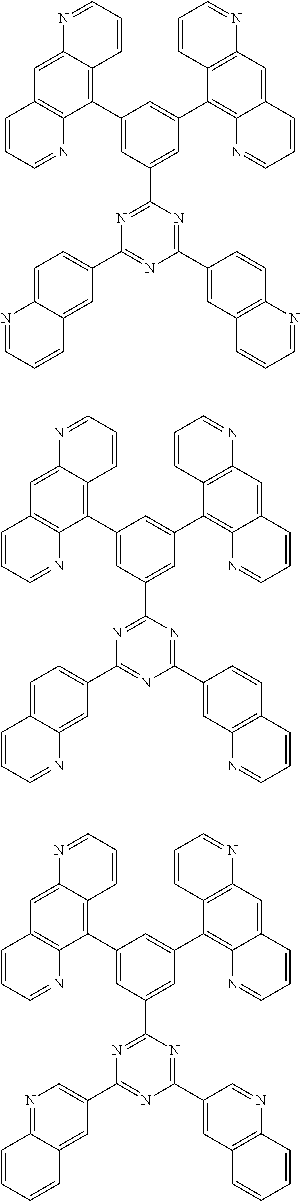 [SMILES: c1cnc2ccc(-c3nc(-c4cc(-c5c6cccnc6cc6cccnc56)cc(-c5c6cccnc6cc6cccnc56)c4)nc(-c4ccc5ncccc5c4)n3)cc2c1 ; c1cnc2cc(-c3nc(-c4cc(-c5c6cccnc6cc6cccnc56)cc(-c5c6cccnc6cc6cccnc56)c4)nc(-c4ccc5cccnc5c4)n3)ccc2c1 ; c1ccc2ncc(-c3nc(-c4cc(-c5c6cccnc6cc6cccnc56)cc(-c5c6cccnc6cc6cccnc56)c4)nc(-c4cnc5ccccc5c4)n3)cc2c1]

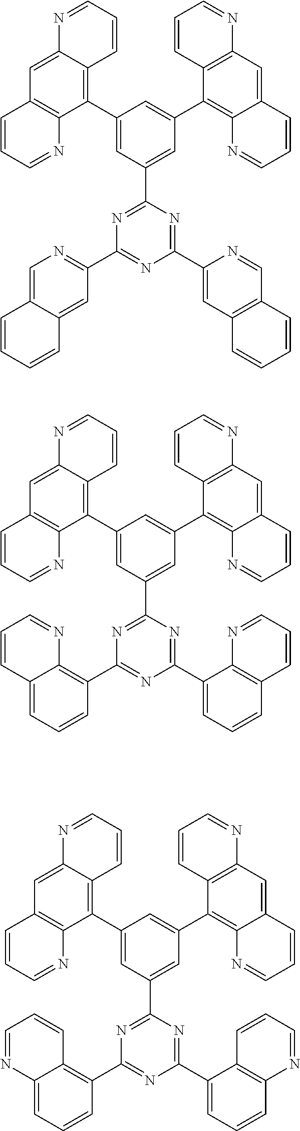 [SMILES: c1ccc2cc(-c3nc(-c4cc(-c5c6cccnc6cc6cccnc56)cc(-c5c6cccnc6cc6cccnc56)c4)nc(-c4cc5ccccc5cn4)n3)ncc2c1 ; c1cnc2c(-c3nc(-c4cc(-c5c6cccnc6cc6cccnc56)cc(-c5c6cccnc6cc6cccnc56)c4)nc(-c4cccc5cccnc45)n3)cccc2c1 ; c1cnc2c(-c3cc(-c4nc(-c5cccc6ncccc56)nc(-c5cccc6ncccc56)n4)cc(-c4c5cccnc5cc5cccnc45)c3)c3cccnc3cc2c1]

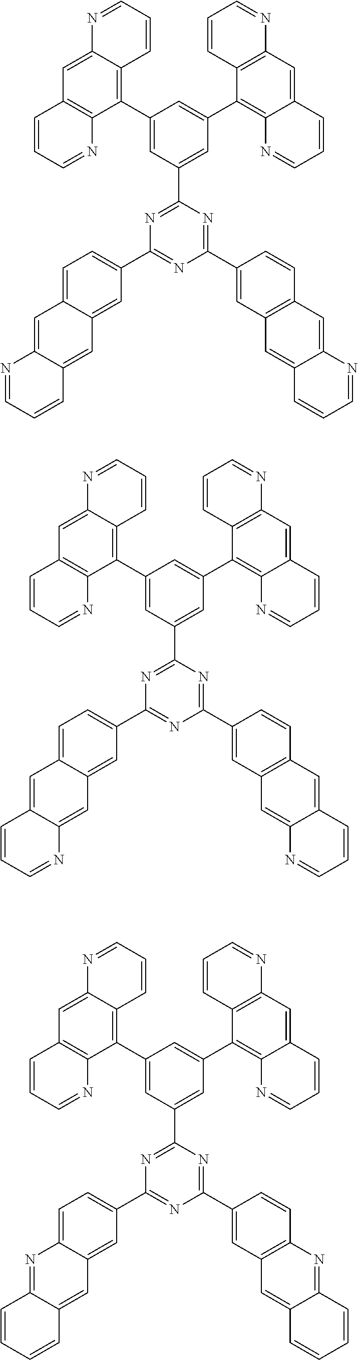 [SMILES: c1cnc2cc3ccc(-c4nc(-c5cc(-c6c7cccnc7cc7cccnc67)cc(-c6c7cccnc7cc7cccnc67)c5)nc(-c5ccc6cc7ncccc7cc6c5)n4)cc3cc2c1 ; c1cnc2cc3cc(-c4nc(-c5cc(-c6c7cccnc7cc7cccnc67)cc(-c6c7cccnc7cc7cccnc67)c5)nc(-c5ccc6cc7cccnc7cc6c5)n4)ccc3cc2c1 ; c1ccc2nc3ccc(-c4nc(-c5cc(-c6c7cccnc7cc7cccnc67)cc(-c6c7cccnc7cc7cccnc67)c5)nc(-c5ccc6nc7ccccc7cc6c5)n4)cc3cc2c1]

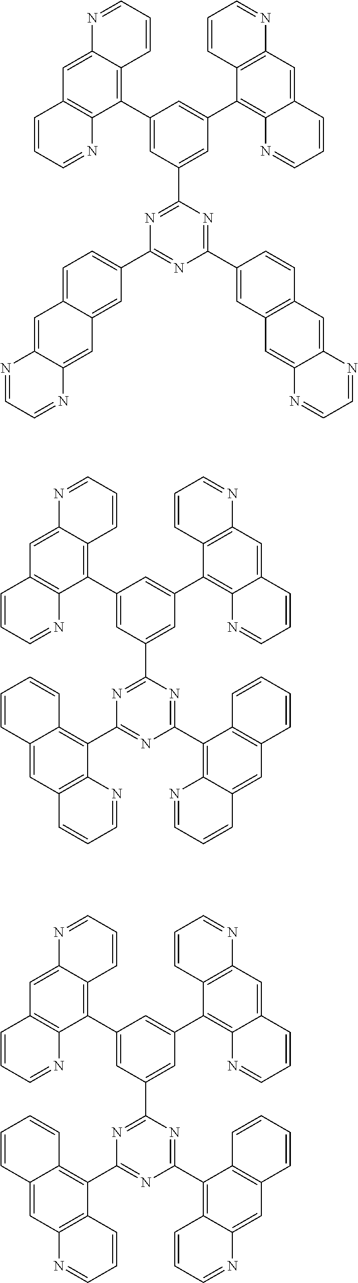 [SMILES: c1cnc2c(-c3cc(-c4nc(-c5ccc6cc7nccnc7cc6c5)nc(-c5ccc6cc7nccnc7cc6c5)n4)cc(-c4c5cccnc5cc5cccnc45)c3)c3cccnc3cc2c1 ; c1ccc2c(-c3nc(-c4cc(-c5c6cccnc6cc6cccnc56)cc(-c5c6cccnc6cc6cccnc56)c4)nc(-c4c5ccccc5cc5cccnc45)n3)c3ncccc3cc2c1 ; c1ccc2c(-c3nc(-c4cc(-c5c6cccnc6cc6cccnc56)cc(-c5c6cccnc6cc6cccnc56)c4)nc(-c4c5ccccc5cc5ncccc45)n3)c3cccnc3cc2c1]

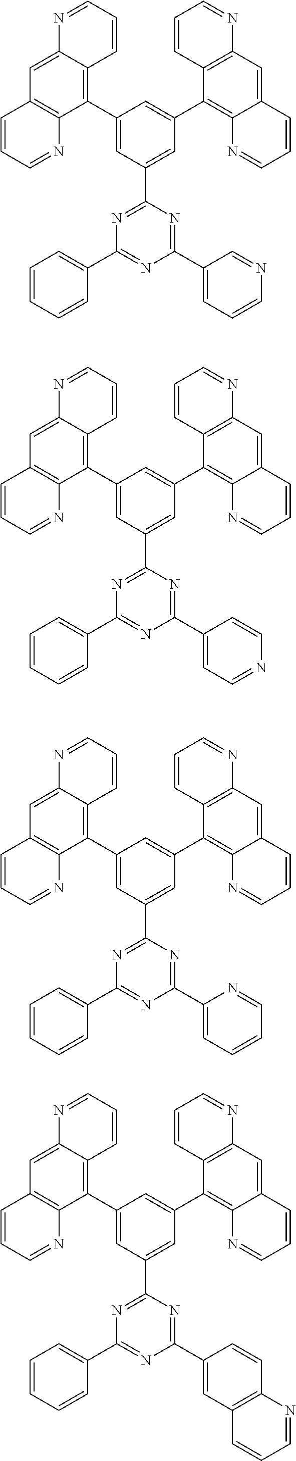 [SMILES: c1ccc(-c2nc(-c3cccnc3)nc(-c3cc(-c4c5cccnc5cc5cccnc45)cc(-c4c5cccnc5cc5cccnc45)c3)n2)cc1 ; c1ccc(-c2nc(-c3ccncc3)nc(-c3cc(-c4c5cccnc5cc5cccnc45)cc(-c4c5cccnc5cc5cccnc45)c3)n2)cc1 ; c1ccc(-c2nc(-c3cc(-c4c5cccnc5cc5cccnc45)cc(-c4c5cccnc5cc5cccnc45)c3)nc(-c3ccccn3)n2)cc1 ; c1ccc(-c2nc(-c3cc(-c4c5cccnc5cc5cccnc45)cc(-c4c5cccnc5cc5cccnc45)c3)nc(-c3ccc4ncccc4c3)n2)cc1]

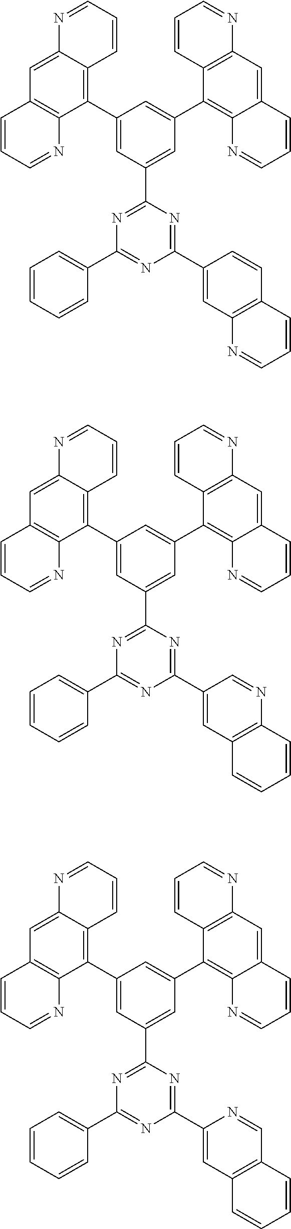 [SMILES: c1ccc(-c2nc(-c3cc(-c4c5cccnc5cc5cccnc45)cc(-c4c5cccnc5cc5cccnc45)c3)nc(-c3ccc4cccnc4c3)n2)cc1 ; c1ccc(-c2nc(-c3cc(-c4c5cccnc5cc5cccnc45)cc(-c4c5cccnc5cc5cccnc45)c3)nc(-c3cnc4ccccc4c3)n2)cc1 ; c1ccc(-c2nc(-c3cc(-c4c5cccnc5cc5cccnc45)cc(-c4c5cccnc5cc5cccnc45)c3)nc(-c3cc4ccccc4cn3)n2)cc1]

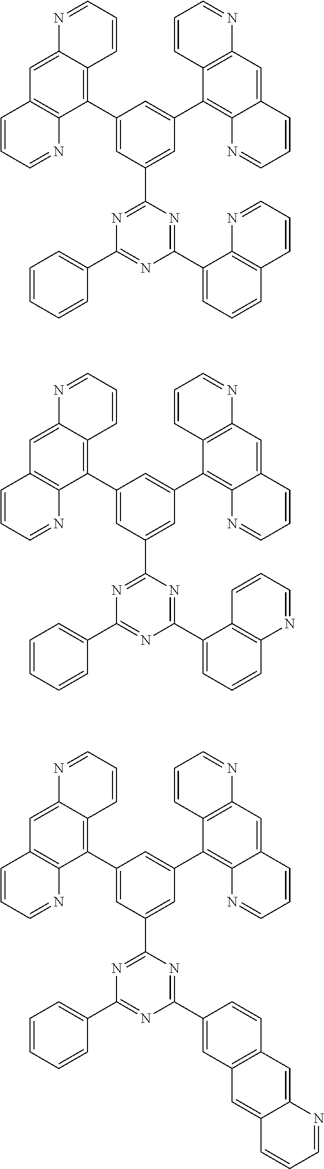 [SMILES: c1ccc(-c2nc(-c3cc(-c4c5cccnc5cc5cccnc45)cc(-c4c5cccnc5cc5cccnc45)c3)nc(-c3cccc4cccnc34)n2)cc1 ; c1ccc(-c2nc(-c3cc(-c4c5cccnc5cc5cccnc45)cc(-c4c5cccnc5cc5cccnc45)c3)nc(-c3cccc4ncccc34)n2)cc1 ; c1ccc(-c2nc(-c3cc(-c4c5cccnc5cc5cccnc45)cc(-c4c5cccnc5cc5cccnc45)c3)nc(-c3ccc4cc5ncccc5cc4c3)n2)cc1]

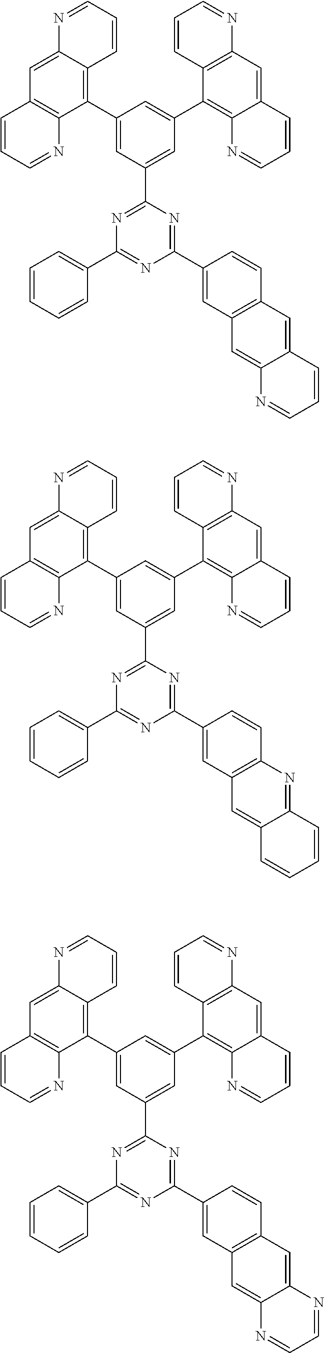 [SMILES: c1ccc(-c2nc(-c3cc(-c4c5cccnc5cc5cccnc45)cc(-c4c5cccnc5cc5cccnc45)c3)nc(-c3ccc4cc5cccnc5cc4c3)n2)cc1 ; c1ccc(-c2nc(-c3cc(-c4c5cccnc5cc5cccnc45)cc(-c4c5cccnc5cc5cccnc45)c3)nc(-c3ccc4nc5ccccc5cc4c3)n2)cc1 ; c1ccc(-c2nc(-c3cc(-c4c5cccnc5cc5cccnc45)cc(-c4c5cccnc5cc5cccnc45)c3)nc(-c3ccc4cc5nccnc5cc4c3)n2)cc1]

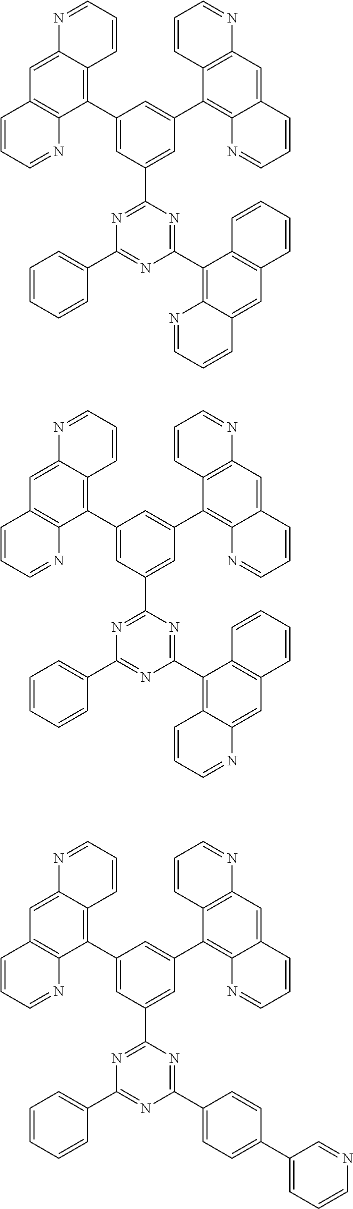

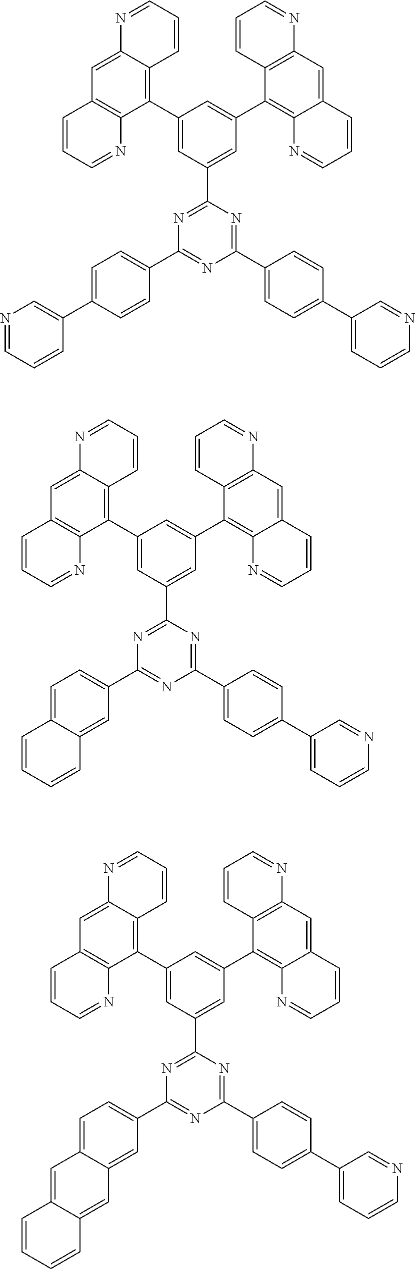 [SMILES: c1cncc(-c2ccc(-c3nc(-c4ccc(-c5cccnc5)cc4)nc(-c4cc(-c5c6cccnc6cc6cccnc56)cc(-c5c6cccnc6cc6cccnc56)c4)n3)cc2)c1 ; c1cncc(-c2ccc(-c3nc(-c4cc(-c5c6cccnc6cc6cccnc56)cc(-c5c6cccnc6cc6cccnc56)c4)nc(-c4ccc5ccccc5c4)n3)cc2)c1 ; c1cncc(-c2ccc(-c3nc(-c4cc(-c5c6cccnc6cc6cccnc56)cc(-c5c6cccnc6cc6cccnc56)c4)nc(-c4ccc5cc6ccccc6cc5c4)n3)cc2)c1]

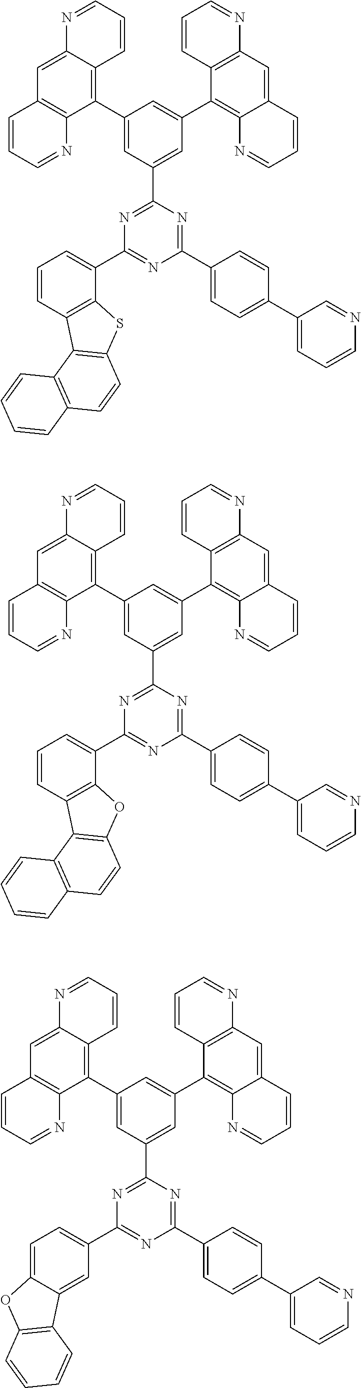 [SMILES: c1cncc(-c2ccc(-c3nc(-c4cc(-c5c6cccnc6cc6cccnc56)cc(-c5c6cccnc6cc6cccnc56)c4)nc(-c4cccc5c4sc4ccc6ccccc6c45)n3)cc2)c1 ; c1cncc(-c2ccc(-c3nc(-c4cc(-c5c6cccnc6cc6cccnc56)cc(-c5c6cccnc6cc6cccnc56)c4)nc(-c4cccc5c4oc4ccc6ccccc6c45)n3)cc2)c1 ; c1cncc(-c2ccc(-c3nc(-c4cc(-c5c6cccnc6cc6cccnc56)cc(-c5c6cccnc6cc6cccnc56)c4)nc(-c4ccc5oc6ccccc6c5c4)n3)cc2)c1]

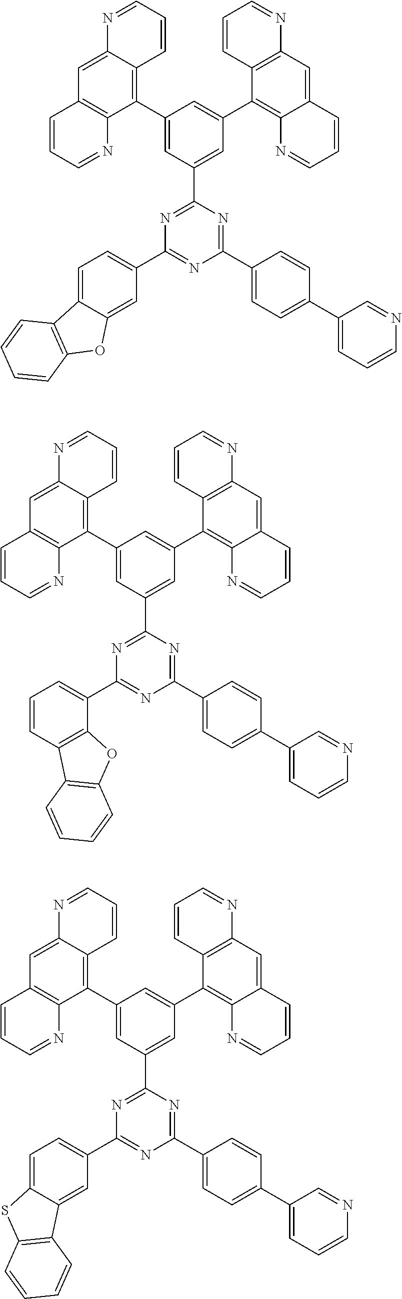 [SMILES: c1cncc(-c2ccc(-c3nc(-c4cc(-c5c6cccnc6cc6cccnc56)cc(-c5c6cccnc6cc6cccnc56)c4)nc(-c4ccc5c(c4)oc4ccccc45)n3)cc2)c1 ; c1cncc(-c2ccc(-c3nc(-c4cc(-c5c6cccnc6cc6cccnc56)cc(-c5c6cccnc6cc6cccnc56)c4)nc(-c4cccc5c4oc4ccccc45)n3)cc2)c1 ; c1cncc(-c2ccc(-c3nc(-c4cc(-c5c6cccnc6cc6cccnc56)cc(-c5c6cccnc6cc6cccnc56)c4)nc(-c4ccc5sc6ccccc6c5c4)n3)cc2)c1]

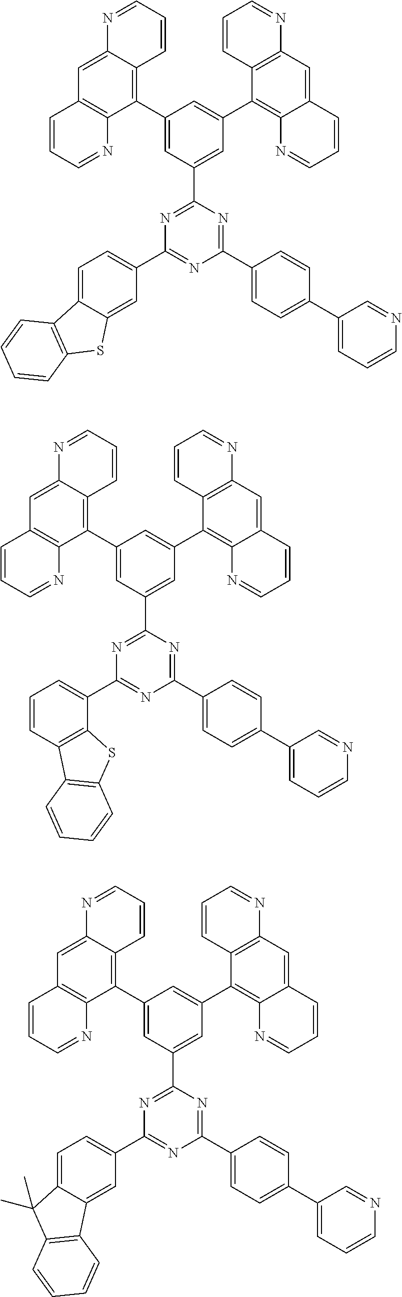 [SMILES: c1cncc(-c2ccc(-c3nc(-c4cc(-c5c6cccnc6cc6cccnc56)cc(-c5c6cccnc6cc6cccnc56)c4)nc(-c4ccc5c(c4)sc4ccccc45)n3)cc2)c1 ; c1cncc(-c2ccc(-c3nc(-c4cc(-c5c6cccnc6cc6cccnc56)cc(-c5c6cccnc6cc6cccnc56)c4)nc(-c4cccc5c4sc4ccccc45)n3)cc2)c1 ; CC1(C)c2ccccc2-c2cc(-c3nc(-c4ccc(-c5cccnc5)cc4)nc(-c4cc(-c5c6cccnc6cc6cccnc56)cc(-c5c6cccnc6cc6cccnc56)c4)n3)ccc21]

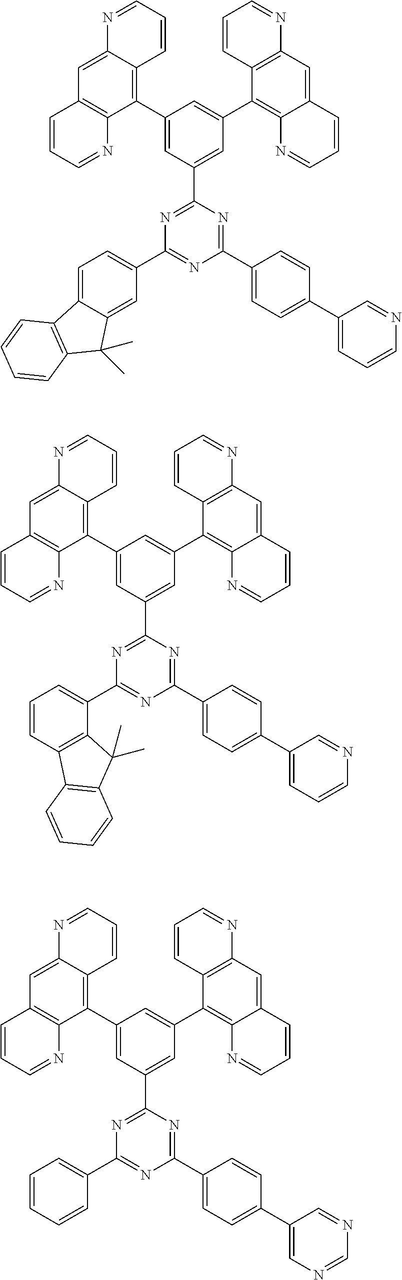 [SMILES: CC1(C)c2ccccc2-c2ccc(-c3nc(-c4ccc(-c5cccnc5)cc4)nc(-c4cc(-c5c6cccnc6cc6cccnc56)cc(-c5c6cccnc6cc6cccnc56)c4)n3)cc21 ; CC1(C)c2ccccc2-c2cccc(-c3nc(-c4ccc(-c5cccnc5)cc4)nc(-c4cc(-c5c6cccnc6cc6cccnc56)cc(-c5c6cccnc6cc6cccnc56)c4)n3)c21 ; c1ccc(-c2nc(-c3ccc(-c4cncnc4)cc3)nc(-c3cc(-c4c5cccnc5cc5cccnc45)cc(-c4c5cccnc5cc5cccnc45)c3)n2)cc1]

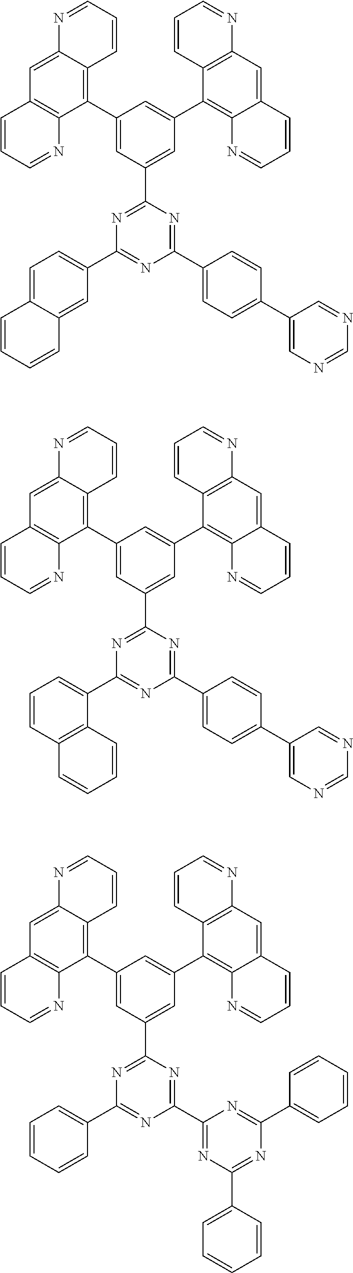 [SMILES: c1ccc2cc(-c3nc(-c4ccc(-c5cncnc5)cc4)nc(-c4cc(-c5c6cccnc6cc6cccnc56)cc(-c5c6cccnc6cc6cccnc56)c4)n3)ccc2c1 ; c1ccc2c(-c3nc(-c4ccc(-c5cncnc5)cc4)nc(-c4cc(-c5c6cccnc6cc6cccnc56)cc(-c5c6cccnc6cc6cccnc56)c4)n3)cccc2c1 ; c1ccc(-c2nc(-c3ccccc3)nc(-c3nc(-c4ccccc4)nc(-c4cc(-c5c6cccnc6cc6cccnc56)cc(-c5c6cccnc6cc6cccnc56)c4)n3)n2)cc1]

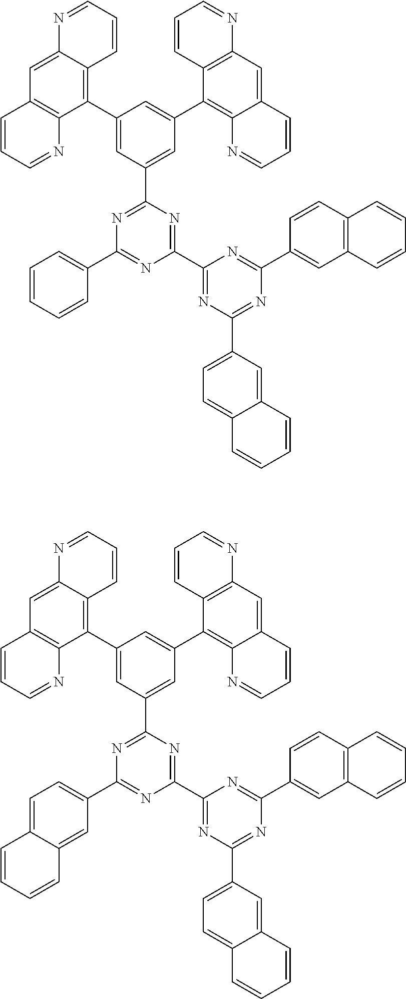 [SMILES: c1ccc(-c2nc(-c3cc(-c4c5cccnc5cc5cccnc45)cc(-c4c5cccnc5cc5cccnc45)c3)nc(-c3nc(-c4ccc5ccccc5c4)nc(-c4ccc5ccccc5c4)n3)n2)cc1 ; c1ccc2cc(-c3nc(-c4cc(-c5c6cccnc6cc6cccnc56)cc(-c5c6cccnc6cc6cccnc56)c4)nc(-c4nc(-c5ccc6ccccc6c5)nc(-c5ccc6ccccc6c5)n4)n3)ccc2c1]

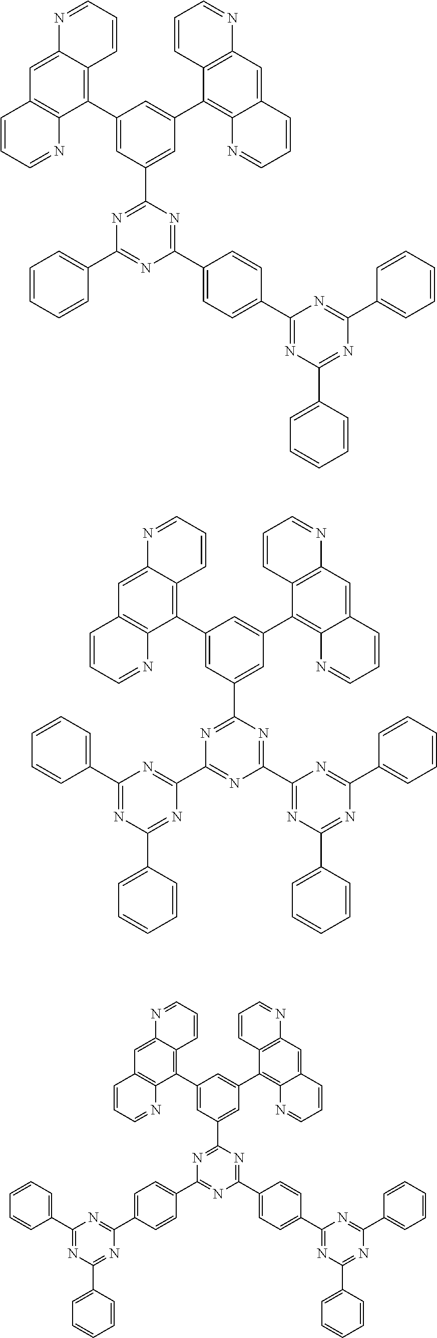 [SMILES: c1ccc(-c2nc(-c3ccccc3)nc(-c3ccc(-c4nc(-c5ccccc5)nc(-c5cc(-c6c7cccnc7cc7cccnc67)cc(-c6c7cccnc7cc7cccnc67)c5)n4)cc3)n2)cc1 ; c1ccc(-c2nc(-c3ccccc3)nc(-c3nc(-c4cc(-c5c6cccnc6cc6cccnc56)cc(-c5c6cccnc6cc6cccnc56)c4)nc(-c4nc(-c5ccccc5)nc(-c5ccccc5)n4)n3)n2)cc1 ; c1ccc(-c2nc(-c3ccccc3)nc(-c3ccc(-c4nc(-c5ccc(-c6nc(-c7ccccc7)nc(-c7ccccc7)n6)cc5)nc(-c5cc(-c6c7cccnc7cc7cccnc67)cc(-c6c7cccnc7cc7cccnc67)c5)n4)cc3)n2)cc1]

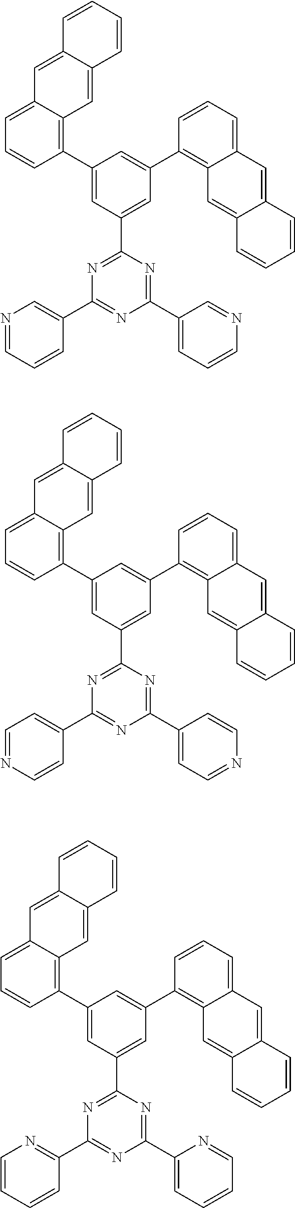 [SMILES: c1cncc(-c2nc(-c3cccnc3)nc(-c3cc(-c4cccc5cc6ccccc6cc45)cc(-c4cccc5cc6ccccc6cc45)c3)n2)c1 ; c1ccc2cc3c(-c4cc(-c5nc(-c6ccncc6)nc(-c6ccncc6)n5)cc(-c5cccc6cc7ccccc7cc56)c4)cccc3cc2c1 ; c1ccc(-c2nc(-c3cc(-c4cccc5cc6ccccc6cc45)cc(-c4cccc5cc6ccccc6cc45)c3)nc(-c3ccccn3)n2)nc1]

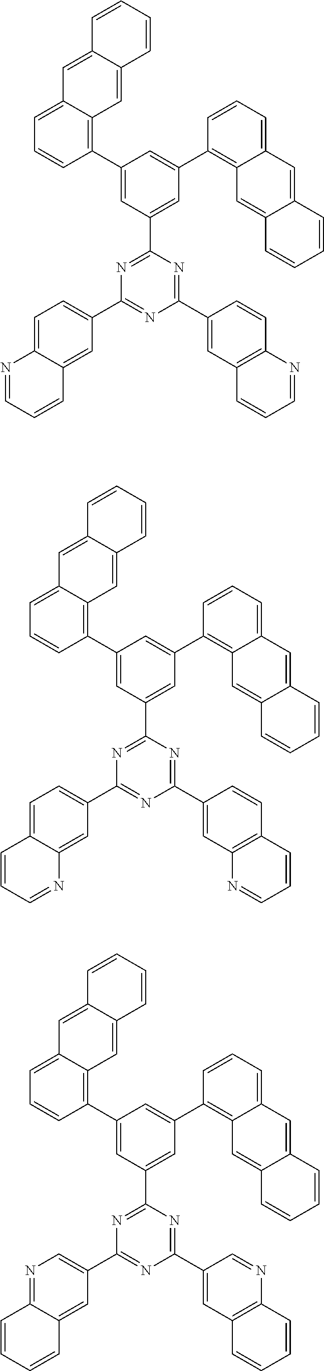 [SMILES: c1ccc2cc3c(-c4cc(-c5nc(-c6ccc7ncccc7c6)nc(-c6ccc7ncccc7c6)n5)cc(-c5cccc6cc7ccccc7cc56)c4)cccc3cc2c1 ; c1ccc2cc3c(-c4cc(-c5nc(-c6ccc7cccnc7c6)nc(-c6ccc7cccnc7c6)n5)cc(-c5cccc6cc7ccccc7cc56)c4)cccc3cc2c1 ; c1ccc2cc3c(-c4cc(-c5nc(-c6cnc7ccccc7c6)nc(-c6cnc7ccccc7c6)n5)cc(-c5cccc6cc7ccccc7cc56)c4)cccc3cc2c1]

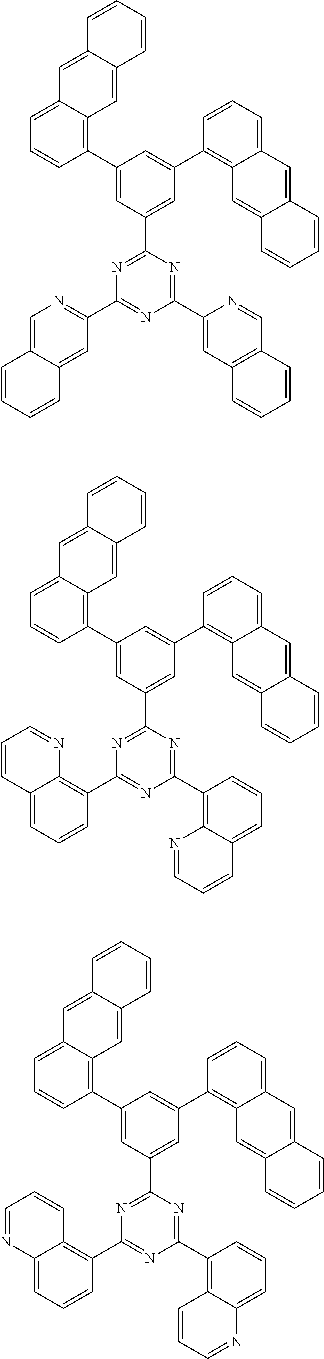 [SMILES: c1ccc2cc(-c3nc(-c4cc(-c5cccc6cc7ccccc7cc56)cc(-c5cccc6cc7ccccc7cc56)c4)nc(-c4cc5ccccc5cn4)n3)ncc2c1 ; c1ccc2cc3c(-c4cc(-c5nc(-c6cccc7cccnc67)nc(-c6cccc7cccnc67)n5)cc(-c5cccc6cc7ccccc7cc56)c4)cccc3cc2c1 ; c1ccc2cc3c(-c4cc(-c5nc(-c6cccc7ncccc67)nc(-c6cccc7ncccc67)n5)cc(-c5cccc6cc7ccccc7cc56)c4)cccc3cc2c1]

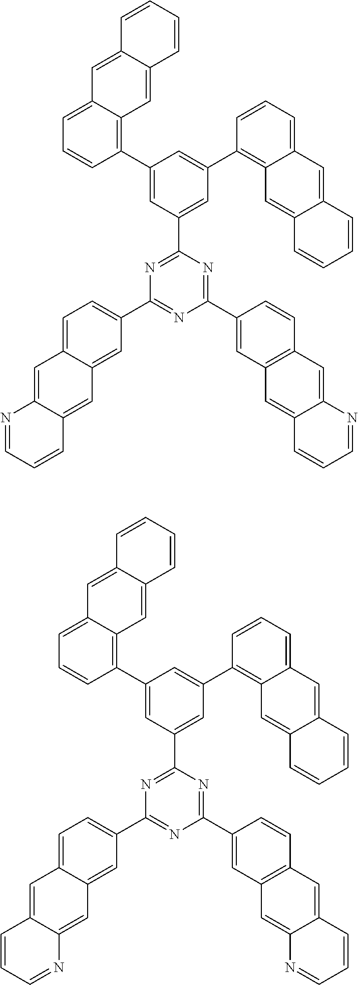 [SMILES: c1ccc2cc3c(-c4cc(-c5nc(-c6ccc7cc8ncccc8cc7c6)nc(-c6ccc7cc8ncccc8cc7c6)n5)cc(-c5cccc6cc7ccccc7cc56)c4)cccc3cc2c1 ; c1ccc2cc3c(-c4cc(-c5nc(-c6ccc7cc8cccnc8cc7c6)nc(-c6ccc7cc8cccnc8cc7c6)n5)cc(-c5cccc6cc7ccccc7cc56)c4)cccc3cc2c1]

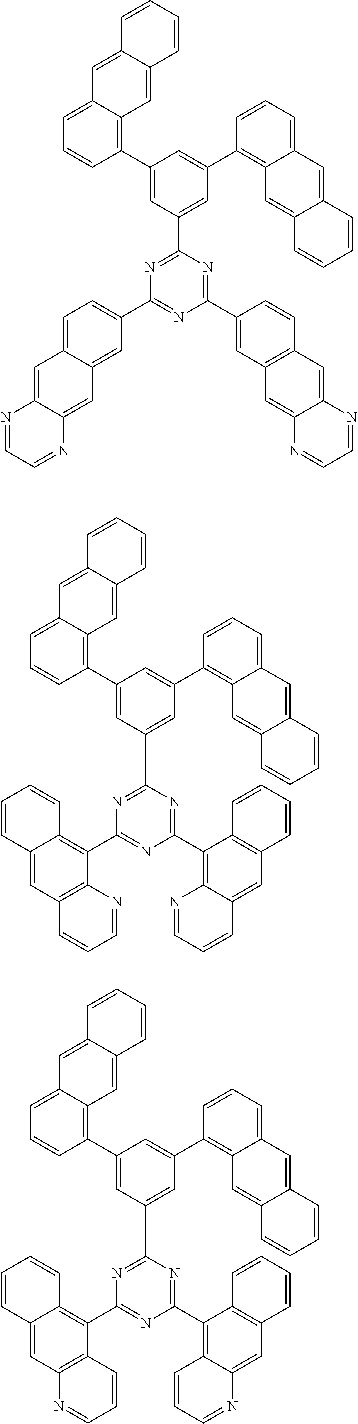 [SMILES: c1ccc2cc3c(-c4cc(-c5nc(-c6ccc7cc8nccnc8cc7c6)nc(-c6ccc7cc8nccnc8cc7c6)n5)cc(-c5cccc6cc7ccccc7cc56)c4)cccc3cc2c1 ; c1ccc2cc3c(-c4cc(-c5nc(-c6c7ccccc7cc7cccnc67)nc(-c6c7ccccc7cc7cccnc67)n5)cc(-c5cccc6cc7ccccc7cc56)c4)cccc3cc2c1 ; c1ccc2cc3c(-c4cc(-c5nc(-c6c7ccccc7cc7ncccc67)nc(-c6c7ccccc7cc7ncccc67)n5)cc(-c5cccc6cc7ccccc7cc56)c4)cccc3cc2c1]

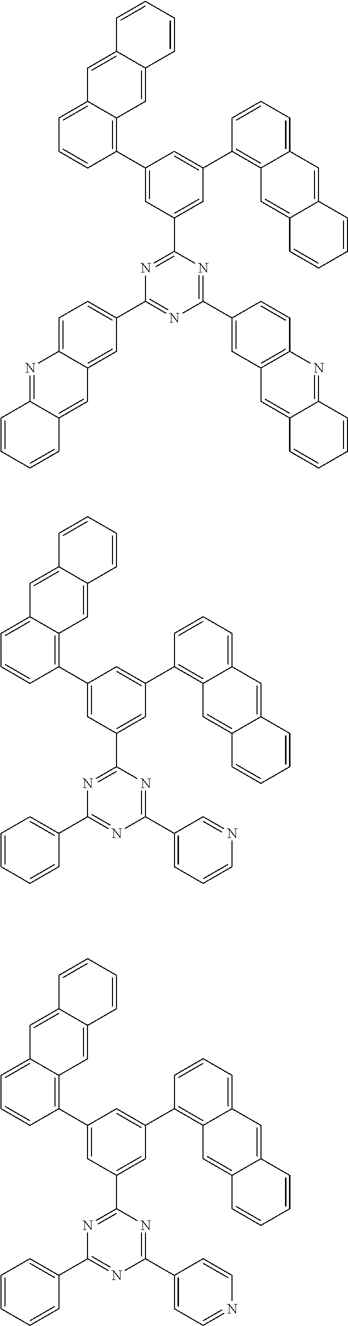 [SMILES: c1ccc2cc3c(-c4cc(-c5nc(-c6ccc7nc8ccccc8cc7c6)nc(-c6ccc7nc8ccccc8cc7c6)n5)cc(-c5cccc6cc7ccccc7cc56)c4)cccc3cc2c1 ; c1ccc(-c2nc(-c3cccnc3)nc(-c3cc(-c4cccc5cc6ccccc6cc45)cc(-c4cccc5cc6ccccc6cc45)c3)n2)cc1 ; c1ccc(-c2nc(-c3ccncc3)nc(-c3cc(-c4cccc5cc6ccccc6cc45)cc(-c4cccc5cc6ccccc6cc45)c3)n2)cc1]

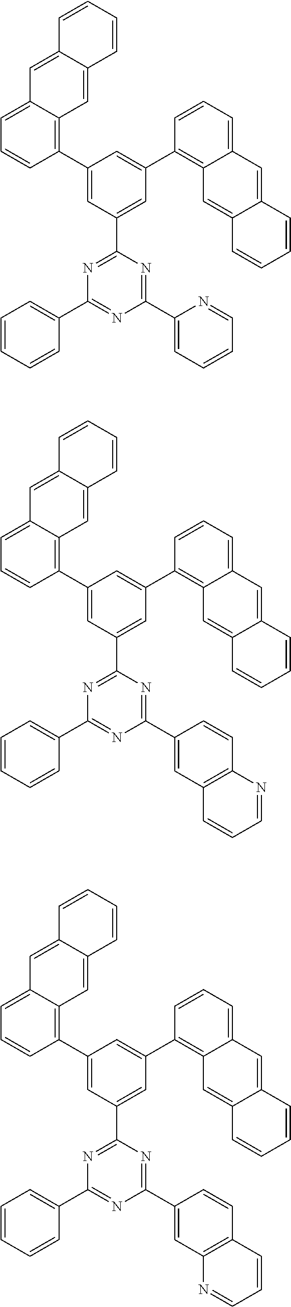 [SMILES: c1ccc(-c2nc(-c3cc(-c4cccc5cc6ccccc6cc45)cc(-c4cccc5cc6ccccc6cc45)c3)nc(-c3ccccn3)n2)cc1 ; c1ccc(-c2nc(-c3cc(-c4cccc5cc6ccccc6cc45)cc(-c4cccc5cc6ccccc6cc45)c3)nc(-c3ccc4ncccc4c3)n2)cc1 ; c1ccc(-c2nc(-c3cc(-c4cccc5cc6ccccc6cc45)cc(-c4cccc5cc6ccccc6cc45)c3)nc(-c3ccc4cccnc4c3)n2)cc1]

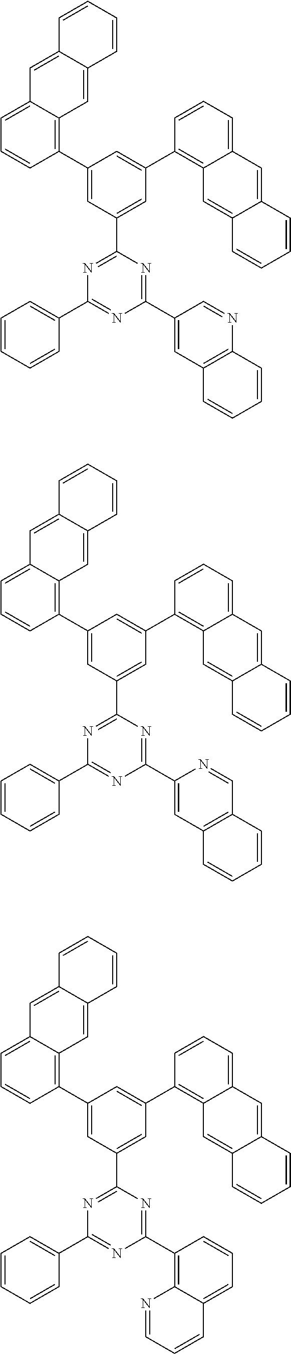 [SMILES: c1ccc(-c2nc(-c3cc(-c4cccc5cc6ccccc6cc45)cc(-c4cccc5cc6ccccc6cc45)c3)nc(-c3cnc4ccccc4c3)n2)cc1 ; c1ccc(-c2nc(-c3cc(-c4cccc5cc6ccccc6cc45)cc(-c4cccc5cc6ccccc6cc45)c3)nc(-c3cc4ccccc4cn3)n2)cc1 ; c1ccc(-c2nc(-c3cc(-c4cccc5cc6ccccc6cc45)cc(-c4cccc5cc6ccccc6cc45)c3)nc(-c3cccc4cccnc34)n2)cc1]

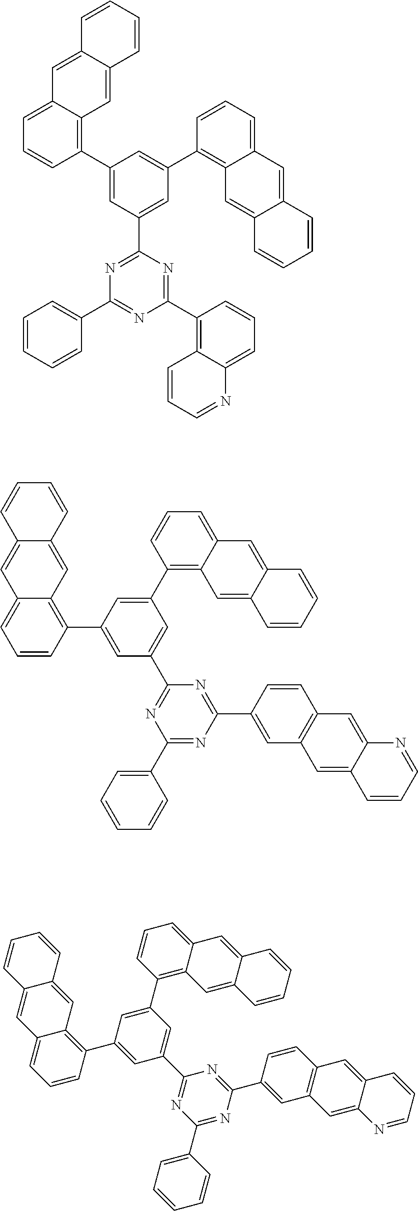 [SMILES: c1ccc(-c2nc(-c3cc(-c4cccc5cc6ccccc6cc45)cc(-c4cccc5cc6ccccc6cc45)c3)nc(-c3cccc4ncccc34)n2)cc1 ; c1ccc(-c2nc(-c3cc(-c4cccc5cc6ccccc6cc45)cc(-c4cccc5cc6ccccc6cc45)c3)nc(-c3ccc4cc5ncccc5cc4c3)n2)cc1 ; c1ccc(-c2nc(-c3cc(-c4cccc5cc6ccccc6cc45)cc(-c4cccc5cc6ccccc6cc45)c3)nc(-c3ccc4cc5cccnc5cc4c3)n2)cc1]

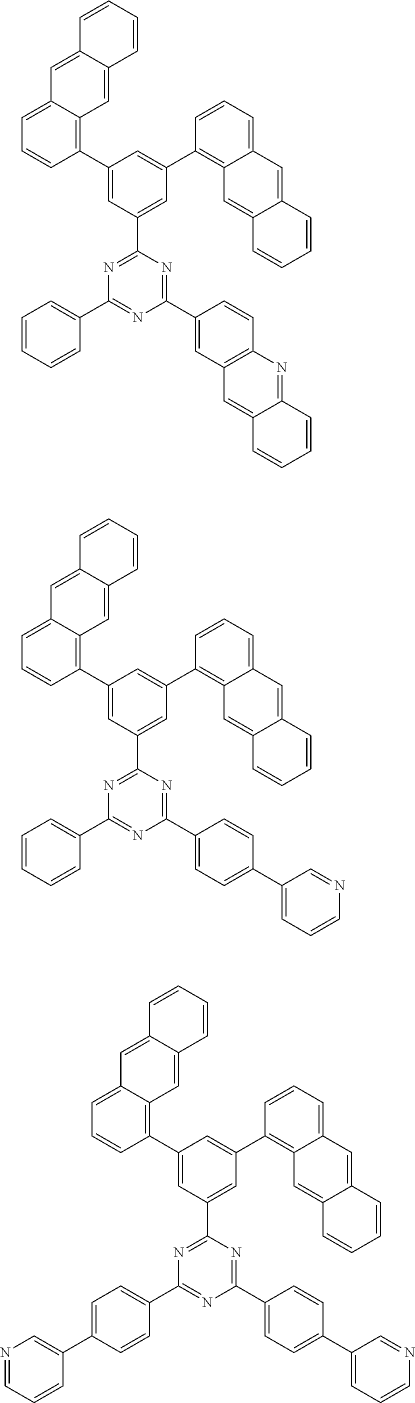 [SMILES: c1ccc(-c2nc(-c3cc(-c4cccc5cc6ccccc6cc45)cc(-c4cccc5cc6ccccc6cc45)c3)nc(-c3ccc4nc5ccccc5cc4c3)n2)cc1 ; c1ccc(-c2nc(-c3ccc(-c4cccnc4)cc3)nc(-c3cc(-c4cccc5cc6ccccc6cc45)cc(-c4cccc5cc6ccccc6cc45)c3)n2)cc1 ; c1cncc(-c2ccc(-c3nc(-c4ccc(-c5cccnc5)cc4)nc(-c4cc(-c5cccc6cc7ccccc7cc56)cc(-c5cccc6cc7ccccc7cc56)c4)n3)cc2)c1]

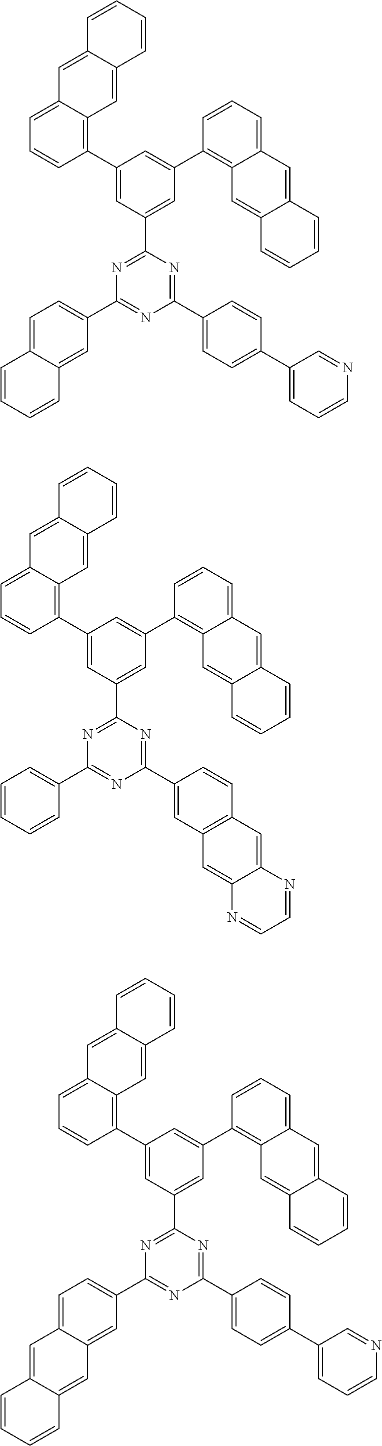 [SMILES: c1cncc(-c2ccc(-c3nc(-c4cc(-c5cccc6cc7ccccc7cc56)cc(-c5cccc6cc7ccccc7cc56)c4)nc(-c4ccc5ccccc5c4)n3)cc2)c1 ; c1ccc(-c2nc(-c3cc(-c4cccc5cc6ccccc6cc45)cc(-c4cccc5cc6ccccc6cc45)c3)nc(-c3ccc4cc5nccnc5cc4c3)n2)cc1 ; c1cncc(-c2ccc(-c3nc(-c4cc(-c5cccc6cc7ccccc7cc56)cc(-c5cccc6cc7ccccc7cc56)c4)nc(-c4ccc5cc6ccccc6cc5c4)n3)cc2)c1]

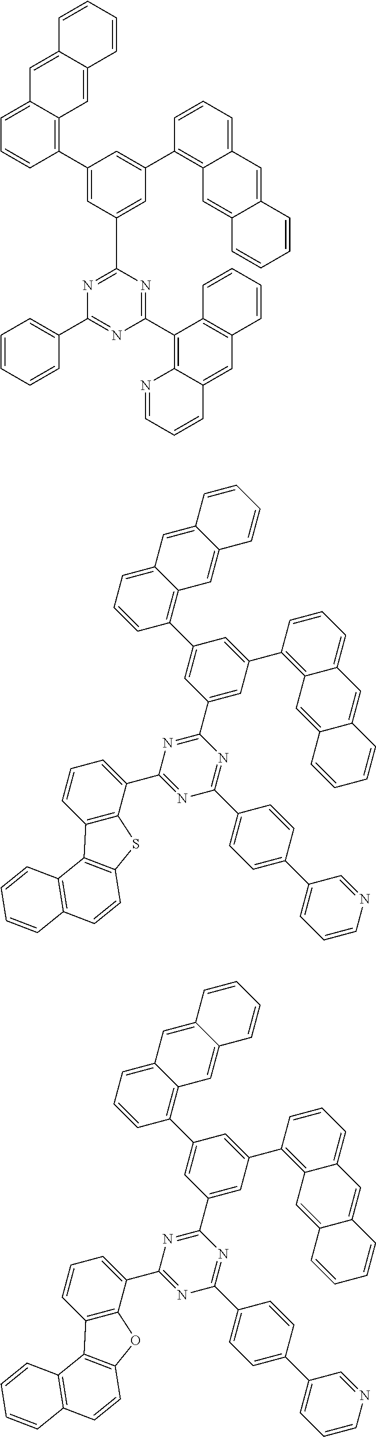 [SMILES: c1ccc(-c2nc(-c3cc(-c4cccc5cc6ccccc6cc45)cc(-c4cccc5cc6ccccc6cc45)c3)nc(-c3c4ccccc4cc4cccnc34)n2)cc1 ; c1cncc(-c2ccc(-c3nc(-c4cc(-c5cccc6cc7ccccc7cc56)cc(-c5cccc6cc7ccccc7cc56)c4)nc(-c4cccc5c4sc4ccc6ccccc6c45)n3)cc2)c1 ; c1cncc(-c2ccc(-c3nc(-c4cc(-c5cccc6cc7ccccc7cc56)cc(-c5cccc6cc7ccccc7cc56)c4)nc(-c4cccc5c4oc4ccc6ccccc6c45)n3)cc2)c1]

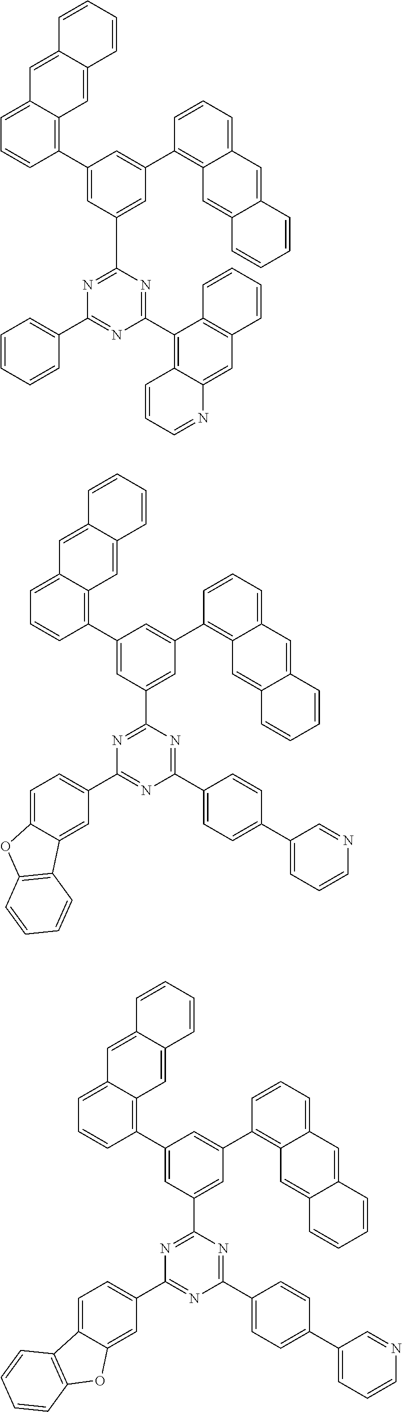 [SMILES: c1ccc(-c2nc(-c3cc(-c4cccc5cc6ccccc6cc45)cc(-c4cccc5cc6ccccc6cc45)c3)nc(-c3c4ccccc4cc4ncccc34)n2)cc1 ; c1cncc(-c2ccc(-c3nc(-c4cc(-c5cccc6cc7ccccc7cc56)cc(-c5cccc6cc7ccccc7cc56)c4)nc(-c4ccc5oc6ccccc6c5c4)n3)cc2)c1 ; c1cncc(-c2ccc(-c3nc(-c4cc(-c5cccc6cc7ccccc7cc56)cc(-c5cccc6cc7ccccc7cc56)c4)nc(-c4ccc5c(c4)oc4ccccc45)n3)cc2)c1]

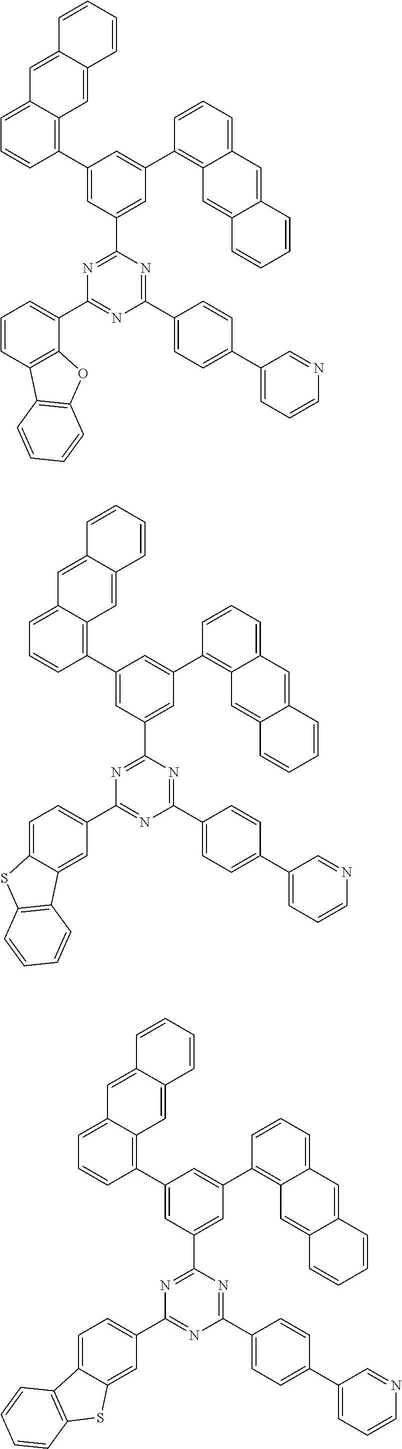 [SMILES: c1cncc(-c2ccc(-c3nc(-c4cc(-c5cccc6cc7ccccc7cc56)cc(-c5cccc6cc7ccccc7cc56)c4)nc(-c4cccc5c4oc4ccccc45)n3)cc2)c1 ; c1cncc(-c2ccc(-c3nc(-c4cc(-c5cccc6cc7ccccc7cc56)cc(-c5cccc6cc7ccccc7cc56)c4)nc(-c4ccc5sc6ccccc6c5c4)n3)cc2)c1 ; c1cncc(-c2ccc(-c3nc(-c4cc(-c5cccc6cc7ccccc7cc56)cc(-c5cccc6cc7ccccc7cc56)c4)nc(-c4ccc5c(c4)sc4ccccc45)n3)cc2)c1]

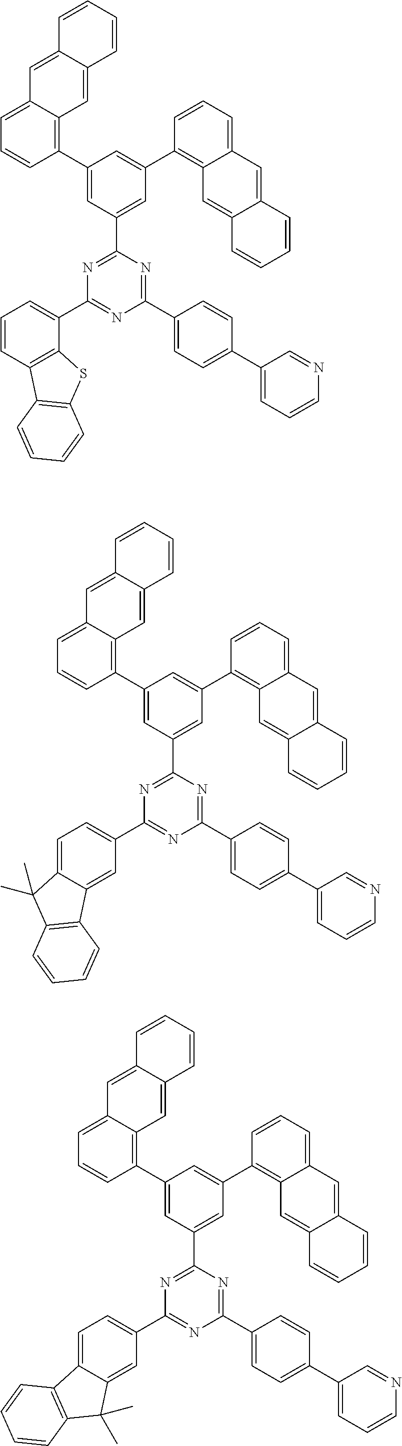 [SMILES: c1cncc(-c2ccc(-c3nc(-c4cc(-c5cccc6cc7ccccc7cc56)cc(-c5cccc6cc7ccccc7cc56)c4)nc(-c4cccc5c4sc4ccccc45)n3)cc2)c1 ; CC1(C)c2ccccc2-c2cc(-c3nc(-c4ccc(-c5cccnc5)cc4)nc(-c4cc(-c5cccc6cc7ccccc7cc56)cc(-c5cccc6cc7ccccc7cc56)c4)n3)ccc21 ; CC1(C)c2ccccc2-c2ccc(-c3nc(-c4ccc(-c5cccnc5)cc4)nc(-c4cc(-c5cccc6cc7ccccc7cc56)cc(-c5cccc6cc7ccccc7cc56)c4)n3)cc21]

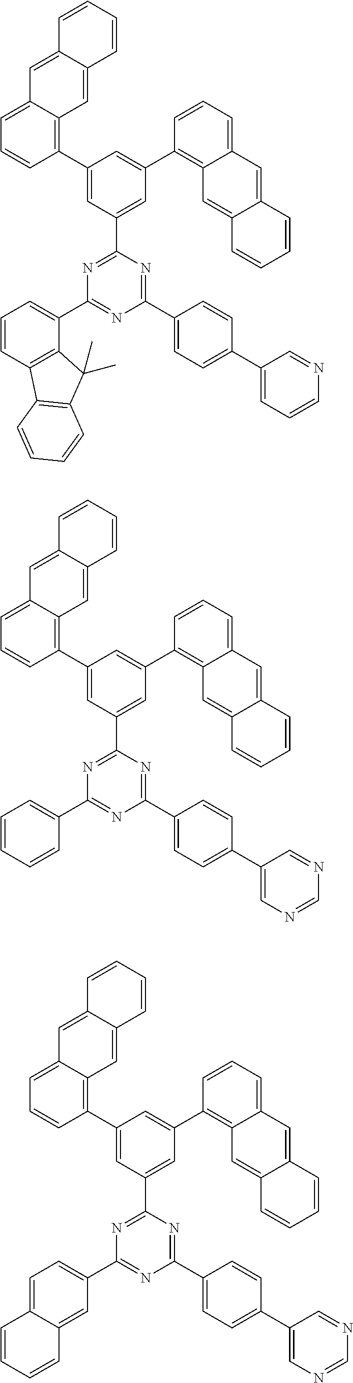

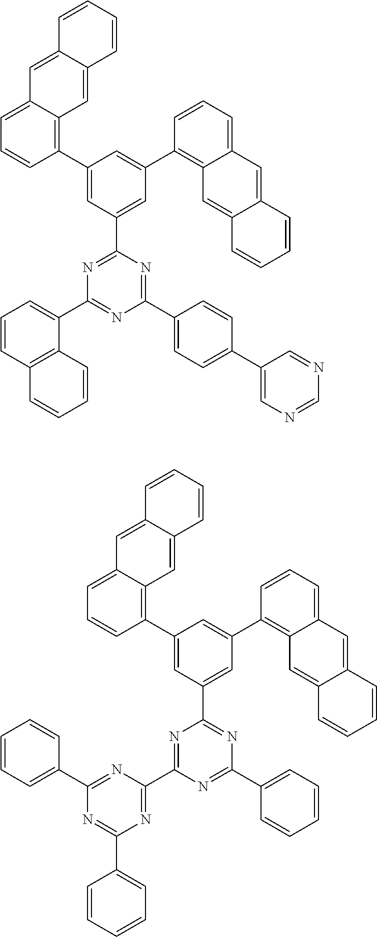 [SMILES: c1ccc2cc3c(-c4cc(-c5nc(-c6ccc(-c7cncnc7)cc6)nc(-c6cccc7ccccc67)n5)cc(-c5cccc6cc7ccccc7cc56)c4)cccc3cc2c1 ; c1ccc(-c2nc(-c3ccccc3)nc(-c3nc(-c4ccccc4)nc(-c4cc(-c5cccc6cc7ccccc7cc56)cc(-c5cccc6cc7ccccc7cc56)c4)n3)n2)cc1]

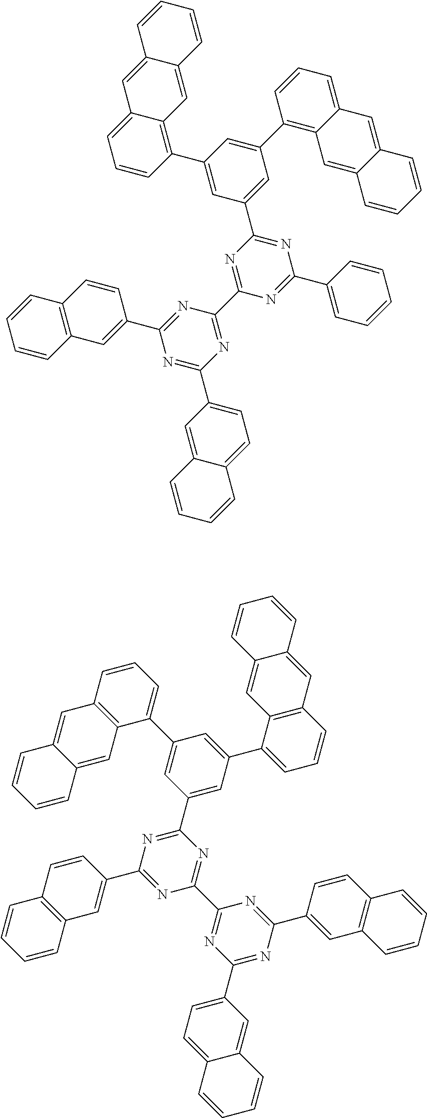 [SMILES: c1ccc(-c2nc(-c3cc(-c4cccc5cc6ccccc6cc45)cc(-c4cccc5cc6ccccc6cc45)c3)nc(-c3nc(-c4ccc5ccccc5c4)nc(-c4ccc5ccccc5c4)n3)n2)cc1 ; c1ccc2cc(-c3nc(-c4cc(-c5cccc6cc7ccccc7cc56)cc(-c5cccc6cc7ccccc7cc56)c4)nc(-c4nc(-c5ccc6ccccc6c5)nc(-c5ccc6ccccc6c5)n4)n3)ccc2c1]

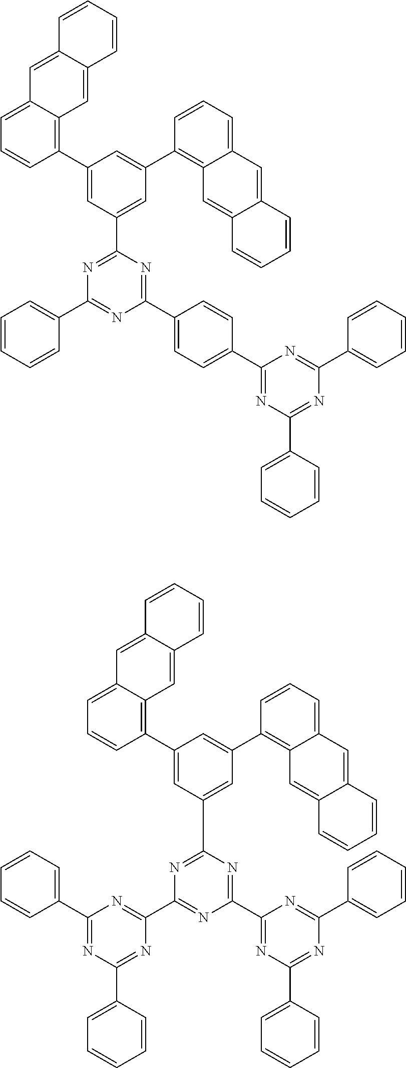 [SMILES: c1ccc(-c2nc(-c3ccccc3)nc(-c3ccc(-c4nc(-c5ccccc5)nc(-c5cc(-c6cccc7cc8ccccc8cc67)cc(-c6cccc7cc8ccccc8cc67)c5)n4)cc3)n2)cc1 ; c1ccc(-c2nc(-c3ccccc3)nc(-c3nc(-c4cc(-c5cccc6cc7ccccc7cc56)cc(-c5cccc6cc7ccccc7cc56)c4)nc(-c4nc(-c5ccccc5)nc(-c5ccccc5)n4)n3)n2)cc1]

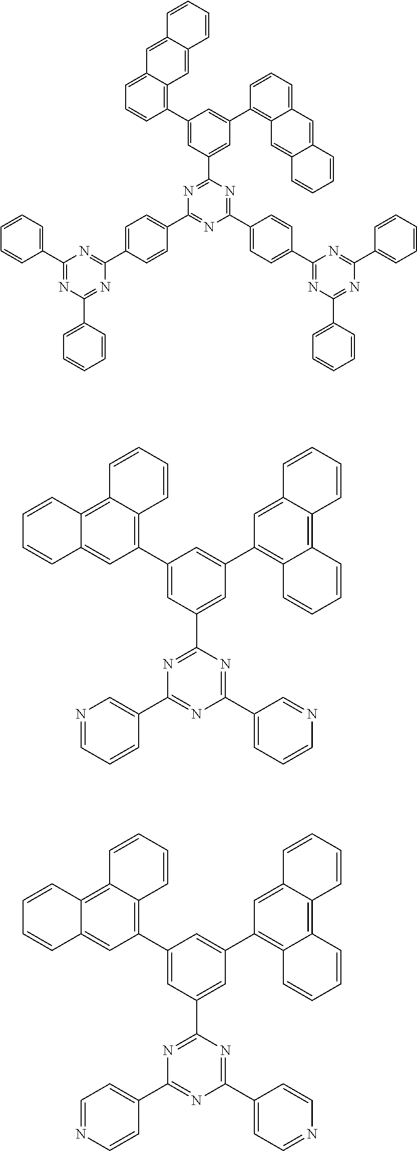 [SMILES: c1ccc(-c2nc(-c3ccccc3)nc(-c3ccc(-c4nc(-c5ccc(-c6nc(-c7ccccc7)nc(-c7ccccc7)n6)cc5)nc(-c5cc(-c6cccc7cc8ccccc8cc67)cc(-c6cccc7cc8ccccc8cc67)c5)n4)cc3)n2)cc1 ; c1cncc(-c2nc(-c3cccnc3)nc(-c3cc(-c4cc5ccccc5c5ccccc45)cc(-c4cc5ccccc5c5ccccc45)c3)n2)c1 ; c1ccc2c(c1)cc(-c1cc(-c3nc(-c4ccncc4)nc(-c4ccncc4)n3)cc(-c3cc4ccccc4c4ccccc34)c1)c1ccccc12]

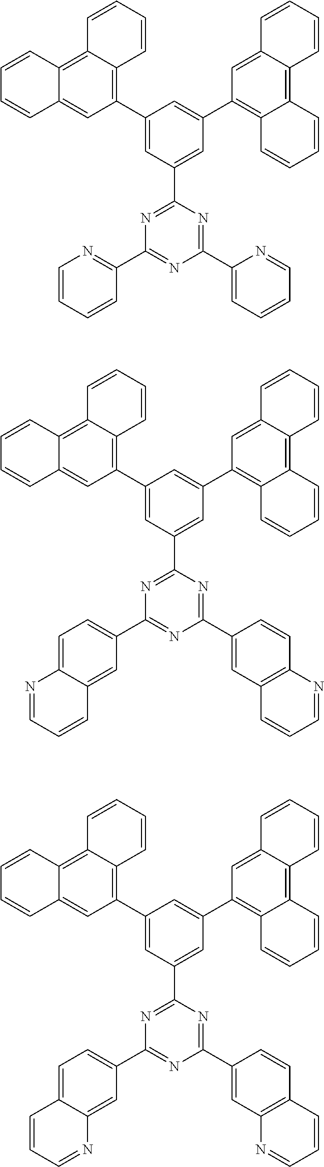 [SMILES: c1ccc(-c2nc(-c3cc(-c4cc5ccccc5c5ccccc45)cc(-c4cc5ccccc5c5ccccc45)c3)nc(-c3ccccn3)n2)nc1 ; c1cnc2ccc(-c3nc(-c4cc(-c5cc6ccccc6c6ccccc56)cc(-c5cc6ccccc6c6ccccc56)c4)nc(-c4ccc5ncccc5c4)n3)cc2c1 ; c1cnc2cc(-c3nc(-c4cc(-c5cc6ccccc6c6ccccc56)cc(-c5cc6ccccc6c6ccccc56)c4)nc(-c4ccc5cccnc5c4)n3)ccc2c1]

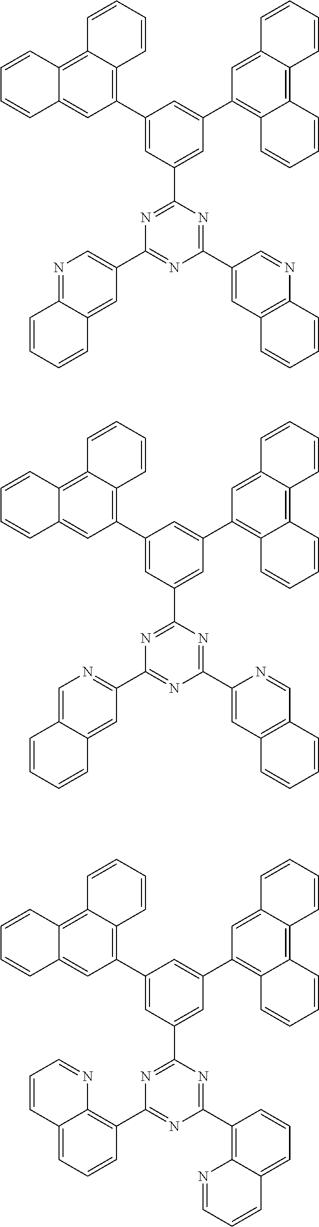 [SMILES: c1ccc2ncc(-c3nc(-c4cc(-c5cc6ccccc6c6ccccc56)cc(-c5cc6ccccc6c6ccccc56)c4)nc(-c4cnc5ccccc5c4)n3)cc2c1 ; c1ccc2cc(-c3nc(-c4cc(-c5cc6ccccc6c6ccccc56)cc(-c5cc6ccccc6c6ccccc56)c4)nc(-c4cc5ccccc5cn4)n3)ncc2c1 ; c1cnc2c(-c3nc(-c4cc(-c5cc6ccccc6c6ccccc56)cc(-c5cc6ccccc6c6ccccc56)c4)nc(-c4cccc5cccnc45)n3)cccc2c1]

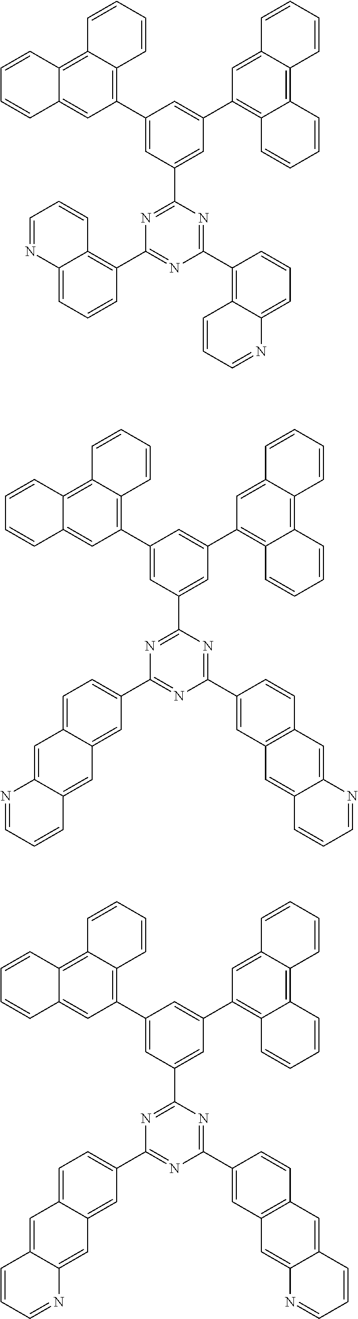 [SMILES: c1ccc2c(c1)cc(-c1cc(-c3nc(-c4cccc5ncccc45)nc(-c4cccc5ncccc45)n3)cc(-c3cc4ccccc4c4ccccc34)c1)c1ccccc12 ; c1cnc2cc3ccc(-c4nc(-c5cc(-c6cc7ccccc7c7ccccc67)cc(-c6cc7ccccc7c7ccccc67)c5)nc(-c5ccc6cc7ncccc7cc6c5)n4)cc3cc2c1 ; c1cnc2cc3cc(-c4nc(-c5cc(-c6cc7ccccc7c7ccccc67)cc(-c6cc7ccccc7c7ccccc67)c5)nc(-c5ccc6cc7cccnc7cc6c5)n4)ccc3cc2c1]

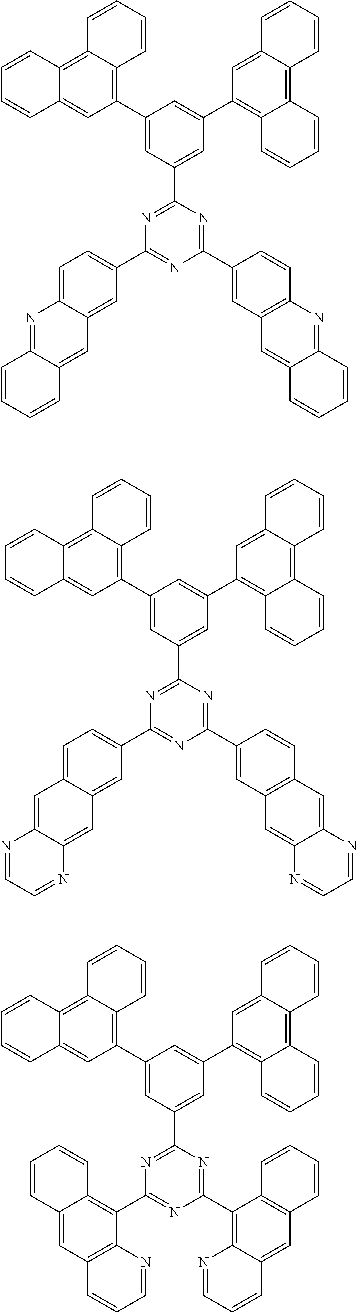 [SMILES: c1ccc2nc3ccc(-c4nc(-c5cc(-c6cc7ccccc7c7ccccc67)cc(-c6cc7ccccc7c7ccccc67)c5)nc(-c5ccc6nc7ccccc7cc6c5)n4)cc3cc2c1 ; c1ccc2c(c1)cc(-c1cc(-c3nc(-c4ccc5cc6nccnc6cc5c4)nc(-c4ccc5cc6nccnc6cc5c4)n3)cc(-c3cc4ccccc4c4ccccc34)c1)c1ccccc12 ; c1ccc2c(-c3nc(-c4cc(-c5cc6ccccc6c6ccccc56)cc(-c5cc6ccccc6c6ccccc56)c4)nc(-c4c5ccccc5cc5cccnc45)n3)c3ncccc3cc2c1]

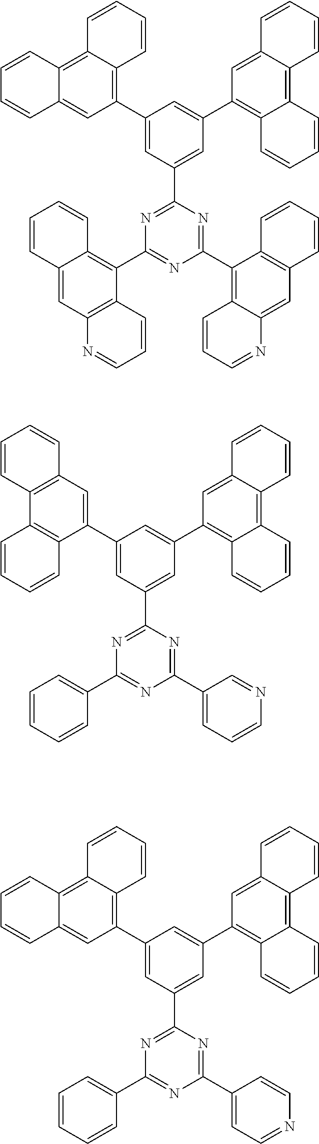 [SMILES: c1ccc2c(-c3nc(-c4cc(-c5cc6ccccc6c6ccccc56)cc(-c5cc6ccccc6c6ccccc56)c4)nc(-c4c5ccccc5cc5ncccc45)n3)c3cccnc3cc2c1 ; c1ccc(-c2nc(-c3cccnc3)nc(-c3cc(-c4cc5ccccc5c5ccccc45)cc(-c4cc5ccccc5c5ccccc45)c3)n2)cc1 ; c1ccc(-c2nc(-c3ccncc3)nc(-c3cc(-c4cc5ccccc5c5ccccc45)cc(-c4cc5ccccc5c5ccccc45)c3)n2)cc1]

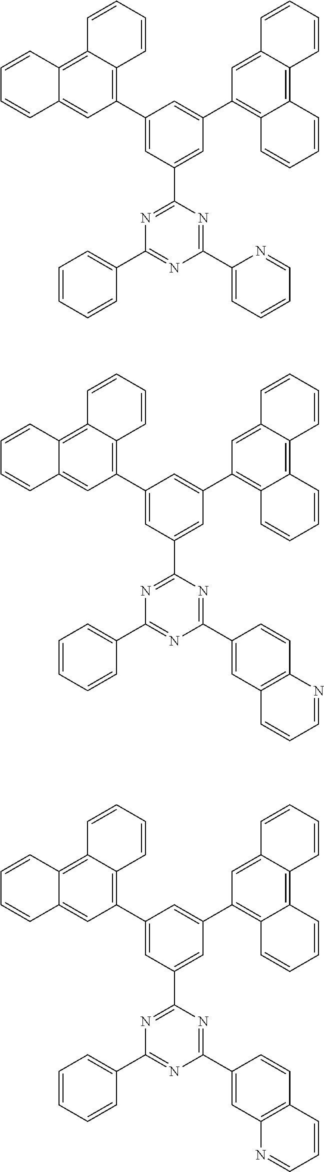 [SMILES: c1ccc(-c2nc(-c3cc(-c4cc5ccccc5c5ccccc45)cc(-c4cc5ccccc5c5ccccc45)c3)nc(-c3ccccn3)n2)cc1 ; c1ccc(-c2nc(-c3cc(-c4cc5ccccc5c5ccccc45)cc(-c4cc5ccccc5c5ccccc45)c3)nc(-c3ccc4ncccc4c3)n2)cc1 ; c1ccc(-c2nc(-c3cc(-c4cc5ccccc5c5ccccc45)cc(-c4cc5ccccc5c5ccccc45)c3)nc(-c3ccc4cccnc4c3)n2)cc1]

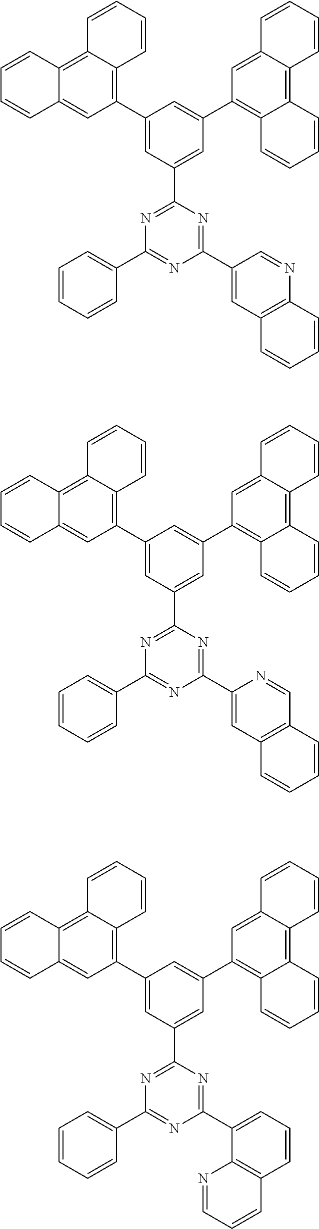 [SMILES: c1ccc(-c2nc(-c3cc(-c4cc5ccccc5c5ccccc45)cc(-c4cc5ccccc5c5ccccc45)c3)nc(-c3cnc4ccccc4c3)n2)cc1 ; c1ccc(-c2nc(-c3cc(-c4cc5ccccc5c5ccccc45)cc(-c4cc5ccccc5c5ccccc45)c3)nc(-c3cc4ccccc4cn3)n2)cc1 ; c1ccc(-c2nc(-c3cc(-c4cc5ccccc5c5ccccc45)cc(-c4cc5ccccc5c5ccccc45)c3)nc(-c3cccc4cccnc34)n2)cc1]

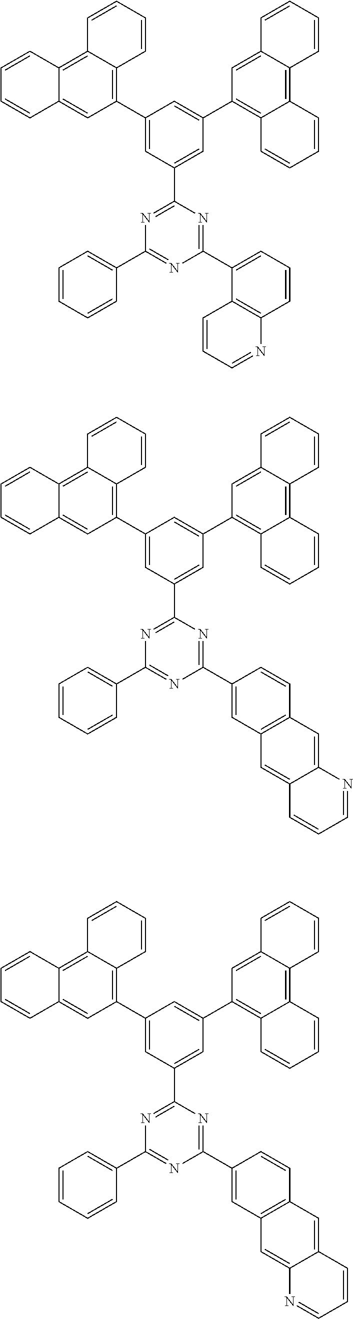 [SMILES: c1ccc(-c2nc(-c3cc(-c4cc5ccccc5c5ccccc45)cc(-c4cc5ccccc5c5ccccc45)c3)nc(-c3cccc4ncccc34)n2)cc1 ; c1ccc(-c2nc(-c3cc(-c4cc5ccccc5c5ccccc45)cc(-c4cc5ccccc5c5ccccc45)c3)nc(-c3ccc4cc5ncccc5cc4c3)n2)cc1 ; c1ccc(-c2nc(-c3cc(-c4cc5ccccc5c5ccccc45)cc(-c4cc5ccccc5c5ccccc45)c3)nc(-c3ccc4cc5cccnc5cc4c3)n2)cc1]

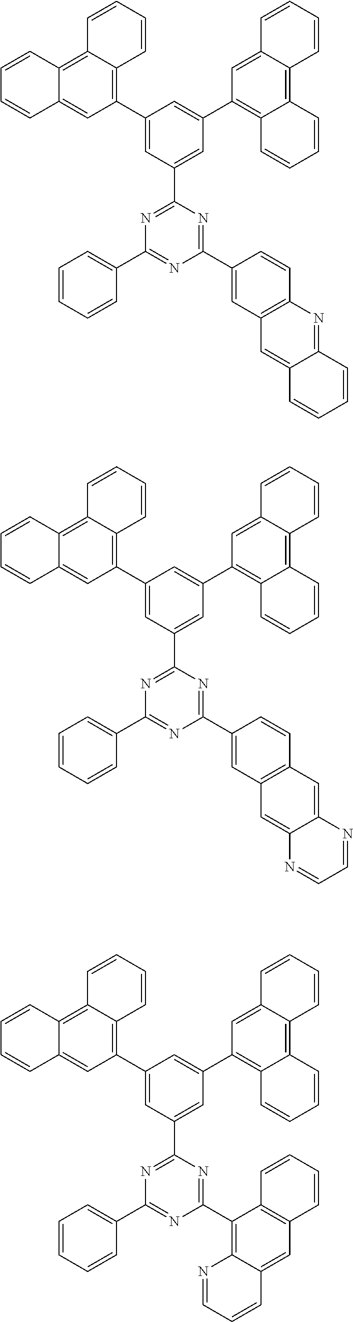 [SMILES: c1ccc(-c2nc(-c3cc(-c4cc5ccccc5c5ccccc45)cc(-c4cc5ccccc5c5ccccc45)c3)nc(-c3ccc4nc5ccccc5cc4c3)n2)cc1 ; c1ccc(-c2nc(-c3cc(-c4cc5ccccc5c5ccccc45)cc(-c4cc5ccccc5c5ccccc45)c3)nc(-c3ccc4cc5nccnc5cc4c3)n2)cc1 ; c1ccc(-c2nc(-c3cc(-c4cc5ccccc5c5ccccc45)cc(-c4cc5ccccc5c5ccccc45)c3)nc(-c3c4ccccc4cc4cccnc34)n2)cc1]

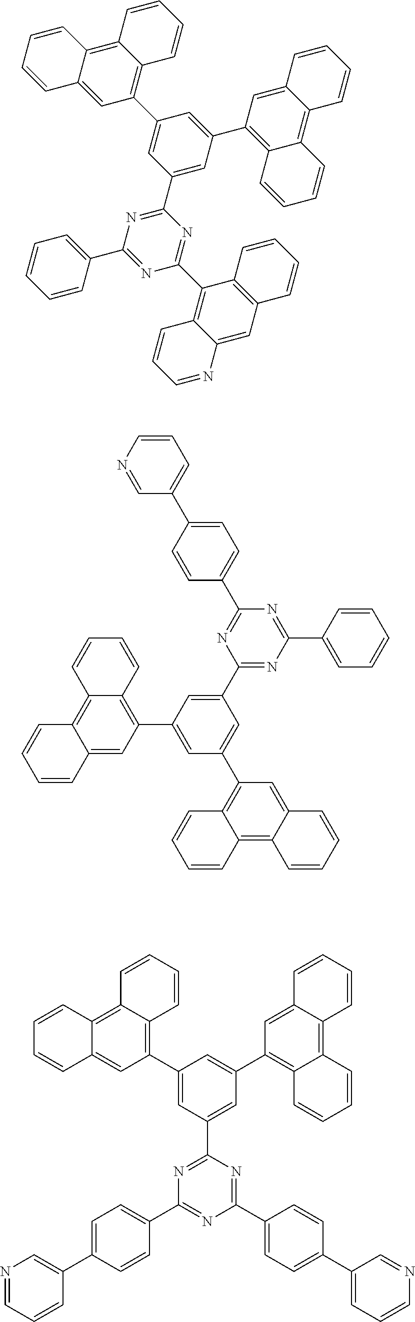 [SMILES: c1ccc(-c2nc(-c3cc(-c4cc5ccccc5c5ccccc45)cc(-c4cc5ccccc5c5ccccc45)c3)nc(-c3c4ccccc4cc4ncccc34)n2)cc1 ; c1ccc(-c2nc(-c3ccc(-c4cccnc4)cc3)nc(-c3cc(-c4cc5ccccc5c5ccccc45)cc(-c4cc5ccccc5c5ccccc45)c3)n2)cc1 ; c1cncc(-c2ccc(-c3nc(-c4ccc(-c5cccnc5)cc4)nc(-c4cc(-c5cc6ccccc6c6ccccc56)cc(-c5cc6ccccc6c6ccccc56)c4)n3)cc2)c1]

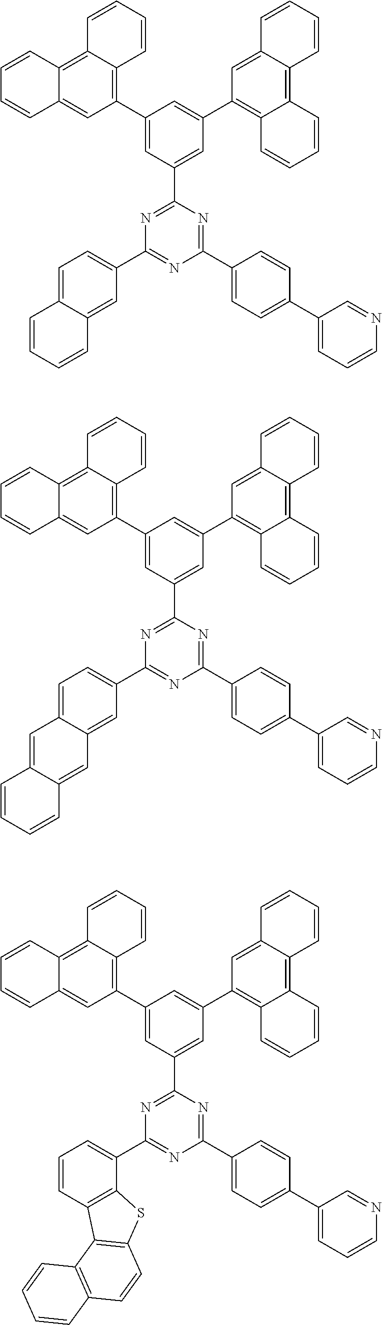 [SMILES: c1cncc(-c2ccc(-c3nc(-c4cc(-c5cc6ccccc6c6ccccc56)cc(-c5cc6ccccc6c6ccccc56)c4)nc(-c4ccc5ccccc5c4)n3)cc2)c1 ; c1cncc(-c2ccc(-c3nc(-c4cc(-c5cc6ccccc6c6ccccc56)cc(-c5cc6ccccc6c6ccccc56)c4)nc(-c4ccc5cc6ccccc6cc5c4)n3)cc2)c1 ; c1cncc(-c2ccc(-c3nc(-c4cc(-c5cc6ccccc6c6ccccc56)cc(-c5cc6ccccc6c6ccccc56)c4)nc(-c4cccc5c4sc4ccc6ccccc6c45)n3)cc2)c1]

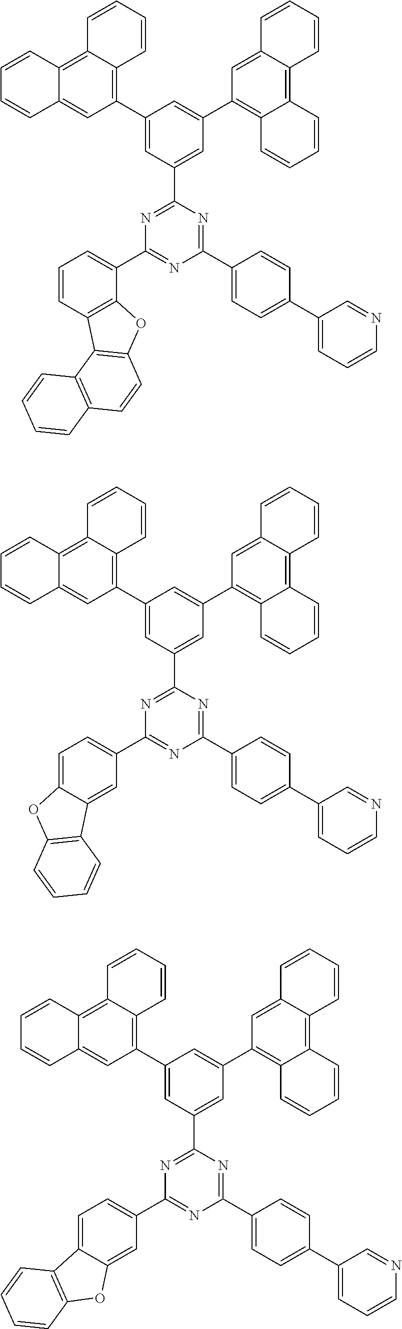 [SMILES: c1cncc(-c2ccc(-c3nc(-c4cc(-c5cc6ccccc6c6ccccc56)cc(-c5cc6ccccc6c6ccccc56)c4)nc(-c4cccc5c4oc4ccc6ccccc6c45)n3)cc2)c1 ; c1cncc(-c2ccc(-c3nc(-c4cc(-c5cc6ccccc6c6ccccc56)cc(-c5cc6ccccc6c6ccccc56)c4)nc(-c4ccc5oc6ccccc6c5c4)n3)cc2)c1 ; c1cncc(-c2ccc(-c3nc(-c4cc(-c5cc6ccccc6c6ccccc56)cc(-c5cc6ccccc6c6ccccc56)c4)nc(-c4ccc5c(c4)oc4ccccc45)n3)cc2)c1]

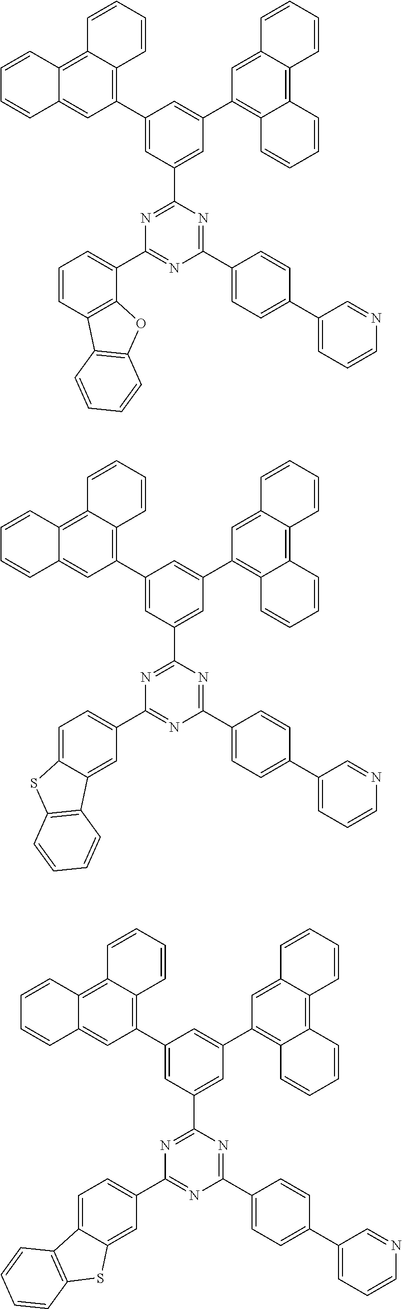 [SMILES: c1cncc(-c2ccc(-c3nc(-c4cc(-c5cc6ccccc6c6ccccc56)cc(-c5cc6ccccc6c6ccccc56)c4)nc(-c4cccc5c4oc4ccccc45)n3)cc2)c1 ; c1cncc(-c2ccc(-c3nc(-c4cc(-c5cc6ccccc6c6ccccc56)cc(-c5cc6ccccc6c6ccccc56)c4)nc(-c4ccc5sc6ccccc6c5c4)n3)cc2)c1 ; c1cncc(-c2ccc(-c3nc(-c4cc(-c5cc6ccccc6c6ccccc56)cc(-c5cc6ccccc6c6ccccc56)c4)nc(-c4ccc5c(c4)sc4ccccc45)n3)cc2)c1]

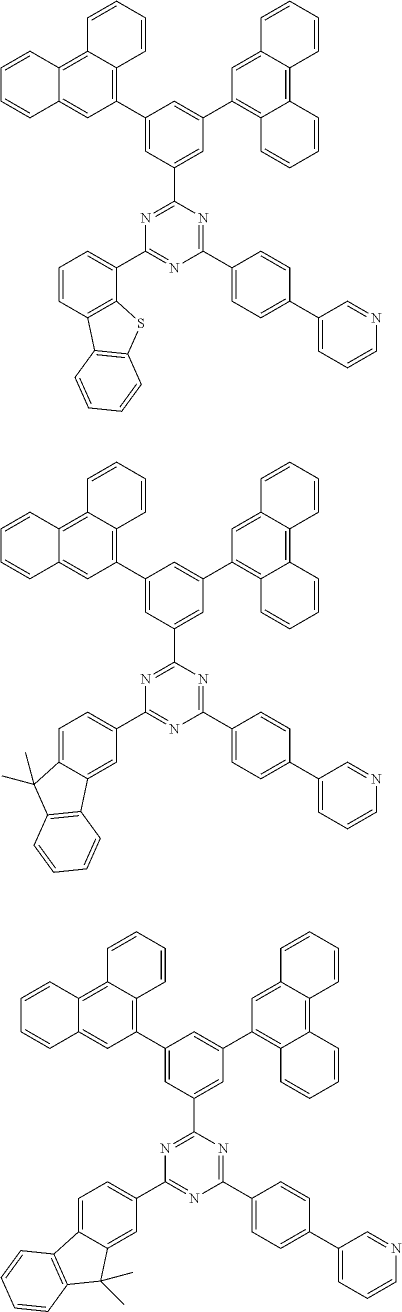 [SMILES: c1cncc(-c2ccc(-c3nc(-c4cc(-c5cc6ccccc6c6ccccc56)cc(-c5cc6ccccc6c6ccccc56)c4)nc(-c4cccc5c4sc4ccccc45)n3)cc2)c1 ; CC1(C)c2ccccc2-c2cc(-c3nc(-c4ccc(-c5cccnc5)cc4)nc(-c4cc(-c5cc6ccccc6c6ccccc56)cc(-c5cc6ccccc6c6ccccc56)c4)n3)ccc21 ; CC1(C)c2ccccc2-c2ccc(-c3nc(-c4ccc(-c5cccnc5)cc4)nc(-c4cc(-c5cc6ccccc6c6ccccc56)cc(-c5cc6ccccc6c6ccccc56)c4)n3)cc21]

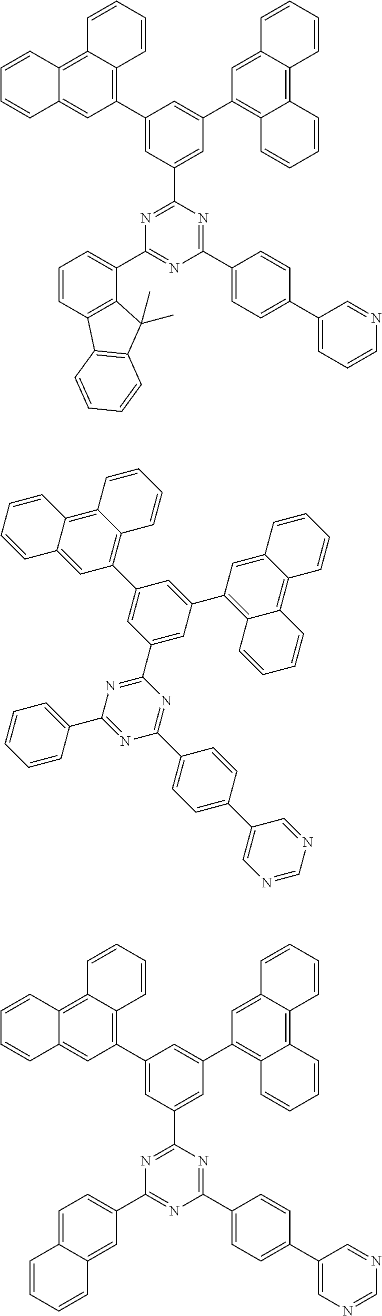 [SMILES: CC1(C)c2ccccc2-c2cccc(-c3nc(-c4ccc(-c5cccnc5)cc4)nc(-c4cc(-c5cc6ccccc6c6ccccc56)cc(-c5cc6ccccc6c6ccccc56)c4)n3)c21 ; c1ccc(-c2nc(-c3ccc(-c4cncnc4)cc3)nc(-c3cc(-c4cc5ccccc5c5ccccc45)cc(-c4cc5ccccc5c5ccccc45)c3)n2)cc1 ; c1ccc2cc(-c3nc(-c4ccc(-c5cncnc5)cc4)nc(-c4cc(-c5cc6ccccc6c6ccccc56)cc(-c5cc6ccccc6c6ccccc56)c4)n3)ccc2c1]

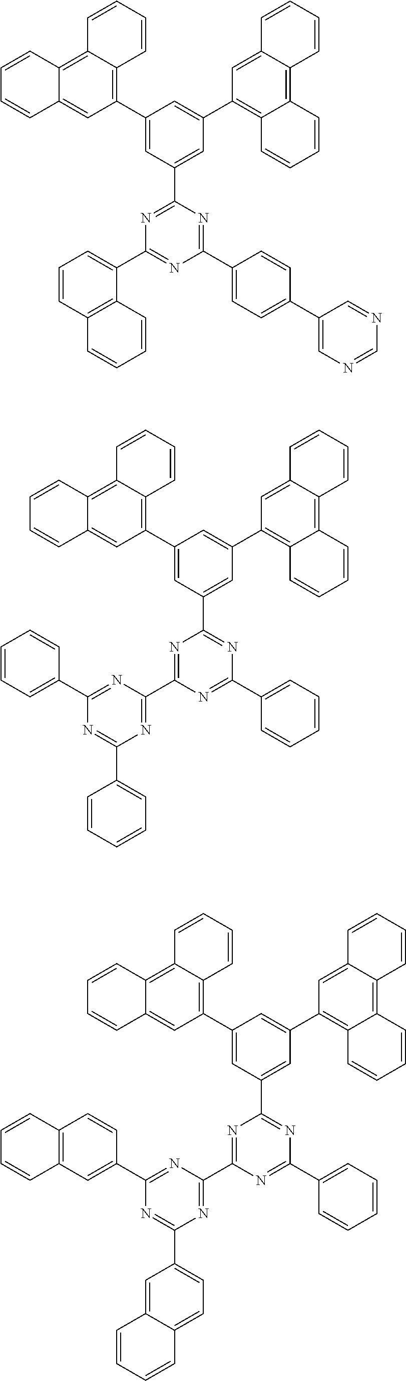 [SMILES: c1ccc2c(-c3nc(-c4ccc(-c5cncnc5)cc4)nc(-c4cc(-c5cc6ccccc6c6ccccc56)cc(-c5cc6ccccc6c6ccccc56)c4)n3)cccc2c1 ; c1ccc(-c2nc(-c3ccccc3)nc(-c3nc(-c4ccccc4)nc(-c4cc(-c5cc6ccccc6c6ccccc56)cc(-c5cc6ccccc6c6ccccc56)c4)n3)n2)cc1 ; c1ccc(-c2nc(-c3cc(-c4cc5ccccc5c5ccccc45)cc(-c4cc5ccccc5c5ccccc45)c3)nc(-c3nc(-c4ccc5ccccc5c4)nc(-c4ccc5ccccc5c4)n3)n2)cc1]

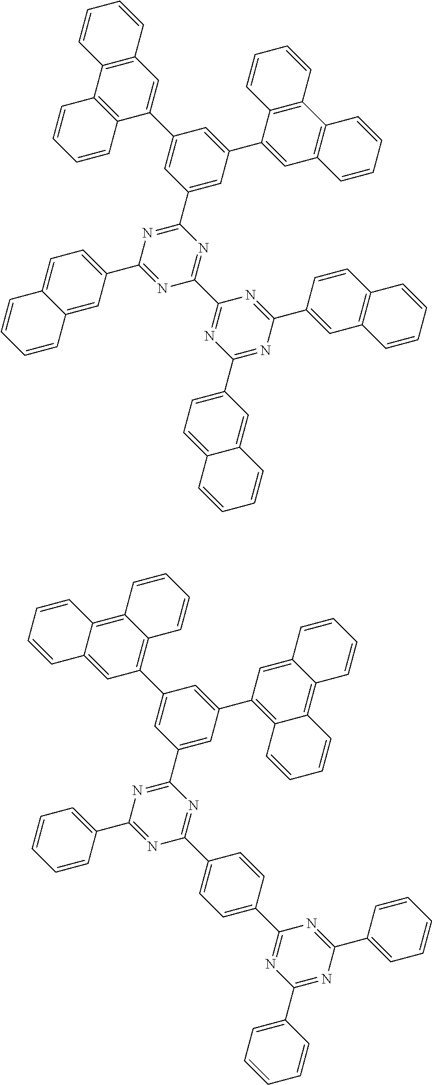 [SMILES: c1ccc2cc(-c3nc(-c4cc(-c5cc6ccccc6c6ccccc56)cc(-c5cc6ccccc6c6ccccc56)c4)nc(-c4nc(-c5ccc6ccccc6c5)nc(-c5ccc6ccccc6c5)n4)n3)ccc2c1 ; c1ccc(-c2nc(-c3ccccc3)nc(-c3ccc(-c4nc(-c5ccccc5)nc(-c5cc(-c6cc7ccccc7c7ccccc67)cc(-c6cc7ccccc7c7ccccc67)c5)n4)cc3)n2)cc1]

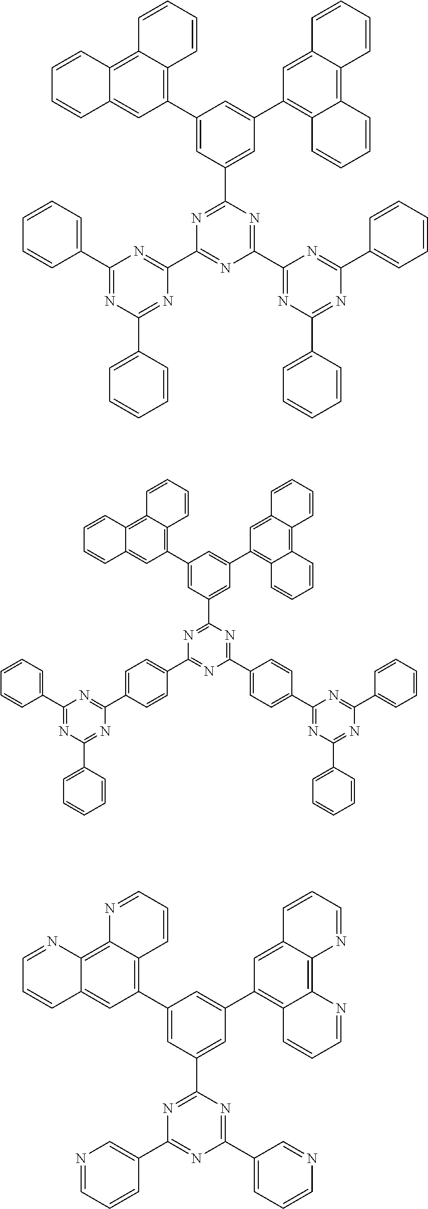 [SMILES: c1ccc(-c2nc(-c3ccccc3)nc(-c3nc(-c4cc(-c5cc6ccccc6c6ccccc56)cc(-c5cc6ccccc6c6ccccc56)c4)nc(-c4nc(-c5ccccc5)nc(-c5ccccc5)n4)n3)n2)cc1 ; c1ccc(-c2nc(-c3ccccc3)nc(-c3ccc(-c4nc(-c5ccc(-c6nc(-c7ccccc7)nc(-c7ccccc7)n6)cc5)nc(-c5cc(-c6cc7ccccc7c7ccccc67)cc(-c6cc7ccccc7c7ccccc67)c5)n4)cc3)n2)cc1 ; c1cncc(-c2nc(-c3cccnc3)nc(-c3cc(-c4cc5cccnc5c5ncccc45)cc(-c4cc5cccnc5c5ncccc45)c3)n2)c1]

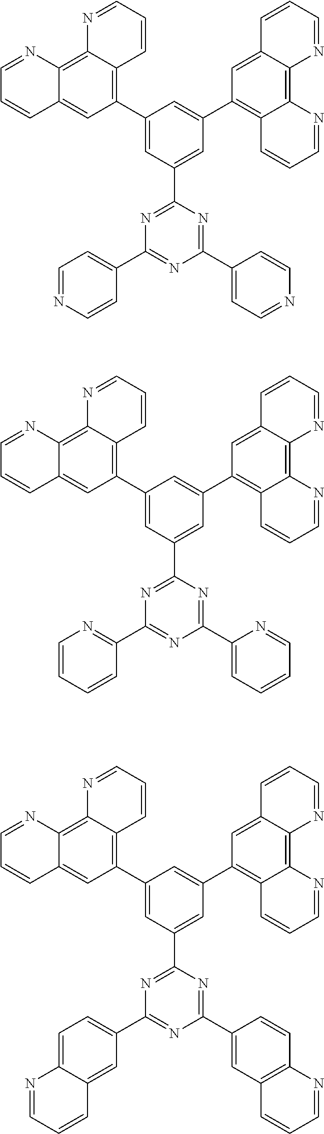 [SMILES: c1cnc2c(c1)cc(-c1cc(-c3nc(-c4ccncc4)nc(-c4ccncc4)n3)cc(-c3cc4cccnc4c4ncccc34)c1)c1cccnc12 ; c1ccc(-c2nc(-c3cc(-c4cc5cccnc5c5ncccc45)cc(-c4cc5cccnc5c5ncccc45)c3)nc(-c3ccccn3)n2)nc1 ; c1cnc2ccc(-c3nc(-c4cc(-c5cc6cccnc6c6ncccc56)cc(-c5cc6cccnc6c6ncccc56)c4)nc(-c4ccc5ncccc5c4)n3)cc2c1]

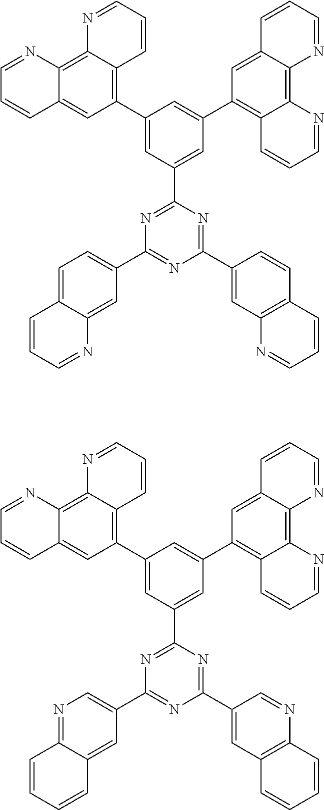 [SMILES: c1cnc2cc(-c3nc(-c4cc(-c5cc6cccnc6c6ncccc56)cc(-c5cc6cccnc6c6ncccc56)c4)nc(-c4ccc5cccnc5c4)n3)ccc2c1 ; c1ccc2ncc(-c3nc(-c4cc(-c5cc6cccnc6c6ncccc56)cc(-c5cc6cccnc6c6ncccc56)c4)nc(-c4cnc5ccccc5c4)n3)cc2c1]

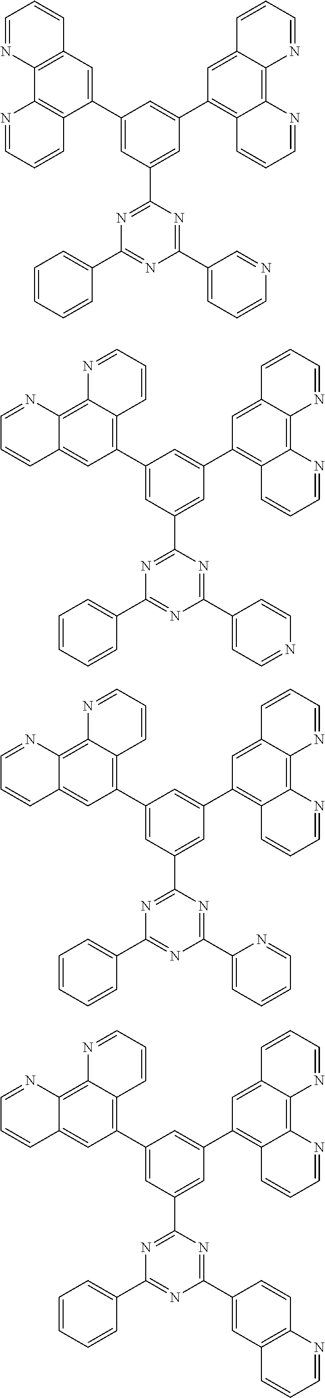

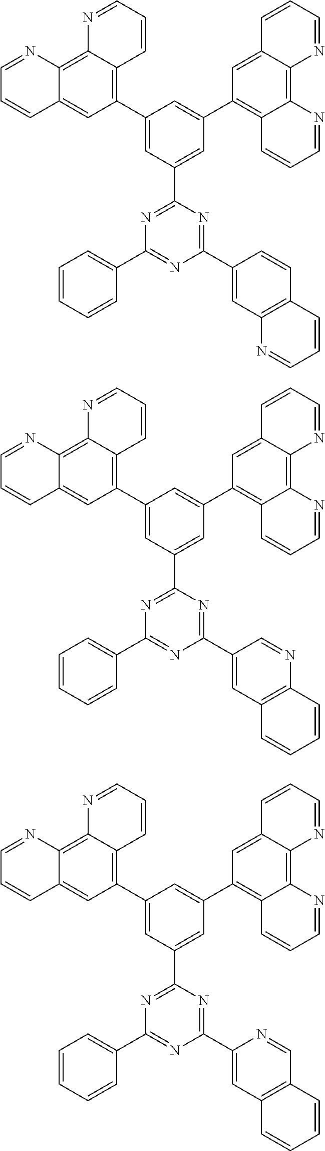

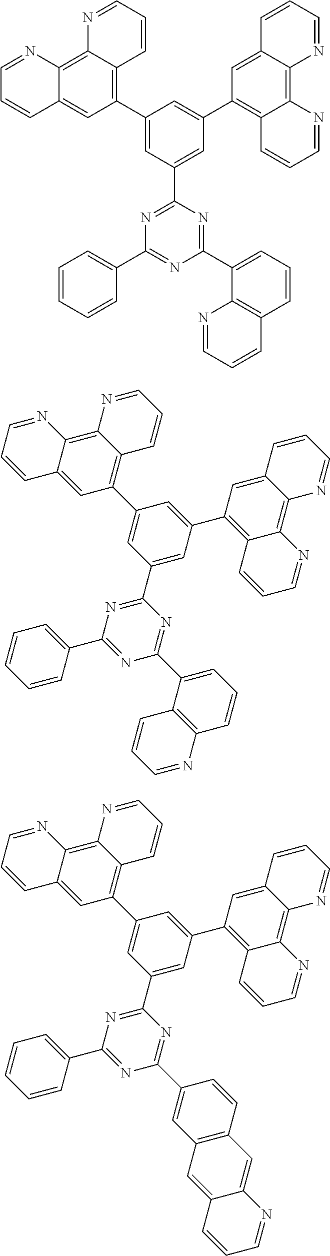

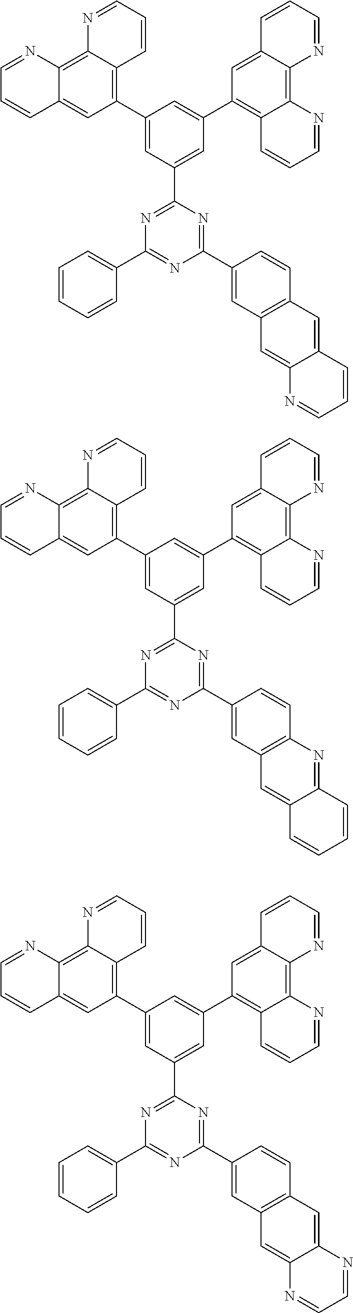

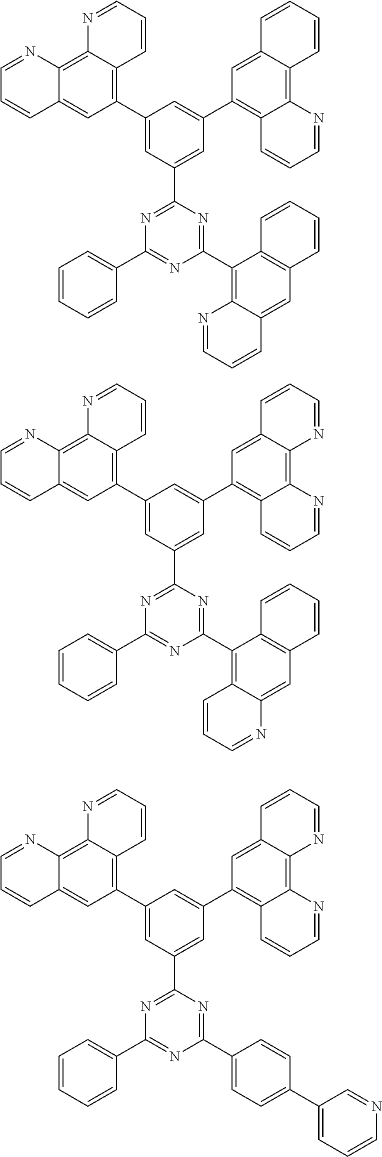

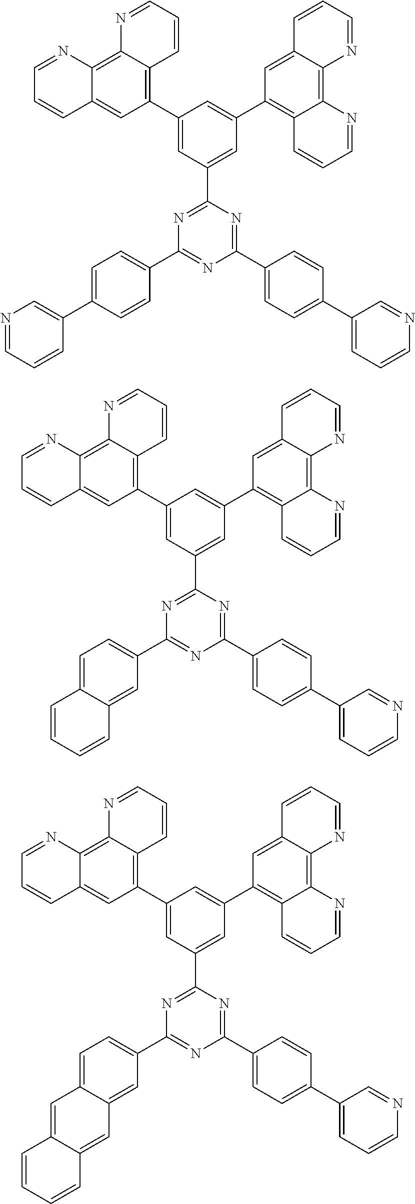 [SMILES: c1cncc(-c2ccc(-c3nc(-c4ccc(-c5cccnc5)cc4)nc(-c4cc(-c5cc6cccnc6c6ncccc56)cc(-c5cc6cccnc6c6ncccc56)c4)n3)cc2)c1 ; c1cncc(-c2ccc(-c3nc(-c4cc(-c5cc6cccnc6c6ncccc56)cc(-c5cc6cccnc6c6ncccc56)c4)nc(-c4ccc5ccccc5c4)n3)cc2)c1 ; c1cncc(-c2ccc(-c3nc(-c4cc(-c5cc6cccnc6c6ncccc56)cc(-c5cc6cccnc6c6ncccc56)c4)nc(-c4ccc5cc6ccccc6cc5c4)n3)cc2)c1]

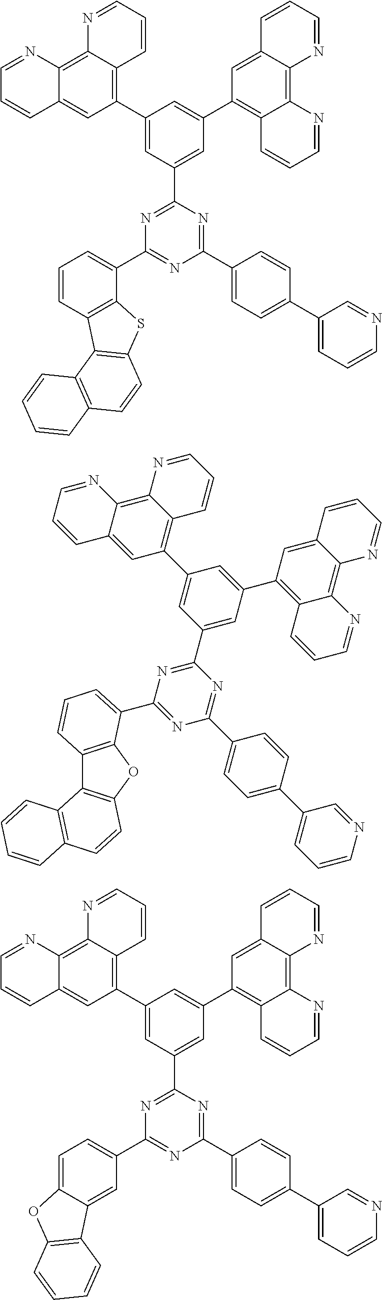

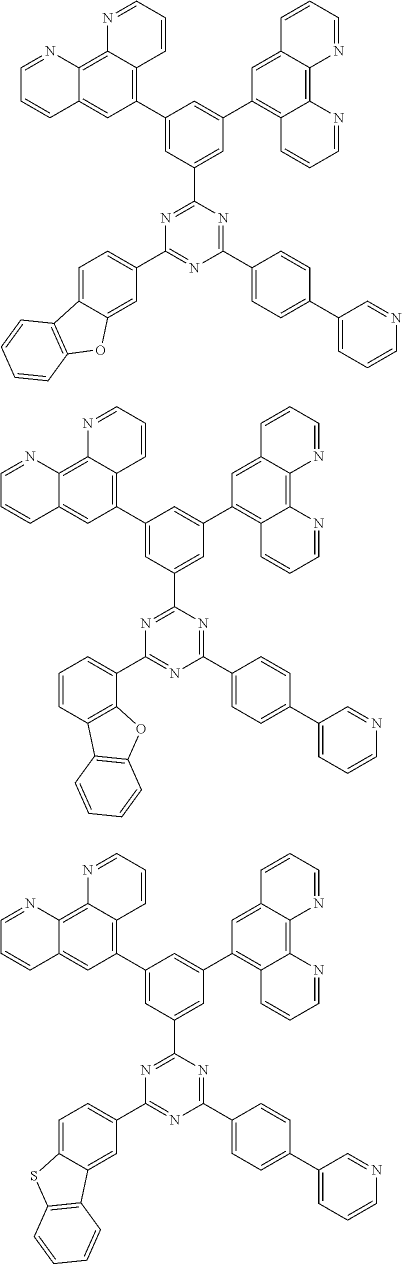

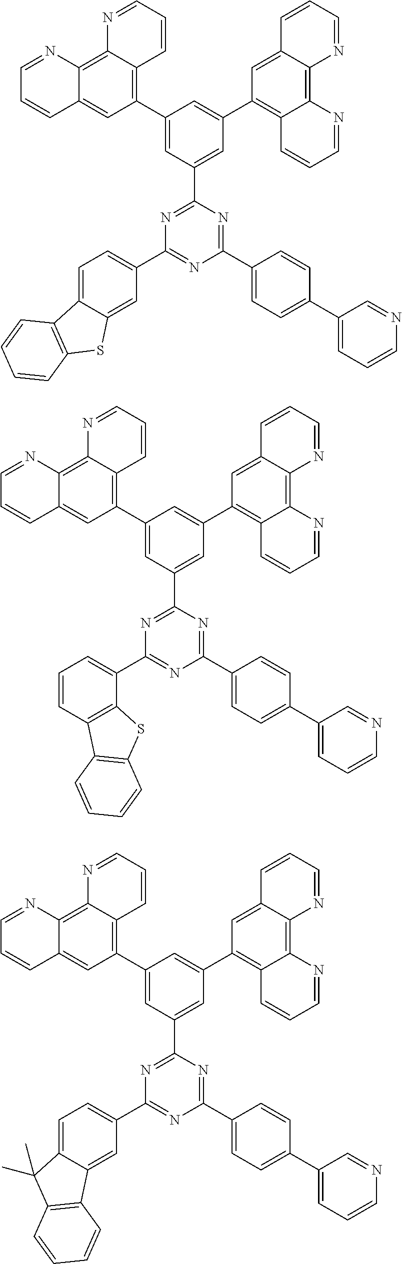

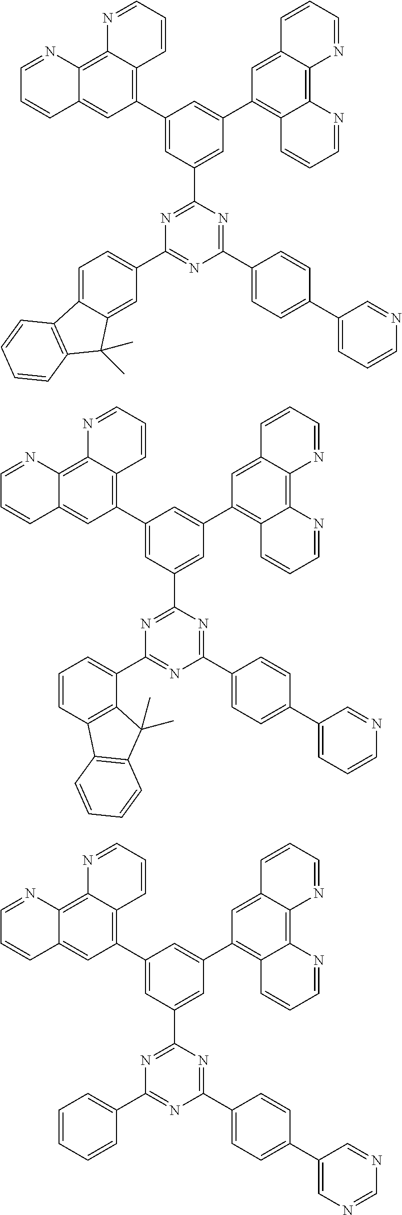

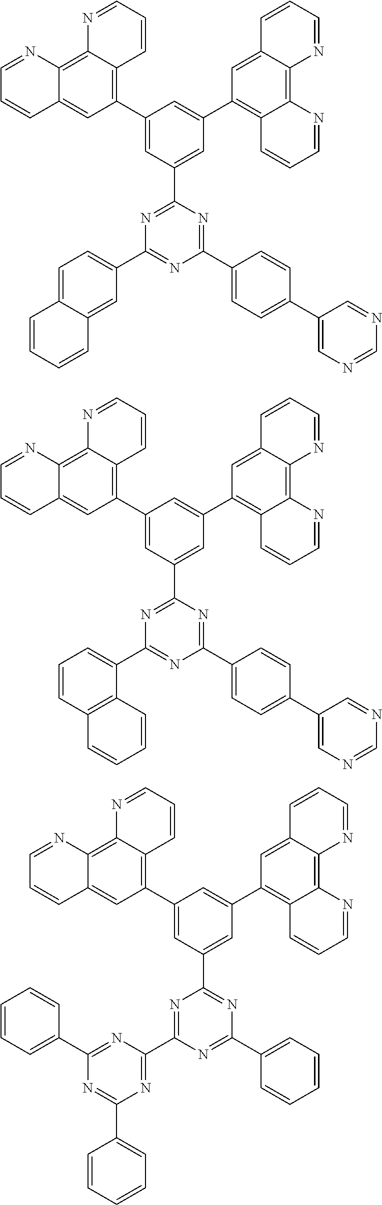

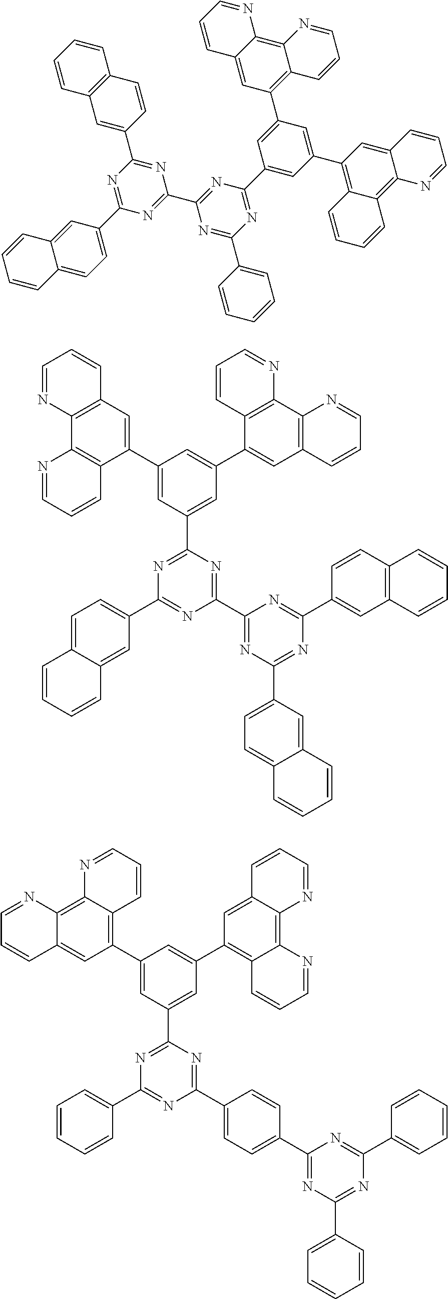

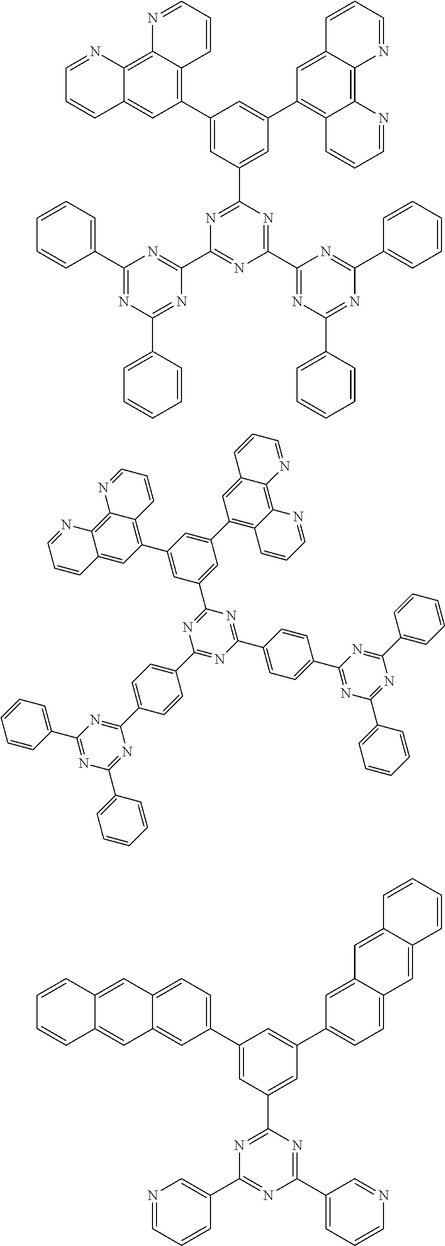

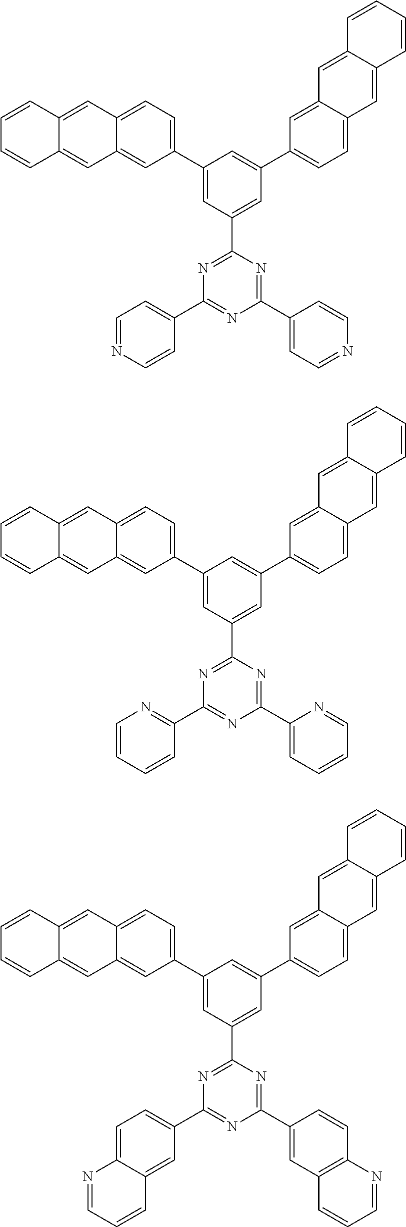 [SMILES: c1ccc2cc3cc(-c4cc(-c5ccc6cc7ccccc7cc6c5)cc(-c5nc(-c6ccncc6)nc(-c6ccncc6)n5)c4)ccc3cc2c1 ; c1ccc(-c2nc(-c3cc(-c4ccc5cc6ccccc6cc5c4)cc(-c4ccc5cc6ccccc6cc5c4)c3)nc(-c3ccccn3)n2)nc1 ; c1ccc2cc3cc(-c4cc(-c5ccc6cc7ccccc7cc6c5)cc(-c5nc(-c6ccc7ncccc7c6)nc(-c6ccc7ncccc7c6)n5)c4)ccc3cc2c1]

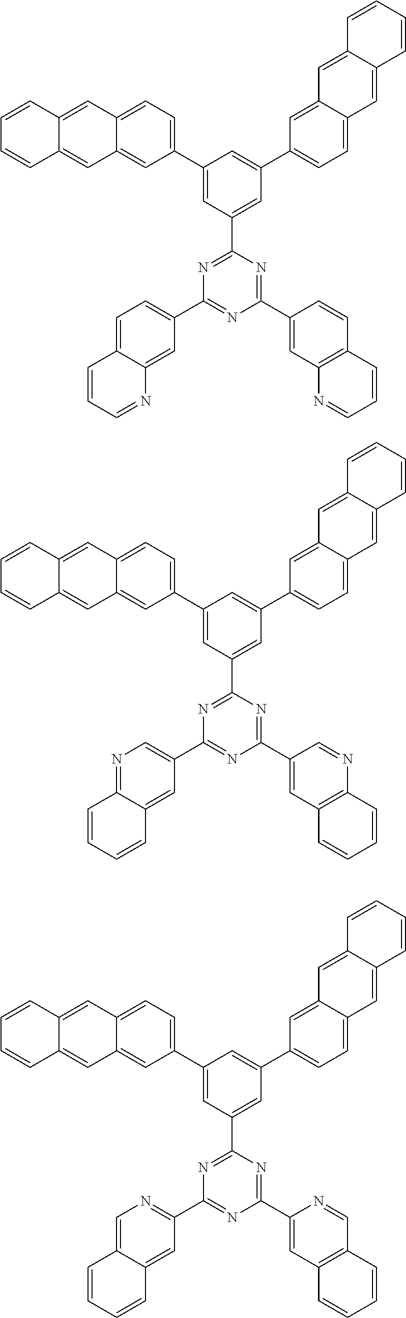

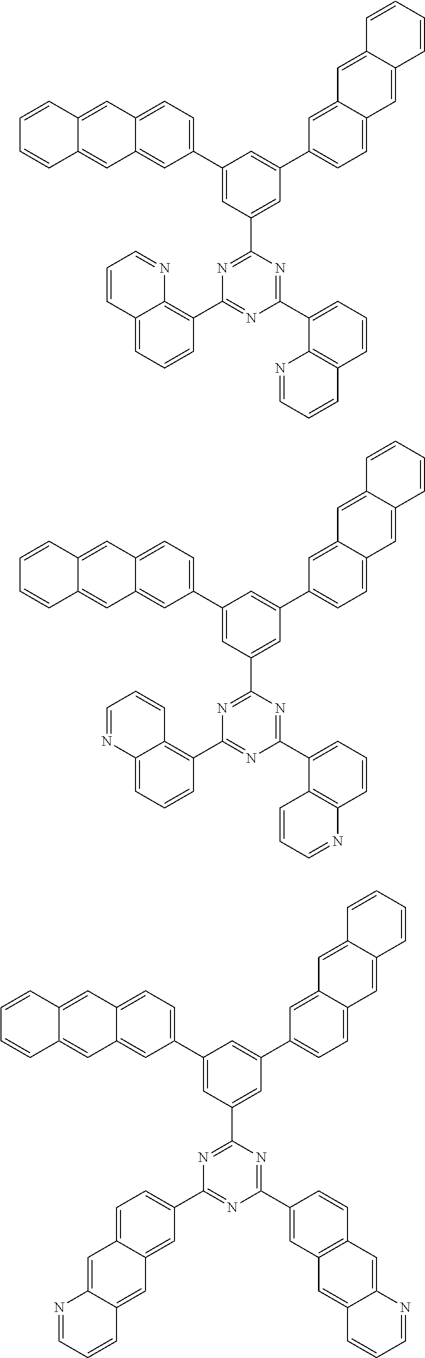 [SMILES: c1ccc2cc3cc(-c4cc(-c5ccc6cc7ccccc7cc6c5)cc(-c5nc(-c6cccc7cccnc67)nc(-c6cccc7cccnc67)n5)c4)ccc3cc2c1 ; c1ccc2cc3cc(-c4cc(-c5ccc6cc7ccccc7cc6c5)cc(-c5nc(-c6cccc7ncccc67)nc(-c6cccc7ncccc67)n5)c4)ccc3cc2c1 ; c1ccc2cc3cc(-c4cc(-c5ccc6cc7ccccc7cc6c5)cc(-c5nc(-c6ccc7cc8ncccc8cc7c6)nc(-c6ccc7cc8ncccc8cc7c6)n5)c4)ccc3cc2c1]

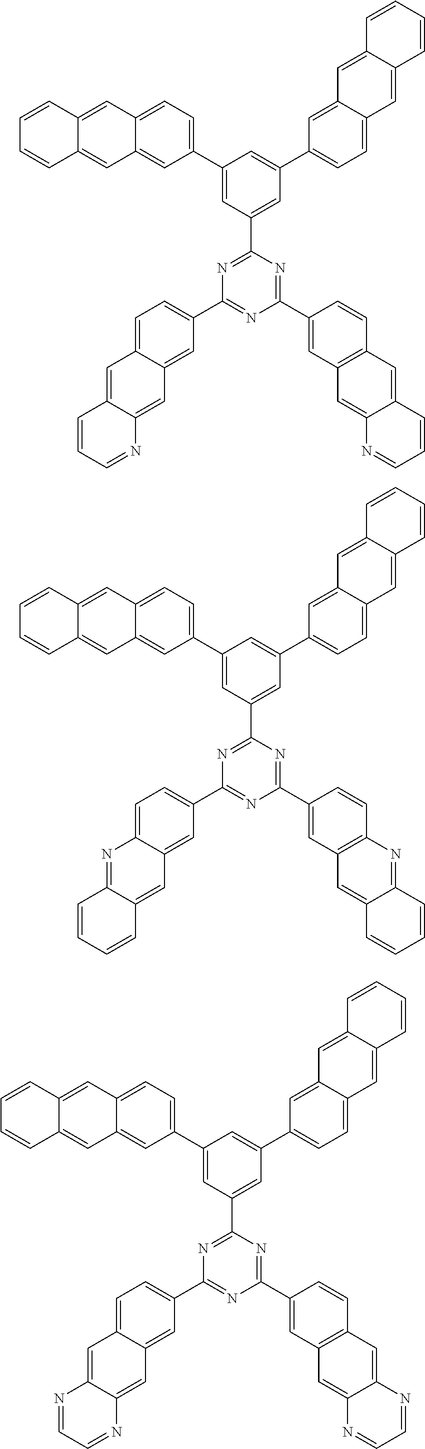

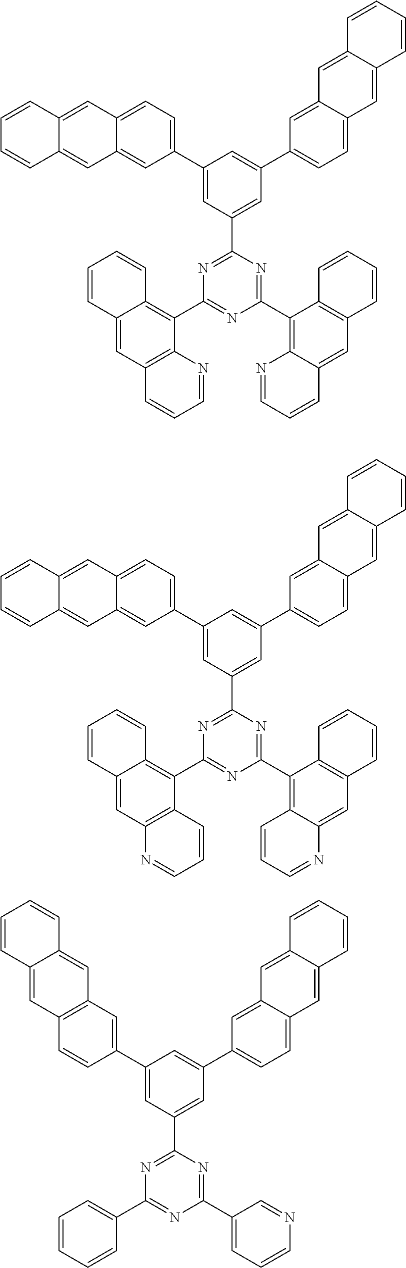

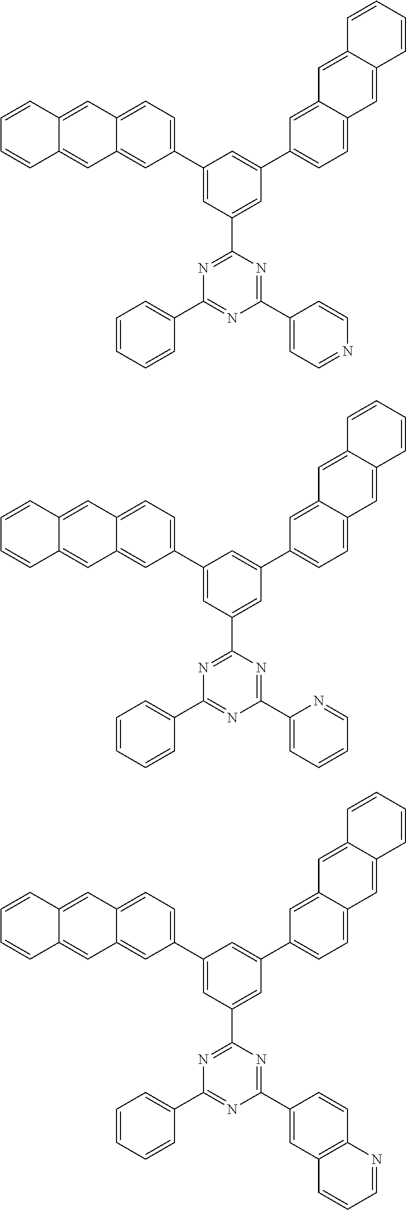 [SMILES: c1ccc(-c2nc(-c3ccncc3)nc(-c3cc(-c4ccc5cc6ccccc6cc5c4)cc(-c4ccc5cc6ccccc6cc5c4)c3)n2)cc1 ; c1ccc(-c2nc(-c3cc(-c4ccc5cc6ccccc6cc5c4)cc(-c4ccc5cc6ccccc6cc5c4)c3)nc(-c3ccccn3)n2)cc1 ; c1ccc(-c2nc(-c3cc(-c4ccc5cc6ccccc6cc5c4)cc(-c4ccc5cc6ccccc6cc5c4)c3)nc(-c3ccc4ncccc4c3)n2)cc1]

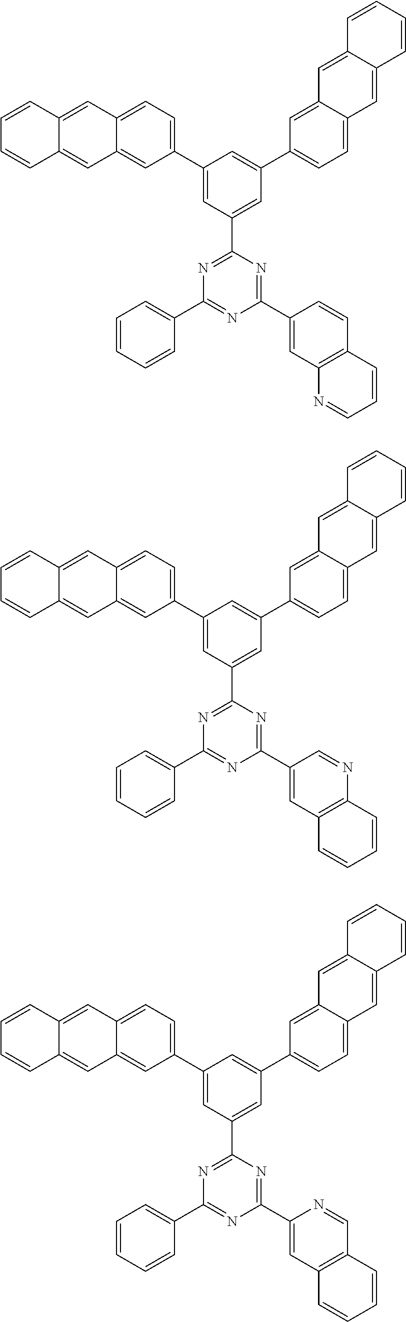

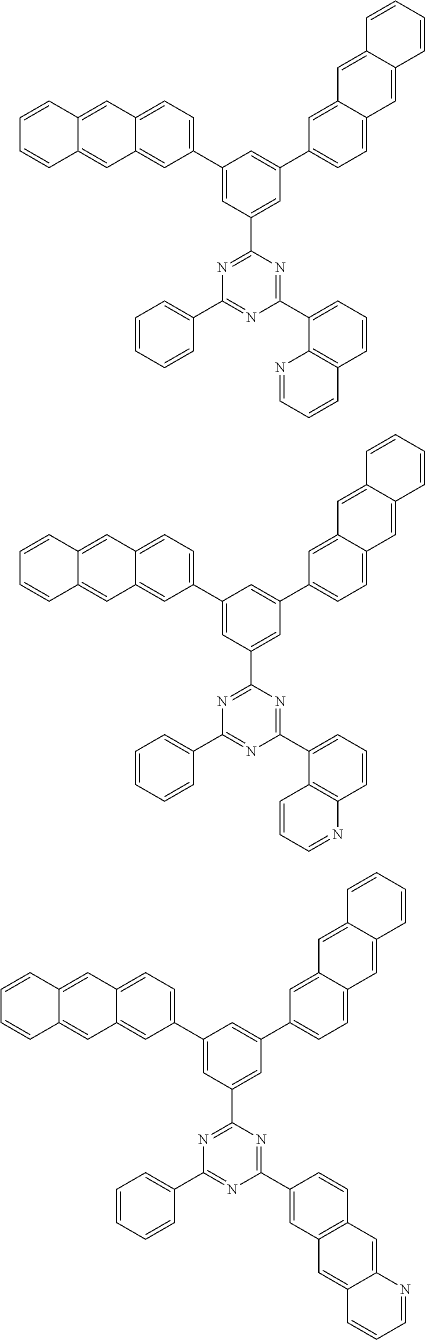 [SMILES: c1ccc(-c2nc(-c3cc(-c4ccc5cc6ccccc6cc5c4)cc(-c4ccc5cc6ccccc6cc5c4)c3)nc(-c3cccc4cccnc34)n2)cc1 ; c1ccc(-c2nc(-c3cc(-c4ccc5cc6ccccc6cc5c4)cc(-c4ccc5cc6ccccc6cc5c4)c3)nc(-c3cccc4ncccc34)n2)cc1 ; c1ccc(-c2nc(-c3cc(-c4ccc5cc6ccccc6cc5c4)cc(-c4ccc5cc6ccccc6cc5c4)c3)nc(-c3ccc4cc5ncccc5cc4c3)n2)cc1]

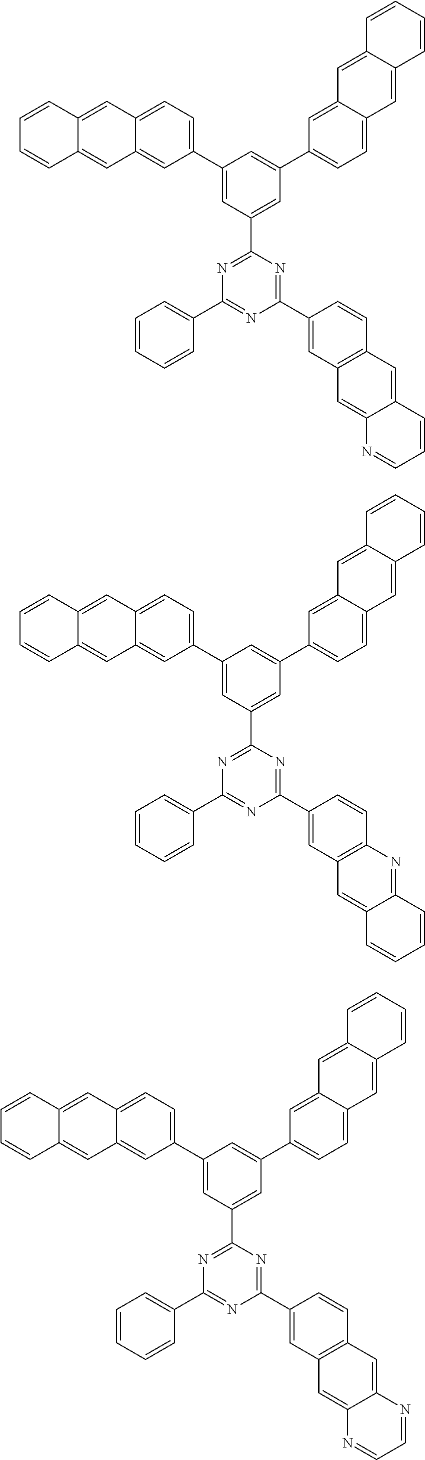

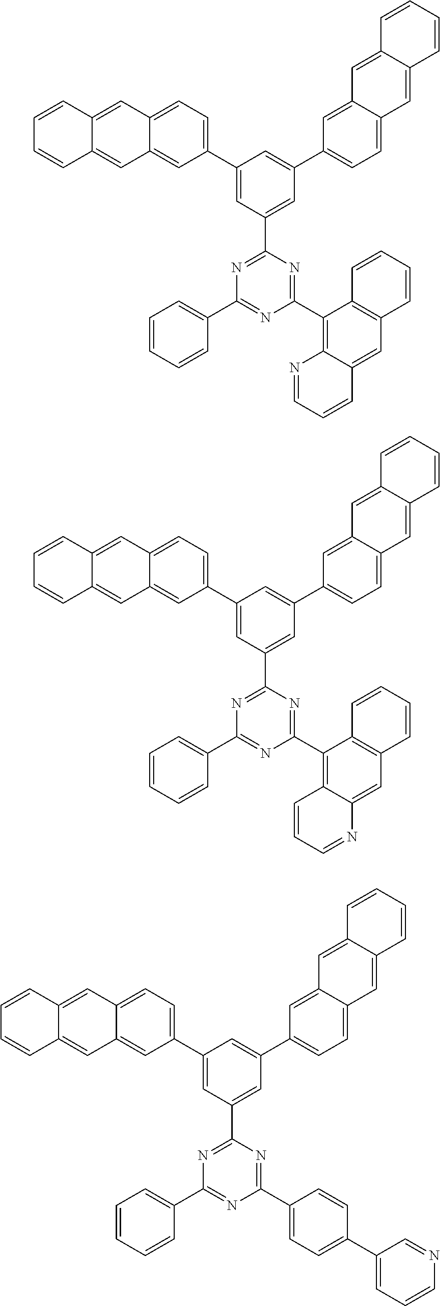 [SMILES: c1ccc(-c2nc(-c3cc(-c4ccc5cc6ccccc6cc5c4)cc(-c4ccc5cc6ccccc6cc5c4)c3)nc(-c3c4ccccc4cc4cccnc34)n2)cc1 ; c1ccc(-c2nc(-c3cc(-c4ccc5cc6ccccc6cc5c4)cc(-c4ccc5cc6ccccc6cc5c4)c3)nc(-c3c4ccccc4cc4ncccc34)n2)cc1 ; c1ccc(-c2nc(-c3ccc(-c4cccnc4)cc3)nc(-c3cc(-c4ccc5cc6ccccc6cc5c4)cc(-c4ccc5cc6ccccc6cc5c4)c3)n2)cc1]

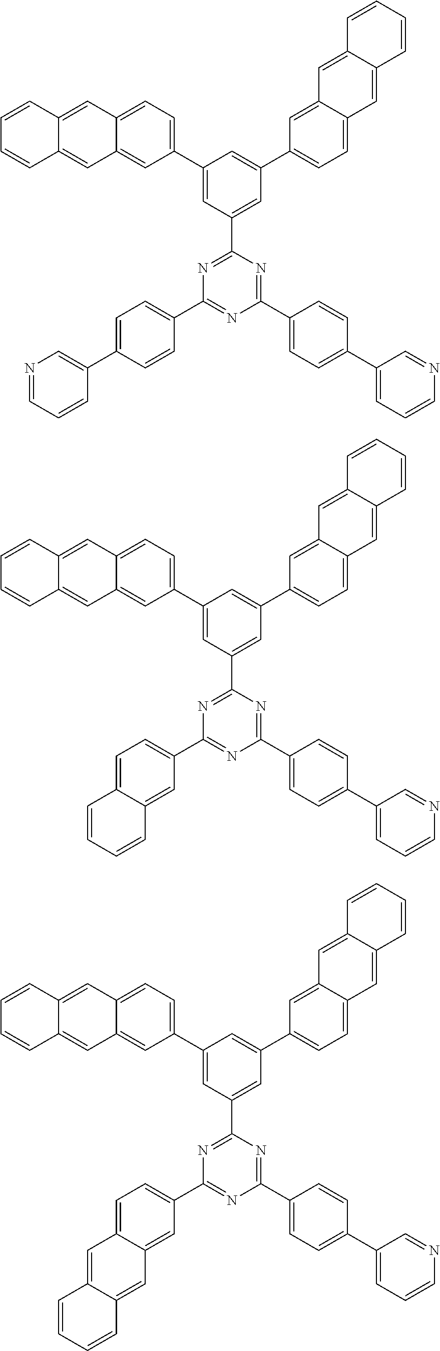

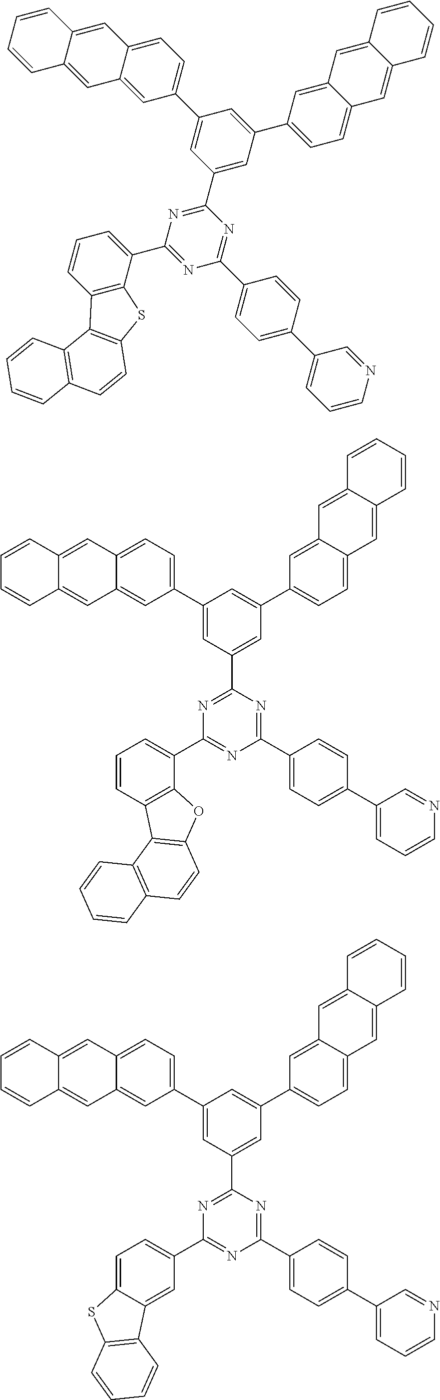

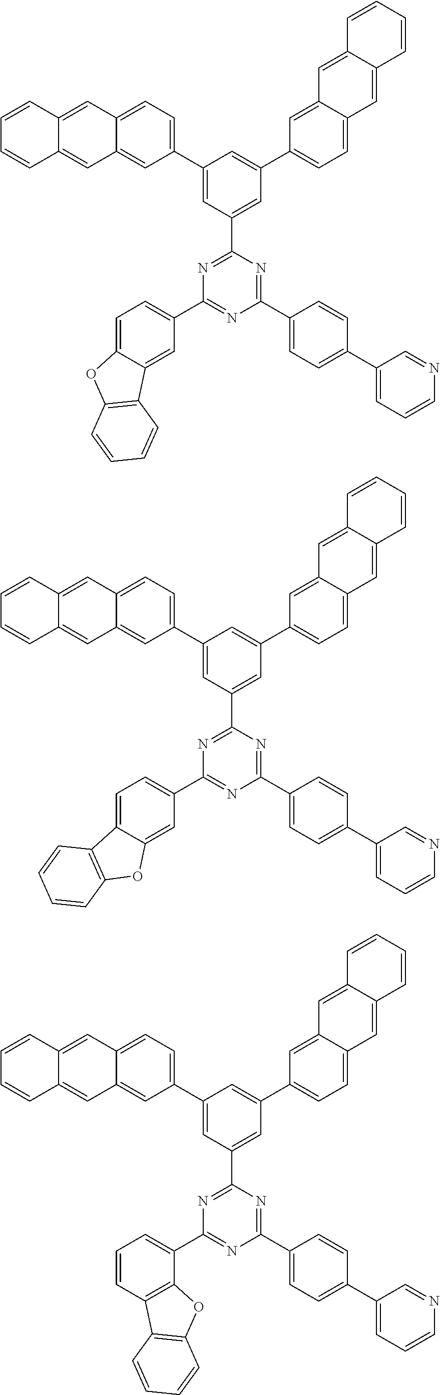 [SMILES: c1cncc(-c2ccc(-c3nc(-c4cc(-c5ccc6cc7ccccc7cc6c5)cc(-c5ccc6cc7ccccc7cc6c5)c4)nc(-c4ccc5oc6ccccc6c5c4)n3)cc2)c1 ; c1cncc(-c2ccc(-c3nc(-c4cc(-c5ccc6cc7ccccc7cc6c5)cc(-c5ccc6cc7ccccc7cc6c5)c4)nc(-c4ccc5c(c4)oc4ccccc45)n3)cc2)c1 ; c1cncc(-c2ccc(-c3nc(-c4cc(-c5ccc6cc7ccccc7cc6c5)cc(-c5ccc6cc7ccccc7cc6c5)c4)nc(-c4cccc5c4oc4ccccc45)n3)cc2)c1]

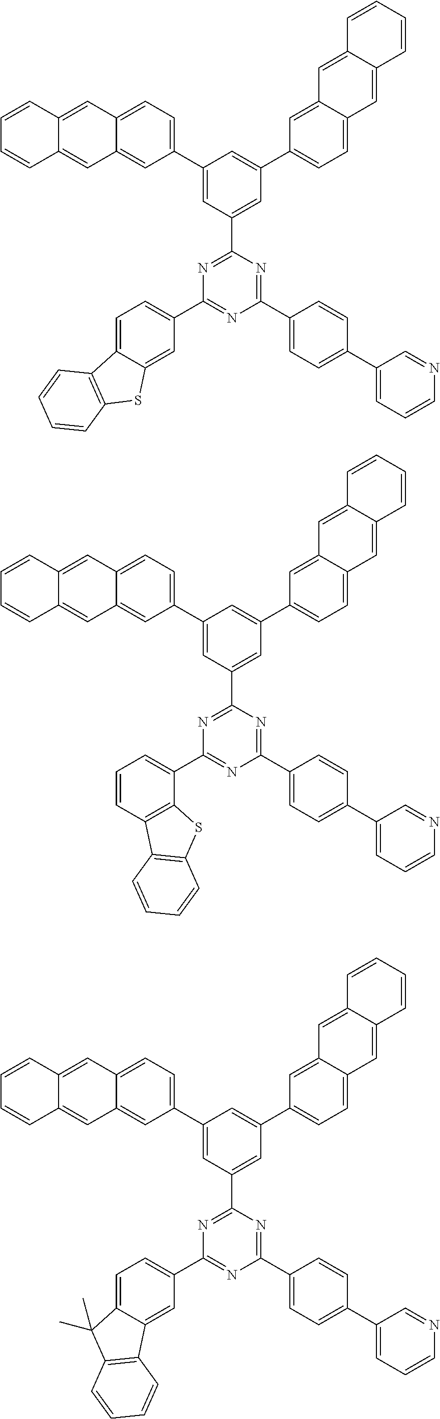 [SMILES: c1cncc(-c2ccc(-c3nc(-c4cc(-c5ccc6cc7ccccc7cc6c5)cc(-c5ccc6cc7ccccc7cc6c5)c4)nc(-c4ccc5c(c4)sc4ccccc45)n3)cc2)c1 ; c1cncc(-c2ccc(-c3nc(-c4cc(-c5ccc6cc7ccccc7cc6c5)cc(-c5ccc6cc7ccccc7cc6c5)c4)nc(-c4cccc5c4sc4ccccc45)n3)cc2)c1 ; CC1(C)c2ccccc2-c2cc(-c3nc(-c4ccc(-c5cccnc5)cc4)nc(-c4cc(-c5ccc6cc7ccccc7cc6c5)cc(-c5ccc6cc7ccccc7cc6c5)c4)n3)ccc21]

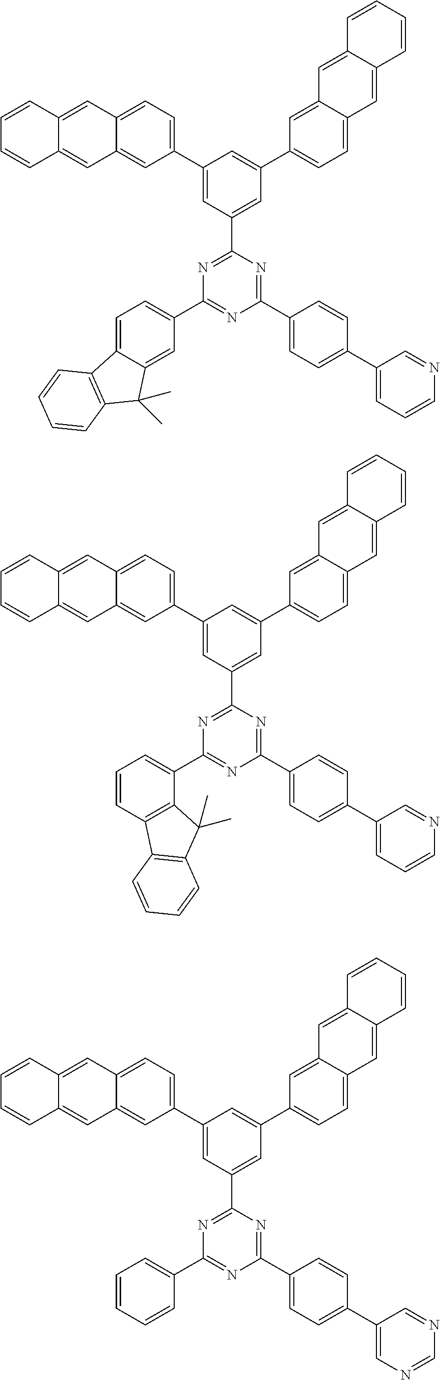 [SMILES: CC1(C)c2ccccc2-c2ccc(-c3nc(-c4ccc(-c5cccnc5)cc4)nc(-c4cc(-c5ccc6cc7ccccc7cc6c5)cc(-c5ccc6cc7ccccc7cc6c5)c4)n3)cc21 ; CC1(C)c2ccccc2-c2cccc(-c3nc(-c4ccc(-c5cccnc5)cc4)nc(-c4cc(-c5ccc6cc7ccccc7cc6c5)cc(-c5ccc6cc7ccccc7cc6c5)c4)n3)c21 ; c1ccc(-c2nc(-c3ccc(-c4cncnc4)cc3)nc(-c3cc(-c4ccc5cc6ccccc6cc5c4)cc(-c4ccc5cc6ccccc6cc5c4)c3)n2)cc1]

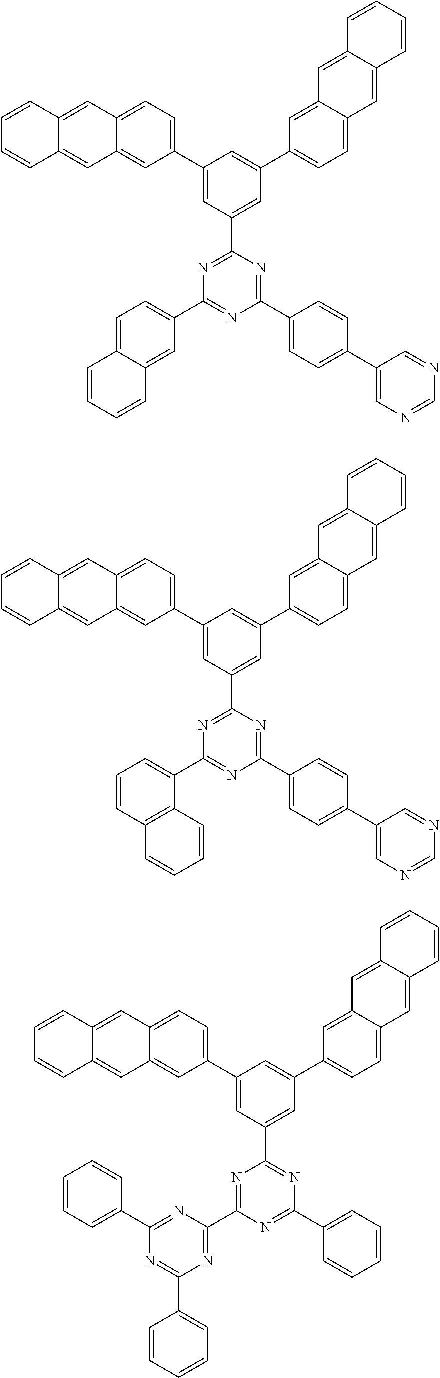

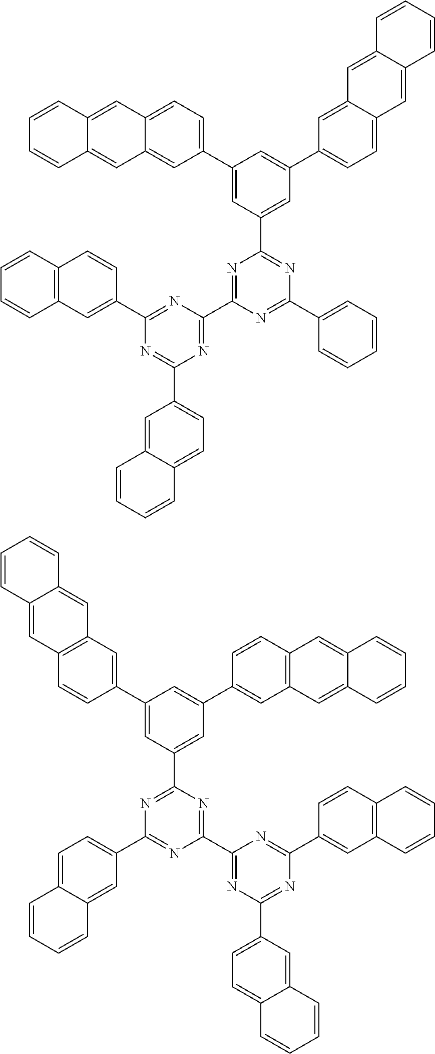 [SMILES: c1ccc(-c2nc(-c3cc(-c4ccc5cc6ccccc6cc5c4)cc(-c4ccc5cc6ccccc6cc5c4)c3)nc(-c3nc(-c4ccc5ccccc5c4)nc(-c4ccc5ccccc5c4)n3)n2)cc1 ; c1ccc2cc(-c3nc(-c4cc(-c5ccc6cc7ccccc7cc6c5)cc(-c5ccc6cc7ccccc7cc6c5)c4)nc(-c4nc(-c5ccc6ccccc6c5)nc(-c5ccc6ccccc6c5)n4)n3)ccc2c1]

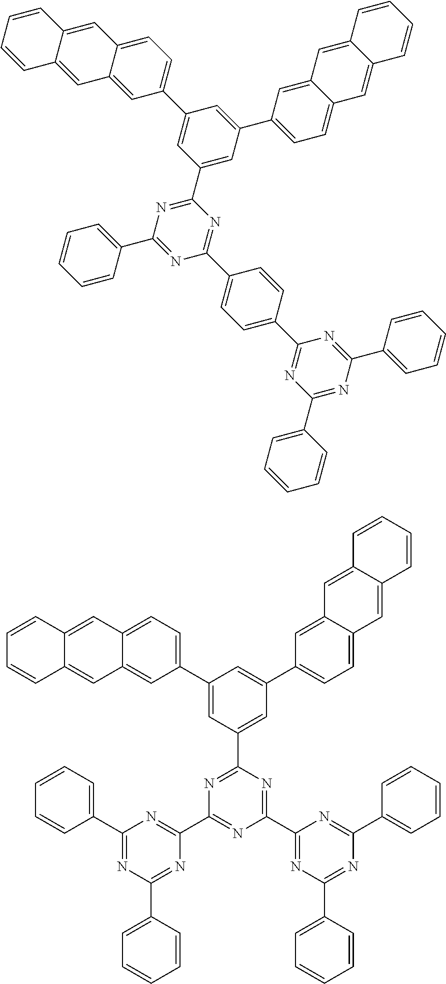 [SMILES: c1ccc(-c2nc(-c3ccccc3)nc(-c3ccc(-c4nc(-c5ccccc5)nc(-c5cc(-c6ccc7cc8ccccc8cc7c6)cc(-c6ccc7cc8ccccc8cc7c6)c5)n4)cc3)n2)cc1 ; c1ccc(-c2nc(-c3ccccc3)nc(-c3nc(-c4cc(-c5ccc6cc7ccccc7cc6c5)cc(-c5ccc6cc7ccccc7cc6c5)c4)nc(-c4nc(-c5ccccc5)nc(-c5ccccc5)n4)n3)n2)cc1]

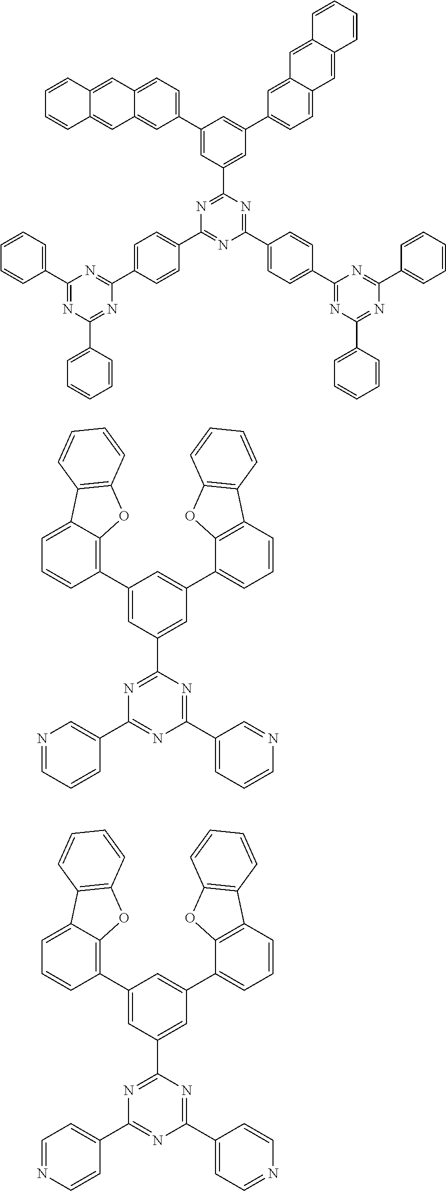

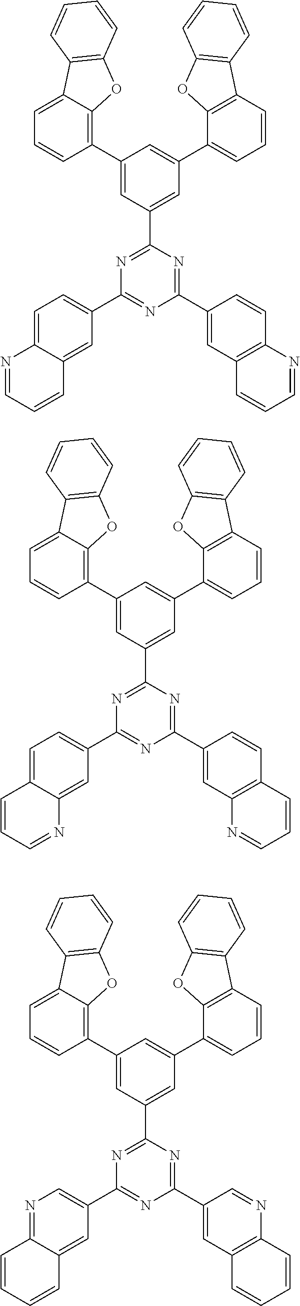

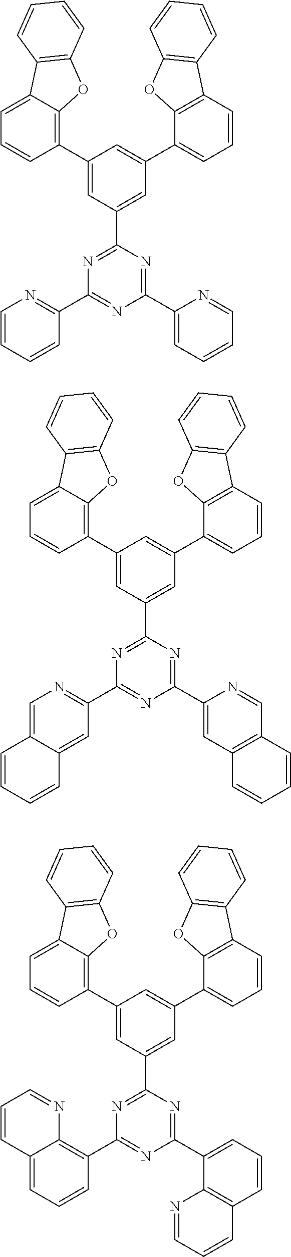

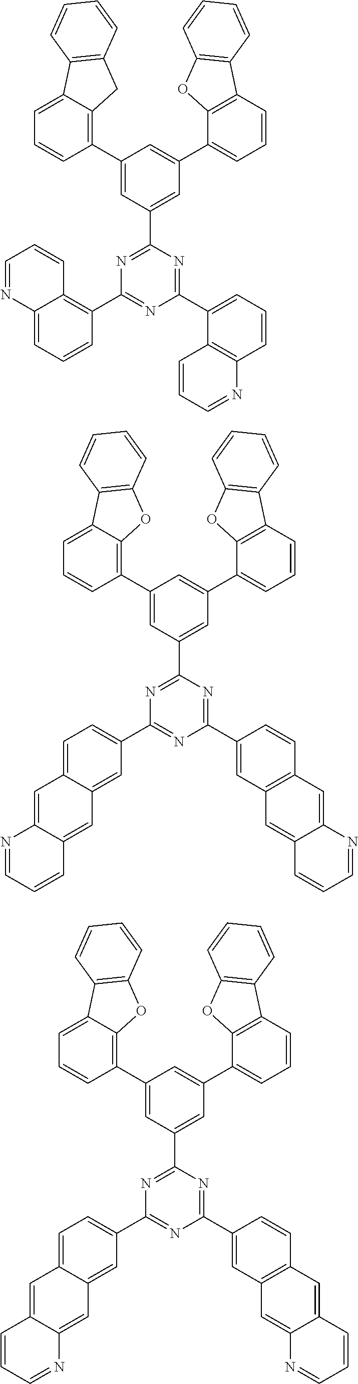

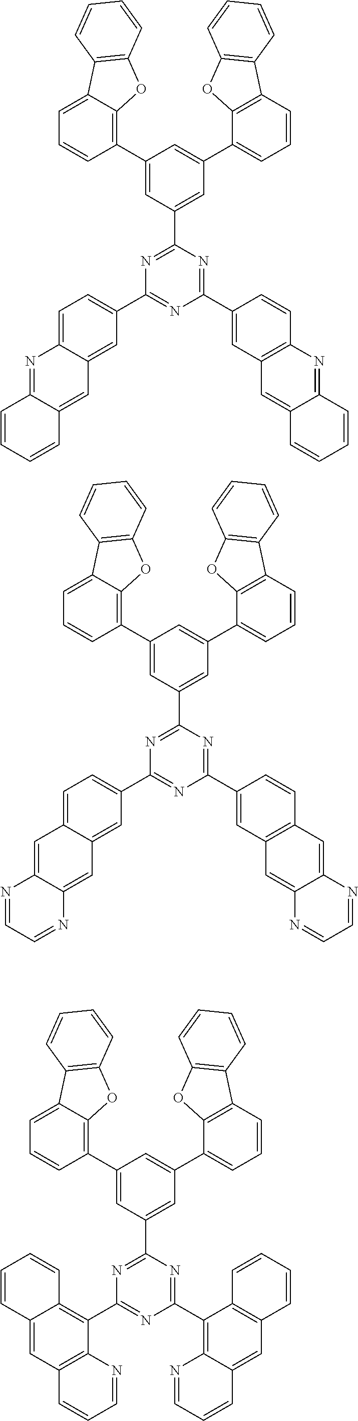 [SMILES: c1ccc2nc3ccc(-c4nc(-c5cc(-c6cccc7c6oc6ccccc67)cc(-c6cccc7c6oc6ccccc67)c5)nc(-c5ccc6nc7ccccc7cc6c5)n4)cc3cc2c1 ; c1ccc2c(c1)oc1c(-c3cc(-c4nc(-c5ccc6cc7nccnc7cc6c5)nc(-c5ccc6cc7nccnc7cc6c5)n4)cc(-c4cccc5c4oc4ccccc45)c3)cccc12 ; c1ccc2c(-c3nc(-c4cc(-c5cccc6c5oc5ccccc56)cc(-c5cccc6c5oc5ccccc56)c4)nc(-c4c5ccccc5cc5cccnc45)n3)c3ncccc3cc2c1]

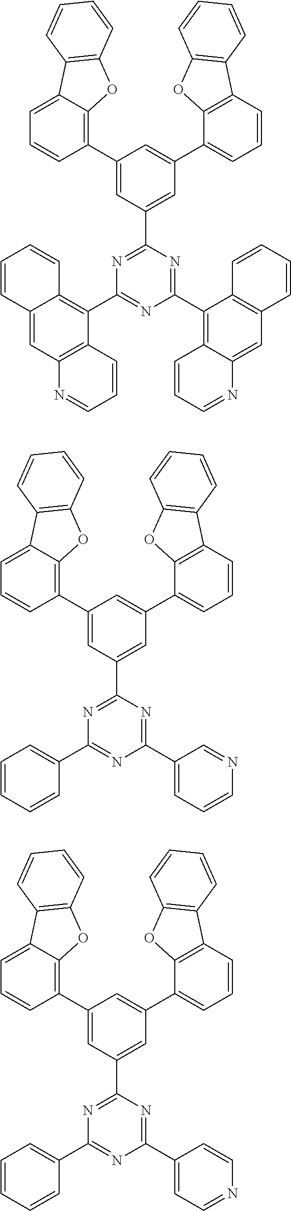

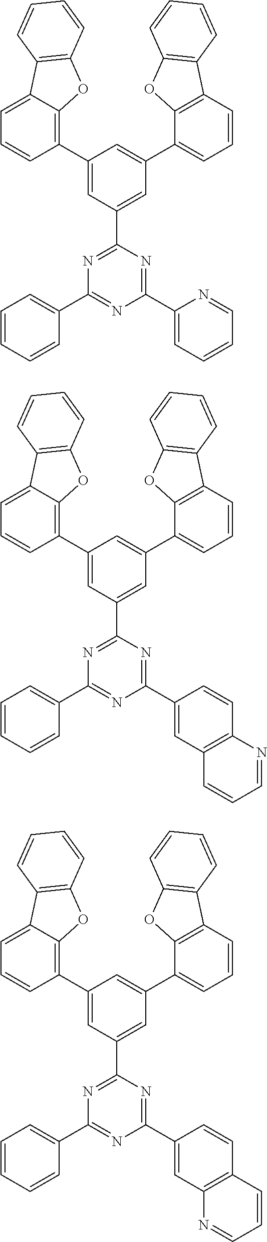

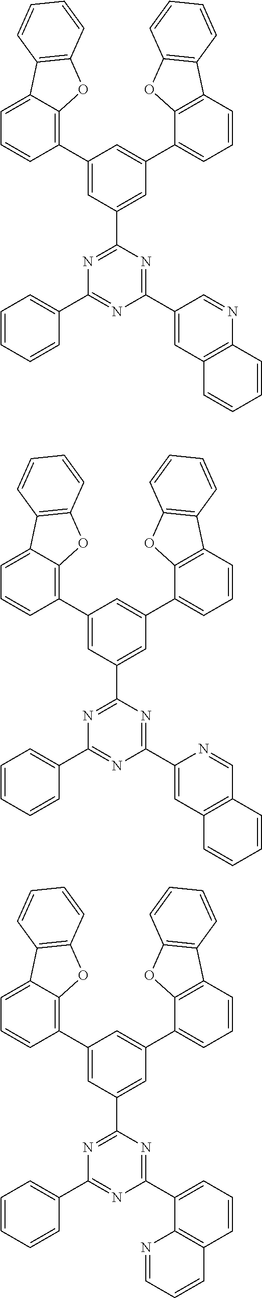

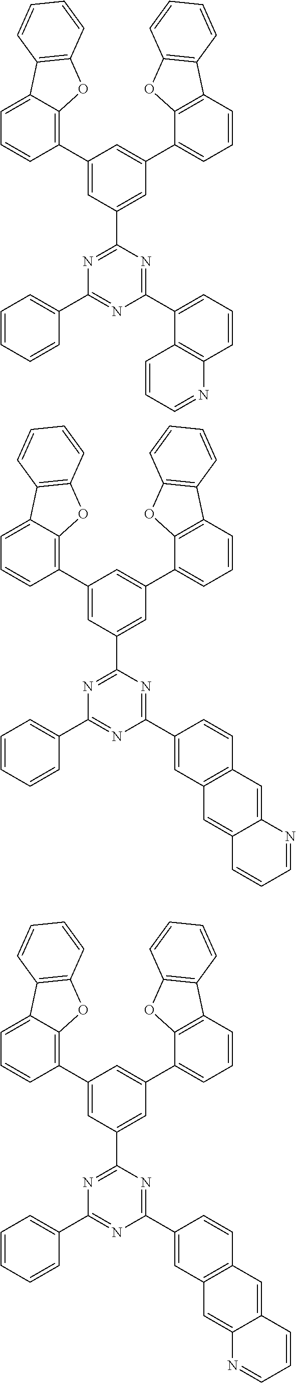

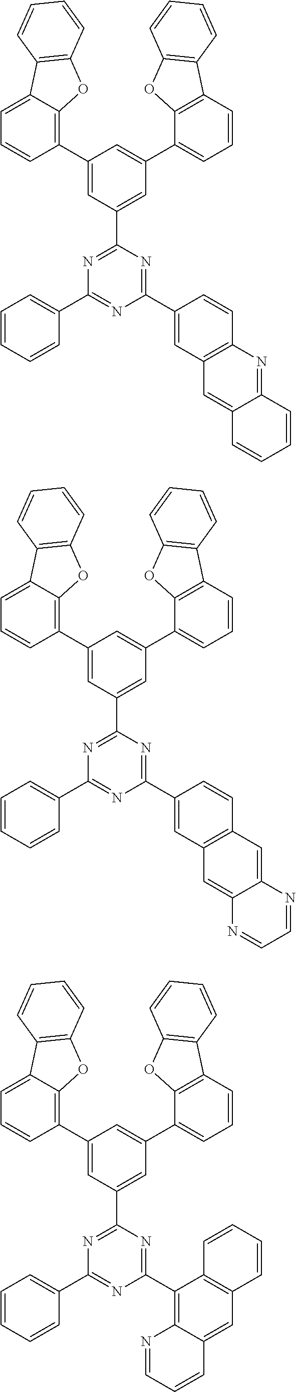 [SMILES: c1ccc(-c2nc(-c3cc(-c4cccc5c4oc4ccccc45)cc(-c4cccc5c4oc4ccccc45)c3)nc(-c3ccc4nc5ccccc5cc4c3)n2)cc1 ; c1ccc(-c2nc(-c3cc(-c4cccc5c4oc4ccccc45)cc(-c4cccc5c4oc4ccccc45)c3)nc(-c3ccc4cc5nccnc5cc4c3)n2)cc1 ; c1ccc(-c2nc(-c3cc(-c4cccc5c4oc4ccccc45)cc(-c4cccc5c4oc4ccccc45)c3)nc(-c3c4ccccc4cc4cccnc34)n2)cc1]

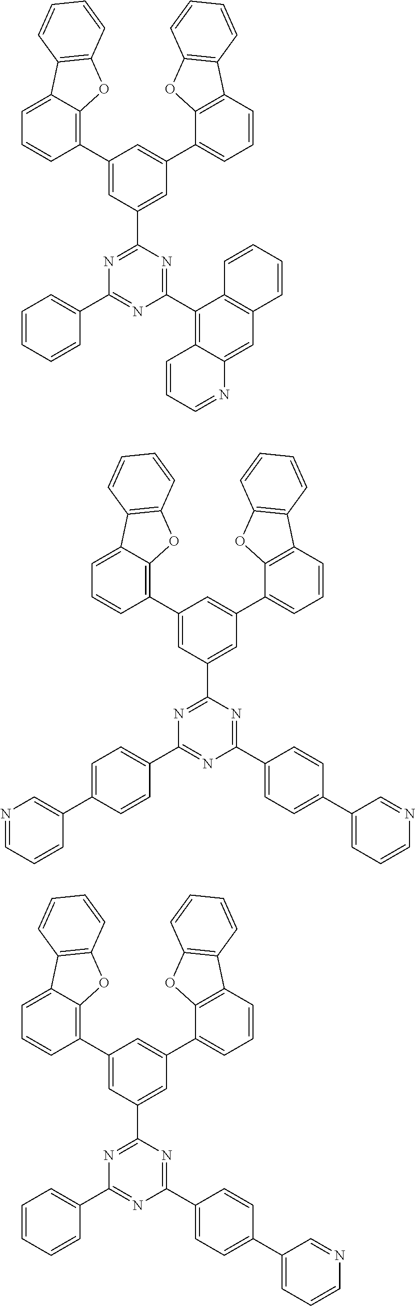

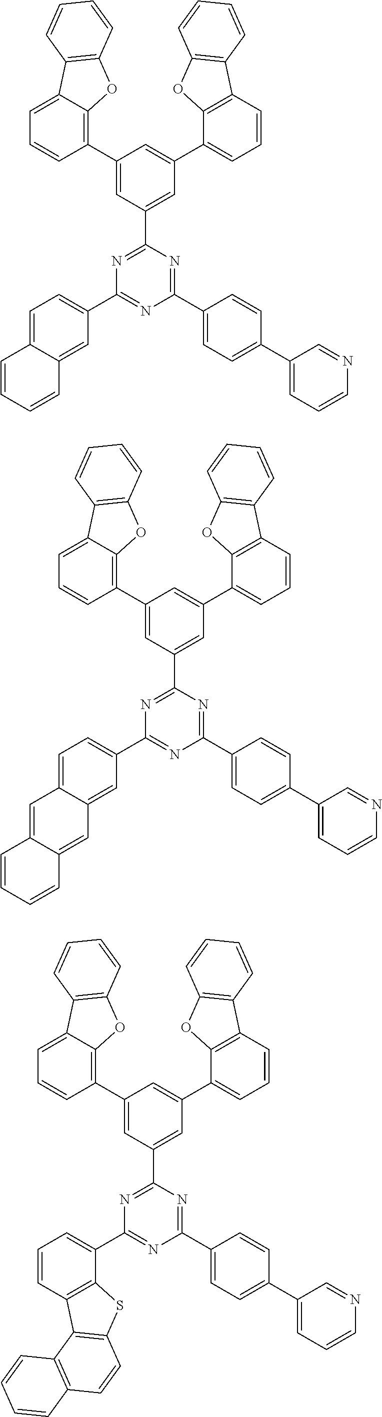 [SMILES: c1cncc(-c2ccc(-c3nc(-c4cc(-c5cccc6c5oc5ccccc56)cc(-c5cccc6c5oc5ccccc56)c4)nc(-c4ccc5ccccc5c4)n3)cc2)c1 ; c1cncc(-c2ccc(-c3nc(-c4cc(-c5cccc6c5oc5ccccc56)cc(-c5cccc6c5oc5ccccc56)c4)nc(-c4ccc5cc6ccccc6cc5c4)n3)cc2)c1 ; c1cncc(-c2ccc(-c3nc(-c4cc(-c5cccc6c5oc5ccccc56)cc(-c5cccc6c5oc5ccccc56)c4)nc(-c4cccc5c4sc4ccc6ccccc6c45)n3)cc2)c1]

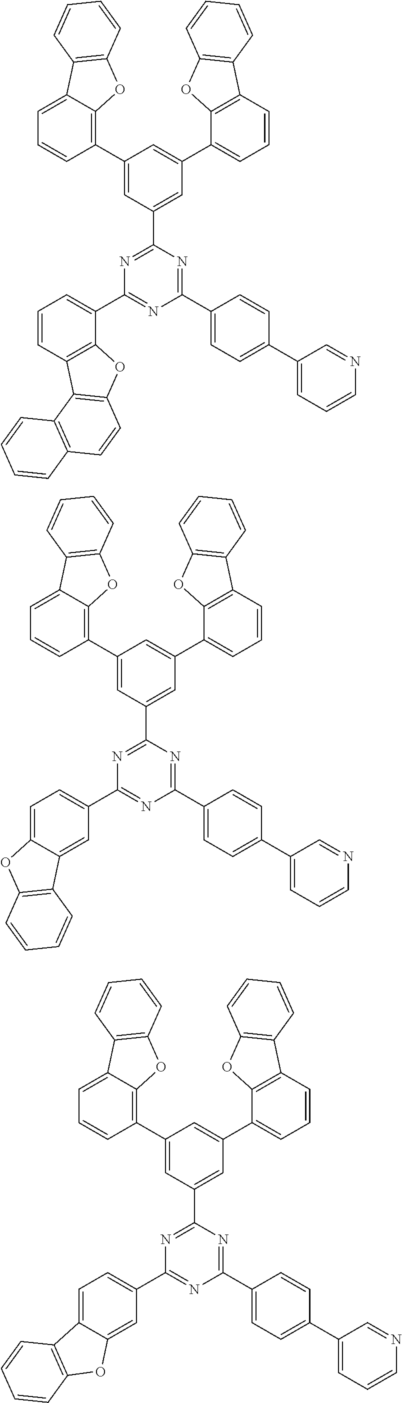

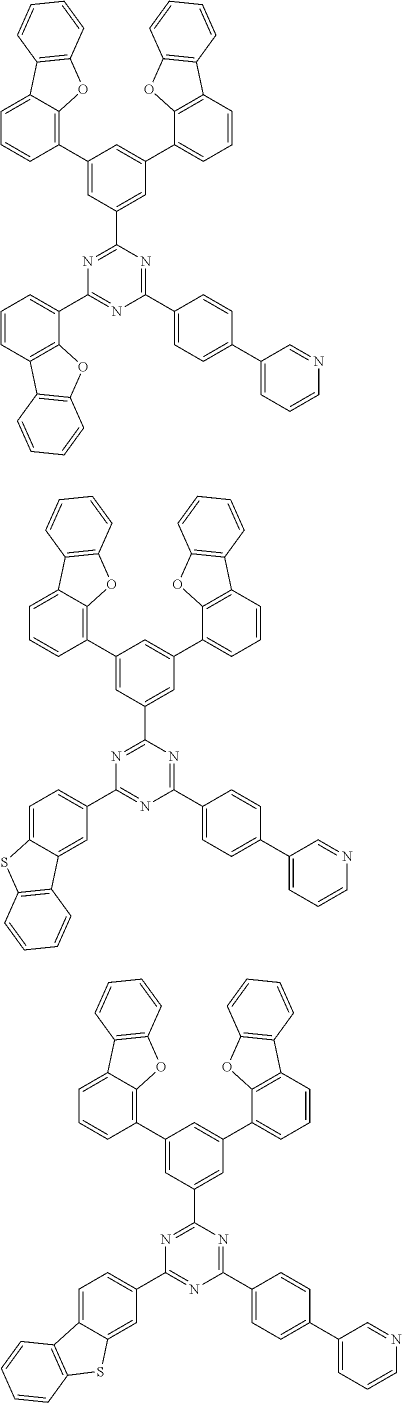

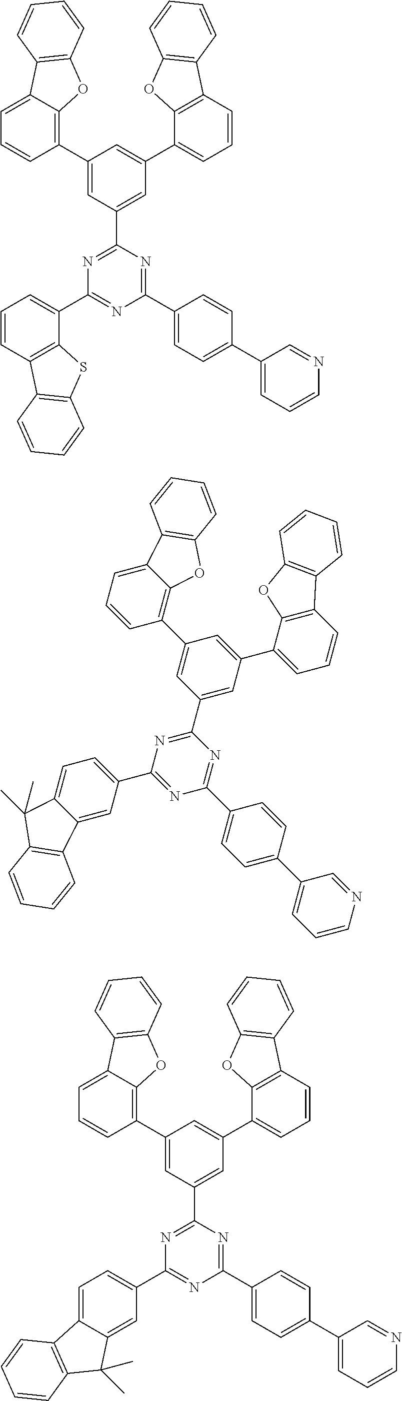 [SMILES: c1cncc(-c2ccc(-c3nc(-c4cc(-c5cccc6c5oc5ccccc56)cc(-c5cccc6c5oc5ccccc56)c4)nc(-c4cccc5c4sc4ccccc45)n3)cc2)c1 ; CC1(C)c2ccccc2-c2cc(-c3nc(-c4ccc(-c5cccnc5)cc4)nc(-c4cc(-c5cccc6c5oc5ccccc56)cc(-c5cccc6c5oc5ccccc56)c4)n3)ccc21 ; CC1(C)c2ccccc2-c2ccc(-c3nc(-c4ccc(-c5cccnc5)cc4)nc(-c4cc(-c5cccc6c5oc5ccccc56)cc(-c5cccc6c5oc5ccccc56)c4)n3)cc21]

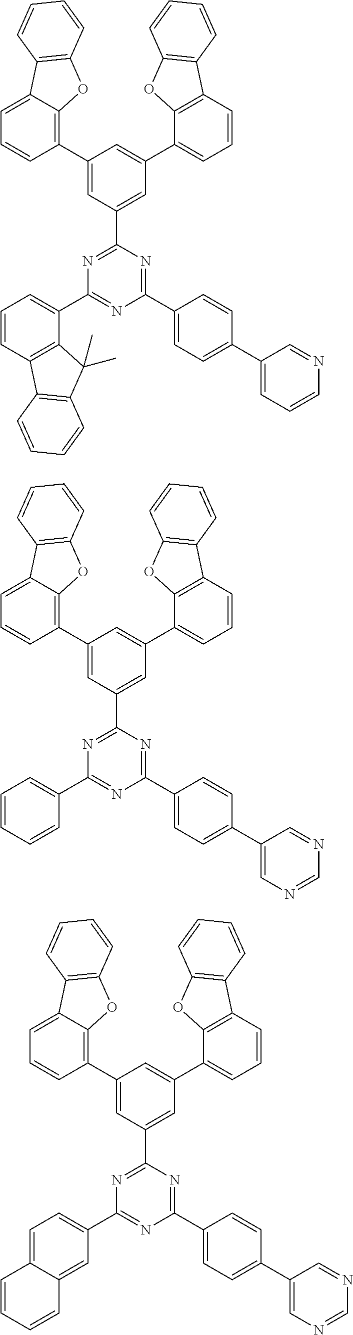

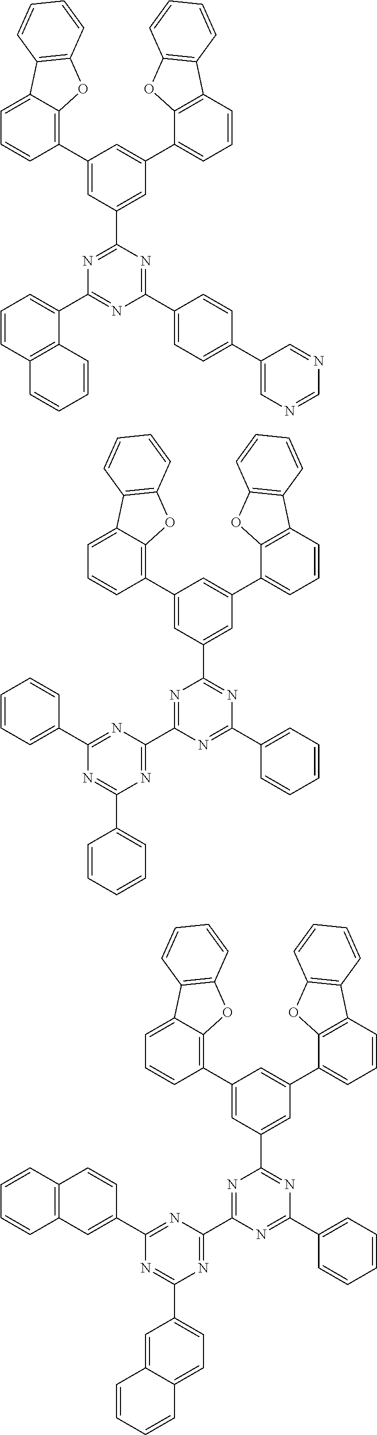

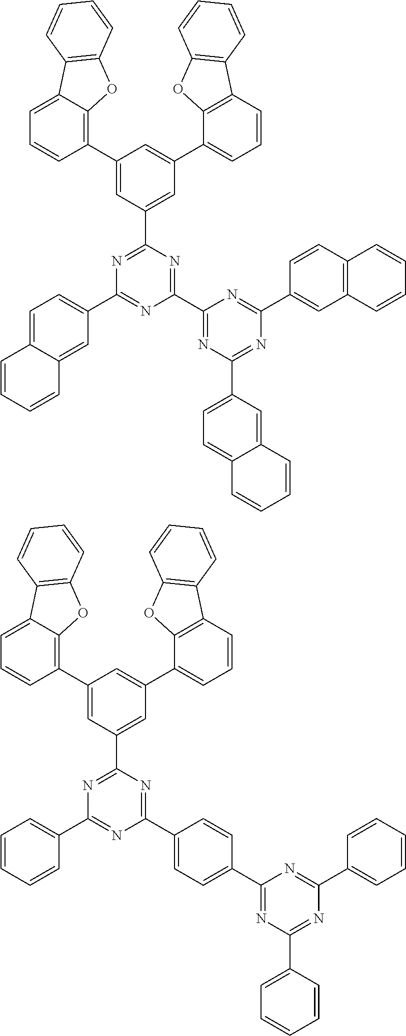

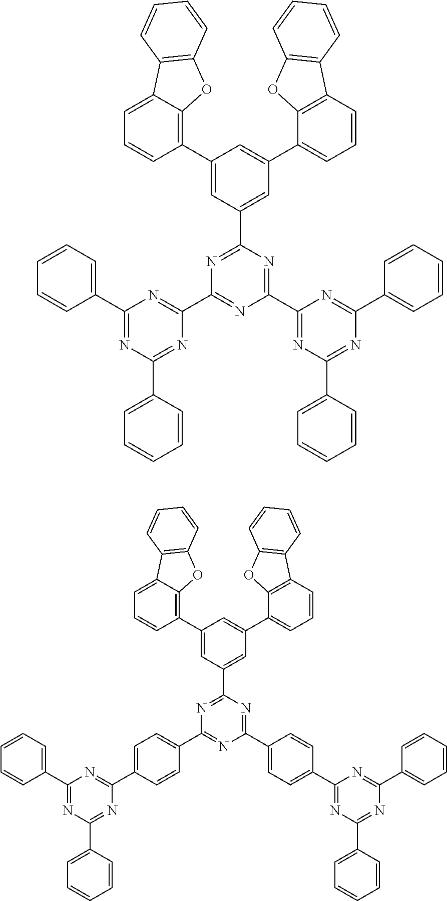 [SMILES: c1ccc(-c2nc(-c3ccccc3)nc(-c3nc(-c4cc(-c5cccc6c5oc5ccccc56)cc(-c5cccc6c5oc5ccccc56)c4)nc(-c4nc(-c5ccccc5)nc(-c5ccccc5)n4)n3)n2)cc1 ; c1ccc(-c2nc(-c3ccccc3)nc(-c3ccc(-c4nc(-c5ccc(-c6nc(-c7ccccc7)nc(-c7ccccc7)n6)cc5)nc(-c5cc(-c6cccc7c6oc6ccccc67)cc(-c6cccc7c6oc6ccccc67)c5)n4)cc3)n2)cc1]

In an embodiment, the organic compound according to the present disclosure is a small molecule material.

The term “small molecule” as defined herein refers to a molecule that is not a polymer, oligomer, dendrimer, or blend. In particular, there are no repeating structures in the small molecule. The molecular weight of the small molecule is less than or equal to 3000 g/mol, further less than or equal to 2000 g/mol, and still further less than or equal to 1500 g/mol.

Polymer includes homopolymer, copolymer, and block copolymer. In addition, in the present disclosure, the polymer also includes dendrimer. The synthesis and application of dendrimers can be found in Dendrimers and Dendrons, Wiley-VCH Verlag GmbH & Co. KGaA, 2002, Ed. George R. Newkome, Charles N. Moorefield, Fritz Vogtle.

Conjugated polymer is a polymer whose backbone is primarily formed by the sp2 hybrid orbital of C atoms. Taking polyacetylene and poly (phenylene vinylene) as famous examples, the C atoms on the backbones of which may also be substituted by other non-C atoms, and which are still considered to be conjugated polymers when the sp2 hybridization on the backbones is interrupted by some natural defects. In addition, the conjugated polymer in the present disclosure may also comprise aryl amine, aryl phosphine and other heteroaromatics, organometallic complexes, and the like on the backbone.

The present disclosure also relates to a polymer comprising a repeating unit in which comprises a structural unit as represented by the general formula (1). In certain embodiments, the polymer is a non-conjugated polymer, wherein the structural unit as represented by the general formula (1) is on the side chain. In another embodiment, the polymer is a conjugated polymer.

The present disclosure further relates to a mixture comprising at least one of at least one of the organic compounds or the polymers according to the present disclosure, and at least another organic functional material.

The organic functional material described herein comprises: hole (also called electronic hole) injection or transport materials (HIM/HTM), hole blocking materials (HBM), electron injection or transport materials (EIM/ETM), electron blocking materials (EBM), organic matrix materials (Host), singlet emitters (fluorescent emitters), thermally activated delayed fluorescent materials (TADF), triplet emitters (phosphorescent emitters), especially light-emitting organometallic complexes and organic dyes. Various organic functional materials are described in detail, for example, in WO2010135519A1, US20090134784A1 and WO 2011110277A1, and the entire contents of these three patent documents are hereby incorporated herein by reference.

The organic functional materials may be small molecules or polymer materials.

In certain embodiments, in the mixture according to the present disclosure, the content of the compound ranges from 50 wt % to 99.9 wt %, further, ranges from 60 wt % to 97 wt %, still further, ranges from 60 wt % to 95 wt %, even further, ranges from 70 wt % to 90 wt %.

In an embodiment, the mixture according to the present disclosure comprises a compound or a polymer according to the present disclosure and a fluorescent emitting material (a singlet emitter).

In another embodiment, the mixture according to the present disclosure comprises a compound or a polymer according to the present disclosure and a thermally activated delayed fluorescent material (TADF).

In another embodiment, the mixture according to the present disclosure comprises a compound or a polymer according to the present disclosure, a fluorescent emitting material and a TADF material.

The fluorescent emitting material or singlet emitter (fluorescent emitting material), and the TADF material are described in more detail below (but not limited thereto).

1. Singlet Emitter

The singlet emitter tends to have a longer conjugated π-electron system. To date, there have been many examples, such as, styrylamine and derivatives thereof disclosed in JP2913116B and WO2001021729A1, and indenofluorene and derivatives thereof disclosed in WO2008/006449 and WO2007/140847.

In an embodiment, the singlet emitter can be selected from the group consisting of mono-styrylamine, di-styrylamine, tri-styrylamine, tetra-styrylamine, styryl phosphine, styryl ether, and arylamine.

A mono-styrylamine is a compound comprising an unsubstituted or substituted styryl group and at least one amine, especially an aromatic amine. A di-styrylamine is a compound comprising two unsubstituted or substituted styryl groups and at least one amine, especially an aromatic amine. A tri-styrylamine is a compound comprising three unsubstituted or substituted styryl groups and at least one amine, especially an aromatic amine. A tetra-styrylamine is a compound comprising four unsubstituted or substituted styryl groups and at least one amine, especially an aromatic amine. In an embodiment, a styrene is stilbene, which may be further substituted. The definitions of the corresponding phosphines and ethers are similar to those of amines. An aryl amine or aromatic amine refers to a compound comprising three unsubstituted or substituted aromatic ring or heterocyclic systems directly coupled to nitrogen. In an embodiment, at least one of such aromatic or heterocyclic ring systems is selected from fused ring system, and especially has at least 14 aromatic ring atoms. Wherein, suitable examples are aromatic anthramine, aromatic anthradiamine, aromatic pyrenamine, aromatic pyrenediamine, aromatic chryseneamine or aromatic chrysenediamine. An aromatic anthramine refers to a compound in which a diarylamino group is directly coupled to anthracene, especially at position 9. An aromatic anthradiamine refers to a compound in which two diarylamino groups are directly coupled to anthracene, especially at positions 9, 10. Aromatic pyrenamine, aromatic pyrenediamine, aromatic chryseneamine and aromatic chrysenediamine are similarly defined, wherein the diarylarylamino group is particularly coupled to position 1 or 1, 6 of pyrene.

The examples of singlet emitters based on vinylamine and arylamine are also found in the following patent documents: WO 2006/000388, WO 2006/058737, WO 2006/000389, WO 2007/065549, WO 2007/115610, U.S. Pat. No. 7,250,532 B2, DE 102005058557 A1, CN 1583691 A, JP 08053397 A, U.S. Pat. No. 6,251,531 B1, US 2006/210830 A, EP 1 957 606 A1 and US 2008/0113101 A1, the entirety of the patent documents listed above are hereby incorporated herein by reference.

Examples of singlet emitters based on stilbene and derivatives thereof can be found in U.S. Pat. No. 5,121,029.

Further, singlet emitters may be selected from indenofluorene-amine and indenofluorene-diamine, as disclosed in WO 2006/122630, benzoindenofluorene-amines and benzoindenofluorene-diamine, as disclosed in WO 2008/006449, dibenzoindenofluorene-amine and dibenzofluorenone-diamine, as disclosed in WO2007/140847.

Other materials that may be used as singlet emitters are polycyclic aromatic hydrocarbon compounds, particularly the derivatives of the following compounds: anthracene such as 9,10-di(2-naphthanthracene), naphthalene, tetracene, xanthene, phenanthrene, pyrene (such as 2,5,8,11-tetra-t-butylperylene), indenopyrene, phenylene (such as 4,4′-bis (9-ethyl-3-carbazovinylene)-1,1′-biphenyl), periflanthene, decacyclene, coronene, fluorene, spirobifluorene, arylpyrene (e.g., US20060222886), arylenevinylene (e.g., U.S. Pat. Nos. 5,121,029, 5,130,603), cyclopentadiene such as tetraphenylcyclopentadiene, rubrene, coumarine, rhodamine, quinacridone, pyrane such as 4(dicyanomethylene)-6-(4-dimethylaminostyryl-2-methyl)-4H-pyrane (DCM), thiapyran, bis (azinyl) imine-boron compound (US 2007/0092753 A1), bis (azinyl) methene compound, carbostyryl compound, oxazone, benzoxazole, benzothiazole, benzimidazole and diketopyrrolopyrrole. Examples of materials of some singlet emitters can be found in the following patent documents: US 20070252517 A1, U.S. Pat. Nos. 4,769,292, 6,020,078, US 2007/0252517 A1, US 2007/0252517 A1. The entirety of the patent documents listed above is hereby incorporated herein by reference.

Some suitable examples of singlet emitters are listed in table below:

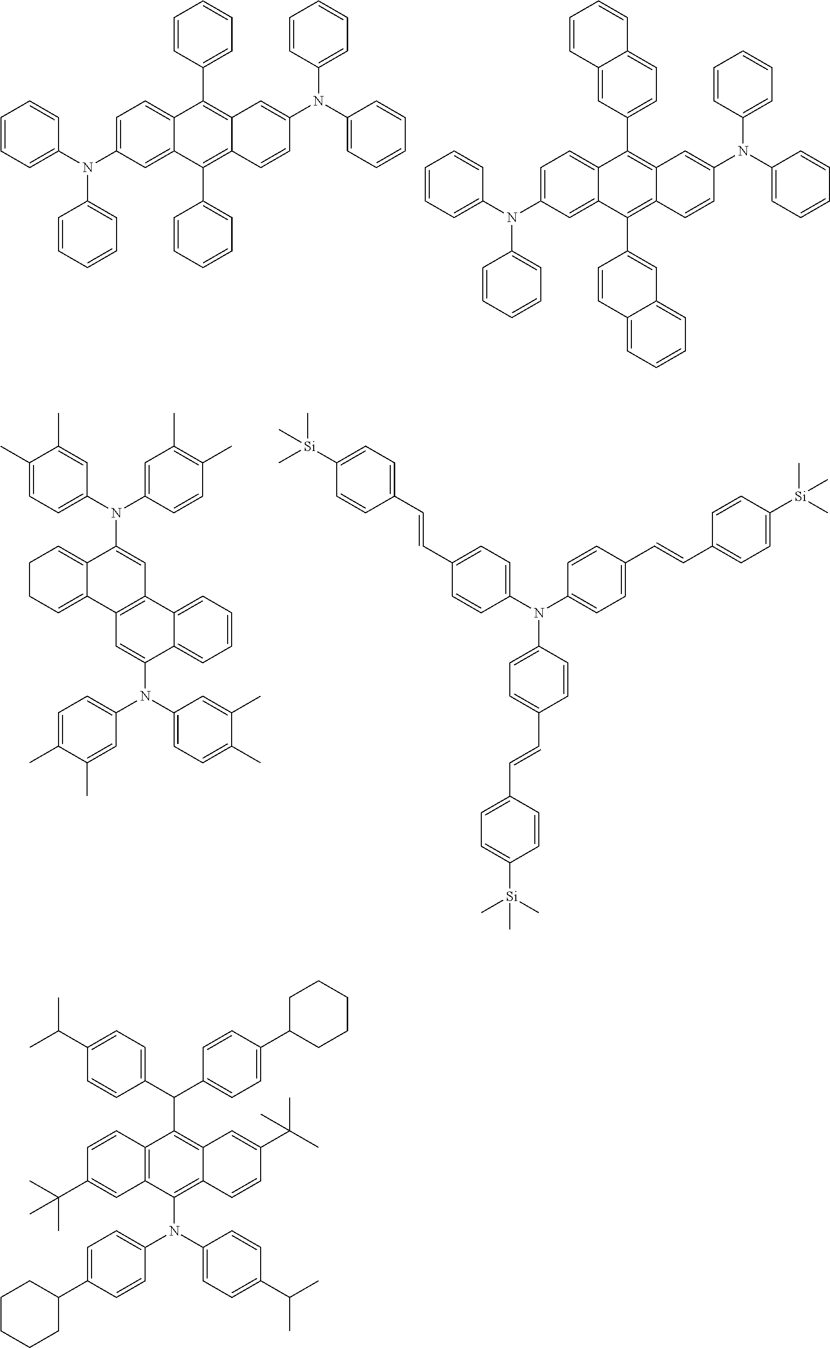

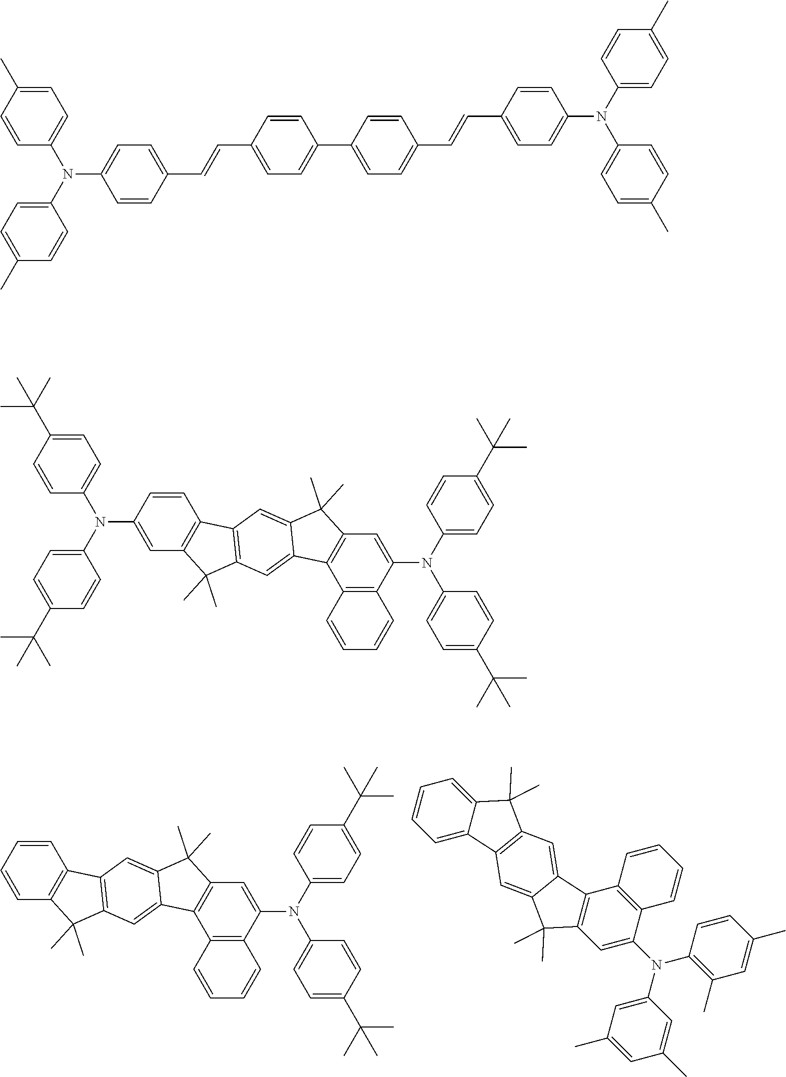

2. Thermally Activated Delayed Fluorescent Materials (TADF):

Traditional organic fluorescent materials can only emit light using 25% singlet exciton formed by electrical excitation and the devices have relatively low internal quantum efficiency (up to 25%). The phosphorescent material enhances the intersystem crossing due to the strong spin-orbit coupling of the heavy atom center, the singlet exciton and the triplet exciton emission formed by the electric excitation can be effectively utilized to emit light, so that the internal quantum efficiency of the devices can reach 100%. However, the phosphor materials are expensive, the material stability is poor, and the device efficiency roll-off is a serious problem, which limit its application in OLED. The thermally activated delayed fluorescent material is the third generation of organic light-emitting material developed after the organic fluorescent material and the organic phosphorescent material. Such materials generally have a small singlet-triplet excited state energy level difference (ΔEst), and triplet excitons can be converted to singlet excitons by intersystem crossing to emit light, which can fully use singlet excitons and triplet excitons formed under electric excitation. The devices can achieve 100% internal quantum efficiency.

The TADF material needs to have a smaller singlet-triplet excited state energy level difference, typically ΔEst <0.3 eV, further ΔEst <0.2 eV, still further ΔEst <0.1 eV, and even further ΔEst <0.05 eV. In an embodiment, TADF has better fluorescence quantum efficiency. Some TADF materials can be found in the following patent documents: CN103483332(A), TW201309696(A), TW201309778(A), TW201343874(A), TW201350558(A), US20120217869(A1), WO2013133359(A1), WO2013154064(A1), Adachi, et. al. Adv. Mater., 21, 2009, 4802; Adachi, et. al. Appl. Phys. Lett., 98, 2011, 083302; Adachi, et. al. Appl. Phys. Lett., 101, 2012, 093306; Adachi, et. al. Chem. Commun., 48, 2012, 11392; Adachi, et. al. Nature Photonics, 6, 2012, 253; Adachi, et. al. Nature, 492, 2012, 234; Adachi, et. al. J. Am. Chem. Soc, 134, 2012, 14706; Adachi, et. al. Angew. Chem. Int. Ed, 51, 2012, 11311; Adachi, et. al. Chem. Commun., 48, 2012, 9580; Adachi, et. al. Chem. Commun., 48, 2013, 10385; Adachi, et. al. Adv. Mater., 25, 2013, 3319; Adachi, et. al. Adv. Mater., 25, 2013, 3707; Adachi, et. al. Chem. Mater., 25, 2013, 3038; Adachi, et. al. Chem. Mater., 25, 2013, 3766; Adachi, et. al. J. Mater. Chem. C., 1, 2013, 4599; Adachi, et. al. J. Phys. Chem. A., 117, 2013, 5607, the contents of the above-listed patents or article documents are hereby incorporated herein by reference in their entirety.

Some suitable examples of TADF light-emitting materials are listed in the table below:

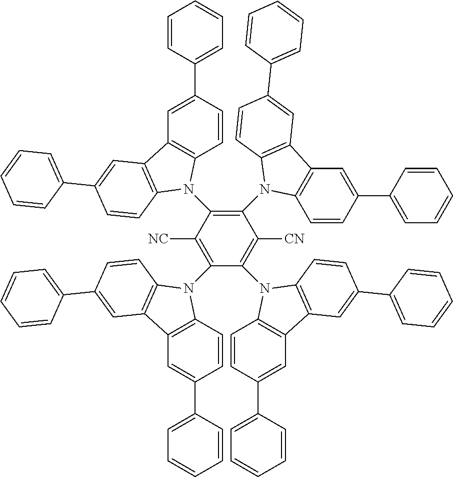 [SMILES: N#Cc1c(-n2c3ccc(-c4ccccc4)cc3c3cc(-c4ccccc4)ccc32)c(-n2c3ccc(-c4ccccc4)cc3c3cc(-c4ccccc4)ccc32)c(C#N)c(-n2c3ccc(-c4ccccc4)cc3c3cc(-c4ccccc4)ccc32)c1-n1c2ccc(-c3ccccc3)cc2c2cc(-c3ccccc3)ccc21]

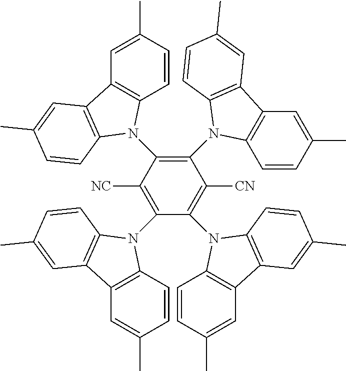

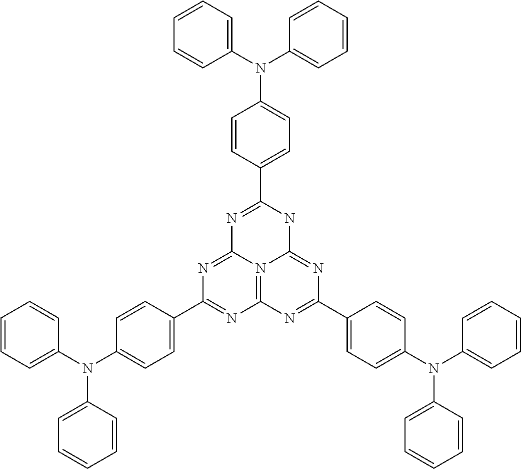

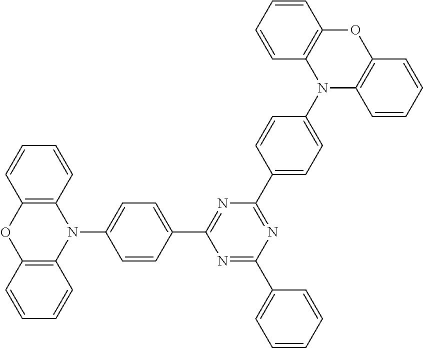 [SMILES: c1ccc(-c2nc(-c3ccc(N4c5ccccc5Oc5ccccc54)cc3)nc(-c3ccc(N4c5ccccc5Oc5ccccc54)cc3)n2)cc1]

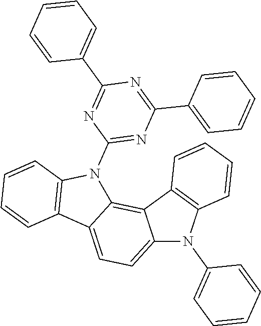 [SMILES: c1ccc(-c2nc(-c3ccccc3)nc(-n3c4ccccc4c4ccc5c(c6ccccc6n5-c5ccccc5)c43)n2)cc1]

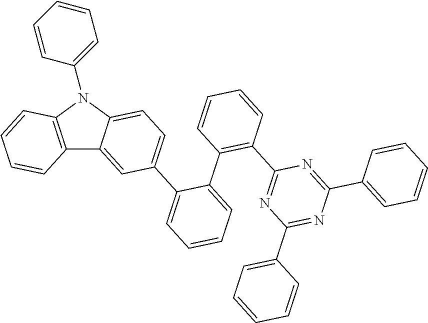 [SMILES: c1ccc(-c2nc(-c3ccccc3)nc(-c3ccccc3-c3ccccc3-c3ccc4c(c3)c3ccccc3n4-c3ccccc3)n2)cc1]

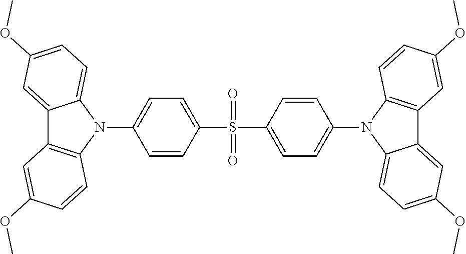 [SMILES: COc1ccc2c(c1)c1cc(OC)ccc1n2-c1ccc(S(=O)(=O)c2ccc(-n3c4ccc(OC)cc4c4cc(OC)ccc43)cc2)cc1]

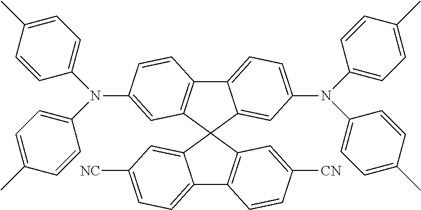

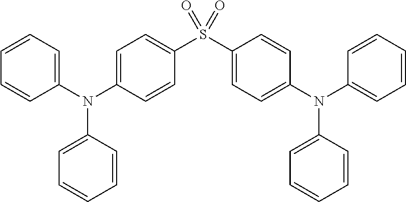 [SMILES: O=S(=O)(c1ccc(N(c2ccccc2)c2ccccc2)cc1)c1ccc(N(c2ccccc2)c2ccccc2)cc1]

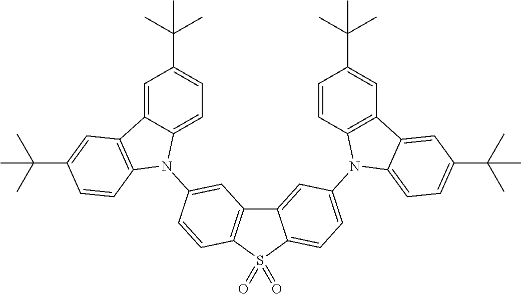 [SMILES: CC(C)(C)c1ccc2c(c1)c1cc(C(C)(C)C)ccc1n2-c1ccc2c(c1)-c1cc(-n3c4ccc(C(C)(C)C)cc4c4cc(C(C)(C)C)ccc43)ccc1S2(=O)=O]

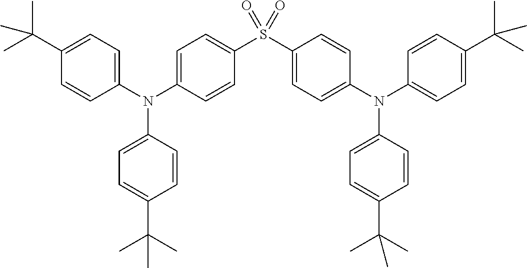 [SMILES: CC(C)(C)c1ccc(N(c2ccc(C(C)(C)C)cc2)c2ccc(S(=O)(=O)c3ccc(N(c4ccc(C(C)(C)C)cc4)c4ccc(C(C)(C)C)cc4)cc3)cc2)cc1]

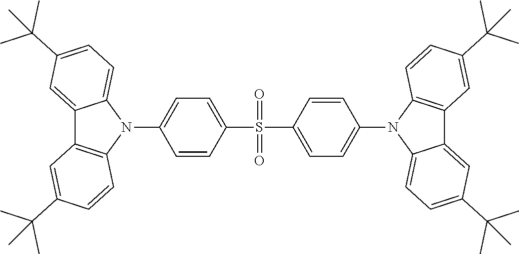 [SMILES: CC(C)(C)c1ccc2c(c1)c1cc(C(C)(C)C)ccc1n2-c1ccc(S(=O)(=O)c2ccc(-n3c4ccc(C(C)(C)C)cc4c4cc(C(C)(C)C)ccc43)cc2)cc1]

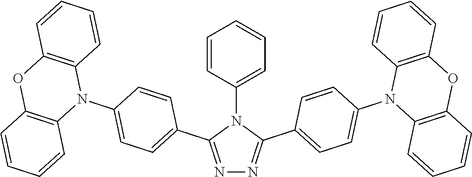 [SMILES: c1ccc(-n2c(-c3ccc(N4c5ccccc5Oc5ccccc54)cc3)nnc2-c2ccc(N3c4ccccc4Oc4ccccc43)cc2)cc1]

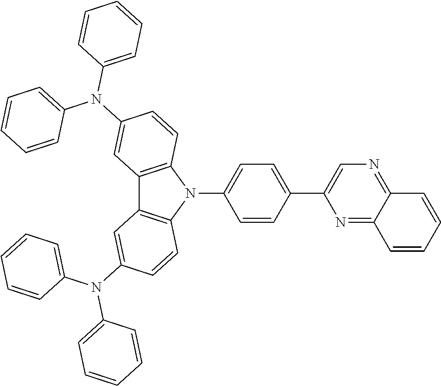 [SMILES: c1ccc(N(c2ccccc2)c2ccc3c(c2)c2cc(N(c4ccccc4)c4ccccc4)ccc2n3-c2ccc(-c3cnc4ccccc4n3)cc2)cc1]

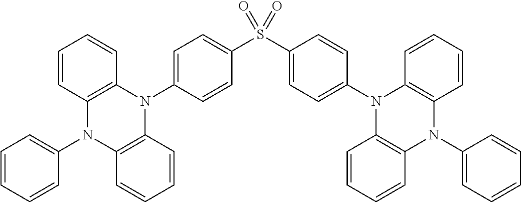 [SMILES: O=S(=O)(c1ccc(N2c3ccccc3N(c3ccccc3)c3ccccc32)cc1)c1ccc(N2c3ccccc3N(c3ccccc3)c3ccccc32)cc1]

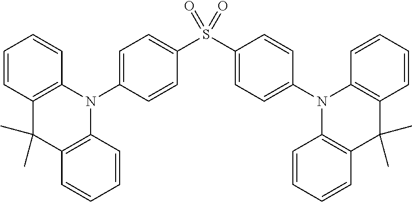

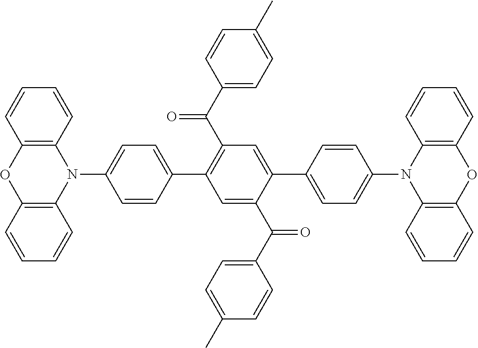 [SMILES: Cc1ccc(C(=O)c2cc(-c3ccc(N4c5ccccc5Oc5ccccc54)cc3)c(C(=O)c3ccc(C)cc3)cc2-c2ccc(N3c4ccccc4Oc4ccccc43)cc2)cc1]

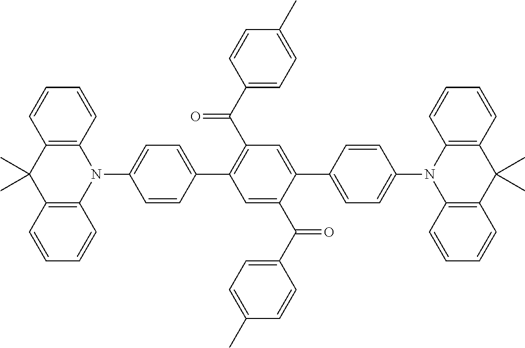

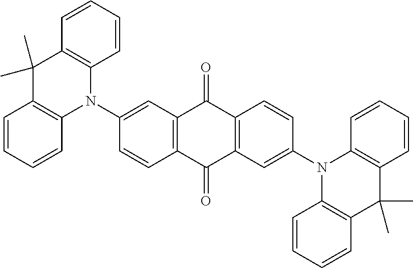

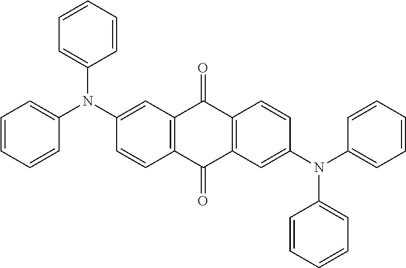 [SMILES: O=C1c2ccc(N(c3ccccc3)c3ccccc3)cc2C(=O)c2ccc(N(c3ccccc3)c3ccccc3)cc21]

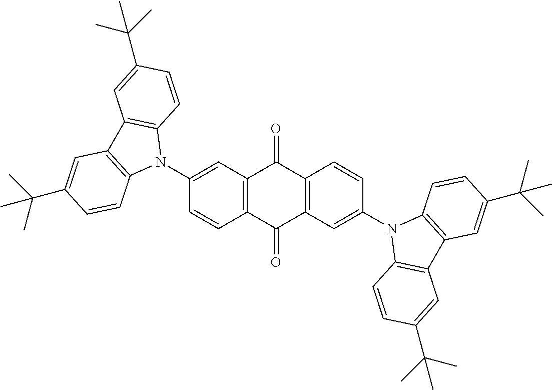 [SMILES: CC(C)(C)c1ccc2c(c1)c1cc(C(C)(C)C)ccc1n2-c1ccc2c(c1)C(=O)c1ccc(-n3c4ccc(C(C)(C)C)cc4c4cc(C(C)(C)C)ccc43)cc1C2=O]

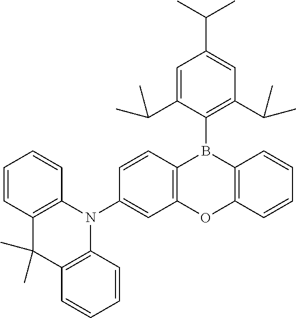 [SMILES: CC(C)c1cc(C(C)C)c(B2c3ccccc3Oc3cc(N4c5ccccc5C(C)(C)c5ccccc54)ccc32)c(C(C)C)c1]

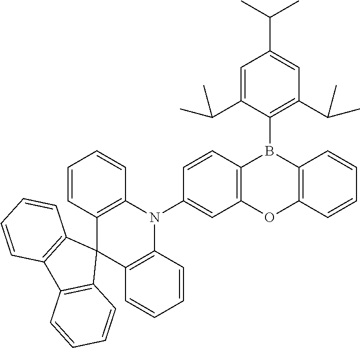 [SMILES: CC(C)c1cc(C(C)C)c(B2c3ccccc3Oc3cc(N4c5ccccc5C5(c6ccccc6-c6ccccc65)c5ccccc54)ccc32)c(C(C)C)c1]

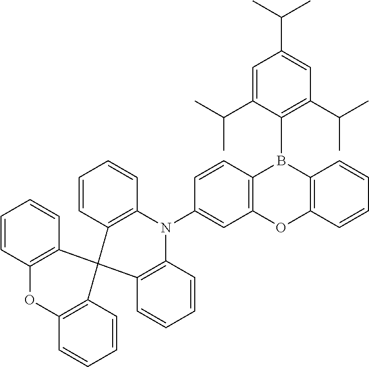 [SMILES: CC(C)c1cc(C(C)C)c(B2c3ccccc3Oc3cc(N4c5ccccc5C5(c6ccccc6Oc6ccccc65)c5ccccc54)ccc32)c(C(C)C)c1]

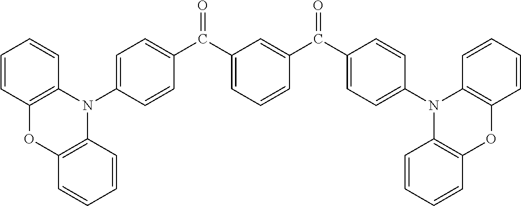 [SMILES: O=C(c1ccc(N2c3ccccc3Oc3ccccc32)cc1)c1cccc(C(=O)c2ccc(N3c4ccccc4Oc4ccccc43)cc2)c1]

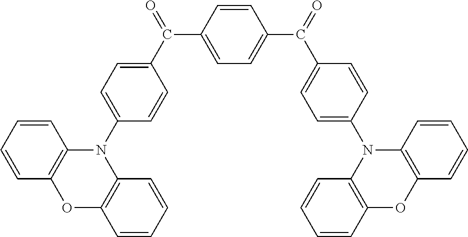 [SMILES: O=C(c1ccc(C(=O)c2ccc(N3c4ccccc4Oc4ccccc43)cc2)cc1)c1ccc(N2c3ccccc3Oc3ccccc32)cc1]

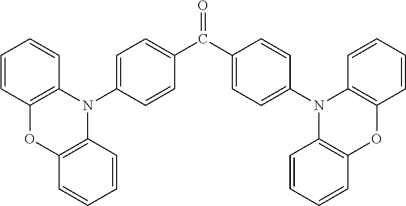 [SMILES: O=C(c1ccc(N2c3ccccc3Oc3ccccc32)cc1)c1ccc(N2c3ccccc3Oc3ccccc32)cc1]

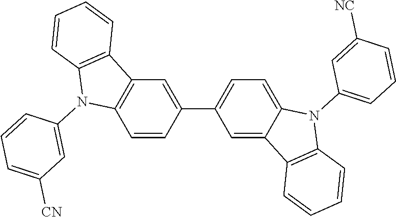 [SMILES: N#Cc1cccc(-n2c3ccccc3c3cc(-c4ccc5c(c4)c4ccccc4n5-c4cccc(C#N)c4)ccc32)c1]

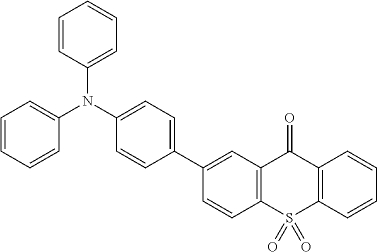 [SMILES: O=C1c2ccccc2S(=O)(=O)c2ccc(-c3ccc(N(c4ccccc4)c4ccccc4)cc3)cc21]

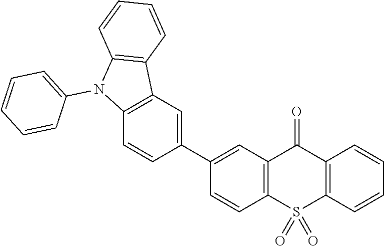 [SMILES: O=C1c2ccccc2S(=O)(=O)c2ccc(-c3ccc4c(c3)c3ccccc3n4-c3ccccc3)cc21]

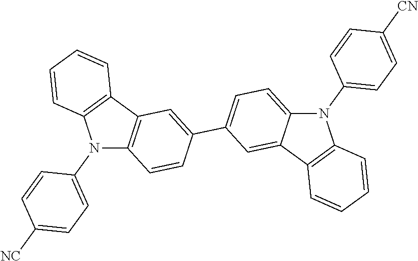 [SMILES: N#Cc1ccc(-n2c3ccccc3c3cc(-c4ccc5c(c4)c4ccccc4n5-c4ccc(C#N)cc4)ccc32)cc1]

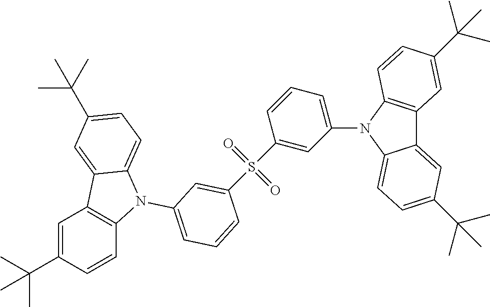 [SMILES: CC(C)(C)c1ccc2c(c1)c1cc(C(C)(C)C)ccc1n2-c1cccc(S(=O)(=O)c2cccc(-n3c4ccc(C(C)(C)C)cc4c4cc(C(C)(C)C)ccc43)c2)c1]

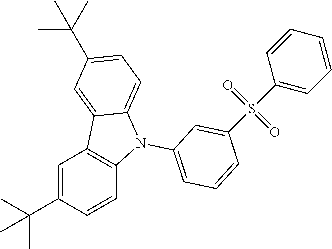 [SMILES: CC(C)(C)c1ccc2c(c1)c1cc(C(C)(C)C)ccc1n2-c1cccc(S(=O)(=O)c2ccccc2)c1]

The publications of organic functional materials mentioned above are incorporated herein by reference for the purpose of disclosure.

In an embodiment, the compound according to the present disclosure is used for evaporated OLED devices. For this purpose, the compound according to the present disclosure has a molecular weight ≤1000 g/mol, further ≤900 g/mol, still further ≤850 g/mol, even further ≤800 g/mol, even further ≤1100 g/mol.

Another purpose of the present disclosure is to provide material solutions for printing OLED.

In certain embodiments, the compound according to the present disclosure has a molecular weight ≥700 g/mol, further ≥800 g/mol, still further ≥900 g/mol, still further ≥1000 g/mol, even further ≥1100 g/mol.

In other embodiments, the compound according to the present disclosure has solubility in toluene ≥210 mg/mL, further ≥215 mg/mL, and still further ≥220 mg/mL at 25° C.

The present disclosure further relates to a formulation or ink in which comprises a compound or a polymer or a mixture according to the present disclosure, and at least one organic solvent. The present disclosure further provides a thin film comprising the compound or the polymer according to the present disclosure prepared from a solution.

The viscosity and surface tension of ink are important parameters when the ink is used in the printing process. The suitable surface tension parameters of ink are suitable for a particular substrate and a particular printing method.

In an embodiment, the surface tension of the ink according to the present disclosure at working temperature or at 25° C. is in the range of about 19 dyne/cm to 50 dyne/cm, further in the range of 22 dyne/cm to 35 dyne/cm, and still further in the range of 25 dyne/cm to 33 dyne/cm.

In another embodiment, the viscosity of the ink according to the present disclosure at the working temperature or at 25° C. is in the range of about 1 cps to 100 cps, further in the range of 1 cps to 50 cps, still further in the range of 1.5 cps to 20 cps, and even further in the range of 4.0 cps to 20 cps. The formulation so formulated will be suitable for inkjet printing.

The viscosity can be adjusted by different methods, such as by the selection of appropriate solvent and the concentration of functional materials in the ink. The ink according to the present disclosure comprising the compound or polymer can facilitate the adjustment of the printing ink in an appropriate range according to the printing method used. In general, the weight ratio of the functional material contained in the formulation according to the present disclosure is in the range of 0.3 wt % to 30 wt %, further in the range of 0.5 wt % to 20 wt %, still further in the range of 0.5 wt % to 15 wt %, still further in the range of 0.5 wt % to 10 wt %, and even further in the range of 1 wt % to 5 wt %.

In some embodiments, according to the ink of the present disclosure, the at least one organic solvent is selected from aromatic or heteroaromatic based solvents, in particular from aromatic solvents or aromatic ketone solvents or aromatic ether solvents substituted by aliphatic chain/ring.

Examples suitable for solvents of the present disclosure include, but not limited to, aromatic or heteroaromatic based solvents: p-diisopropylbenzene, pentylbenzene, tetrahydronaphthalene, cyclohexyl benzene, chloronaphthalene, 1,4-dimethylnaphthalene, 3-isopropylbiphenyl, p-cymene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diisopropylbenzene, 1-methoxynaphthalene, cyclohexylbenzene, dimethylnaphthalene, 3-isopropylbiphenyl, p-cymene, 1-methylnaphthalene, 1,2,4-trichlorobenzene, 1,3-dipropoxybenzene, 4,4-difluorodiphenylmethane, 1,2-dimethoxy-4-(1-propenyl)benzene, diphenylmethane, 2-phenylpyridine, 3-phenylpyridine, N-methyldiphenylamine, 4-isopropylbiphenyl, α,α-dichlorodiphenylmethane, 4-(3-phenylpropyl)pyridine, benzylbenzoate, 1,1-di(3,4-dimethylphenyl)ethane, 2-isopropylnaphthalene, dibenzylether, and the like; solvents based on ketones: 1-tetralone, 2-tetralone, 2-(phenylepoxy)tetralone, 6-(methoxyl)tetralone, acetophenone, phenylacetone, benzophenone, and derivatives thereof, such as 4-methylacetophenone, 3-methylacetophenone, 2-methylacetophenone, 4-methylphenylacetone, 3-methylphenylacetone, 2-methylphenylacetone, isophorone, 2,6,8-trimethyl-4-nonanone, fenchone, 2-nonanone, 3-nonanone, 5-nonanone, 2-decanone, 2,5-hexanedione, phorone, 6-undecanone; aromatic ether solvents: 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylphenetole, 1,2,4-trimethoxybenzene, 4-(1-propenyl)-1,2-dimethoxybenzene, 1,3-dimethoxybenzene, glycidyl phenyl ether, dibenzyl ether, 4-tert-butylanisole, trans-p-propenylanisole, 1,2-dimethoxybenzene, 1-methoxynaphthalene, diphenyl ether, 2-phenoxymethyl ether, 2-phenoxytetrahydrofuran, ethyl-2-naphthyl ether, pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether; and ester solvents: alkyl octoate, alkyl sebacate, alkyl stearate, alkyl benzoate, alkyl phenylacetate, alkyl cinnamate, alkyl oxalate, alkyl maleate, alkyl lactone, alkyl oleate, and the like.

Further, according to the ink of the present disclosure, the at least one solvent can be selected from: aliphatic ketones, such as 2-nonanone, 3-nonanone, 5-nonanone, 2-decanone, 2,5-hexanedione, 2,6,8-trimethyl-4-nonanone, phorone, 6-undecanone, and the like, or aliphatic ethers, such as pentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, and the like.

In other embodiments, the printing ink further comprises another organic solvent. Examples of another organic solvent include, but not limited to, methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxy toluene, 1,1,1-trichloroethane, 1,1,2,2-tetrachloroethane, ethyl acetate, butyl acetate, dimethylformamide, dimethylacetamide, dimethyl sulfoxide, tetrahydronaphthalene, decalin, indene, and/or mixtures thereof.

In an embodiment, the formulation according to the present disclosure is a solution.

In another embodiment, the formulation according to the present disclosure is a suspension.

The present disclosure also relates to the use of the formulation as printing ink during the preparation of organic electronic devices, particularly by the preparation method of printing or coating.

Suitable printing or coating techniques include, but are not limited to, inkjet printing, nozzle printing, letterpress printing, screen printing, dip coating, spin coating, blade coating, roller printing, torsion roll printing, lithography, flexography, rotary printing, spraying, brushing or pad printing, nozzle printing, slot die coating, etc, preferably inkjet printing, slot die coating, nozzle printing and gravure printing.

The solution or suspension may additionally comprise one or more components such as surface-active compound, lubricant, wetting agent, dispersant, hydrophobic agent, binder, etc., for adjusting viscosity and film-forming performance, enhancing adhesion, and the like. For more information about printing technologies and relevant requirements thereof on related solutions, such as solvents and concentration, viscosity, etc., see Handbook of Print Media: Technologies and Production Methods, ISBN 3-540-67326-1, edited by Helmut Kipphan.

Based on the above compound, the present disclosure also provides an application of the compound or the polymer as described above in organic electronic devices. The organic electronic devices may be selected from, but not limited to, an organic light-emitting diode (OLED), an organic photovoltaic cell (OPV), an organic light-emitting electrochemical cell (OLEEC), an organic field effect transistor (OFET), an organic light-emitting field effect transistor, organic laser, an organic spintronic device, an organic sensor, and organic plasmon emitting diode, and the like, specially OLED. In an embodiment of the present disclosure, the organic compound is used in the electron transport layer, the electron injection layer or the light-emitting layer of the OLED devices.

The present disclosure further relates to an organic electronic device comprising at least one of the compounds or the polymers as described above. Generally, such organic electronic device comprises at least one cathode, one anode, and one functional layer located between the cathode and the anode, wherein the functional layer comprises at least one compounds or the polymers as described above. The organic electronic devices may be selected from, but not limited to, an organic light-emitting diode (OLED), an organic photovoltaic cell (OPV), an organic light-emitting electrochemical cell (OLEEC), an organic field effect transistor (OFET), an organic light-emitting field effect transistor, an organic laser, an organic spintronic device, an organic sensor, and an organic plasmon emitting diode.

In an embodiment, the organic electronic device is an electroluminescent device, in particular an OLED, comprising a substrate, an anode, a cathode, and at least one light-emitting layer located between the anode and the cathode, optionally may also comprise a hole transport layer or an electron transport layer. In an embodiment, the organic electronic device comprises an electron transport layer or an electron injection layer in which comprises the compound or the polymer according to the present disclosure. In another embodiment, the organic electronic device comprises a light-emitting layer in which comprises the compound or the polymer according to the present disclosure, further, the light-emitting layer comprises the compound or the polymer according to the present disclosure and at least one light-emitting material which may be selected from fluorescent emitter, or TADF material.

The device structure of the electroluminescent device is described below, but it is not limited thereto.

The substrate can be opaque or transparent. A transparent substrate can be used to fabricate a transparent light-emitting device. For example, see Bulovic et al. Nature 1996, 380, p 29 and Gu et al. Appl. Phys. Lett. 1996, 68, p 2606. The substrate may be rigid or elastic. The substrate may be plastic, metal, semiconductor wafer or glass. In one embodiment, the substrate has a smooth surface. The substrate without any surface defects is a particularly desirable choice. In an embodiment, the substrate is flexible and may be selected from polymer thin film or plastic which has the glass transition temperature T_{g} of greater than 150° C., further greater than 200° C., still further greater than 250° C., even further greater than 300° C. Suitable examples of the flexible substrate are poly(ethylene terephthalate) (PET) and polyethylene(2,6-naphthalate) (PEN).

The anode may comprise a conductive metal or a metal oxide, or a conductive polymer. The anode can inject holes easily into the hole injection layer (HIL), or the hole transport layer (HTL), or the light-emitting layer. In one embodiment, the absolute value of the difference between the work function of the anode and the HOMO energy level or the valence band energy level of the emitter in the light-emitting layer or of the p-type semiconductor material as the HIL or HTL or the electron blocking layer (EBL) is less than 0.5 eV, further less than 0.3 eV, even further less than 0.2 eV. Examples of anode materials include, but not limited to, Al, Cu, Au, Ag, Mg, Fe, Co, Ni, Mn, Pd, Pt, ITO, aluminum doped zinc oxide (AZO), and the like. Other suitable anode materials are known and may be easily selected by one of ordinary skilled in the art. The anode material may be deposited using any suitable technique, such as a suitable physical vapor deposition method, including radio frequency magnetron sputtering, vacuum thermal evaporation, e-beam, and the like. In some embodiments, the anode is patterned. Patterned ITO conductive substrates are commercially available and can be used to prepare the device according to the present disclosure.

The cathode may comprise a conductive metal or a metal oxide. The cathode can inject electrons easily into the EIL or ETL, or directly injected into the light-emitting layer. In one embodiment, the absolute value of the difference between the work function of the cathode and the LUMO energy level or the conduction band energy level of the emitter in the light-emitting layer or of the n type semiconductor material as the electron injection layer (EIL) or the electron transport layer (ETL) or the hole blocking layer (HBL) is less than 0.5 eV, further less than 0.3 eV, even further less than 0.2 eV. In principle, all materials that can be used as cathodes for OLED can be used as cathode materials for the devices of the present disclosure. Examples of the cathode materials include, but not limited to: Al, Au, Ag, Ca, Ba, Mg, LiF/Al, MgAg alloy, BaF₂/Al, Cu, Fe, Co, Ni, Mn, Pd, Pt, ITO, and the like. The cathode material may be deposited using any suitable technique, such as a suitable physical vapor deposition method, including radio frequency magnetron sputtering, vacuum thermal evaporation, e-beam, and the like.

OLED can also comprise other functional layers such as a hole injection layer (HIL), a hole transport layer (HTL), an electron blocking layer (EBL), an electron injection layer (EIL), an electron transport layer (ETL), and a hole blocking layer (HBL). Materials suitable for use in these functional layers are well known to those skilled in the art and are readily found in the literatures.

In an embodiment, in the light-emitting device according to the present disclosure, the electron transport layer or the electron injection layer thereof comprises the organic compound or the polymer of the present disclosure.

According to the light-emitting device of the present disclosure, the light-emitting wavelength thereof is between 300 and 1000 nm, further between 350 and 900 nm, and even further between 400 and 800 nm.

The present disclosure also relates to the application of the organic electronic device according to the present disclosure in various electronic equipments, comprising, but not limited to display equipment, lighting equipment, light source, sensor, and the like.

The present disclosure will be described below with reference to the preferred embodiments, but the present disclosure is not limited to the following embodiments. It should be understood that the appended claims summarize the scope of the present disclosure. Those skilled in the art should realize that certain changes to the embodiments of the present disclosure that are made under the guidance of the concept of the present disclosure will be covered by the spirit and scope of the claims of the present disclosure.

### DETAILED EXAMPLES

1. Examples are given according to the method of synthesizing the compound of the present disclosure, but the present disclosure is not limited to the following examples.

### Example 1: Synthesis of 2-(3,5-bis(3-fluoranthenyl)phenyl)4,6-bis(3-pyridyl)-1,3,5-triazine (1)

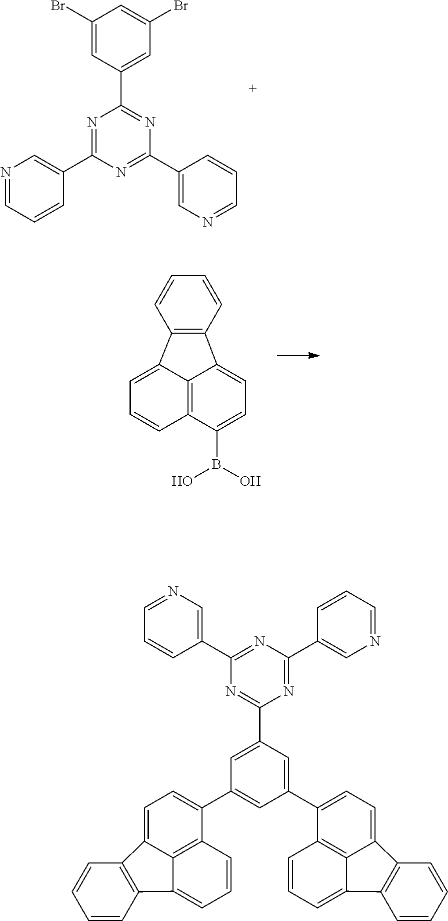 [SMILES: Brc1cc(Br)cc(-c2nc(-c3cccnc3)nc(-c3cccnc3)n2)c1 ; OB(O)c1ccc2c3c(cccc13)-c1ccccc1-2 ; c1cncc(-c2nc(-c3cccnc3)nc(-c3cc(-c4ccc5c6c(cccc46)-c4ccccc4-5)cc(-c4ccc5c6c(cccc46)-c4ccccc4-5)c3)n2)c1]

To a 500 mL double-necked round-bottom flask, 2-(4,5-dibromophenyl)-4,6-bis(3-pyridyl)-1,3,5-triazine (10 g, 21.32 mmol) and fluoranthene-3-boronic acid (11.54 g, 46.89 mmol) were added, followed by 250 mL of toluene as a solvent. The equipment was installed. Then, potassium carbonate (12.06 g, 87.41 mmol) was completely dissolved in 40 mL of water, and added into the round-bottom flask, and finally Pd(PPh₃)₄ (1.48 g, 1.28 mmol) was put into the flask. Air in the flask was evacuated by an oil pump, and nitrogen gas was introduced thereto. The solution was heated at a constant temperature under reflux for 12 hours, cooled, extracted, dried, concentrated, and purified to give 2-(3,5-bis(3-fluoranthenyl)phenyl)4,6-bis(3-pyridyl)-1,3,5-triazine with the yield of 65%.

### Example 2: Synthesis of 2-(3,5-difluoranthenylphenyl)-4-phenyl-6(4-(3-pyridyl)phenyl)-1,3,5-triazine (2)

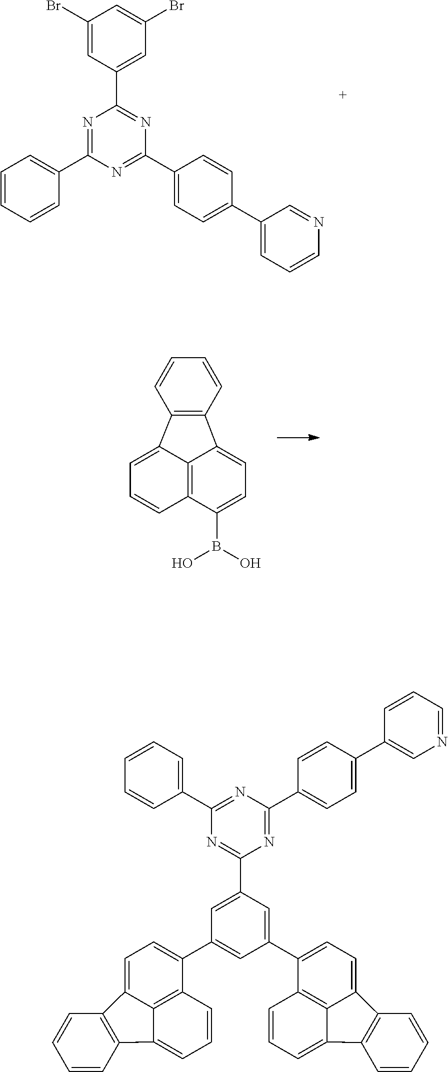 [SMILES: Brc1cc(Br)cc(-c2nc(-c3ccccc3)nc(-c3ccc(-c4cccnc4)cc3)n2)c1 ; OB(O)c1ccc2c3c(cccc13)-c1ccccc1-2 ; c1ccc(-c2nc(-c3ccc(-c4cccnc4)cc3)nc(-c3cc(-c4ccc5c6c(cccc46)-c4ccccc4-5)cc(-c4ccc5c6c(cccc46)-c4ccccc4-5)c3)n2)cc1]

To a 500 mL double-necked round-bottom flask, 2-(3,5-dibromophenyl)-4-phenyl-6(4-(3-pyridyl)phenyl)-1,3,5-triazine (10 g, 18.37 mmol) and fluoranthene-3-boronic acid (9.95 g, 40.4 mmol) were added, followed by 250 mL of toluene as a solvent. The equipment was installed. Then, potassium carbonate (10.4 g, 75.32 mmol) was completely dissolved in 35 mL of water, and added into the round-bottom flask, and finally Pd(PPh₃)₃ (1.27 g, 1.1 mmol) was put into the flask. Air in the flask was evacuated by an oil pump, and nitrogen gas was introduced thereto. The solution was heated at a constant temperature under reflux for 12 hours, cooled, extracted, dried, concentrated, and purified to give 2-(3,5-difluoranthenylphenyl)-4-phenyl-6(4-(3-pyridyl)phenyl)-1,3,5-triazine with the yield of 70%.

### Example 3: Synthesis of 2-(3,5-difluoranthenylphenyl)-4-phenyl-6(6-quinolyl)-1,3,5-triazine (3)

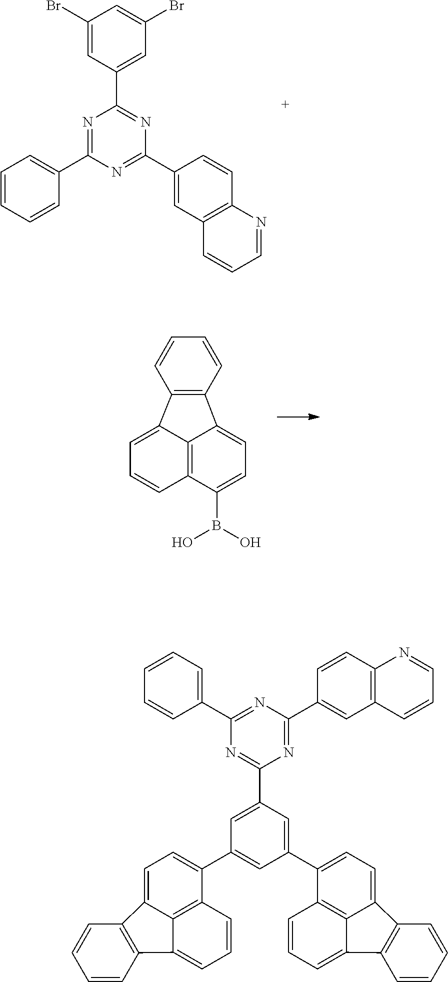 [SMILES: Brc1cc(Br)cc(-c2nc(-c3ccccc3)nc(-c3ccc4ncccc4c3)n2)c1 ; OB(O)c1ccc2c3c(cccc13)-c1ccccc1-2 ; c1ccc(-c2nc(-c3cc(-c4ccc5c6c(cccc46)-c4ccccc4-5)cc(-c4ccc5c6c(cccc46)-c4ccccc4-5)c3)nc(-c3ccc4ncccc4c3)n2)cc1]

To a 500 mL double-necked round-bottom flask, 2-(3,5-dibromophenyl)-4-phenyl-6(4-(6-quinolyl))-1,3,5-triazine (10 g, 19.3 mmol) and fluoranthene-3-boronic acid (10.45 g, 42.45 mmol) were added, followed by 250 mL of toluene as a solvent. The equipment was installed. Then, potassium carbonate (10.92 g, 79.13 mmol) was completely dissolved in 40 mL of water, and added into the round-bottom flask, and finally Pd(PPh₆)₃ (1.34 g, 1.16 mmol) was put into the flask. Air in the flask was evacuated by an oil pump, and nitrogen gas was introduced thereto. The solution was heated at a constant temperature under reflux for 12 hours, cooled, extracted, dried, concentrated, and purified to give 2-(3,5-difluoranthenylphenyl)-4-phenyl-6(6-quinolyl)-1,3,5-triazine with the yield of 75%.

### Example 4: Synthesis of 2-(3,5-dianthrylphenyl)-4-phenyl-6(4-(3-pyridyl)phenyl)-1,3,5-triazine (4)

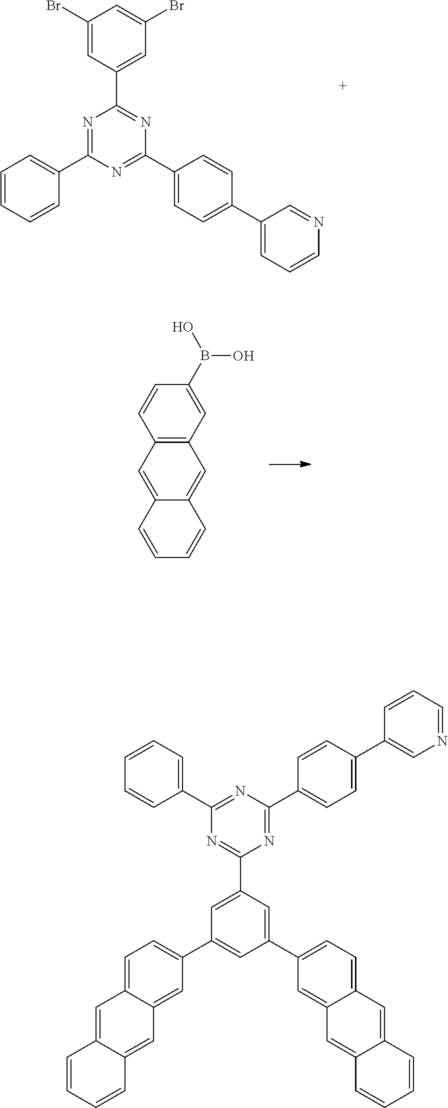 [SMILES: Brc1cc(Br)cc(-c2nc(-c3ccccc3)nc(-c3ccc(-c4cccnc4)cc3)n2)c1 ; OB(O)c1ccc2cc3ccccc3cc2c1 ; c1ccc(-c2nc(-c3ccc(-c4cccnc4)cc3)nc(-c3cc(-c4ccc5cc6ccccc6cc5c4)cc(-c4ccc5cc6ccccc6cc5c4)c3)n2)cc1]

To a 500 mL double-necked round-bottom flask, 2-(3,5-dibromophenyl)-4-phenyl-6(4-(3-pyridyl)phenyl)-1,3,5-triazine (10 g, 18.37 mmol) and anthraceneboronic acid (8.95 g, 40.42 mmol) were added, followed by 250 mL of toluene as a solvent. The equipment was installed. Then, potassium carbonate (10.4 g, 75.32 mmol) was completely dissolved in 35 mL of water, and added into the round-bottom flask, and finally Pd(PPh₃)₃ (1.27 g, 1.1 mmol) was put into the flask. Air in the flask was evacuated by an oil pump, and nitrogen gas was introduced thereto. The solution was heated at a constant temperature under reflux for 12 hours, cooled, extracted, dried, concentrated, and purified to give 2-(3,5-dianthrylphenyl)-4-phenyl-6(4-(3-pyridyl)phenyl)-1,3,5-triazine with the yield of 60%.

### Example 5: Synthesis of 2-(3,5-bis(3-fluoranthenyl)phenyl)4-phenyl-6-(3-pyridyl)-1,3,5-triazine (5)

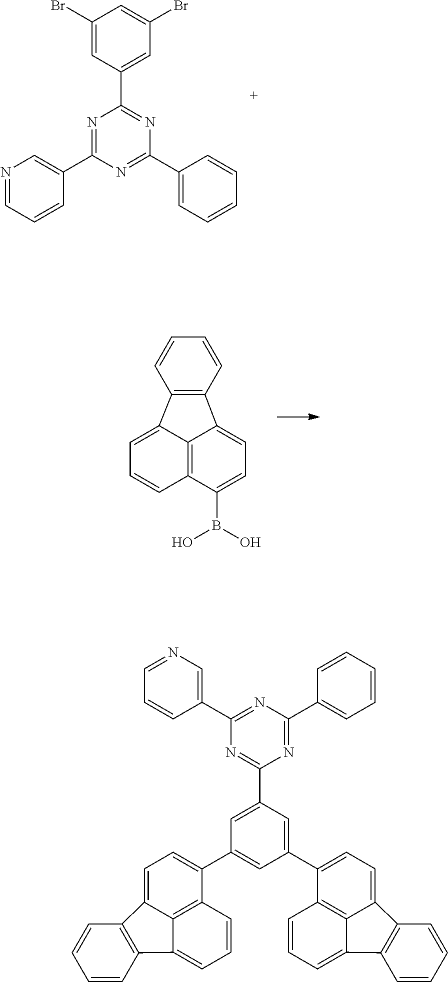 [SMILES: Brc1cc(Br)cc(-c2nc(-c3ccccc3)nc(-c3cccnc3)n2)c1 ; OB(O)c1ccc2c3c(cccc13)-c1ccccc1-2 ; c1ccc(-c2nc(-c3cccnc3)nc(-c3cc(-c4ccc5c6c(cccc46)-c4ccccc4-5)cc(-c4ccc5c6c(cccc46)-c4ccccc4-5)c3)n2)cc1]

To a 500 mL double-necked round-bottom flask, 2-(3,5-dibromophenyl)-4-phenyl-6-(3-pyridyl)-1,3,5-triazine (10 g, 21.32 mmol) and fluoranthene-3-boronic acid (11.54 g, 46.89 mmol) were added, followed by 250 mL of toluene as a solvent. The equipment was installed. Then, potassium carbonate (12.06 g, 87.41 mmol) was completely dissolved in 40 mL of water, and added into the round-bottom flask, and finally Pd(PPh₃)₃ (1.48 g, 1.28 mmol) was put into the flask. Air in the flask was evacuated by an oil pump, and nitrogen gas was introduced thereto. The solution was heated at a constant temperature under reflux for 12 hours, cooled, extracted, dried, concentrated, and purified to give 2-(3,5-bis(3-fluoranthenyl)phenyl)4-phenyl-6-(3-pyridyl)-1,3,5-triazine with the yield of 65%.

### Example 6: Synthesis of 2-(3,5-bis(3-anthryl)phenyl)4-phenyl-6-(3-pyridyl)-1,3,5-triazine (5)

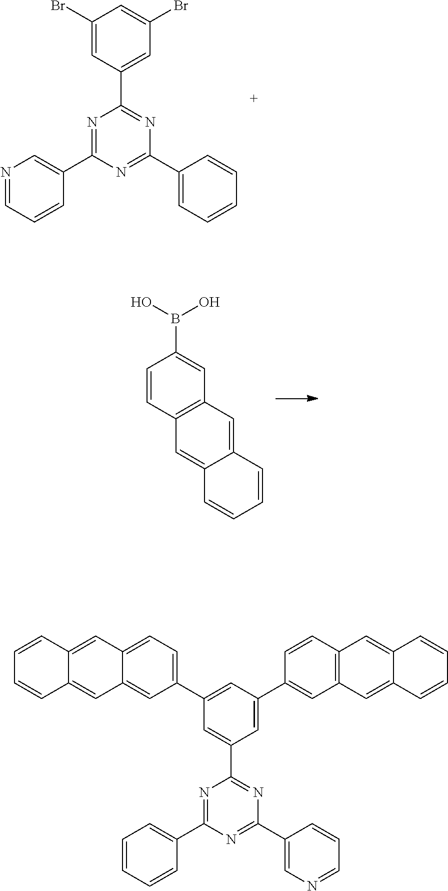 [SMILES: Brc1cc(Br)cc(-c2nc(-c3ccccc3)nc(-c3cccnc3)n2)c1 ; OB(O)c1ccc2cc3ccccc3cc2c1 ; c1ccc(-c2nc(-c3cccnc3)nc(-c3cc(-c4ccc5cc6ccccc6cc5c4)cc(-c4ccc5cc6ccccc6cc5c4)c3)n2)cc1]

To a 500 mL double-necked round-bottom flask, 2-(3,5-dibromophenyl)₄-phenyl-6-(3-pyridyl)-1,3,5-triazine (10 g, 21.32 mmol) and anthraceneboronic acid (10.43 g, 46.89 mmol) were added, and followed by 250 mL of toluene as a solvent. The equipment was installed. Then, potassium carbonate (12.06 g, 87.41 mmol) was completely dissolved in 40 mL of water, and added into the round-bottom flask, and finally Pd(PPh₃)₃ (1.48 g, 1.28 mmol) was put into the flask. Air in the flask was evacuated by an oil pump, and nitrogen gas was introduced thereto. The solution was heated at a constant temperature under reflux for 12 hours, cooled, extracted, dried, concentrated, and purified to give 2-(3,5-bis(3-anthryl)phenyl)4-phenyl-6-(3-pyridyl)-1,3,5-triazine with the yield of 65%.

### Comparative Example 1: Synthesis of the Comparative Compound 2-(4-(9,10-bis(2-naphthalene)anthracene-2-)phenyl)-1-phenyl-1-H-benzimidazole (Comparative 1)

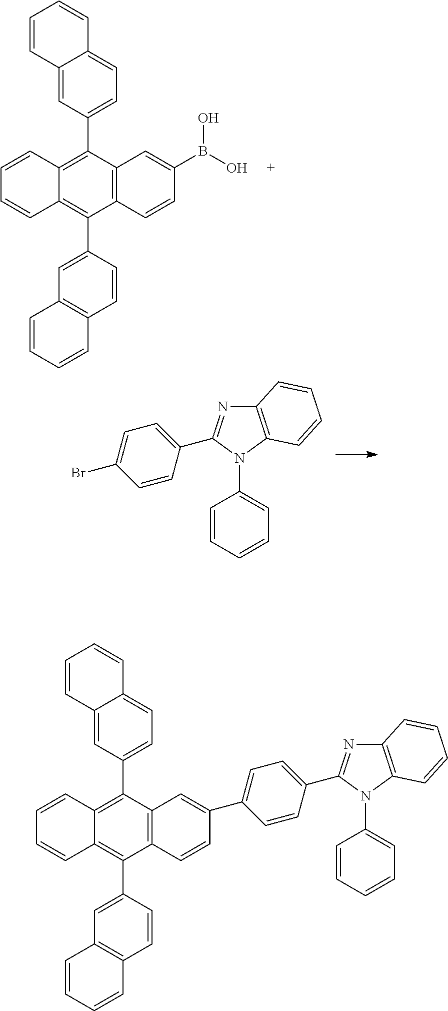 [SMILES: OB(O)c1ccc2c(-c3ccc4ccccc4c3)c3ccccc3c(-c3ccc4ccccc4c3)c2c1 ; Brc1ccc(-c2nc3ccccc3n2-c2ccccc2)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

To a 1 L double-necked round-bottom flask, 9,10-bis(2-naphthalene)anthracene-2-boronic acid (47.4 g, 0.1 mol) and 2-(4-bromobenzene)-1-phenyl-1-H-benzimidazole (34.9 g, 0.1 mol) were added, followed by 500 mL of toluene as a solvent. The equipment was installed. Then, potassium carbonate (20.7 g, 0.15 mol) was completely dissolved in 100 mL of water, and added into the round-bottom flask, and finally Pd(PPh₃)₄ (3.4 g, 0.003 mol) was put into the flask. Air in the flask was evacuated by an oil pump, and nitrogen gas was introduced thereto. The solution was heated at a constant temperature under reflux for 12 hours, and cooled. The reaction solution was transferred to a rotary evaporator flask, evaporated most of the solvent away by rotary evaporation, extracted with dichloromethane, washed three times with water, dried over anhydrous magnesium sulfate, filtered, spin-dried, and purified to give 2-(4-(9,10-bis(2-naphthalene)anthracene-2-)phenyl)-1-phenyl-1-H-benzimidazole, with the yield of 74%.

2. Energy Structure of Organic Compounds

The energy levels of organic materials can be obtained by quantum calculations, such as using TD-DFT (Time Dependent-Density Functional Theory) by Gaussian03W (Gaussian Inc.), and the specific simulation methods can be found in WO2011141110. Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-density functional theory) for “TD-SCF/DFT/Default Spin/B3PW91” and the basis set “6-31G (d)” (Charge 0/Spin Singlet). The HOMO and LUMO levels are calculated according to the following calibration formulas, and S1 and T1 are used directly.

HOMO(eV)=((HOMO(G)×27.212)−0.9899)/1.1206

LUMO(eV)=((LUMO(G)×27.212)−2.0041)/1.385

wherein HOMO(G) and LUMO(G) in the unit of Hartree are the direct calculation results of Gaussian 03W. The results were shown in Table 1:

TABLE 1

| | HOMO [eV] | LUMO [eV] | T1 [eV] | S1 [eV] |
|---|---|---|---|---|
| Example 1 | −5.84 | −3.00 | 2.28 | 2.51 |
| Example 2 | −6.06 | −2.95 | 2.28 | 2.52 |
| Example 3 | −6.08 | −2.97 | 2.28 | 2.52 |
| Example 4 | −5.68 | −2.92 | 1.74 | 2.93 |
| Example 5 | −6.10 | −3.00 | 2.28 | 2.52 |
| Example 6 | −5.69 | −2.97 | 1.74 | 2.87 |
| Comparative | −5.56 | −2.83 | 1.66 | 2.83 |
| Example 1 | | | | |

3. Preparation and Characterization of OLED Devices:

HIL: a triarylamine derivative;

HTL: a triarylamine derivative;

Host: anthracene derivative;

Dopant: a triarylamine derivative;

ETL: Compound 1-Compound 6, or Comparative Compound 1.

The preparation steps of an OLED device having ITO/HIL (50 nm)/HTL (35 nm)/Host: 5% Dopant (25 nm)/ETL (28 nm)/LiQ (1 nm)/Al (150 nm)/cathode are as follows:

a. cleaning of conductive glass substrate: when the conductive glass substrate is used for the first time, various solvents such as chloroform, ketone, and isopropanol can be used for cleaning, then treating with UV and ozone plasma;

b. HIL (50 nm), HTL (35 nm), EML (25 nm), ETL (28 nm): formed by thermal evaporation in high vacuum (1×10⁻⁶ mbar).

c. cathode: LiQ/Al (1 nm/150 nm) was formed by thermal evaporation in high vacuum (1×10⁻⁶ mbar);

d. encapsulation: the device was encapsulated with ultraviolet curable resin in a nitrogen glove box.

The current-voltage (J-V) characteristics of each OLED device were characterized by characterization equipment while important parameters such as efficiency, lifetime and external quantum efficiency were recorded. After testing, the blue light-emitting device prepared by using the compound 1 to compound 6 as the electron transfer layer ETL has a better color coordinate than that prepared by using the comparative compound 1. For example, the device prepared by using the compound 1 to compound 6 has a color coordinate with X<0.15 and Y<0.10. In addition, the blue light-emitting device prepared by using the compound 1 to compound 6 as the ETL layer has luminous efficiency in the range of 5-8 cd/A, which is more excellent luminous efficiency; in terms of lifetime of the device, the blue light-emitting device prepared by using the compound 1 to compound 6 as the ETL layer has a much better lifetime than that prepared by using the comparative compound 1. For example, the T₉₅ lifetime of the device prepared by using the compound 1 to compound 6 is at least twice that prepared by using the comparative compound 2.2 at 1000 nits. The test results are shown in table 2.

TABLE 2

| | | Lifetime (T95,1000 nits) | |
|---|---|---|---|
| | Efficiency | relative to the comparative | Color coordinate |
| | (cd/A) | example 1 | (CIE, 1931) |
| Example 1 | 5.7 | 3.35 | (0.12, 0.08) |
| Example 2 | 5.3 | 3.75 | (0.10, 0.06) |
| Example 3 | 5.5 | 3.125 | (0.14, 0.08) |
| Example 4 | 4.7 | 2.375 | (0.12, 0.08) |
| Example 5 | 5.8 | 2.55 | (0.14, 0.07) |
| Example 6 | 4.5 | 2.225 | (0.13, 0.09) |
| Comparative | 3.6 | 1 | (0.18, 0.15) |
| Example 1 | | | |

In the present disclosure, a triazine structure containing three strong electron-accepting nitrogen atoms is linked to a planar pyrene aromatic fused ring, achieving better carrier transport performance and optoelectronic response, and thus achieving higher efficiency, longer lifetime and bluer color coordinates due to the large planar conjugated structure of the molecule.

The technical features of the above-described embodiments may be combined arbitrarily. To simplify the description, not all the possible combinations of the technical features in the above embodiments are described. However, all of the combinations of these technical features should be considered as within the scope of the present disclosure, as long as such combinations do not contradict with each other.

The above-described embodiments merely represent several embodiments of the present disclosure, and the description thereof is more specific and detailed, but it should not be construed as limiting the scope of the present disclosure. It should be noted that, for those skilled in the art, several variations and improvements may be made without departing from the concept of the present disclosure, and these are all within the protection scope of the present disclosure. Therefore, the scope of the present disclosure shall be defined by the appended claims.

1. A triazine fused ring compound represented by general formula (1): 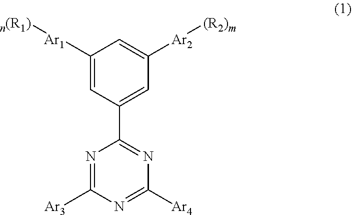
wherein
Ar₁ and Ar₂ are aromatic or heteroaromatic groups, and at least one of Ar₁ and Ar₂ is a fused aromatic ring or fused heteroaromatic ring containing 13 to 60 ring atoms,
Ar₃ and Ar₄ are selected from the group consisting of H, D, F, —CN, —NO₂, —CF₃, alkenyl, alkynyl, amino, acyl, amide group, cyano, isocyano, alkoxy, hydroxy, carbonyl, sulfonyl, a alkyl group containing 1 to 60 carbon atoms, an aromatic group containing 6 to 60 carbon atoms, and a heterocyclic aromatic group containing 3 to 60 carbon atoms, or Ar₃ and Ar₄ form a monocyclic or polycyclic aliphatic or aromatic ring system with each other;
at least one of Ar₃ and Ar₄ comprises an aromatic heterocyclic ring having an N atom;
R₁ and R₂ are selected from the group consisting of H, D, F, —CN, —NO₂, —CF₃, alkenyl, alkynyl, amino, acyl, amide group, cyano, isocyano, alkoxy, hydroxy, carbonyl, sulfonyl, a alkyl group containing 1 to 60 carbon atoms, an aromatic group containing 6 to 60 carbon atoms, and a heterocyclic aromatic group containing 3 to 60 carbon atoms, or R₁ and R₂ form a monocyclic or polycyclic aliphatic or aromatic ring system with each other;
n is an integer from 0 to 20; and m is an integer from 0 to 20.

2. The triazine fused ring compound according to claim 1, wherein at least one of Ar₃ and Ar₄ comprises the following structures Ts: 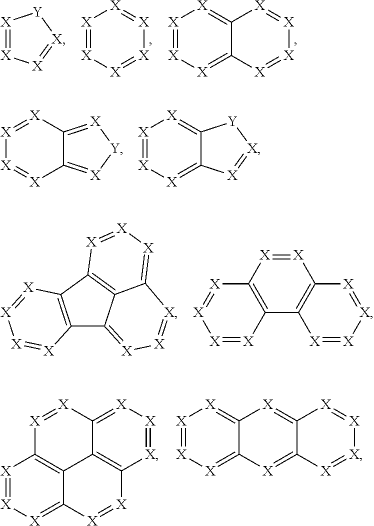
wherein
X is CR3 or N, at least one of the Xs in the structures Ts is N, and two adjacent Xs are not simultaneously N;
Y is selected from the group consisting of CR4R5, SiR6R7, NR8, C(═O), S(═O)2, O, S;
R3 to R8 are selected from the group consisting of H, D, F, —CN, —NO2, —CF3, alkenyl, alkynyl, amino, acyl, amide group, cyano, isocyano, alkoxy, hydroxy, carbonyl, sulfonyl, alkyl groups containing 1 to 60 carbon atoms, aromatic groups containing 6 to 60 carbon atoms, and heterocyclic aromatic groups containing 3 to 60 carbon atoms, or R₃ to R₈ form a monocyclic or polycyclic aliphatic or aromatic ring system with each other.

3. The triazine fused ring compound according to claim 1, wherein at least one of Ar₁ and Ar₂ is selected from the following structures Ds: 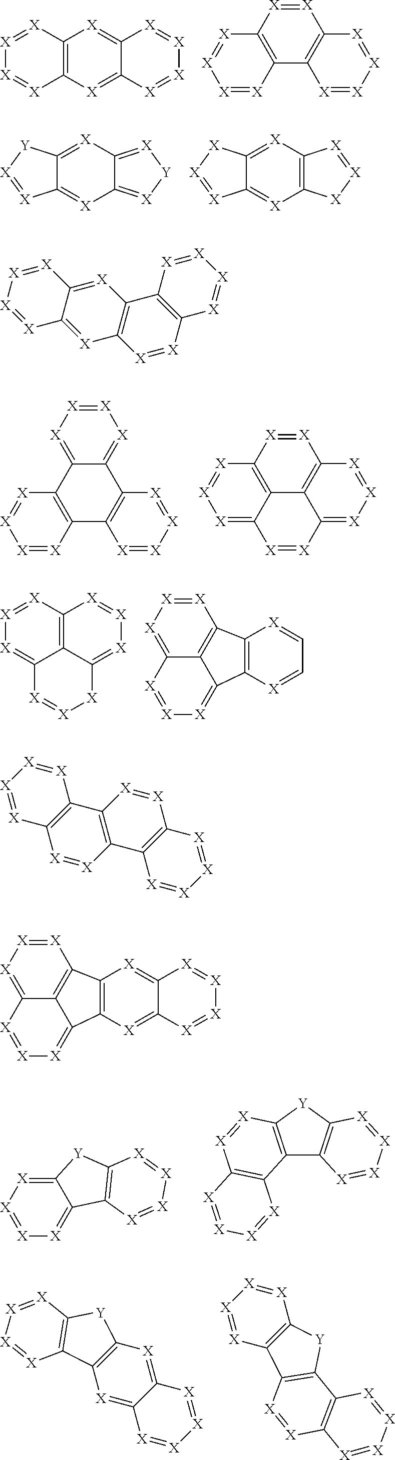 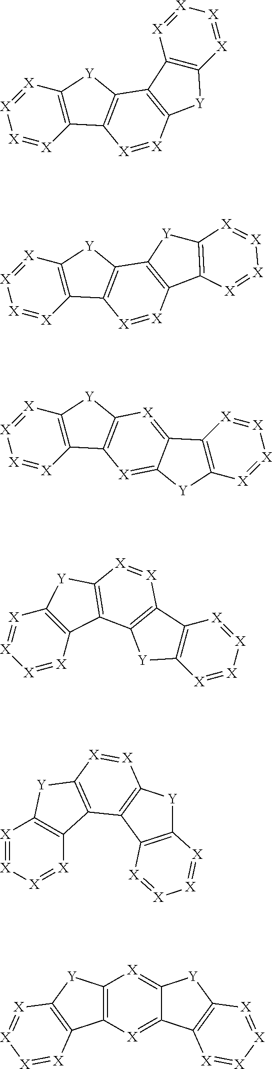 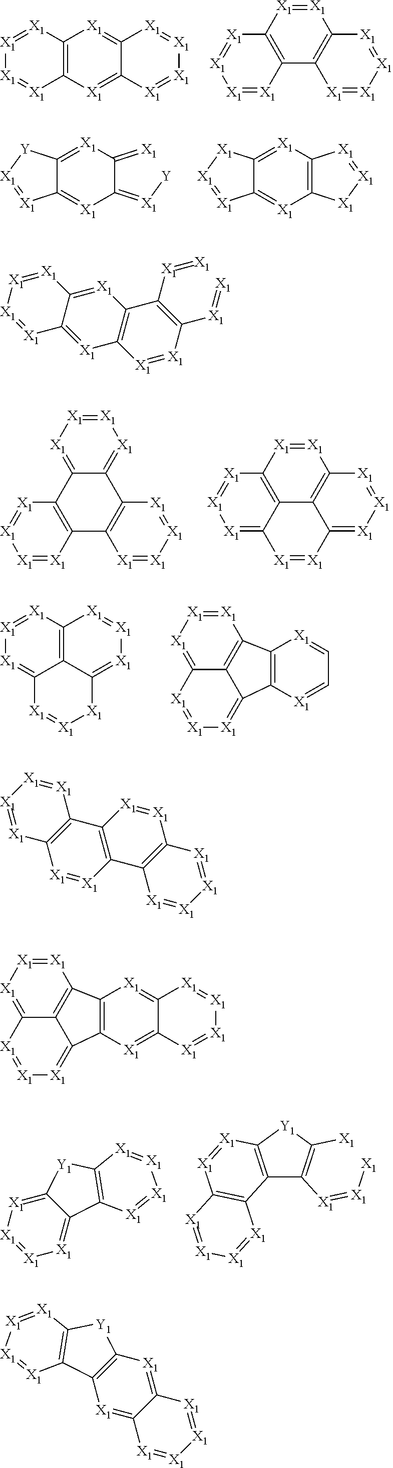 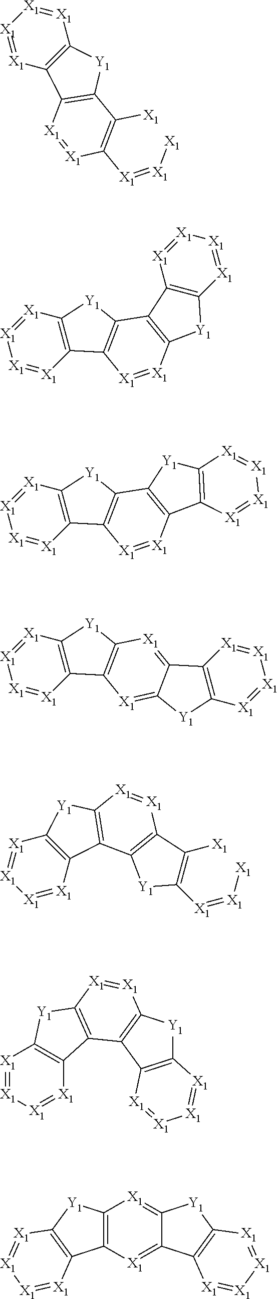
wherein
X₁ is CR₉ or N, but two adjacent X1s are not N simultaneously;
Y1 is selected from the group consisting of CR10R11, SiR12R13, NR14, C(═O), S(═O)2, O, S;
R9 to R14 are selected from the group consisting of H, D, F, —CN, —NO2, —CF3, alkenyl, alkynyl, amino, acyl, amide group, cyano, isocyano, alkoxy, hydroxy, carbonyl, sulfonyl, alkyl groups containing 1 to 60 carbon atoms, aromatic groups containing 6 to 60 carbon atoms, and heterocyclic aromatic groups containing 3 to 60 carbon atoms, or R₉ to R₁₄ form a monocyclic or polycyclic aliphatic or aromatic ring system with each other.

4. (canceled)

5. A mixture comprising at least one of the triazine fused ring compound of claim 1, and at least one organic solvent or at least one organic functional material, the organic functional material being a hole injection material, a hole transport material, a hole blocking material, an electron injection material, an electron transport material, an electron blocking material, a fluorescent emitter, a phosphorescent emitter, a thermally activated delayed fluorescent material or an organic dye.

6. (canceled)

7. An organic electronic device comprising the triazine fused ring compound of claim 1.

8. The organic electronic device according to claim 7, wherein the organic electronic device is an organic light emitting diode, an organic photovoltaic cell, an organic light-emitting electrochemical cell, an organic field effect transistor, an organic light emitting field effect transistor, an organic laser, an organic spintronic device, an organic sensor or an organic plasmon emitting diode.

9. The organic electronic device according to claim 7, wherein the organic electronic device is an organic electroluminescence device, and the organic electronic device comprises an electron transport layer, an electron injection layer or a light-emitting layer, the electron transport layer, the electron injection layer or the light-emitting layer comprising the triazine fused ring compound.

10. (canceled)

11. The triazine fused ring compound according to claim 1, wherein as least one of Ar₁ and Ar₂ is selected from the following structures Ds: 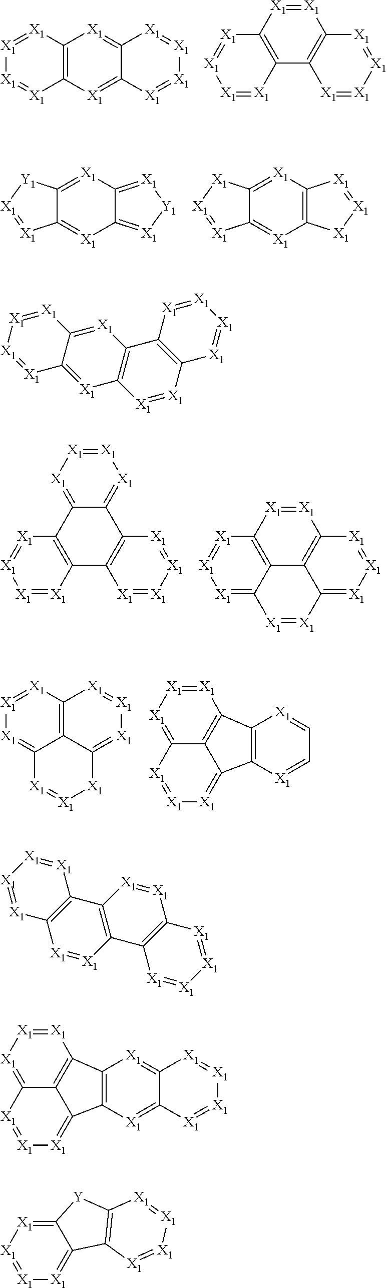 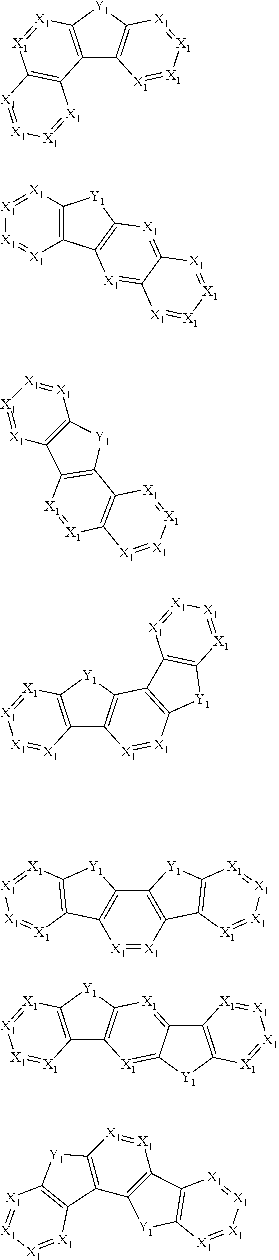 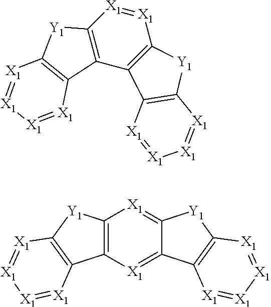
wherein
X1 is CR9 or N, but two adjacent X1s are not N simultaneously;
Y1 is selected from the group consisting of CR10R11, SiR12R13, NR14, C(═O), S(═O)2, O, S;
R9 to R14 are selected from the group consisting of H, D, F, —CN, —NO2, —CF3, alkenyl, alkynyl, amino, acyl, amide group, cyano, isocyano, alkoxy, hydroxy, carbonyl, sulfonyl, alkyl groups containing 1 to 60 carbon atoms, aromatic groups containing 6 to 60 carbon atoms, and heterocyclic aromatic groups containing 3 to 60 carbon atoms, or R₉ to R₁₄ form a monocyclic or polycyclic aliphatic or aromatic ring system with each other.

12. The triazine fused ring compound according to claim 1, wherein Ar₁ and Ar₂ in the general formula (1) are each independently selected from the following structures: 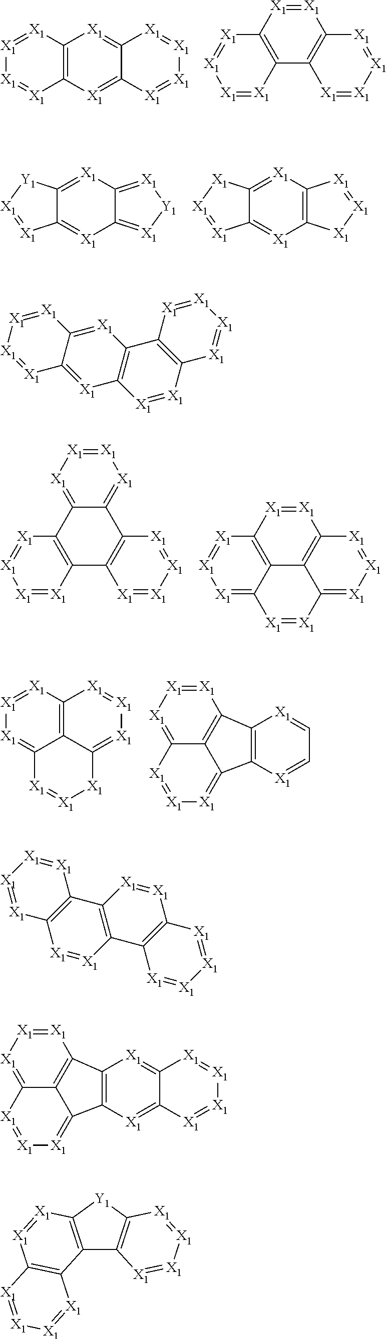 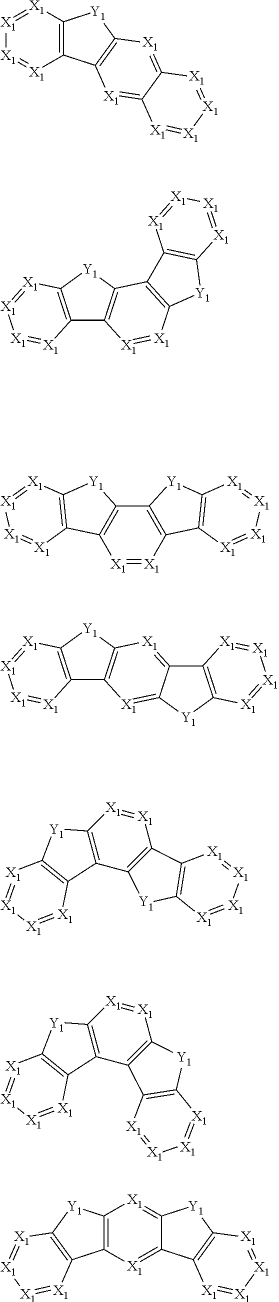
wherein
X1 is CR9 or N, but two adjacent X1s are not simultaneously N;
Y1 is selected from the group consisting of CR10R11, SiR12R13, NR14, C(═O), S(═O)2, O, and S;
R9 to R14 are selected from the group consisting of H, D, F, —CN, —NO2, —CF3, alkenyl, alkynyl, amino, acyl, amide group, cyano, isocyano, alkoxy, hydroxy, carbonyl, sulfonyl, alkyl groups containing 1 to 60 carbon atoms, aromatic groups containing 6 to 60 carbon atoms, heterocyclic aromatic groups containing 3 to 60 carbon atoms, or R₉ to R₁₄ form a monocyclic or polycyclic aliphatic or aromatic ring system with each other.

13. The triazine fused ring compound according to claim 1, wherein at least one of Ar₃ and Ar₄ comprises an aromatic heterocyclic ring having an N atom, and the aromatic heterocyclic ring is selected from the following structures:

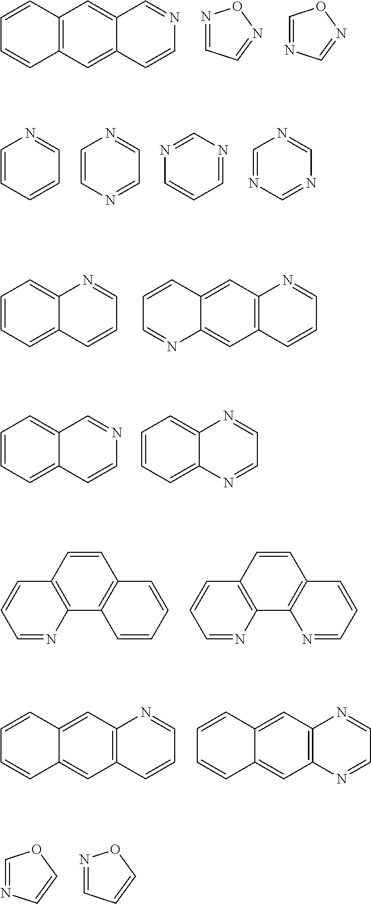

14. The triazine fused ring compound according to claim 1, wherein Ar₁ or Ar₂ is independently selected from the following structures:

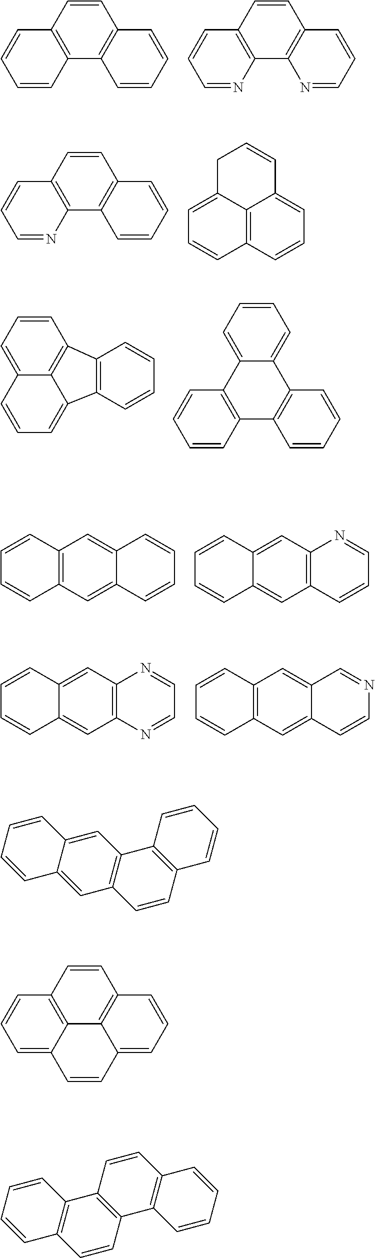

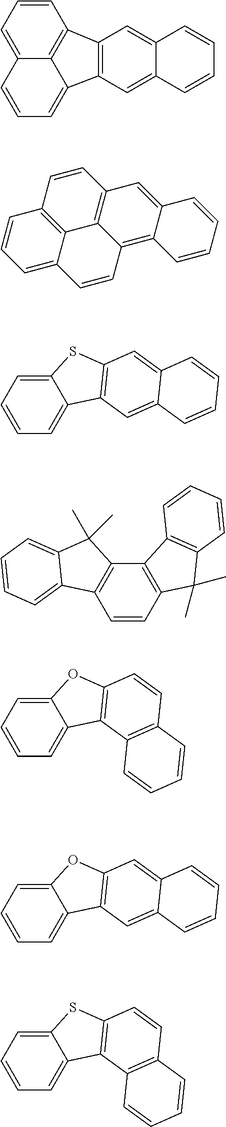

15. The triazine fused ring compound according to claim 1, wherein Ar₁ and Ar₂ are each independently selected from the following structures:

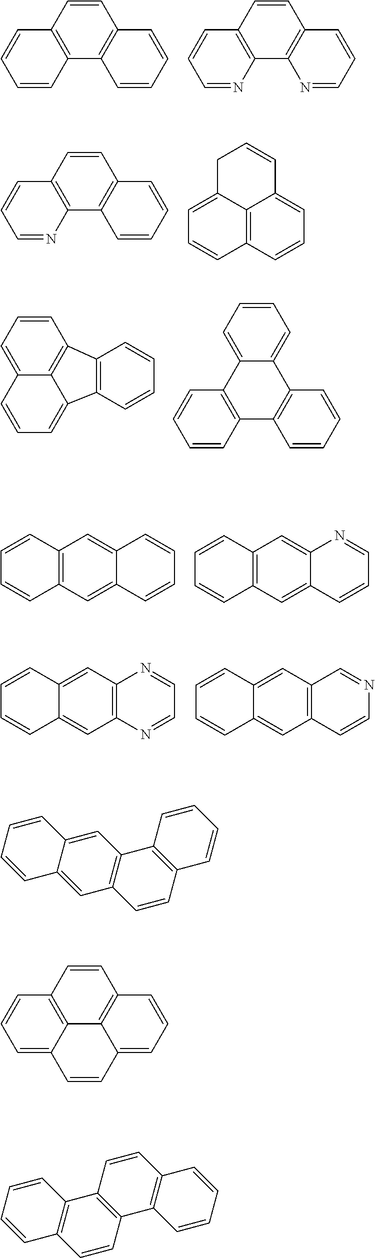

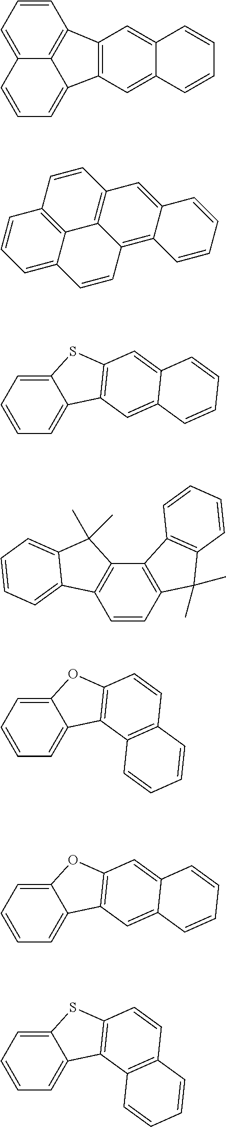

16. The triazine fused ring compound according to claim 1, wherein Ar₁ or Ar₂ is independently selected from the following structures: 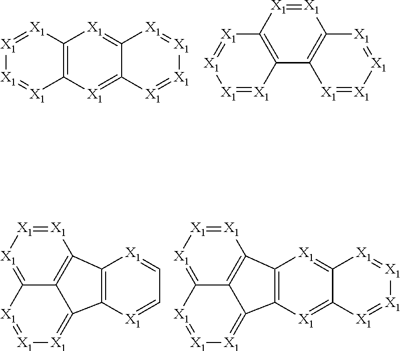
wherein
X1 is CR9 or N, but two adjacent X1s are not simultaneously N;
R9 is selected from the group consisting of H, D, F, —CN, —NO2, —CF3, alkenyl, alkynyl, amino, acyl, amide group, cyano, isocyano, alkoxy, hydroxy, carbonyl, sulfonyl, alkyl groups containing 1 to 60 carbon atoms, aromatic groups containing 6 to 60 carbon atoms, heterocyclic aromatic groups containing 3 to 60 carbon atoms.

17. The triazine fused ring compound according to claim 1, wherein Ar₁ or Ar₂ is independently selected from the following structures: 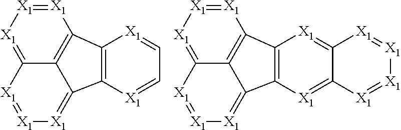
wherein
X1 is CR9 or N, but two adjacent X1s are not simultaneously N;
R9 is selected from the group consisting of H, D, F, CN, —NO2, —CF3, alkenyl, alkynyl, amino, acyl, amide group, cyano, isocyano, alkoxy, hydroxy, carbonyl, sulfonyl, alkyl groups containing 1 to 60 carbon atoms, aromatic groups containing 6 to 60 carbon atoms, heterocyclic aromatic groups containing 3 to 60 carbon atoms.

18. The triazine fused ring compound according to claim 1, wherein Ar₁ and Ar₂ are each independently selected from the following structures:

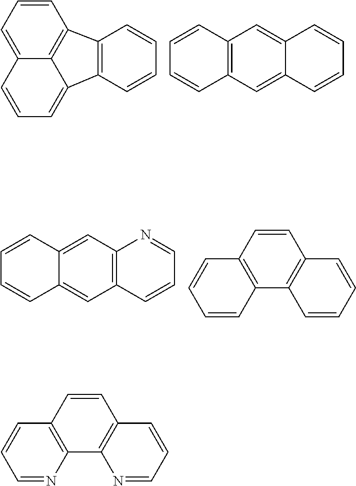

19. The triazine fused ring compound according to claim 1, the lowest unoccupied molecular orbital energy level (LUMO) is less than or equal to −2.7 eV.

20. The triazine fused ring compound according to claim 1, the highest occupied molecular orbital energy level (HOMO) is less than or equal to −5.65 eV.

21. The triazine fused ring compound according to claim 1, the triplet excited state energy level (T1) is greater than or equal to 1.70 eV.
